SEQUENCE LISTING (221 sequences)

SEQ ID NO: 1 - DNA, 12225 bp, Homo sapien
atgactgcctggctgatctctctgatgagtattgaagtactacttttggcagtacgtcacctgagtttacatattgaacctgaagaaggtagccttgcagggggaacgtggatcacagtcatttttgatggtttggagttgggtgttctttaccccaacaatggctctcaattggagatacacctggtgaacgtgaacatggtggtgcccgcactgcggagtgttccctgtgacgtctttcctgttttcttggatttgcctgtggtgacatgccggaccagatctgtgctgtctgaagcacatgagggtctgtacttcctggaagcatacttcgggggacagctggtaagcagtccaaatccaggaccacgagatagctgtactttcaagttttccaaggcgcagacacccatcgttcaccaagtttatccaccaagtggtgttccaggaaaactaatacatgtatatggctggattatcactggaagattggaaacttttgattttgatgctgagtacattgatagcccagtgatcttggaagctcaaggagacaaatgggttactccttgctctcttataaataggcagatgggaagctgttatcctattcaggaggaccatggtcttgggactctgcagtgccatgtggaaggcgactacatcggctcccagaatgttagcttctcagtatttaacaaaggaaagtcaatggtccacaagaaggcatggctgatcagtgctaaacaggatcttttcctataccagacacactcagaaatattatctgtgtttccagaaactgggagccttgggggaagaacaaacatcacaattacaggagacttttttgacaattctgcccaggttaccattgcaggcattccatgtgatattagacacgtgtctcccaggaagattgagtgcaccactcgggctccaggaaaagatgtgaggctcaccacccctcagccaggcaatcgagggcttctttttgaagttggagatgctgttgagggactggaactgactgaagccaccccagggtacaggtggcagattgtccctaatgccagttctccatttgggttttggtcacaggaaggacaacctttcagagcacggctcagtgggttctttgtggctccagagacaaataattacactttctggattcaggcagatagccaagcttccttgcatttcagttggtcagaggaaccaaggactaaggtgaaagtggcctccatcagcgtcggcactgctgactggtttgactcctgggagcagaatagggatgaagggacctggcagcagaagactcccaagttggagctgttgggtggagccatgtactacctggaagcagagcatcatgggatagccccaagcagggggatgaggattggtgtccagattcacaacacctggctgaatcctgatgtggtcaccacttacctacgggagaagcaccagatccgagtccgagcccagaggcttccagaagtacaggtgctgaatgtatcaggcagaggaaacttcttccttacttgggacaatgtctctagtcagccaatccctgcaaatgccacagcccatctgattcaaacaaccattgaggagttacttgcagtaaaatgcaaactggaacccctttggtctaacatccttctccggcttggatttgaacgaggcccagaagtttccaactctgatggggacctcaccagtgggacggagcccttctgtggcaggttcagcctccgtcagcctcgacaccttgtccttactcccccggctgcccagaagggctatcggctagatcagtatacacacctgtgtcttgcatacaaaggccacatgaacaagatcctgaagatgattgtgtccttcacaatcggctttcaaaacatggtaaagaataccacctgtgactggagtctcacgaggaccagccccgagagctggcagttcgattgcactgacctctgggagacttgtgtgcgttgcttcggggatctccagccccctccggcaaactccccagtgctggttcatcagatcaaccttctccctctggcccaggagacgggcctgttctatgtggatgaaattattattgcagacacaaacgtaacagtttctcaagctgattctggaacggctcgcccagggggcaatctggtggaatcagtctctgtggtgggatcccctccggtctacagtgtcacctcctggctggcggggtgtggcacggagctcccgctcatcactgcacgctctgtgcccactgaaggaacagaagagggatctggactggtcctggtgacgacacagagacgacagcggacaagtccacctctaggaggacactttcgcatccagcttcctaatacagtgatttctgatgtccctgtacaaatttctgctcatcaccttcaccagctcttacagaataatgccgatgacttcacatccaggtacctcaatgccagtgacttcactgtgaaggaggatctatacacttgctacgaacacgtgtggaccttgtcctggtccactcagattggggatttgcccaattttatcagggtctctgatgaaaaccttactggagtgaatcctgctgcagccacgcgtgtggtatatgatggtggagtttttcttggacccatatttggagacatgttggctactgccaaccagcatactcaggtggttgtgcgagtgaatgatgtaccagctcattgcccaggttcctgctctttccagtacctccaagggtcaactccctgtgtccattctgtgtggtactccattgatggtgacatcaacctaatgatttacattaccggaactggtttctctggtgactcccagttcttgcaggttacagtgaacaaaacgagttgcaaagttattttctcaaaccagaccaatgtagtctgtcagacagatttgctacctgttggaatgcatcggatcttgatgttggtgagaccctctggtcttgccatcagtgccactggagaagacctcttcctaaatgtgaaacctagactggatatggtggagccttccagagctgcggatattggagggctctgggccaccatccgaggctctagtttggaaggtgttagcctgatattatttggatcttactcgtgtgccatcaatgtcgctacaagcaattcaagcagaattcagtgcaaagttccacccagggggaaagatggacgcattgtgaatgtgactgtgatcagaggggactattctgcagttcttcccagagcatttacatatgtctcttccttaaatccagttattgtgactctgagcagaaacataagcaatatagcaggcggtgagaccctggtcattggagtggcgaggctgatgaactatacggatttggatgtggaagtccacgtccaggatgccttggctccggttcacacacagtcggcttggggcctggaggtggcactgcccccactgccagctggtctccacagaatttccgtctctatcaatggggtcagcattcactcacaaggggttgatctccacatccagtacctcacagaagttttcagcatcgagccttgctgtgggtccctgctgggagggaccatcctcagcatctcaggaataggcttcagcagggacccagctttggtttgggtacttgtgggcaatcggtcctgtgacattgtgaacttaacggaggcgagcatctggtgtgaaaccctgccagccccccagatacccgatgcgggcgctcccactgttccagctgccgtggaggtctgggctggcaacaggttcttcgcccgtggtccttcaccaagcttggtggggaaaggcttcaccttcatgtatgaagcggcagcaacaccagtagtcactgccatgcaaggagaaatcacaaatagcagcctgagcctgcatgtgggaggaagtaacctctccaactcagtcatccttctggggaacctgaactgtgatgttgagacacagtccttccagggcaacgtgagcctgtctggatgctccatccctcttcacagtctggaggctggcatctatcctctccaagtacgtcagaagcagatgggatttgctaatatgtctgtggtgctccagcaatttgcagtgatgcctcggataatggccatcttcccatcgcagggttcggcatgtggtgggaccatacttactgtgagggggttgcttcttaactctagaaggaggtcagttcgggttgacctctcgggtccttttacttgtgtgattttgagtttgggagaccacaccattctctgccaggttagcctggagggtgaccccttgcctggagcttccttctccctgaacgtcacagtcctggtcaatgggctaaccagcgagtgtcaggggaattgcactcttttcataagggaagaggcaagtcctgtcatggatgccttgtccacaaacaccagtgggtctctgaccactgtgctgattaggggtcagaggttagccaccacagctgatgagccgatggtatttgtggatgatcaacttccttgcaatgtaactttttttaatgcaagccacgttgtgtgccagacaagagacttggccccaggaccccactacctgtcagttttttatacaagaaatgggtatgcttgttctggtaatgtttccagacacttctacattatgccccaagtgtttcattattttcctaagaatttcagcttacatggtggaagcctcttgaccatagagggcacaggcctgagaggacagaacaccacgtcagtctatattgaccagcagacctgcctgacggtgaacatcggtgctgagctcatccggtgcattgttcccacagggaatggctctgttgccctggaaatagaggtagatggactttggtatcacataggagtcattggttataacaaggcctttaccccagaattgatctctatttctcagagcgatgacatcttaacctttgcagtggcccagatctcaggagctgcaaacattgacatttttataggaatgtcaccctgtgtgggtgtctctggtaaccacaccgttcttcagtgcgtggtcccttcccttccggccggggagtaccacgtcagaggctatgactgcatcagagggtgggcctcatctgccctggtgttcacctcaagagttattattacagcagtgacggagaacttcggctgcctgggtggaaggctggtgcatgtgtttggagcgggattttctccagggaatgtctcagctgctgtgtgtggtgctccctgccgagtcctggctaatgctacagtgtctgccttcagctgcttggttctgcccctggatgtgtccttggccttcctgtgtggcctgaagcgtgaggaggacagctgtgaggctgccagacacacctatgtgcagtgtgatttgacagttgccatggcgacagagcaactgcttgaatcgtggccttacctctacatttgcgaggaaagttcccaatgcctctttgtgccagatcattgggcagagtcaatgtttccatcattctcgggcctctttatcagccctaaattggaaagagatgaagttctcatctataatagctcctgtaacattaccatggaaactgaggcagagatggagtgtgagacgcccaatcagccaattaccgtcaagattactgagatacggaaacgctggggccagaacactcagggcaacttttctttacagttctgccggagatggtccaggactcacagctggtttcctgaaaggctgccacaagatggcgacaacgtcacagtggagaatggccaattgcttctgctggacactaacacaagcatcctcaacttactgcacattaaagggggcaagctgattttcatggccccaggacccatcgagctcagggcacacgccatccttgtttctgatggtggagagctccggattggatccgaagacaagcccttccaaggcagagctcagatcacactctacgggagttcctactcaactcccttctttccctatggagtcaagttcctggctgtgaggaatggaactctttctctgcacggttcactaccagaagtaattgtcacctgtcttagagcaactgcccatgccctagacacagtgctggctttagaagatgctgtggactggaaccctggggatgaagttgtcatcatcagtggaacaggtgttaaaggtgccaaaccgatggaagagattgtcactgtggaaactgtgcaggatacagacctctatcttaagtcacctttgagatattctcacaactttacagagaattgggtggctggagagcaccatattttaaaggccactgtggctctgctcagcaggagtattaccatacaaggaaatctcactaatgagagggagaagctgcttgtttcatgccaggaggccaatgctccagaaggtaatctgcagcactgtttgtattccatgagtgagaagatgctaggatccagggatatgggagccagagtgatcgttcagtccttcccagaagagcccagccaggtccagttgaagggagtgcagtttcaagtcttggggcaagccttccataagcatctgagctcactcactctggtgggagctatgagagagtctttcatacagggctgcacagtgaggaactccttcagtagaggcctcagcatgtgcgggaccttgggcctgaaggtggacagtaatgtattctacaatattttaggtcatgcgctgctagttgggacatgcacggagatgagatatatctcctgggaggcaattcatggaaggaaagatgactggtcaggacatggaaatataataagaaacaacgtgatcatccaggtttctggtgccgagggactctccaatcctgaaatgttgacaccatctggcatctatatctgcagtcccaccaatgttatagaggggaacagagtgtgtggtgctggctatggctactttttccatctcatgaccaaccaaacatcacaagctccgcttctttccttcactcagaacattgcacattcttgtaccaggtatggtctctttgtataccctaaatttcagccaccttgggataatgtcactggcaccactctgttccagagcttcacagtttgggaaagtgcaggtggtgcccagatttttagaagtagcaatcttcgcctgaaaaacttcaaagtttattcatgcagagattttggaattgacgtcttggaaagtgatgcaaatacttcagttactgacagcttattacttggtcattttgcccacaagggaagtctgtgtatgtcatctgggattaaaactcctaaaagatgggaactgatggtgtctaacacaacctttgttaattttgatctcatcaactgtgtggccattagaacctgttcagactgttcccaaggacaaggtggatttactgtgaagaccagccagttgaagtttacaaactcttcaaacttagtggcatttccatttcctcatgcagcaattttggaagacttggatgggtctctgtctgggaaaaacagaagtcacattcttgcttctatggaaaccctttcagcttcttgtttggtcaattcaagctttggtcgggttgtccatggcagtgcctgtggaggaggtgttctttttcatcgtatgtctattggtttagcgaatactcctgaagtttcttatgatttaaccatgactgacagcagaaataaaacaaccactgtcaattatgtacgtgatacattgtctaaccctcgtggctggatggctctgctcttggaccaagagacctactcattgcaatctgagaacctttggatcaacagatctctgcagtactcagcaacctttgacaactttgctcctggtaattacctactgctggtgcacacagatttgccgccttaccctgacatcctcctaagatgtgggagtcgagtgggtctgtcttttccatttcttccatcaccaggtcagaaccaaggctgtgactggttcttcaatagccagctgaggcaactcacctatctggtttcaggtgaaggccaagttcaagtcattctccgggtgaaggaaggtatgcccccaactatttcagcttctacctctgcccctgaatcagctttaaaatggtccctccctgaaacatggcaaggtgttgaagaaggctggggaggatacaacaataccattccaggccctggggatgacgttctcattttacccaacagaactgtccttgtggatacagatcttccattcttcaaagggctgtatgtgatggggaccttagacttccctgtggacagaagcaatgttctgagtgtggcatgcatggtcattgcaggcggggagctgaaagttggtactttagaaaatcccttagaaaaggaacaaaagcttctgattctccttagagcctcagagggagtcttttgtgaccgtatgaatggaattcatattgacccaggaacaattggggtttatgggaaagttcatctttacagtgcttatcctaagaactcctggacacatcttggagctgatattgcctcaggaaatgagagaattatagtagaagatgcagtggattggcgcccccatgacaaaatagtccttagctcctcttcttatgagcctcatgaagcagaggtcctcactgtgaaagaagtcaagggccaccatgtgaggatctatgaacggctcaaacaccggcatattggaagtgtacatgtcacggaggatggccgacacattcgtttggctgctgaggttggactgttgacccgaaatatacaaattcagcctgacgtatcatgtagggggagactgtttgtggggtccttcaggaagtccagccgagaagaattttcaggtgtccttcaacttcttaatgtggaaattcagaacttcgggtcaccattgtactcatctgttgaattcagtaatgtgtcagcaggatcctggatcatatcatctactctgcaccagagctgtggcgggggcattcatgcagctgccagtcatggagtacttttaaatgacaatattgtgtttggcacagctggccatggcatagatttagagggtcaggcctatactgtcactaataaccttgtggttctgatgacacagccagcgtggtccaccatttgggtggcgggaatcaaagtgaaccaggtaaaggacatcaacctccatggcaacgttgtggcaggatcagagagacttggctttcacatccgaggccacaagtgctcctcttgtgaactgctttggtctgacaatgtggcgcattcaagtcttcatggccttcatctctataaggaaagtggacttgacaactgtaccagaatctctggcttcttggctttcaagaactttgactatggtgccatgttacatgtagagaacagcgtggagatagagaacattactctggtagacaatactattggtcttttggcagtagtgtatgtattttctgctccacaaaattccgtcaaaaaagtgcagattgtgcttaggaattcagtcattgtggccaccagctcttcttttgactgcattcaggacaaagtgaagccgcactcagccaacttgacatcaacagatagagctccctccaatccaagaggaggtcgaattggtattctgtggcctgtattcacctcagaaccaaatcagtggcctcaggagccatggcacaaagtgaggaatgatcattcaatttcaggaatcatgaaacttcaagatgttaccttttctagttttgtgaagagttgctatagcgatgacctggatgtctgcattctaccaaatgcagagaacagtggaattatgcacccaataacagcagagaggaccaggatgctaaagataaaagataaaaacaagttctactttccttcattacaacccaggaaagatttaggaaaagtagtctgtcctgaattagactgtgcaagtccaagaaaatatctcttcaaggatctggatgggagagccctgggtctgcctccaccagtttctgtatttcctaaaacagaggcagaatggactgcatccttcttcaacgcaggtacatttagagaagaacagaaatgtacataccaatttctgatgcaaggattcatctgcaaacagactgaccaagtggtcctaattcttgatagcgctgatgccatttgggcaattcagaagttatatccagttgtatctgtgactagtggttttgttgatgtctttagcagtgtaaatgccaatattccctgctctacttctgggtcagtgtctactttctattctatcttacccatcaggcaaatcaccaaagtctgcttcatggatcaaactcctcaagttttgcgcttttttctattggggaacaaaagtacctccaagcttctcttggctgtattctaccatgagctccagagcccccacgtcttcttaggggaaagttttattccacccactctggttcagtcagcttccttattgctgaatgaatctattggtgccaactatttcaacatcatggataacctcttgtatgttgtcctacaaggagaggagcccattgaaatacgctcaggtgtttccattcacttggccctcactgtgatggtttcagtcttagaaaaaggctgggaaatagtaatactcgaaagactaactaacttcttacagattggccaaaaccaaatcaggtttattcacgagatgcctggccatgaagagaccttaaaggccattgctgacagtagagcaaaaagaaagcgcaattgccctactgtgacttgcactagtcattatagaagagttggtcaacgtaggcctctcatgatggaaatgaactcacatagggcttcacccccaatgactgtggaaactatctcaaaagtgattgtcattgaaattggtgattcgccaacagtaaggagcactggaatgatttcatccttatcaagtaacaaattacagaatttggctcatcgagtcatcactgctcaacagactggggtactagagaatgttctgaatatgactatcggggccttactagttactcagtcaaagggagtcattggctatggaaatacaagcagttttaaaactgggaacttgatatatattcggccctatgcactttccatcctagtccagccttcagatggagaagtgggaaatgagcttccagtgcagccacaattggtatttttggatgagcagaatcgaagagtagagtccctgggacctccttcagagccatggacaatttcagcttccctggaaggagcatcagactcagtgctaaaagggtgcacccaggcagaaactcaagatggttatgttagcttctacaacttggcagtcttgatctctgggtcaaactggcactttatttttactgtcacttctcctccaggagtcaattttacagctcgatccaagccatttgctgtcttgcctgtgactaggaaggagaagtcgaccatcatcctggctgcttccctgtcctctgtggcctcatggctggctctgagctgtctggtgtgctgttggcttaaaagaagcaaaagcagaaaaacaaaacctgaagagattcctgaatcccagactaataatcaaaatattcatatccacatctcatccaaacgccgagaatcacaagggcccaaaaaagaagacactgtggtgggagaagatatgagaatgaaggtcatgctgggcaaggtgaaccagtgcccccaccagttgatgaatggagtgtccagaaggaaagttagccgccacattgtccgagaggaagaggctgctgtgcctgctcctggtactactggcatcacatcccatgggcacatctgtgctccaggtgctcctgctcagcaggtgtacctgcaagagactgggaactggaaggagggccaagagcagttgctcagataccagctggcaggccaaaatcagctgctgctgctatgcccagacttcagacaagagaggcagcagttgccagggcaaagtcggctgagtaagcaaagtggcagcttggggctttcccaagagaagaaagcctcctgcggggccactgaggcattctgccttcattcagtacacccggaaactattcaggagcaactgtga

SEQ ID NO: 2 - protein, 4074 aa, Homo sapien
MTAWLISLMSIEVLLLAVRHLSLHIEPEEGSLAGGTWITVIFDGLELGVLYPNNGSQLEIHLVNVNMVVPALRSVPCDVFPVFLDLPVVTCRTRSVLSEAHEGLYFLEAYFGGQLVSSPNPGPRDSCTFKFSKAQTPIVHQVYPPSGVPGKLIHVYGWIITGRLETFDFDAEYIDSPVILEAQGDKWVTPCSLINRQMGSCYPIQEDHGLGTLQCHVEGDYIGSQNVSFSVFNKGKSMVHKKAWLISAKQDLFLYQTHSEILSVFPETGSLGGRTNITITGDFFDNSAQVTIAGIPCDIRHVSPRKIECTTRAPGKDVRLTTPQPGNRGLLFEVGDAVEGLELTEATPGYRWQIVPNASSPFGFWSQEGQPFRARLSGFFVAPETNNYTFWIQADSQASLHFSWSEEPRTKVKVASISVGTADWFDSWEQNRDEGTWQQKTPKLELLGGAMYYLEAEHHGIAPSRGMRIGVQIHNTWLNPDVVTTYLREKHQIRVRAQRLPEVQVLNVSGRGNFFLTWDNVSSQPIPANATAHLIQTTIEELLAVKCKLEPLWSNILLRLGFERGPEVSNSDGDLTSGTEPFCGRFSLRQPRHLVLTPPAAQKGYRLDQYTHLCLAYKGHMNKILKMIVSFTIGFQNMVKNTTCDWSLTRTSPESWQFDCTDLWETCVRCFGDLQPPPANSPVLVHQINLLPLAQETGLFYVDEIIIADTNVTVSQADSGTARPGGNLVESVSVVGSPPVYSVTSWLAGCGTELPLITARSVPTEGTEEGSGLVLVTTQRRQRTSPPLGGHFRIQLPNTVISDVPVQISAHHLHQLLQNNADDFTSRYLNASDFTVKEDLYTCYEHVWTLSWSTQIGDLPNFIRVSDENLTGVNPAAATRVVYDGGVFLGPIFGDMLATANQHTQVVVRVNDVPAHCPGSCSFQYLQGSTPCVHSVWYSIDGDINLMIYITGTGFSGDSQFLQVTVNKTSCKVIFSNQTNVVCQTDLLPVGMHRILMLVRPSGLAISATGEDLFLNVKPRLDMVEPSRAADIGGLWATIRGSSLEGVSLILFGSYSCAINVATSNSSRIQCKVPPRGKDGRIVNVTVIRGDYSAVLPRAFTYVSSLNPVIVTLSRNISNIAGGETLVIGVARLMNYTDLDVEVHVQDALAPVHTQSAWGLEVALPPLPAGLHRISVSINGVSIHSQGVDLHIQYLTEVFSIEPCCGSLLGGTILSISGIGFSRDPALVWVLVGNRSCDIVNLTEASIWCETLPAPQIPDAGAPTVPAAVEVWAGNRFFARGPSPSLVGKGFTFMYEAAATPVVTAMQGEITNSSLSLHVGGSNLSNSVILLGNLNCDVETQSFQGNVSLSGCSIPLHSLEAGIYPLQVRQKQMGFANMSVVLQQFAVMPRIMAIFPSQGSACGGTILTVRGLLLNSRRRSVRVDLSGPFTCVILSLGDHTILCQVSLEGDPLPGASFSLNVTVLVNGLTSECQGNCTLFIREEASPVMDALSTNTSGSLTTVLIRGQRLATTADEPMVFVDDQLPCNVTFFNASHVVCQTRDLAPGPHYLSVFYTRNGYACSGNVSRHFYIMPQVFHYFPKNFSLHGGSLLTIEGTGLRGQNTTSVYIDQQTCLTVNIGAELIRCIVPTGNGSVALEIEVDGLWYHIGVIGYNKAFTPELISISQSDDILTFAVAQISGAANIDIFIGMSPCVGVSGNHTVLQCVVPSLPAGEYHVRGYDCIRGWASSALVFTSRVIITAVTENFGCLGGRLVHVFGAGFSPGNVSAAVCGAPCRVLANATVSAFSCLVLPLDVSLAFLCGLKREEDSCEAARHTYVQCDLTVAMATEQLLESWPYLYICEESSQCLFVPDHWAESMFPSFSGLFISPKLERDEVLIYNSSCNITMETEAEMECETPNQPITVKITEIRKRWGQNTQGNFSLQFCRRWSRTHSWFPERLPQDGDNVTVENGQLLLLDTNTSILNLLHIKGGKLIFMAPGPIELRAHAILVSDGGELRIGSEDKPFQGRAQITLYGSSYSTPFFPYGVKFLAVRNGTLSLHGSLPEVIVTCLRATAHALDTVLALEDAVDWNPGDEVVIISGTGVKGAKPMEEIVTVETVQDTDLYLKSPLRYSHNFTENWVAGEHHILKATVALLSRSITIQGNLTNEREKLLVSCQEANAPEGNLQHCLYSMSEKMLGSRDMGARVIVQSFPEEPSQVQLKGVQFQVLGQAFHKHLSSLTLVGAMRESFIQGCTVRNSFSRGLSMCGTLGLKVDSNVFYNILGHALLVGTCTEMRYISWEAIHGRKDDWSGHGNIIRNNVIIQVSGAEGLSNPEMLTPSGIYICSPTNVIEGNRVCGAGYGYFFHLMTNQTSQAPLLSFTQNIAHSCTRYGLFVYPKFQPPWDNVTGTTLFQSFTVWESAGGAQIFRSSNLRLKNFKVYSCRDFGIDVLESDANTSVTDSLLLGHFAHKGSLCMSSGIKTPKRWELMVSNTTFVNFDLINCVAIRTCSDCSQGQGGFTVKTSQLKFTNSSNLVAFPFPHAAILEDLDGSLSGKNRSHILASMETLSASCLVNSSFGRVVHGSACGGGVLFHRMSIGLANTPEVSYDLTMTDSRNKTTTVNYVRDTLSNPRGWMALLLDQETYSLQSENLWINRSLQYSATFDNFAPGNYLLLVHTDLPPYPDILLRCGSRVGLSFPFLPSPGQNQGCDWFFNSQLRQLTYLVSGEGQVQVILRVKEGMPPTISASTSAPESALKWSLPETWQGVEEGWGGYNNTIPGPGDDVLILPNRTVLVDTDLPFFKGLYVMGTLDFPVDRSNVLSVACMVIAGGELKVGTLENPLEKEQKLLILLRASEGVFCDRMNGIHIDPGTIGVYGKVHLYSAYPKNSWTHLGADIASGNERIIVEDAVDWRPHDKIVLSSSSYEPHEAEVLTVKEVKGHHVRIYERLKHRHIGSVHVTEDGRHIRLAAEVGLLTRNIQIQPDVSCRGRLFVGSFRKSSREEFSGVLQLLNVEIQNFGSPLYSSVEFSNVSAGSWIISSTLHQSCGGGIHAAASHGVLLNDNIVFGTAGHGIDLEGQAYTVTNNLVVLMTQPAWSTIWVAGIKVNQVKDINLHGNVVAGSERLGFHIRGHKCSSCELLWSDNVAHSSLHGLHLYKESGLDNCTRISGFLAFKNFDYGAMLHVENSVEIENITLVDNTIGLLAVVYVFSAPQNSVKKVQIVLRNSVIVATSSSFDCIQDKVKPHSANLTSTDRAPSNPRGGRIGILWPVFTSEPNQWPQEPWHKVRNDHSISGIMKLQDVTFSSFVKSCYSDDLDVCILPNAENSGIMHPITAERTRMLKIKDKNKFYFPSLQPRKDLGKVVCPELDCASPRKYLFKDLDGRALGLPPPVSVFPKTEAEWTASFFNAGTFREEQKCTYQFLMQGFICKQTDQVVLILDSADAIWAIQKLYPVVSVTSGFVDVFSSVNANIPCSTSGSVSTFYSILPIRQITKVCFMDQTPQVLRFFLLGNKSTSKLLLAVFYHELQSPHVFLGESFIPPTLVQSASLLLNESIGANYFNIMDNLLYVVLQGEEPIEIRSGVSIHLALTVMVSVLEKGWEIVILERLTNFLQIGQNQIRFIHEMPGHEETLKAIADSRAKRKRNCPTVTCTSHYRRVGQRRPLMMEMNSHRASPPMTVETISKVIVIEIGDSPTVRSTGMISSLSSNKLQNLAHRVITAQQTGVLENVLNMTIGALLVTQSKGVIGYGNTSSFKTGNLIYIRPYALSILVQPSDGEVGNELPVQPQLVFLDEQNRRVESLGPPSEPWTISASLEGASDSVLKGCTQAETQDGYVSFYNLAVLISGSNWHFIFTVTSPPGVNFTARSKPFAVLPVTRKEKSTIILAASLSSVASWLALSCLVCCWLKRSKSRKTKPEEIPESQTNNQNIHIHISSKRRESQGPKKEDTVVGEDMRMKVMLGKVNQCPHQLMNGVSRRKVSRHIVREEEAAVPAPGTTGITSHGHICAPGAPAQQVYLQETGNWKEGQEQLLRYQLAGQNQLLLLCPDFRQERQQLPGQSRLSKQSGSLGLSQEKKASCGATEAFCLHSVHPETIQEQL

SEQ ID NO: 3 - DNA, 13971 bp, Rattus norvegicus
agcgagctacctgcatggtagctagctagctgcaggctggcccatcaccaaataacgcggcgtcattttgaagggaccggtttctctcatttgaagcacaaagcggacacagcatcaggcacaaaagctacatcatgccagtcgaccctaaaaatgtgcttctaacatatgagaggtctcaagagagctgagctaccctgatgtgatgcctgcctggcggatctctctgttgagtgtggaactactacttttagcaaatgcttactcaagtttccggattgaaccgacagaaggtagccttgcagggggaacatggatcacacttgtttttgacggtttggaaacaagtattctttaccccaacaatggctctcagctggcgatagacctggtgaatgtggcaacacccgccttgaagatcccttgtgacatctctcccctcttcgtggatttgcccgtggtgacgtgccagacgaggtctcttccatcagaagcacctgaaggtctttattctctggagatacgctctggggatcaactgctaggcagcccgtgtgccagattactggacaactgtactttcaagttttccaaggagcagacacctgttttatatcaagttaacccatcaagtggagttccaggagaagtagtgcatgtgtatggctgggtcattactgatcggatggaaatttttgatcctgatgtggactacatggagagcccattaatcctggaagctggaggagacaaatggcttactccttgttctcttataaacaggaagacaggaatctgttttcccatccaggaggaacatggccttgggactgtgcagtgtcgcgtggaaggcagttatattggttcccagaatgttagtttctcagtatttaacaaaggaaggtcgatggtgcataaggaagcctggctgatcagtgctaaacaggaactgttcctgtaccagacatacccagaaatccactctgtgtttccaaaagttgggagccttgggggaagaacagacatcactattactggagatttctttgacccttctgcccgggttaccattgcaggcattccatgtgatattagacatgtgtctcccaagaagattgagtgcaccaccagggctccaagaaatggagcaaggctcactgctccccaggcaagtaatcgaggactcctttttgaagttggaaatgctgttaaggatgaggagctgactgaagccaccccagggtacaggtggcagatagttcctaatgctagttctccatctggcttttggtccaaggaagggagacctttcagagcacggctcagcgggttcttcgtggctccagagaccaacaattacacattctggatccaggcagacagccaagctttcttgcgtttcagttcttcagaggagccaaggatgaaggtagaagtggcttccattgcagttggcactgctgactggtttgactcttgggagcagaacgggaatgaagggagctggcaacagaagacaaataagctagaactgcagggcggagccaagtactacttggaagcagagcagcatgggatagctcccagcagggggatgagaattgctgtccagatccacaacacctggctgaatccagatgtggtcaacacttaccttctggagaaacaccagatccgagcccaagcccagagacttccagaaatacaggtgttgaacatgtcaggtaaaggaaactttttccttacttggggcaatgtctccagccaaccagttcctgcaaatgccacagcccaacagatccaaaccatcatcgaggagttgctggtggtgaaatgcaatctggtacccctttcagctcgtgttctcctctggcttggatttgagcaaggcttagatggctccaggtctgatggggtcctcaccagttcaaccgaaccattctgtggcagattcagccttggtcaacttcgacatcttatcctaagccctggggctgtcagtaagggctatcagctggatcgatacccatatttgtgctttgcatacagaggtcatatgaacaggaccctggacatgactgtttctttcctatttggcttccaaaatgtcatgaagaatatcacctgtgactggagtctgatggaaccccaccctgagagctggcactttacttgcaataacctctgggacacatgtgtacgccattctgaggagctccagtcttctctggcaaacaccccattgctggttcatcggatcgacatcttccctgtggttccagaggcagacttgctctatgtggatgaaattattcttgcagataccaacgtaacagtttctcaagccgattctggagtagcccgcccaggtgggaatgtggtggagtcagtgtctgtggtgggagtccctccagtctacagcatagcctcttggatggcaggatgtggctcagaacttcctctcatcactgcatgctatgtgcccacccaggggacaggcgaaggatcggaactgattgaggtgacagcgaaaagactccagaggacaagcccacctttgggaggacacttctccctccacctctctgatacagtgatacctgatgttccagtgcatgtgtctgccagacagcttcataagctactgcaggacaatgctgatgagtccacatctggatatctcaatgccgatgacttcattgtgacaaaggatcaaaattcctgctatgaacatgtgtggactctttcctggaccactcagattggggatttgcccaatttcatcagggtctccgatcaaaatcttacggggttgaatcccaccataactgctcgtgtagtatacgatggcggagttttccttgggcccattcttggagacacactggctactgccaaccagcagactcaggtgactgtacaagtgaatgacataccagcccattgttcaggctcctgctccttccaataccagcgagcgtcaactcccagcgtggactgtgtgtggtattcccttggtagtgatgtcaacctgctggttcattttactggaaatggtttccctagagatgcccagttcttacaggtcacggtgatcaacacgagttgtgaagttattttctcaaatgaaaccaacgttgcctgtgagcttgctctactaccagttggagtgcaccgggtttttatgctggtgagaccttctggccttgctcttaatgccagtggagaaggcctcttcctacgtgtggaacccaggctggatgctgtggagccttctacaggcgcagagattggaggatggtgggttacactccgaggctctagtctagaaggcgttagcctggtgttatttggaactcggtcgtgtgtcattgatgtcattagaagcaattcgcaacagattcaatgcaaagtcccacctagggggaaagatggatacactgtgaatgtgactgtgatcaatggggaccactccacagttcttgccagagcatttacttatgtctcctctttaaatccagtgattgtgtctttgagcagaaatagaagcaacatagcaggaggtgagattctgttccttgggatgacactgatggtgaactatacagatctgaatgtgcaaatctacattcagaacacttctgctcaggtccttgcaaagacggcatggggactggaggtgctgttgcctccgttgctgcctggcatctatgtgatttcagcattcatcaatggaatcagcattcgttcacaaggggttgatctccacatccagtacctcactgaagttttcagcatggaaccttgctccgggtctctcttgggtggaactatcctcagactctcaggagcaggactgggcagagacccagctctgattcgggtgcttgtggacaaccgtccttgtgatactgtgaagttaacagaagtgaacatttggtgtgagactccccgagtcctactaccacccagagcagatgttctcactgtcccagcatctgtggagatctgggctggcaacacttccttcgtggggaaggccttcatcttcacatatgaagctgcagcaactccagtggtcactgctatgtggggtgaactcatgaacaacagtgtaaggttttatgtggaaggaaataacctctccgactcagtcattcttttgggatccttgaaatgtgaccttcaagtgcaattgtttggtgataacatgaacctgtctgggtgctcccttcttctccacagtttggaagctggggtctatgctctccaagttcgtcacaagaggatggggtttgccaatatgtctgcggtgcctcagaagtttgagctgtcaccgcagattatcgacatcttcccaacacatggttctatatatggtgggacagtgcttactgtgaagggcacggctttcaggtccagaaggaggtctgttcatgttgacctttcaggtccttttacttgcgtgattttgagtttgggagaccacacagtcctatgccagaccaagtttgtgggtgatcagttttctgaaacatcactggctctaaacatcacagttctggtcaatgggctgaccagcaagtgtgaggggaactgtactctcttcctggaggaagcagcaactcctattgtggatgctttgactataagtatcagtggctctctaaccacagtgctgatgagaggacagaggttaggctctgctggtgagccaacagcctttgtggatggtcaacttctttgccatacaactttctttaataccagctatgtggcatgccagatgagagatttggccccggggttccactatctgtcagctgttcgtacaagtgctggatatgcttgcctcaatagtgtttctagaaacttcttcgttgtgcctcaggtgttcgattattttcctaaggattttagcatccatggtggaagtctcttgactataaaaggcacaacccttcgaggatggaaaactacatttgtctatgttggccagcaggcttgtctaacagtgaacatcagttctgagttcatccagtgcattgttcccgcaggaaatggctctgctgctctggaaatagatgtagatggagttttataccacatgggatttgttgactacagcagcatctttaccccagaattgctttctgtttcacggagtcatgacatcttgacctttacagtggcccggatctcaggggctgcaaatgttgacattcttattgggacgtcaccgtgtctagatgttgctggtaaccgtacgattctccggtgcatggtccctctgcttcctgctggggagtatgctgtcacaggttatgaccacagcagaggatgggtctcatccactctcattctcgtgctgagagccactgtgacctcagtgactgagaactatggctgcctgggtggaaggcttttgcatgtgctcggagcaggattttctccagggaacatctcagctgctgtatgtggtgctccgtgccaagtcttggctaatgcgacagtgtctgccttcagctgcttggttctgcccctggatgcgtccttggctttcctctgtaacctgaggcatgcagaagacagctgtggagttagcaggcccacctacttgcaatgtgacttgactgtctccgtggggatggagagactgcctggatcctggccttacgtctacctttgtgaagagagttcccagggcctcttcgtaccagatcaccagatcttcctttccttctcaggcctcttcctcagccctaaagtggaaagagatgaagttctcatctataatagctcctgtaacattaccatggaaactgaggcagagatggagtgcgggacacctaatcagccaattaccgccaagattactgaaatacagaaaagccggggccagaacactcagggcaacttttccttccaattctgccgaaggtggtccaggcctcacagttggtttcctcacagagtgccacaagatggcgacaatgtcacagtggagaccagtcacctgctactgctagatgcgaatacaagtctcctgaactccctgcacattaaaggtggcaagctgattttcatggagcccggacccattgatctcagagcccattctatcctaattacagacggtggagagctccatattggatctgaggacaagcctttccaaggcaaagctcggatcaaactctatggaagtgtccactccacttccttctttccctatggagtcaagttcctggctgtgaggaatggaactctttctctgcatggttctgttccagaagttacttttacctatcttcgagcagctgcatgtgcaggagacaccgtgttggctctggaagaggccgtggcttggcaccctggggatgaggctgtcattagcagtggagcgactgtagaaggtgctgaagcaacagaagaggtggttgttgtagaaactgtccacgatgcggacctccatctcaggaaccccctgagatactcctacaacttcacagaaaactgggtaactggagtgaatcatattttgaaggctacagtggctcttctcagcaggaacattattgtccaaggaaatctcactctcgagagggtaaagcttcttgattcatgccaggaggccggtgctgctgaaggaaacctgaagcattgtttatattctaagagtgagaagatgctgggagccaggaatctgggggccagagttatcattcagtccttcccagaggaacccagcttggtcaagctgaagggagtgcagttccgagacctgggacaagccttccataagcatctaagctcactcaccctggtgggagctatgagaggttcttatctccacagctgttcaatgtggggctccttcagcagaggcctcagcatacacaggacctggggtctgaaggtggaaaataatatattctataagattgtaggtcatgccctactggtggggtcctacatggacagaagttttagcactagtgaggctgttattggaagaaaaaatggttggtgggaagagggaagtatgataagaaacaatgtgatcatcaatgtttctggggctgagggactgtccagttctgaaatgttggcaccagcaggcatctacactttcagtcccaccagtgcgattgagggcaacagagtgtgtgcagctggttatggctatgttctccaccttgtgaccagccaaacagtgcaagctccactactctcattcaactggaatactgcacattcttgtacaagatatggtctccttgtatatcctgaatttcaaccgccttggaataatgacactggcttcactctgtttcaaaacttcatggtttggggaagtgctggtggtgcccagatttttagaagtaacaatctgcacctgagaaacttccaagtttatgcatgcagagactttgggattgacattttggaaagtgacacgaacactttggttactgacagcttcttactaggtcacttcacccacgagggaagtctatgtatgtcagttggcatcaaaactcccaaaagatggggactgacgatttctaacacaactttcgttaattttgatctcaactgtgtggccatcagaacctgttctggctgttcccaaggacagggtggatttcctgtcaagaccaggcaattgaagtttgtcaactcttcaaatttagtagcatttccttttcctcatgcagcagttttgcaagacttagatgggtccctgtctgggaaaaatggatctcatgttcttgcttctatggaaaccctctcagacacatgtttgaccaatgcaagcttcagccagattgtctctggcagcgtctgtggggaagctgttctcttccatcgtatgtctattggtttagctaagagcctcgacgttcctaagaatttaaccatgactgacatcagaaataggacaatcactatcaattatgtggatgacacgttgtctaactcttatggctggatggctctactcttggaccaagagacctacttgctgcagtttgagggtccttggacggacggatctttgcagtactcagcaacctttgacaactttgcccctggaaattacctcctgctaatgcacagggacctactaccttatcccgacattctcctcagatgtgggagtcgggtgggccagtcacttccctctcatcctttgccgagtcgagacggagcctgtgattggttcttccacaggcagttaaggcagctcacctacctggtttcaggggaaggtcaagttcacgtattcctccaggtgaagcatggtgcacctccaactgtttcagcttctacatctgtatctgaatcagctttaaaatggtcccttcctgaaacatggcaagatgttgaaaaaggctggggaggatacaaccacaccatcccaggacccggtgatgacgtcttgattttacccaacaagacggttcttgtggatactgatctccccgtgcttagatgcctctatgtgatgggtaccttagaattccctgtggacagaagcaacgttctgagtgtggcgtgcttactcattgcaggaggggagctgaaagtaggtactttggaaaaccccttaggaaaagaccaaagacttctgatactcctcagagcctcagaagaaatcttctgtgaccgttttgacggaatacgtgttgaccctggaacaattggagtttatgggaaacttcgccttcacagcgcatatcctaagaaatcctgggtacaccttggagctgacattgccccgggaaatgagaggattatagtacataatgcagtggattggcagccccatgacaaaatcgtcctcagctcttcttcttatgagcctcatgaggcagaggtcctcactgtgaaggaagtcaaggaccatcacatcaggatctatgaacgtctaaaacacaggcacattggaagtgcccacaccctggaggatagtcgacagatttgtttggctgctgaggttggattgttgaccagaaacattagaattcagtctgactcatcctgtagagggagactccttgtggggtccttcaggaaatctagcggagaagagttttctggcattcttcaacttctaaatgtagaaattcaaaacatggggtcaccactgtatgcatctattgaattccccggtgtgtcagctggatcctgggttatatcttctactgtacatcaaagctgcactgtgggcatccatgcatcttctagtcgtgggatggtcttaaatgacaacatagtgtttggcacaaaaggccatggaatcgatgtggagggtcagaactattctctcaccaataaccttgtcattctgtcaatgcagtcagcaaactcccttccttgggtggcaggaatcaaagtgaactatgccgaagatatcattctccacggcaatgtggtggcaggatctgagagacttggctttcatgtccgtggccatgggtgctcctctgaagtgctttggtctgacaatgtggtccactcaagcctccatggcattcacctctacaagaaacgtgaacctaataactgtactggtgtctctggatttctggcttttaagaactttgactatggtgccatggttcagacagagaatagtgtggacatacagaatatcactctggtggataatactgttggtcttttggccatctcgtatgtatcttcggctccactgagatctggcagcactctacagattactcttaggaattcagtcatcgtagccactagctcctcttttgactgcatccaagacagaaaggctcctcagtcggccaactggacctccacagatagagcgccttccaatcccagaggaggccggatcggtattctgtggcctgtatctgcctcagaaccaaatggatggccccaggagccatggcacaaagtaaggggcggtcattcagttccaggaattatgaagcttcaagatgtcaccttttctagtttcgtgaagagttgctatagcgacgatctggatgtctgcatcctgactaatgagcacagcactggaatcatgtacccaataacagcagagaggaccagaatgctgaagataaaggacaaaaacaagttctactttcttccattacagtctgggaaagacttagtgggaaccatctgccctgcatcatcggactgtgaaattccaagaaaatacctcttcacagatctggatgggagaaccctgggtctgtccccaccagtttctgtgtttccaaggatagaggaaggatggactggatccttcttcaacacaggtatattcagagaagaacagaaatgcacatttcgagccatgatccagggcttcttctgtaagcagactgagcatgcagtcctaattcttgataatgttgatgtaacttggacaatccccaaattctacccactcgtatccattactaatggttttgtggacacgttcagtatcgtgaaggacagtggtctatgcttccccacaagctccccgtctactttttattccatcttacccaccagccaaatgaccaaagtatgctttccagagcgaactcccccattcctgcacttttttctactggcaaacagaagagcgtccaagctcatcttggctgtgttctacaatgagattcagagcccctacgttttcttagacaagagctttattccacctacaccagtagagtcagcattttcactgttggatgagcctgttggtgccaactatttcgacatcatgaataacctcttgtatgttgtcctgcaaggagaggaggctgttgaaatacgttcaagtgtttccattcatttggctttgacagtgacattttcagtcctagaaaagggctgggagaaagcagtgcttgaaagtctaagaggcttcttccagattgaccaaaaccaaatcagactcactcttgagatgcctggcaacaaagagaccttaaaggccattgcaaacagtgaaggaaaacgaaagcgcagttgcccaactgtgacttgtgctgtcccttctagcagatatgttcaacgcagacctctcatggcagaaatggcatcatttacaatcacaccagcaaccactctggaaactttctcaaaggtgattgtctttgaaatcggtgatctgccaaatgcaagggacagtgaattcattcagtccttaccaagtaacagattacagaaattggctcaccaggttatcactgctcaacagactggagttctggaaaatgtccttggtatgactgttggggccctactagtggctcagtcagagggagtcacaggatatagaaatgcaagtagtttaataactggaaaccagatatatacccggccctctgtggtttccatcctggtgcagccttcagatggggaagtgggaatagaattaccgattcagccacggctcatcttcctggatgagaagaatgagagagtggaagctttgggtctcccctcagaaccttggattatttcagtttccctggaagggacatctgaatcaatgctaaaagggtgtacccaggcagaaacacaggatggctatgtgaccttctccagattggctgtcttgacctctgggtcaaactggcacttagtttttactgtcactttccctccaggtactaatttaacagctcgatccaagacctttgctgtcctgcctgtggccggcaaggagaggtcaactatcatcctggccttgtccctgtgctcagtggcgtcttgggtggctctaagttgtctcatttgctgctggtttaagaaaagcaaaaccagaaaaataaaaccagaagacatatctgaatcccaggctaaggaacaaaagaagaatacccatatctcttccaaacccagagaactacaagtaaagaccgcaaaagaagacgctatgatgggagaagacatgagaatgaaggtcatggagggaaggcagaaccagttttcccaacactcaatggatggagtgtcaaaaagaaaagttagccgccgtgctgtcccagaggaaagaacagctacacctgccctgaagattcccagaaccacctgtgtcccaggagctcttcctcagcagttgactctgccggagcctgggaactggcaggaggcccaacagcagctgcttagataccagctggcaggccgcgatcagctgcttctgttatgcccagacctcaggcaagagaggcagcagggtcaggagcctagccagctgaacaaagggagtggccgctgcatggggctgtctcaagagaaggccacttgcgttcccactgagaccttctgccttcacacagctccaccagaaactatccagtaacagctgtgggcgcggagcactgtgggcgtttgtatgaaaaggagaaactgttcaaaattatttctacatggtgaacgggggaggagaggtctgatctgtttggacaactaagggagaatggggacgactgtaattaagcttctcttagagccaacactcaggatgcttcaggcttgtcttatggaagacctgtgatagatttagttagctgatgttgtatctgcaggactacagaaatactcaggagatatcttacaccaaagcttatcccccaaacagaggtaacrtaataagagttacttgtagattttatttcgtggtttaaatttagtaattattgggagctttctgcattgcaagcattattatttgcacctcatgagtatttgtacatgaaagatgatggtyacataggctttgttgggatttcagttaagaaaaagatatgaaaacactaaatttaggcaatttttatgtactataggtacatttcacaccaggtatttatatgaaaaggggaaatattcaaagctacttctctgtggtaagtgggaaaagagaggtctgatctgtttggacaactaagggaaaaatgtggactctcaatattttgacttaatagttctaataatttcttctagttgggtaaaatttacaactaccagggaatttttctagaagactaaacataatctaatggtaaagtggactaatggctctcttatttatcagagaactgatttttgtttcccacaaaaagcaagtataaggagtgcccaagagactagaaaagaaatcttaggtatggtcttttgaagctgtgtttagggagatgaagcaaagaggtagctgatgctttaagaaagaaagtgcatcaatcaccctctctaaactgctgaaaattttataaggaatagatagatgccaaaggattttcctgaactgattgatatatttatgtgaccttttatcttctttagtttactgatacagtaatgcattccgtgacttggattcaaacgctgaaccaaatttacataccttaaataaatcctgctggcatattttttgatatactaacaggtttagtttgctgatattttattgtattttatatctaaatgatataaaatattataaaataatataaaatgatatgagatattcgatcataatttcagtttattttttcaattaactttagccatgcatagtttgaatgcctgtagcctaagctttcccaaaagagacggccggaaaatgctttagcacgggaattcaaggccagtctgaaccccatagcaagacaaaataatttctttaaaacgtgaattaggtgtagggcatctggtgacacatgcctctggccccagtactcaagagatagaatagcaggcatcacaaatttaaaggtagcctgggatccacaatatgctgcaagccaacatgacttatccacaaagaacttgtctcaatagagaaaaaagaattaggaatgctctgtattttctggaagaaattctgtgaagttggaggggggaaatggaacttttgttgttgttttctgtagtctgtaaaaacattgtatggaataaaaaaaaaaaaaaaaa

SEQ ID NO: 4 - DNA, 12918 bp, Mus musculus
cactgttggcctactggtagctagctagctgcaggctggcctgtcaccgaatagcgcggcttcgtttagaaggggacggtttctctcatttgaggcacaaggctgacacagcatcaggcacaaaaactacatggcacaaatcaaccctaaaaatgcttctaatatctgaggaatctcaagagagctgagctaactggacatgatgcttgcctggctggtctctctgctgagtatggaagtactactgttagcaaagccttactcgagtttccagtttgaacccgcagaaggtagccttgcaggaggaacatggatcacagttgtatttgacggtttggatagaagtattctttaccccaacaatggctctcagctgcagatagacctggtgagtgtggcaatacctaccttgaggatcccctgtgacgtctctccggccttcgtggatttgcctgtggtgacatgccagacgaggtctcttccatcagaagcagatgcagggccatactccctggaaatgcgctctggggagcaagtgctaggcactccatgtcctggatcactagatagctgtactttcaagttttccagggatcagacacctgttttatatcaagtttacccagcaagtggagttccaggagaagtagtgtccgtgtacggccgggtcatcactacctggctggagaccttcgatcctgatgtggactacatagagagcccattgatcctagaagctagagaagacaaatggcttactccctgttcccttataaacaggcagacaggaagctgttttcccatccaggaggagcatggccttgggaatgtgcagtgtcgtgtggaaggcgattatattggttctcagaatgttagtttttcagtatttaacaaggggaggtcgatggtgcataaggaagcctggctcatcagtgctaaacaggaactgttcctgtaccagacatacccagaaatcttatccgtgtttccaaaagttgggagccttgggggaagaacggacatcatcattacgggagatttctttgacccttctgcccgggttaccattgcaggcatcccatgtgatattagatatgtgtctcccaggaagattgagtgcactaccagggctccaggaaatgaagcaaggctcactgctcctcaggcaggcaatcgaggactccgttttgaagttggagatgctactaaggatgtggagctgactgaagccaccccagggtacaggtggcagatagttcctaatgccagttctccatctggcttttggtcaaaggaagggagacctttcagagcacggctcagtgggttcttcgtggcgccacagacaaacaattacacattctggatccaggcagacagccaagcttccttgtgtttcagttcttcagaggagccaaggaccaaggtagaagtggcctctgttggggttggcactgctgactggtttgactcttgggagcagatcgggaatgaagggagctggcaccagaagacaaccaagctagaactgcagggcggagccaagtactacttggaagcagagcaacatgggatagctcccagcagagggatgagaattggtgtccagatccacaacacctggttgaatccagatgtggtcaacacttaccttctggagaaacaccagatccgagctcgagcccagagacttccagaaatacaggtgttgcatgtgtcaggtaaaggaaactttttccttacttggggcaatgtctccagccaaccagttcctgcaaatgccacagcccaacagatccaaaccaccattgaagagttgcttgtggtgaaatgcaatctggcaccgttttcggcccatgttcttctccggcttggatttgagcaaggtttagaaggctccaggtctgatggggtccgcaccagttcaactgaacccttctgtggcagattcagccttggtcaacttggacatcttatcctaattccagaggctgccgacaagggctatcagctggatcgatacccgtatctgtgccttgcgtacagaggccatatgaacaagaccctggacatgactgtttctttcctctttggcttccaaactatcatgaagaatatcacctgtgattggagtcttaccgatccccaccctgagagctggcagttcacttgcattaacctctgggacacatgtctatgtcactctgaggatatccagtcttctctggcaaacaccccattgctggctcatcggattgacatccgccccgtggttccggaggcaggcctgctctatgtggatgaaattattcttgcagataccaacgtaacagtttctcaagctgattctggaagagcctgcccaggtgggaatgtggtggagtcagtatcggtggtgggagtccctccggtctacagcataagctcctggttggcaggatgtggctcagagctccctctcatcactgcatgctctgtgtccacggagggaacaggagatggatctgaattgattgaggtgacagctcaaagactccagaggacaagcccacctttgggaggacacttcttcctttacctctctgacacagtgatacctgatgttccggtgcgcatgtctgcccgacagctgcataagctactgcaggacagtgctgatgagtccacatctggatatctcaatgccggtgacttcactgtgacagaggatctgaattcctgctatgaacatgtgtggactctttcttggaccactcagactggggatttgcccaattttatcagggtctctgatcaaaatcttactggggtgaatcccactgtaactgctcgcgtggtatatgatggtggagttttccttggacccatctttggagacatgttggctactgccaaccagcaaactcaggtggctgtacaggtgaatgacataccagcctattgttcaggctcctgttctttccaataccagcaagagtcaactcccagtgtggatcatgtgtggtactcccttggaagcgatgtcaacttgctggttcattttactggaactggtttccctagagacacccagttcttacaggtcacggtgaacaaaactagctgtgaagttcttttctcaaatgaaaccaatgtggcctgtgagctggctctgctaccagttggagtgcaccagatttttatgctggtgataccgtcaggccttgctgttcatgccagtggagaagacctcctcctgcatgtggaacccaggctggatgctgtggagccttctacagctgcagagattggaggacggtgggttactctccgaggctctagtttggaaggtgttagcctggtgttatttggaactcagtcatgtgtcattgatgccattagaagcaattcacaacaaattcaatgcaaagtcccacctagggggaaagatggatacactgtgaatgtgactgtgatcagtggcgaccactctacagttcttgccagagcatttacatatgtctcctctttaaatccagtgattgtgtcattgagcagaaacagaagcagcatagcaggaggtgagattctgttccttgggatgtcactgctggtgaactacacagatctggatgtgcaaatccatgtgcaggatacttctgctcaggtcctttcacagacagcgtggggactggaggtagtgttgcctccactagtacctggcatccatgtgatttcagcattcatcaatggagtcagtattcgttcacaaggggttgatctctatatccagtacctcactgaagttttcagcgtggaaccttgctctgggtctctcttgggtgtttttctcctcagtctcttaagaacaggactgggcagagacccagctctgattcgggtgcttgtggacaatcatccttgtgatattgtgaacttaacggaagtgaacatttggtgtgagactcctccagctgtactaccacccagggcagatgttctcactgtcctagcctctgtggagatctgggctggcaacacttacttcttccatggaccaagcttggtggggaagggctttaccttcacatatgaagcagcagcgacaccagtggtcactgctatgtggggagaattcaagaacaacagtgtgaggttttatgtggaaggaagtaacatctctgactcagtcattcttttggggtccttgaaatgtgaacttgaagtacaattttttggtgatagcatgaacttgtctgggtgcttttttcctctccatagtttggaagccggggtctatactctccaagttcgtcacaagaggatggggtttgccaatatgtctgtggtgcctcaraaatttgagttgtcacctcagattattgccatcttcccaacacatgggtctaaatgtggtgggacagtacttactgtgaagggcatggccttcagttccagaaagaggtcagttcatgttgacatttcaggcccttttgcttgcatgattttgagtttggaagaccacacagtcctatgccagaccagatttgtgggtgaccaattttctgaagcatcactggctctaaacatcacagttctggtcaatgggctgaccagcaagtgtaaggggaactgtacactcttcatagaggaagcagcaactcctattgtggatgctttgactataagcatcagtgggtctctaaccatggtgctgatgagaggccggaggttagctaccactgctgatgagccaatagcatttgtggatgatcaacttccctgccacacaacattcttcaataccagccatgtggcatgccagataagagatttggccccaggcttccactatctgtcagctgttcatacaagtgctggatatgcttgcctcaatagtgtttctagaaacttcttcatcgtgcctcaggtgcttgattattttcctaaggactttagcatccatggtggaagtctcttgacgataaaaggcacagccctgcgaggatggaaagctacagttgtctatgttggccggcaggcttgtctaacagtgaacttcagctctgacttcatccagtgcattgttcctgcaggaaatggctctgctgctctggaaattgatgtgaatggagttttataccacataggacttgttgattacagcagtatcttcaccccagaattgctttctgtttcacggagccaagacatcttaacctttacagtggcccggatctcaggggctgcaaatgttgacatttttattgggacatcaccgtgtctaggtgttgctggcaaccgtacagttctccagtgcatggtccctctgcttcctgctggggagtatcttgtcacaggttatgatcacagccgagggtgggcctcatccactctcattcttgtgctgagagccactgtgacctcagtgaccaagaactatggttgcctgggtggaaggcttttgcatgtgctcggagcaggattttctccagggaacatctcagctgccgtatgtggtgctccatgccaagtcttggctaatgcgacagtgtctgccttcagctgcttggttctgcccctgcatgtgtccttggctttcctatgtgacctgaggcatgcagaagacagctgtaaagtcaggagctccacctacttgcgatgtgatttgactgtctccatggggacagagagactgcctggatcctggccttatgtctacctttgtgaagagagttccctgtgcctcttcgaaccagatcactggacagagtcagtctttccatcgttctcaggcctcttcctcagccctaaagtggaaagagatgaagttctcatctataatagctcctgtaacattaccatggaaactgaggcagagatggagtgtgagatgcctaatcagccaattaccgccaagattactgaaatacagaaaagctggggccagaacactcagggcaacttttccttccaattctgccgaaggtggtccaggcctcacagttggtttcctcaaagagtgccacacgatggcgacagtgtcacagtggagaccggtcacctgctactgcttgatgcgaacactagcttcctgaactccctgcacattaaaggtggcaagctgatcttcatggatccaggacccattgagctcagagcccactccatccttattacagatggtggagagctccatattggatctgaggaaaagcctttccaaggcaaagctcggatcaaaatctatggaagtgtccattccactcccttctttccctatggagtcaagttcctagctgtgaggaatggaactctttccctgcatggttcagttccagaggttactgtcacctatcttcaagcagctgcacatgcaggagacaaagtgttgactctgggggaagctgtggactggaagcctggggatgaggctgtcattaccagtgggatgactgtagcaggagctgaagcaacagaagttgttgttgtagaaactgtccacaatgcagacctccatctcaggaaccccctgagatattcctatgatttcagagagaactgggtagctggagagaatcctattttgaagccaacagttgctctcctcagcaggaacattatcatccaaggaaacttcacacttgagagggtaaagcttctcaattcatgccaggaggccaacactgctaaaggaaacctgaagcattgtttatattctaagagtgagaagatgctgggagccaggaatctgggggccagagttatcattcagtccttcccagaggaacccagcttggtcaagctgaagggagtgcagttccgagacctgggacaagccttccataagcatctaagctcactcaccctggtgggagctatgagaggctcttatatccaaagctgttcagtgtggaactccttcagcagaggccttagcatgcacaggacctggggtctgaaggtggacagcaatgtattctataagattgtagggcatgccctgctgctggggtcctacctggacggaaggtttagcactagtgagactgttactggaagaaaaaatggttggtgggaacagggaagtacaataagaaacaatgtgatcatcagtgtttctgcagctgagggactgtccggttctgaaatgttggcaccagctggcatctacactttcagtcccaccaatgtgatggagggcaacagagtgtgtgcagctggctatggatatgtcttccaccttgtgaccagccaaacattacaagctccactcctctcattcaattggaatactgctcattcttgtacaagatatggtctccttgtatatcctaaatttcaaccaccttggaataatgacactggcttcactctgttccaaaacttcatggtttggggaagtgctggtggtgcccagatttttagaagtaacaatctacacctgaaaaacttccaagtttatgcatgcagagattttggaattgacattttggaaagtgatgcgaacactttgattaccgacagctttttacttggtcatttcacccacaagggaagtctatgtatgtcagctgggatcaaaactccccaaagatgggaactgaccatttcgaacacaacttttgttaattttgatggcaactgtgtggccatcagaacctgttctggctgtttccaaggacagggtggctatactgtgaagaccaggcaattgaagtttgtaaactcttcaaatttagtwgcattyycatttcctcatgcagcagttctggaagacttagacgggtccctgtctgggaaaaatgggtctcatgttcttgcatctatggaaaccctctcagacacatgcttgaccaatgcaagcttcagtcagattgtccctggcagcgtctgtggcgaagctgttctcttccatcgtatgtctattgctctagccaatagccttgatgttcctaagaatttaaccattactgacatcagtaataagacaatcactgtcaattatgtggaagacaccctgtctaactactacggctggatggctctgctcttggatcaagagacctactcgctgcagtttgagagcccttggatgaacagatctctgcagtactcagcaacgtttgacagctttgctcctggaaattacctcctgataatgcacagggacctaccaccttatcctgacatcctcctcagatgtgggagtcaggtgggccattcacttccatttcatcctttgcctagtcaagacagagcctgtgattggttcttcaataggcaattgaggcagctcacctacctggtttcaggtgaaggtcaagttaaggtatttctccaactgaagcctggtgtacctccaagtgtttcagcttctacatcagtacctgaatcagcttcaagatggtctcttcctgaaacatggcaagatgttgaaaaaggctggggaggatacaaccacaccatcccaggacccggtgatgatgtcttgattttacccaacaagactgttcttgtggatactgatctcccagtgcttcgatgcctctatgtgatgggtaccttagaattccctgtggacagaagcaacgttctgagtgtggcatgcttactcattgcaggaggggagctgaaagtaggcactttggaaaaccccttagaaaaggaccaaagacttctgatattccttagagcctcagaagaagtcgtctgtgactattttgaaggaattcatgttgacccaggaacaattggagtttatgggaaacttcgccttcacagtgcttatcctaagaaatcctgggtacaccttggagctgacattgcaccgggaaatgagaggattattgtacacaatgcagtggattggcagcctcatgacacaattgtcctcagctcttcttcttatgaggctcatgaggcagaggtcctcactgtgaaggaagtcaagggccatcacatcaggatctatgaacgtctaaagcacaggcacattggaagtacccacaccatggaggatggtcaacaggttcatttggctgctgaggttgggctgttgaccagaaacattagaattcagcctgactcatcctgtagagggagactccttgtggggtccttcaggaagtctagcggagaagacttttctggtgttcttcracttctcaatgtagaaattcagaacatggggttgccattgtattcatctattgaattcactggtgtatcagctggatcctgggtaatatcttctactgtgcatcaaagttgcagtgtgggcatccatgcatcttccagccatggcgtgattttaactgataatgtagtgtttggcacaaatggccatggcatcgatgtggagggtcagaactattctctcaccaataaccttgtcattctgacaatgcagtcagcaaactcatctccttgggtggcaggaatcaaagtgaactatgcagaggacatcattctccatggcaatgtggtggcaggatctgagagacttggctttcatgtcggtggtcatgggtgctcctctgaagtgctttggtctgataatgtggtccactcaagcctccatggccttcacctctacaagaaacatgaatccaataactgtactggtgtctctggatttatggcttttaagaactttgactatggtgccatggttcagacagagaatagtgtggacatacagaatatcactctggtagacaatactgttggtcttttggctatcacatatgtatcttctgctctcctgagctctgtcagtactgtacagattacacttaggaattcagtcattgtggccactagctcctcttttgactgcatccacgacagaaaggctcctcagtcagccaactggacatcaacagatagagcaccttccaatcccagaggaggccgaatcggtattctgtggcctgtttctgcctcagaaccaaatgcatggccccaggagccatggcacaaagtaaggagccgtcattcagtcccaggaattatgaagcttcaagatgtcaccttttctagttttgtgaagagttgctatagcaacgacctggatgtctgcatcctgcctaatgagtatagcactggagtcatgtacccaataacagcagagaggaccagaatgctggggataaaggacaaaaacaagttctactttcctgtattacagtccagcaaagacttagtgggaaccatttgtcccacattggtctgtgaatatccaagaaaatacctcttcacagatcttgatgggagaacactgggtctacccccaccagtttctgtgtttccaaggacagaggaagagtggactggatcgttcctcaatacaggtatattcagagaagaacagaaatgcacattccgagcgatgaaccagggcttcttctgtaagcagactgagcatgcggtcctaattctcgataatgttgatgcaacttggacaatccccaaatcacacccacttgtatctgttactaatggctttgtggacacatttagcattgtgaaggacagtgatttatgccctcccacaagctctctgtctactttttattccatcttgcccaccagacaaatgaccaaggtatgctttccagagcaaactcctccattcctgcgttttcttctattgggaaaccaaagagcctccaagctcatcttggctgtattctacaatgagattcagagccctcatgttttcttagacaagagctttattccacctactccattagagtcagcattttcattgttggctgagccctctggtgccaactattttgacatcatgaataacctcttgtatgttgtcctgcaaggagaggagcctgttgaaatacattcaagtgtttccattcatttggctttgactgtgacattttcagtcctagaaaagggctgggagagagcaatgcttgaaagcctaagtgacttctttcagattgacccaaaccaaatcagactcactcttgagatgcctggcaacaaagagaccttagaggccattgcaaacagtgaaagaaaacgaaagcgcaattgcccatctgtaacttgtggtggcccttctatcagatatggtcaacgtagacctctcatggcagaaatgacatcacttaagatcacaccagcaacaactctggaaactttctcaaaggtgattgtcattgaagttggtgacctgccaaacataaggaacagtgaacccattcagtccttaccaagtaacagattacagagattggtgaaccaggttatcactgctcaacagactggagctctagaaaatgtcctaggtatgactgttggggccctactagtgactcagtccaagggagtcacaggatatagaaatgcaagtagtttaataactgggaacctgatatacacccggccctcagagctttccatcctggtgcagccttctgatggagaagtgggaatagaattgccagttcagccacggcttgtcttcctggatgagaagaatgagagagtagagtctttgggtctcccctcagaaccctggattatttcagtttctctagagggagcatctgaatcagtgcttaaagggtgtaccctggcagaaacacgggatggctatgtgaccttttctagattggctgtcttgatctctgggtcaaactggcacttgttttttactgttatatcccctccaggtactaattttacagctcgatcaaggaccttcgttgtcttgcctgtggctagcaaggagagatcaactatcatcttggccttgtccctgtgctcagtggcatcatgggtggctctgagctgtctcgtttgctgctggtttaagaaaagcaaaaccagaaaaataaaaccagaagacatatctgaatcccaggctaaggaacaaaagaagaatacccataattcctccaaacccagaggactacaagcaaagacagcaaaagagaacactttgatgggagaagatatgagaatgaaggtcatgcagggaatgcagagccagtttccccaacactcaatggatggagtgtccaaaaggaaagttagccgccttgctgtcacaggggaaagaacagctacacctgccccaaagattcccagaatcacctgtgttccaggatctcttgctcagcagctgacactgcaggagcctgggaactggcaggaggcccaacagcagttgctcagataccagctggcaggccgcaatcagctgctcctgttacgcccagacctcaggcaagagaggaaacagggtcaggagcctagccagctggacaaagggagtgactgcactggactgtctcaagagaaggccacttgcattcccactgagactttcggcctccacactactccaccagaaaccatccagtaacagctgtgggcatggagcactgtgggcatttgtatgaaaaggagaaaatgttcaaaattatttctgcattgtaaatgggagaggagaggtctgatctgtttggacaaataaggagaatgtgaactctaaatctttaaactttaatatggaagacagtcaggcaaatgcttgtaaactgagattgtaatcaatcatgtctcttgtacctctcattgatcctacccaattccagcaaataataggacctcaaaaacttgctgatattttctggggttcaaactggttacgttgcagccaacttaatacctgtgctataaatactgaatttcagtaagcaggaactgtgattaagattccctatagtcaacactcaggatgctttaggcttgtcttatggaagacctgttatagctttagttagccgatgttgtatctgcagtgctatagaaatacccaggagatattttacaccaatgtttacccttcaaacagagttaacttaataaaagttacttgtagattttaaaaaaaaaaaaaaa

SEQ ID NO: 5 - DNA, 2416 bp, Homo sapien
gtactgtttgatccagaaaagtctctgctctctgttttctaggtgttgtatctcttgttaacgttctggtgaacaaaaggaagaaatgggagatctatagtagtgccttgcaactttgtccctcgatgtcccaaaggagaagctgagagggaagggaggaagaagggaagagggagtgagagcagagagaggagagagagagaggagaacaaaaacatgactaaataaatgcacacagctctcctctgtggttgaaaaattttagttatgagaaataaagagagtctgagtttatttttaggaagaattagtggatagactaataaaaaattacatttataattgacatgaaaaagatgcagttccaaatgtagggttttaagaaaaccagtgtgcgaatgtttatttctattgttctgagcttgggagtaggcattaagaagaaatgttaaaatcacgtagccagattgaaaatagactctcctgtgctgagtatattttgtctgtgtaacaagcaagtcagatctcatgcttttggactaagcagcaaatacgcttgggtacttttccctatgtggtagatgtattctgaaagttgtatatttataagttgaattctatttcaaatagacgtaaaagtttgggaattttgcaacgaaaaggaacacttgctgaatccacttgaaaatactagaattcctttcttaaatccaagtagcctccacacttaccctatctggtaatgactcgggggcaggtacatagattgtttgttttcccaacttttagtatagtgcttagcacgtagtgggcatacaataaatgtttgcagtgttgaattaaatagaaatgattatagatttccagctgtctcaatcagaacatgcccgtaaggtgagtttattgcaaagtttttgcagattttttttttgagggggggagtatattgtagcttttatattttcagaaatgatgtaactacttctgctaatctcagtgttttcttaaaaatataaattaaatggccaggtgtggtggttcatgcctgtaatcccagcactttgggaggctgaggcagggagatcacttgaggccaggagttcaagaccagtctggctatcatggtgaaaacctgtctctacaaaaaattcaaaaattagccgggtgtggtggcgcacacctgtaatcccagctactcaggaggctgaggcataagaatcacttgaacctgggagatggaggttgcagtgaactgacatcatgccactgcactccagcccaaatgacagaatgagaccctgccacacacacacacatacacacacactttgtgtgtgtgtatgtgtgtgtgtgtatgtaggtatgtatatatatattaaatatatttatatatttatatgtaataaacataaacataattaaatatatattaaacgtgtatttagtagtacatgtgggtgtttaattctgtaattttcaaactttcagaaaatttcagaggcactatttttaagaggattttgtcgatgccctctgccatgaggtgctggagagcagctcagcactttccttagagcaggcatgagagtgaaagctgttcatctcagattaggtaaacccagatttagggtattcaaatatgtcttcatccactccaatgtactgagcgctgtgtgggaccttcacactttcacactaggaaggctgtggaaatagaagactggttcctgggtctcttcaaggaacgtgccgtctggaagaagaggcctatgtgtgccacattatctggtctaggctctacagcacggaagttagaggttcagggggaaggtggtgagcactggaatggcggaggaaagaatcagctgaggaggggctcctgagcagcaccacgaaggtgcgccaggatctcctgaggccacggagagagttgagggcatccaggaagggaccagcaggggccaaggcagaggctgatgtgtggaaggcttgtctgagtgacggtgagaacggcgtgtggctgcagtgtagggttcaaactggagagcagcacgagggaaagctggacgccagcacagggcaaagcaaaacagacttcaccctatcacgaactagaattttaatttctagaattctttttaaaactcagcttctgttcaataatgagttaatgaaagaggttattatttatccagcttaaaataagttggtgggacaaggagggcagagtggggatgattttatgcaaggttctcagatgatccttttctcctgagcttggaaaaatggtggctccatttgaagacactaaggccatgtttatctcatctccttaatttcccaggtttcagaacagcaaaataatcgtatgtaaacaaaatgataatttttttttgctttttttgtttttataaag

SEQ ID NO: 6 - protein, 4059 aa, Mus musculus
  VARIANT 2511: Xaa = Pro or Ser
MMLAWLVSLLSMEVLLLAKPYSSFQFEPAEGSLAGGTWITVVFDGLDRSILYPNNGSQLQIDLVSVAIPTLRIPCDVSPAFVDLPVVTCQTRSLPSEADAGPYSLEMRSGEQVLGTPCPGSLDSCTFKFSRDQTPVLYQVYPASGVPGEVVSVYGRVITTWLETFDPDVDYIESPLILEAREDKWLTPCSLINRQTGSCFPIQEEHGLGNVQCRVEGDYIGSQNVSFSVFNKGRSMVHKEAWLISAKQELFLYQTYPEILSVFPKVGSLGGRTDIIITGDFFDPSARVTIAGIPCDIRYVSPRKIECTTRAPGNEARLTAPQAGNRGLRFEVGDATKDVELTEATPGYRWQIVPNASSPSGFWSKEGRPFRARLSGFFVAPQTNNYTFWIQADSQASLCFSSSEEPRTKVEVASVGVGTADWFDSWEQIGNEGSWHQKTTKLELQGGAKYYLEAEQHGIAPSRGMRIGVQIHNTWLNPDVVNTYLLEKHQIRARAQRLPEIQVLHVSGKGNFFLTWGNVSSQPVPANATAQQIQTTIEELLVVKCNLAPFSAHVLLRLGFEQGLEGSRSDGVRTSSTEPFCGRFSLGQLGHLILIPEAADKGYQLDRYPYLCLAYRGHMNKTLDMTVSFLFGFQTIMKNITCDWSLTDPHPESWQFTCINLWDTCLCHSEDIQSSLANTPLLAHRIDIRPVVPEAGLLYVDEIILADTNVTVSQADSGRACPGGNVVESVSVVGVPPVYSISSWLAGCGSELPLITACSVSTEGTGDGSELIEVTAQRLQRTSPPLGGHFFLYLSDTVIPDVPVRMSARQLHKLLQDSADESTSGYLNAGDFTVTEDLNSCYEHVWTLSWTTQTGDLPNFIRVSDQNLTGVNPTVTARVVYDGGVFLGPIFGDMLATANQQTQVAVQVNDIPAYCSGSCSFQYQQESTPSVDHVWYSLGSDVNLLVHFTGTGFPRDTQFLQVTVNKTSCEVLFSNETNVACELALLPVGVHQIFMLVIPSGLAVHASGEDLLLHVEPRLDAVEPSTAAEIGGRWVTLRGSSLEGVSLVLFGTQSCVIDAIRSNSQQIQCKVPPRGKDGYTVNVTVISGDHSTVLARAFTYVSSLNPVIVSLSRNRSSIAGGEILFLGMSLLVNYTDLDVQIHVQDTSAQVLSQTAWGLEVVLPPLVPGIHVISAFINGVSIRSQGVDLYIQYLTEVFSVEPCSGSLLGVFLLSLLRTGLGRDPALIRVLVDNHPCDIVNLTEVNIWCETPPAVLPPRADVLTVLASVEIWAGNTYFFHGPSLVGKGFTFTYEAAATPVVTAMWGEFKNNSVRFYVEGSNISDSVILLGSLKCELEVQFFGDSMNLSGCFFPLHSLEAGVYTLQVRHKRMGFANMSVVPQKFELSPQIIAIFPTHGSKCGGTVLTVKGMAFSSRKRSVHVDISGPFACMILSLEDHTVLCQTRFVGDQFSEASLALNITVLVNGLTSKCKGNCTLFIEEAATPIVDALTISISGSLTMVLMRGRRLATTADEPIAFVDDQLPCHTTFFNTSHVACQIRDLAPGFHYLSAVHTSAGYACLNSVSRNFFIVPQVLDYFPKDFSIHGGSLLTIKGTALRGWKATVVYVGRQACLTVNFSSDFIQCIVPAGNGSAALEIDVNGVLYHIGLVDYSSIFTPELLSVSRSQDILTFTVARISGAANVDIFIGTSPCLGVAGNRTVLQCMVPLLPAGEYLVTGYDHSRGWASSTLILVLRATVTSVTKNYGCLGGRLLHVLGAGFSPGNISAAVCGAPCQVLANATVSAFSCLVLPLHVSLAFLCDLRHAEDSCKVRSSTYLRCDLTVSMGTERLPGSWPYVYLCEESSLCLFEPDHWTESVFPSFSGLFLSPKVERDEVLIYNSSCNITMETEAEMECEMPNQPITAKITEIQKSWGQNTQGNFSFQFCRRWSRPHSWFPQRVPHDGDSVTVETGHLLLLDANTSFLNSLHIKGGKLIFMDPGPIELRAHSILITDGGELHIGSEEKPFQGKARIKIYGSVHSTPFFPYGVKFLAVRNGTLSLHGSVPEVTVTYLQAAAHAGDKVLTLGEAVDWKPGDEAVITSGMTVAGAEATEVVVVETVHNADLHLRNPLRYSYDFRENWVAGENPILKPTVALLSRNIIIQGNFTLERVKLLNSCQEANTAKGNLKHCLYSKSEKMLGARNLGARVIIQSFPEEPSLVKLKGVQFRDLGQAFHKHLSSLTLVGAMRGSYIQSCSVWNSFSRGLSMHRTWGLKVDSNVFYKIVGHALLLGSYLDGRFSTSETVTGRKNGWWEQGSTIRNNVIISVSAAEGLSGSEMLAPAGIYTFSPTNVMEGNRVCAAGYGYVFHLVTSQTLQAPLLSFNWNTAHSCTRYGLLVYPKFQPPWNNDTGFTLFQNFMVWGSAGGAQIFRSNNLHLKNFQVYACRDFGIDILESDANTLITDSFLLGHFTHKGSLCMSAGIKTPQRWELTISNTTFVNFDGNCVAIRTCSGCFQGQGGYTVKTRQLKFVNSSNLVAFXFPHAAVLEDLDGSLSGKNGSHVLASMETLSDTCLTNASFSQIVPGSVCGEAVLFHRMSIALANSLDVPKNLTITDISNKTITVNYVEDTLSNYYGWMALLLDQETYSLQFESPWMNRSLQYSATFDSFAPGNYLLIMHRDLPPYPDILLRCGSQVGHSLPFHPLPSQDRACDWFFNRQLRQLTYLVSGEGQVKVFLQLKPGVPPSVSASTSVPESASRWSLPETWQDVEKGWGGYNHTIPGPGDDVLILPNKTVLVDTDLPVLRCLYVMGTLEFPVDRSNVLSVACLLIAGGELKVGTLENPLEKDQRLLIFLRASEEVVCDYFEGIHVDPGTIGVYGKLRLHSAYPKKSWVHLGADIAPGNERIIVHNAVDWQPHDTIVLSSSSYEAHEAEVLTVKEVKGHHIRIYERLKHRHIGSTHTMEDGQQVHLAAEVGLLTRNIRIQPDSSCRGRLLVGSFRKSSGEDFSGVLXLLNVEIQNMGLPLYSSIEFTGVSAGSWVISSTVHQSCSVGIHASSSHGVILTDNVVFGTNGHGIDVEGQNYSLTNNLVILTMQSANSSPWVAGIKVNYAEDIILHGNVVAGSERLGFHVGGHGCSSEVLWSDNVVHSSLHGLHLYKKHESNNCTGVSGFMAFKNFDYGAMVQTENSVDIQNITLVDNTVGLLAITYVSSALLSSVSTVQITLRNSVIVATSSSFDCIHDRKAPQSANWTSTDRAPSNPRGGRIGILWPVSASEPNAWPQEPWHKVRSRHSVPGIMKLQDVTFSSFVKSCYSNDLDVCILPNEYSTGVMYPITAERTRMLGIKDKNKFYFPVLQSSKDLVGTICPTLVCEYPRKYLFTDLDGRTLGLPPPVSVFPRTEEEWTGSFLNTGIFREEQKCTFRAMNQGFFCKQTEHAVLILDNVDATWTIPKSHPLVSVTNGFVDTFSIVKDSDLCPPTSSLSTFYSILPTRQMTKVCFPEQTPPFLRFLLLGNQRASKLILAVFYNEIQSPHVFLDKSFIPPTPLESAFSLLAEPSGANYFDIMNNLLYVVLQGEEPVEIHSSVSIHLALTVTFSVLEKGWERAMLESLSDFFQIDPNQIRLTLEMPGNKETLEAIANSERKRKRNCPSVTCGGPSIRYGQRRPLMAEMTSLKITPATTLETFSKVIVIEVGDLPNIRNSEPIQSLPSNRLQRLVNQVITAQQTGALENVLGMTVGALLVTQSKGVTGYRNASSLITGNLIYTRPSELSILVQPSDGEVGIELPVQPRLVFLDEKNERVESLGLPSEPWIISVSLEGASESVLKGCTLAETRDGYVTFSRLAVLISGSNWHLFFTVISPPGTNFTARSRTFVVLPVASKERSTIILALSLCSVASWVALSCLVCCWFKKSKTRKIKPEDISESQAKEQKKNTHNSSKPRGLQAKTAKENTLMGEDMRMKVMQGMQSQFPQHSMDGVSKRKVSRLAVTGERTATPAPKIPRITCVPGSLAQQLTLQEPGNWQEAQQQLLRYQLAGRNQLLLLRPDLRQERKQGQEPSQLDKGSDCTGLSQEKATCIPTETFGLHTTPPETIQ

SEQ ID NO: 7 - protein, 4051 aa, Rattus norvegicus
MPAWRISLLSVELLLLANAYSSFRIEPTEGSLAGGTWITLVFDGLETSILYPNNGSQLAIDLVNVATPALKIPCDISPLFVDLPVVTCQTRSLPSEAPEGLYSLEIRSGDQLLGSPCARLLDNCTFKFSKEQTPVLYQVNPSSGVPGEVVHVYGWVITDRMEIFDPDVDYMESPLILEAGGDKWLTPCSLINRKTGICFPIQEEHGLGTVQCRVEGSYIGSQNVSFSVFNKGRSMVHKEAWLISAKQELFLYQTYPEIHSVFPKVGSLGGRTDITITGDFFDPSARVTIAGIPCDIRHVSPKKIECTTRAPRNGARLTAPQASNRGLLFEVGNAVKDEELTEATPGYRWQIVPNASSPSGFWSKEGRPFRARLSGFFVAPETNNYTFWIQADSQAFLRFSSSEEPRMKVEVASIAVGTADWFDSWEQNGNEGSWQQKTNKLELQGGAKYYLEAEQHGIAPSRGMRIAVQIHNTWLNPDVVNTYLLEKHQIRAQAQRLPEIQVLNMSGKGNFFLTWGNVSSQPVPANATAQQIQTIIEELLVVKCNLVPLSARVLLWLGFEQGLDGSRSDGVLTSSTEPFCGRFSLGQLRHLILSPGAVSKGYQLDRYPYLCFAYRGHMNRTLDMTVSFLFGFQNVMKNITCDWSLMEPHPESWHFTCNNLWDTCVRHSEELQSSLANTPLLVHRIDIFPVVPEADLLYVDEIILADTNVTVSQADSGVARPGGNVVESVSVVGVPPVYSIASWMAGCGSELPLITACYVPTQGTGEGSELIEVTAKRLQRTSPPLGGHFSLHLSDTVIPDVPVHVSARQLHKLLQDNADESTSGYLNADDFIVTKDQNSCYEHVWTLSWTTQIGDLPNFIRVSDQNLTGLNPTITARVVYDGGVFLGPILGDTLATANQQTQVTVQVNDIPAHCSGSCSFQYQRASTPSVDCVWYSLGSDVNLLVHFTGNGFPRDAQFLQVTVINTSCEVIFSNETNVACELALLPVGVHRVFMLVRPSGLALNASGEGLFLRVEPRLDAVEPSTGAEIGGWWVTLRGSSLEGVSLVLFGTRSCVIDVIRSNSQQIQCKVPPRGKDGYTVNVTVINGDHSTVLARAFTYVSSLNPVIVSLSRNRSNIAGGEILFLGMTLMVNYTDLNVQIYIQNTSAQVLAKTAWGLEVLLPPLLPGIYVISAFINGISIRSQGVDLHIQYLTEVFSMEPCSGSLLGGTILRLSGAGLGRDPALIRVLVDNRPCDTVKLTEVNIWCETPRVLLPPRADVLTVPASVEIWAGNTSFVGKAFIFTYEAAATPVVTAMWGELMNNSVRFYVEGNNLSDSVILLGSLKCDLQVQLFGDNMNLSGCSLLLHSLEAGVYALQVRHKRMGFANMSAVPQKFELSPQIIDIFPTHGSIYGGTVLTVKGTAFRSRRRSVHVDLSGPFTCVILSLGDHTVLCQTKFVGDQFSETSLALNITVLVNGLTSKCEGNCTLFLEEAATPIVDALTISISGSLTTVLMRGQRLGSAGEPTAFVDGQLLCHTTFFNTSYVACQMRDLAPGFHYLSAVRTSAGYACLNSVSRNFFVVPQVFDYFPKDFSIHGGSLLTIKGTTLRGWKTTFVYVGQQACLTVNISSEFIQCIVPAGNGSAALEIDVDGVLYHMGFVDYSSIFTPELLSVSRSHDILTFTVARISGAANVDILIGTSPCLDVAGNRTILRCMVPLLPAGEYAVTGYDHSRGWVSSTLILVLRATVTSVTENYGCLGGRLLHVLGAGFSPGNISAAVCGAPCQVLANATVSAFSCLVLPLDASLAFLCNLRHAEDSCGVSRPTYLQCDLTVSVGMERLPGSWPYVYLCEESSQGLFVPDHQIFLSFSGLFLSPKVERDEVLIYNSSCNITMETEAEMECGTPNQPITAKITEIQKSRGQNTQGNFSFQFCRRWSRPHSWFPHRVPQDGDNVTVETSHLLLLDANTSLLNSLHIKGGKLIFMEPGPIDLRAHSILITDGGELHIGSEDKPFQGKARIKLYGSVHSTSFFPYGVKFLAVRNGTLSLHGSVPEVTFTYLRAAACAGDTVLALEEAVAWHPGDEAVISSGATVEGAEATEEVVVVETVHDADLHLRNPLRYSYNFTENWVTGVNHILKATVALLSRNIIVQGNLTLERVKLLDSCQEAGAAEGNLKHCLYSKSEKMLGARNLGARVIIQSFPEEPSLVKLKGVQFRDLGQAFHKHLSSLTLVGAMRGSYLHSCSMWGSFSRGLSIHRTWGLKVENNIFYKIVGHALLVGSYMDRSFSTSEAVIGRKNGWWEEGSMIRNNVIINVSGAEGLSSSEMLAPAGIYTFSPTSAIEGNRVCAAGYGYVLHLVTSQTVQAPLLSFNWNTAHSCTRYGLLVYPEFQPPWNNDTGFTLFQNFMVWGSAGGAQIFRSNNLHLRNFQVYACRDFGIDILESDTNTLVTDSFLLGHFTHEGSLCMSVGIKTPKRWGLTISNTTFVNFDLNCVAIRTCSGCSQGQGGFPVKTRQLKFVNSSNLVAFPFPHAAVLQDLDGSLSGKNGSHVLASMETLSDTCLTNASFSQIVSGSVCGEAVLFHRMSIGLAKSLDVPKNLTMTDIRNRTITINYVDDTLSNSYGWMALLLDQETYLLQFEGPWTDGSLQYSATFDNFAPGNYLLLMHRDLLPYPDILLRCGSRVGQSLPSHPLPSRDGACDWFFHRQLRQLTYLVSGEGQVHVFLQVKHGAPPTVSASTSVSESALKWSLPETWQDVEKGWGGYNHTIPGPGDDVLILPNKTVLVDTDLPVLRCLYVMGTLEFPVDRSNVLSVACLLIAGGELKVGTLENPLGKDQRLLILLRASEEIFCDRFDGIRVDPGTIGVYGKLRLHSAYPKKSWVHLGADIAPGNERIIVHNAVDWQPHDKIVLSSSSYEPHEAEVLTVKEVKDHHIRIYERLKHRHIGSAHTLEDSRQICLAAEVGLLTRNIRIQSDSSCRGRLLVGSFRKSSGEEFSGILQLLNVEIQNMGSPLYASIEFPGVSAGSWVISSTVHQSCTVGIHASSSRGMVLNDNIVFGTKGHGIDVEGQNYSLTNNLVILSMQSANSLPWVAGIKVNYAEDIILHGNVVAGSERLGFHVRGHGCSSEVLWSDNVVHSSLHGIHLYKKREPNNCTGVSGFLAFKNFDYGAMVQTENSVDIQNITLVDNTVGLLAISYVSSAPLRSGSTLQITLRNSVIVATSSSFDCIQDRKAPQSANWTSTDRAPSNPRGGRIGILWPVSASEPNGWPQEPWHKVRGGHSVPGIMKLQDVTFSSFVKSCYSDDLDVCILTNEHSTGIMYPITAERTRMLKIKDKNKFYFLPLQSGKDLVGTICPASSDCEIPRKYLFTDLDGRTLGLSPPVSVFPRIEEGWTGSFFNTGIFREEQKCTFRAMIQGFFCKQTEHAVLILDNVDVTWTIPKFYPLVSITNGFVDTFSIVKDSGLCFPTSSPSTFYSILPTSQMTKVCFPERTPPFLHFFLLANRRASKLILAVFYNEIQSPYVFLDKSFIPPTPVESAFSLLDEPVGANYFDIMNNLLYVVLQGEEAVEIRSSVSIHLALTVTFSVLEKGWEKAVLESLRGFFQIDQNQIRLTLEMPGNKETLKAIANSEGKRKRSCPTVTCAVPSSRYVQRRPLMAEMASFTITPATTLETFSKVIVFEIGDLPNARDSEFIQSLPSNRLQKLAHQVITAQQTGVLENVLGMTVGALLVAQSEGVTGYRNASSLITGNQIYTRPSVVSILVQPSDGEVGIELPIQPRLIFLDEKNERVEALGLPSEPWIISVSLEGTSESMLKGCTQAETQDGYVTFSRLAVLTSGSNWHLVFTVTFPPGTNLTARSKTFAVLPVAGKERSTIILALSLCSVASWVALSCLICCWFKKSKTRKIKPEDISESQAKEQKKNTHISSKPRELQVKTAKEDAMMGEDMRMKVMEGRQNQFSQHSMDGVSKRKVSRRAVPEERTATPALKIPRTTCVPGALPQQLTLPEPGNWQEAQQQLLRYQLAGRDQLLLLCPDLRQERQQGQEPSQLNKGSGRCMGLSQEKATCVPTETFCLHTAPPETIQ

SEQ ID NO: 8 - protein, 77 aa, Homo sapien
SEILSVFPETGSLGGRTNITITGDFFDNSAQVTIAGIPCDIRHVSPRKIECTTRAPGKDVRLTTPQPGNRGLLFEVG

SEQ ID NO: 9 - protein, 85 aa, Homo sapien
PCVHSVWYSIDGDINLMIYITGTGFSGDSQFLQVTVNKTSCKVIFSNQTNVVCQTDLLPVGMHRILMLVRPSGLAISATGEDLFL

SEQ ID NO: 10 - protein, 85 aa, Homo sapien
PRLDMVEPSRAADIGGLWATIRGSSLEGVSLILFGSYSCAINVATSNSSRIQCKVPPRGKDGRIVNVTVIRGDYSAVLPRAFTYV

SEQ ID NO: 11 - protein, 92 aa, Homo sapien
TEVFSIEPCCGSLLGGTILSISGIGFSRDPALVWVLVGNRSCDIVNLTEASIWCETLPAPQIPDAGAPTVPAAVEVWAGNRFFARGPSPSLV

SEQ ID NO: 12 - protein, 91 aa, Homo sapien
PRIMAIFPSQGSACGGTILTVRGLLLNSRRRSVRVDLSGPFTCVILSLGDHTILCQVSLEGDPLPGASFSLNVTVLVNGLTSECQGNCTLF

SEQ ID NO: 13 - protein, 85 aa, Homo sapien
PVMDALSTNTSGSLTTVLIRGQRLATTADEPMVFVDDQLPCNVTFFNASHVVCQTRDLAPGPHYLSVFYTRNGYACSGNVSRHFY

SEQ ID NO: 14 - protein, 87 aa, Homo sapien
PQVFHYFPKNFSLHGGSLLTIEGTGLRGQNTTSVYIDQQTCLTVNIGAELIRCIVPTGNGSVALEIEVDGLWYHIGVIGYNKAFTPE

SEQ ID NO: 15 - protein, 92 aa, Homo sapien
HLSLHIEPEEGSLAGGTWITVIFDGLELGVLYPNNGSQLEIHLVNVNMVVPALRSVPCDVFPVFLDLPVVTCRTRSVLSEAHEGLYFLEAYF

SEQ ID NO: 16 - protein, 109 aa, Homo sapien
PIVHQVYPPSGVPGKLIHVYGWIITGRLETFDFDAEYIDSPVILEAQGDKWVTPCSLINRQMGSCYPIQEDHGLGTLQCHVEGDYIGSQNVSFSVFNKGKSMVHKKAWL

SEQ ID NO: 17 - protein, 84 aa, Homo sapien
PVIVTLSRNISNIAGGETLVIGVARLMNYTDLDVEVHVDALAPVHTQSAWGLEVALPPLPAGLHRISVSINGVSIHSQGVDLHI

SEQ ID NO: 18 - protein, 82 aa, Homo sapien
PVVTAMQGEITNSSLSLHVGGSNLSNSVILLGNLNCDVETQSFQGNVSLSGCSIPLHSLEAGIYPLQVRQKQMGFANMSVVL

SEQ ID NO: 19 - protein, 85 aa, Homo sapien
PELISISQSDDILTFAVAQISGAANIDIFIGMSPCVGVSGNHTVLQCVVPSLPAGEYHVRGYDCIRGWASSALVFTSRVIITAVT

SEQ ID NO: 20 - protein, 81 aa, Mus musculus
PAVASVSPTSGSIAGGTTLMITGNGFSPGNTTVTVGDQPCQITFISSSEVYCSTPAGRAGTANLKISVNAIIYPPLSFTYA

SEQ ID NO: 21 - protein, 83 aa, Mus musculus
PVITSISPRYGPQAGGTLLTLTGKYLNSGNSRHISIGGKTCTLKSVSDSILECYTPAQTTSDEFPVKLKIDLANRETSSFSYR

SEQ ID NO: 22 - protein, 85 aa, Mus musculus
PTFYRVSPSRGPLSGGTWIGIEGSHLNAGSDVAVSIGGRPCSFSWRNSREIRCLTPPGHTPGSAPIVININRAQLSNPEVKYNYT

SEQ ID NO: 23 - protein, 86 aa, Homo sapien
PVLIAVQPLFGPRAGGTCLTLEGQSLSVGTSRAVLVNGTECLLARVSEGQLLCATPPGATVASVPLSLQVGGAQVPGSWTFQYRED

SEQ ID NO: 24 - protein, 88 aa, Artificial Sequence
  Consensus
PVITSISPSSGPLSGGTEITITGSNLGSGSEDIKVTFGGTECDVISSSSSTQIVCKTPPVAGGPSPPVVTVSLDGDGLSSSPVTFTYV

SEQ ID NO: 25 - DNA, 1776 bp, Homo sapien
atgactgcctggctgatctctctgatgagtattgaagtactacttttggcagtacgtcacctgagtttacatattgaacctgaagaaggtagccttgcagggggaacgtggatcacagtcatttttgatggtttggagttgggtgttctttaccccaacaatggctctcaattggagatacacctggtgaacgtgaacatggtggtgcccgcactgcggagtgttccctgtgacgtctttcctgttttcttggatttgcctgtggtgacatgccggaccagatctgtgctgtctgaagcacatgagggtctgtacttcctggaagcatacttcgggggacagctggtaagcagtccaaatccaggaccacgagatagctgtactttcaagttttccaaggcgcagacacccatcgttcaccaagtttatccaccaagtggtgttccaggaaaactaatacatgtatatggctggattatcactggaagattggaaacttttgattttgatgctgagtacattgatagcccagtgatcttggaagctcaaggagacaaatgggttactccttgctctcttataaataggcagatgggaagctgttatcctattcaggaggaccatggtcttgggactctgcagtgccatgtggaaggcgactacatcggctcccagaatgttagcttctcagtatttaacaaaggaaagtcaatggtccacaagaaggcatggctgatcagtgctaaacaggatcttttcctataccagacacactcagaaatattatctgtgtttccagaaactgggagccttgggggaagaacaaacatcacaattacaggagacttttttgacaattctgcccaggttaccattgcaggcattccatgtgatattagacacgtgtctcccaggaagattgagtgcaccactcgggctccaggaaaagatgtgaggctcaccacccctcagccaggcaatcgagggcttctttttgaagttggagatgctgttgagggactggaactgactgaagccaccccagggtacaggtggcagattgtccctaatgccagttctccatttgggttttggtcacaggaaggacaacctttcagagcacggctcagtgggttctttgtggctccagagacaaataattacactttctggattcaggcagatagccaagcttccttgcatttcagttggtcagaggaaccaaggactaaggtgaaagtggcctccatcagcgtcggcactgctgactggtttgactcctgggagcagaatagggatgaagggacctggcagcagaagactcccaagttggagctgttgggtggagccatgtactacctggaagcagagcatcatgggatagccccaagcagggggatgaggattggtgtccagattcacaacacctggctgaatcctgatgtggtcaccacttacctacgggagaagcaccagatccgagtccgagcccagaggcttccagaagtacaggtgctgaatgtatcaggcagaggaaacttcttccttacttgggacaatgtctctagtcagccaatccctgcaaatgccacagcccatctgattcaaacaaccattgaggagttacttgcagtaaaatgcaaactggaacccctttggtctaacatccttctccggcttggatttgaacgaggcccagaagtttccaactctgatggggacctcaccagtgggacggagcccttctgtggcaggttcagcctccgtcagcctcga

SEQ ID NO: 26 - protein, 1871 aa, Mus musculus
MGHLWLSGTWFLFGLLWCAADSHKGSSETIPKVTEVIPKYGSINGATRLTIKGEGFSQASQFNYGADNTELGNHVQLVSSFQSITCDVEKDSSHSTQITCYTRAMPEDTYSVRVSVDGVPVAENNTCKGVASSWACSFSTKSFRTPTIRSITPLSGTPGTLITIKGRLFTDVYGSNTALSSNGRNVRILRIYIGGMPCELLIPHSDDLYGLKLDHANGDTGSVTCKTTGTYIGHHNVSFILDSDYGRSFPEKMTYFVSSLNKISMFQTYPEVVMVSPSKGSTEGGTLLTIHGHFFDQTDLPVRVLVGGQACAILNVTENTIYCKTPPKPHILKATYPGGRGLKVEVWNNSRPAHLEDILEYNEHTPGYMGATWTDSASYVWPIEQGTFVARISGFLVPPDSDVYRFYIRGDDRYAIYFSQTGRTEDKVRIAYYSGNANTYFSNSTQRSDEIHLQKGKEYYIEILLQEYTLSAFVDVGLYQYKNVFTEQQTGDALNEEQVIKSQSTVVPEVQIITLENWETADVTNEVQQVTVTSPCVGANSCSLSQYRFIYNMEKTVWLPADASDFTLKSALNDLWSIKPDSVQVTSKRDLQSYIYTITFVSTRGDFDLLGYEVFEGSNVTLSITEQTKGKPNLETFTLNWDGIASKPLTPESSEAEFQVAVEEMVSAKCPPEIAHLEEGFLVKYFRDYETDFELEHINRGQKTAETDAYCGRYSLKNPAVLFDSTDVKPNKSPYGDILLFPYNQLCLAYKGSLANFIDLKFKYQDSGKIIRSADVQFEYNFASGNKWTYTCIDLLDFLQTKYAGTSFSLQRITLQKSSEFQSIYVDAVYIGQTPTVSVLDDMPKRRPPALANKGIFLKHFQVNRTKLNGSAMTIQYSVTITSYNCSHNIPMMAVSFGQIITNETKNELVYRGNNWPGESKIRIQKIQEASPPISGSFDVQAYGHTLKGIPAAVPAADLQFALQSLEEIEQVSVNREGTCAGYSWSIRWTSPRGKQPLLQINDSNIIGEKANVTVTTIKEGGLFRQRIPGDMLRTLNQQPQVEVYLNGIPAKCSGDCGFTWDAMITPLILTTTPSEGSYAESTILTIAGSGFSPTSAVSVSVGSTRCSLLSVEENEIKCQIVNGSAGHVPVAVSIADVGLAQNLEGEGSHFIYRSQISHVWPDSGSLAGGTLLTISGFGFSENSTVLVGNETCNVIEGDLNRITCRTSKRIEGTVDISVITNGIQVTAKDSFSYSCLQTPVVTDFSPKERTVLGKVNLTIKGYNFGNELAQNTVYVGRKHCQVLHSNFTDITCLLPTLPPGKHDIYVKVRNWGLASTRNKLNASILYILEVIHMFPQRGSLYGGTEITIMGFGFSTIPTENSVLLGSFPCDITSSSENVIKCTLHSTGTVFRITNNGSHLVHGLGYAWSPSVLNVTVGDTVVWSWQAHPFLRGIGYRIFSVSSPGSVTYDDKGFTNGRQKSASGSFSYQFTSPGIYYYSSGYVDEAHSISLQGVINVFPAEARHIPLYLFVGNIEATYVPAGPAHLQLASTAAGCLATEPLCGLNDTRVKHSNKLFFELSNCISPSIINITPSTGTANELITIIGHGFSSLPCANKVTIGSYPCVVEESSENSIICHIDPQNSMNVGIREIVTLIVYNLGTAINTLTKAFDRRFVLLPNIDMVMPKAGSTTGMTRVTIQGSGFMSSPEGVEVFMGDFPCKVLSVTYTALNVETSPAPQQLVLVDILIHGVPAQCQSNCSFSYLENIAPYVTGIFPNSIQGYGNVLIKGERFGTVLEEISIFIGSQQFRVIDVNENNITVLMTPLEAGLHSLSVVVGSKGLALGNLTISSPAVASVSPTSGSIAGGTTLMITGNGFSPGNTTVTVGDQPCQI

SEQ ID NO: 27 - DNA, 446 bp, Homo sapien
tgtgactggagtctcacgaggaccagccccgagagctggcagttcgattgcactgacctctgggagacttgtgtgcgttgcttcggggatctccagccccctccggcaaactccccagtgctggttcatcagatcaaccttctccctctggcccaggagacgggcctgttctatgtggatgaaattattattgcagacacaaacgtaacagtttctcaagctgattctggaacggctcgcccagggggcaatctggtggaatcagtctctgtggtgggatcccctccggtctacagtgtcacctcctggctggcggggtgtggcacggagctcccgctcatcactgcacgctctgtgcccactgaaggaacagaagagggatctggactggtcctggtgacgacacagagacgacagcggacaagtccacctctaggaggacactt

SEQ ID NO: 28 - DNA, 188 bp, Homo sapien
tcttgcaggttacagtgaacaaaacgagttgcaaagttattttctcaaaccagaccaatgtagtctgtcagacagatttgctacctgttggaatgcatcggatcttgatgttggtgagaccctctggtcttgccatcagtgccactggagaagacctcttcctaaatgtgaaacctagactggatatg

SEQ ID NO: 29 - protein, 116 aa, Homo sapien
  VARIANT (1)...(433): Xaa = Any Amino Acid
RLRNWDPGQDSAKQVVIKEGDMLRLTSDATVHSIVIQDGGLLVFGDNKDGSRNITLRTHYILIQDGGALHIGAEKCRYKSKATITLYGKSDEGESMPTFGKKFIGVEAGGTLELHG

SEQ ID NO: 30 - protein, 114 aa, Homo sapien
  VARIANT (1)...(433): Xaa = Any Amino Acid
ELQPWNPGHDQDHHVHIGQGKTLLLTSSATVYSIHISEGGKLVIKDHDEPIVLRTRHILIDNGGELHAGSALCPFQGNFTIILYGRADEGIQPDPYYGLKYIGVGKGGALELHG

SEQ ID NO: 31 - DNA, 489 bp, Homo sapien
cacagtcggcttggggcctggaggtggcactgcccccactgccagctggtctccacagaatttccgtctctatcaatggggtcagcattcactcacaaggggttgatctccacatccagtacctcacagaagttttcagcatcgagccttgctgtgggtccctgctgggagggaccatcctcagcatctcaggaataggcttcagcagggacccagctttggtttgggtacttgtgggcaatcggtcctgtgacattgtgaacttaacggaggcgagcatctggtgtgaaaccctgccagccccccagatacccgatgcgggcgctcccactgttccagctgccgtggaggtctgggctggcaacaggttcttcgcccgtggtccttcaccaagcttggtggggaaaggcttcaccttcatgtatgaagcggcagcaacaccagtagtcactgccatgcaaggagaaatcacaaatagcagcctgagcc

SEQ ID NO: 32 - protein, 366 aa, Homo sapien
SIVALNKSYEVYFTGTSPQNLRLMLLNVDHNKAVLVGIFFSTLQRLDVYVNNLLVCPKTTIWNAQQKHCELNNHLYKDQFLPNLDSTVLGENYFDGTYQMLYLLVKGTIPVEIHTATVIFVSFQLSVATEDDFYTSHNLVKNLALFLKIPSDKIRISKIRGKSLRRKRSMGFIIEIEIGDPPIQFISNGTTGQMQLSELQEIAGSLGQAVILGNISSILGFNISSMSITNPLPSPSDSGWINVTAQPVERSAFPVHHVAFVSSLLVITQPVAAQPGQPFPQQPSVKATDSDGNCVSVGITALTLRAILKDSNNNQVNGLSGNTTIPFSSCWANYTDLTPLRTGKNYKIEFILDNVVGVESRTFSLL

SEQ ID NO: 33 - DNA, 30 bp, Artificial Sequence
  Primer
gccttcttgtggaccattgactttcctttg

SEQ ID NO: 34 - DNA, 20 bp, Artificial Sequence
  Primer
gccagcatttccgagtggtg

SEQ ID NO: 35 - DNA, 23 bp, Artificial Sequence
  Primer
tgggtaagcaaggtcaaacttcc

SEQ ID NO: 36 - DNA, 24 bp, Artificial Sequence
  Primer
accctcaatacaagaaggaaccac

SEQ ID NO: 37 - DNA, 20 bp, Artificial Sequence
  Primer
ggtgctcccgtctcccagcg

SEQ ID NO: 38 - DNA, 18 bp, Artificial Sequence
  Primer
ggtctttgaaagaaggaa

SEQ ID NO: 39 - DNA, 21 bp, Artificial Sequence
  Primer
caatcaattcaaaggtctcag

SEQ ID NO: 40 - DNA, 22 bp, Artificial Sequence
  Primer
gcaggactacagaaatactcag

SEQ ID NO: 41 - DNA, 20 bp, Artificial Sequence
  Primer
tcccacttaccacagagaag

SEQ ID NO: 42 - DNA, 21 bp, Artificial Sequence
  Primer
aggaggaacatggatcacagt

SEQ ID NO: 43 - DNA, 21 bp, Artificial Sequence
  Primer
agccattgttggggtaaagaa

SEQ ID NO: 44 - DNA, 21 bp, Artificial Sequence
  Primer
ttataatcatgcaaaatgggc

SEQ ID NO: 45 - DNA, 21 bp, Artificial Sequence
  Primer
acatcattttcatgaggcaat

SEQ ID NO: 46 - DNA, 21 bp, Artificial Sequence
  Primer
ggctgtgaggaatggaactct

SEQ ID NO: 47 - DNA, 23 bp, Artificial Sequence
  Primer
atggtcattgtcctcatatttgc

SEQ ID NO: 48 - DNA, 25 bp, Artificial Sequence
  Primer
aggtttcagaacagcaaaataatcg

SEQ ID NO: 49 - DNA, 27 bp, Artificial Sequence
  Primer
ttctcaaggtaacctattgtgttctta

SEQ ID NO: 50 - DNA, 25 bp, Artificial Sequence
  Primer
tggtttgaatctgaccttcaaaacc

SEQ ID NO: 51 - DNA, 23 bp, Artificial Sequence
  Primer
aaatgtgcacttggtaaaacccc

SEQ ID NO: 52 - DNA, 25 bp, Artificial Sequence
  Primer
tttcacactgtcctgtgtcaatgac

SEQ ID NO: 53 - DNA, 23 bp, Artificial Sequence
  Primer
aaaatccctcatcctgtctggtc

SEQ ID NO: 54 - DNA, 24 bp, Artificial Sequence
  Primer
ttgggaattcatggtttttgattc

SEQ ID NO: 55 - DNA, 25 bp, Artificial Sequence
  Primer
acataccttcctccagccttagaac

SEQ ID NO: 56 - DNA, 21 bp, Artificial Sequence
  Primer
gaaaggcttgtgcctcctgtg

SEQ ID NO: 57 - DNA, 25 bp, Artificial Sequence
  Primer
tggcaaacagattcacaattattcc

SEQ ID NO: 58 - DNA, 21 bp, Artificial Sequence
  Primer
cattgagtttgagctaagtcc

SEQ ID NO: 59 - DNA, 22 bp, Artificial Sequence
  Primer
catgcagcatgtatgtaactag

SEQ ID NO: 60 - DNA, 18 bp, Artificial Sequence
  Primer
gtttattgggagtattgc

SEQ ID NO: 61 - DNA, 18 bp, Artificial Sequence
  Primer
gtggacgaacttacaagc

SEQ ID NO: 62 - DNA, 18 bp, Artificial Sequence
  Primer
tgagttgtctggtcattc

SEQ ID NO: 63 - DNA, 20 bp, Artificial Sequence
  Primer
gagaaagaaatggataagac

SEQ ID NO: 64 - DNA, 19 bp, Artificial Sequence
  Primer
actcgtgcagattcctgag

SEQ ID NO: 65 - DNA, 18 bp, Artificial Sequence
  Primer
caagatgagagagatagg

SEQ ID NO: 66 - DNA, 20 bp, Artificial Sequence
  Primer
caatcccagttgatattttc

SEQ ID NO: 67 - DNA, 19 bp, Artificial Sequence
  Primer
acaagggaaggggtacttg

SEQ ID NO: 68 - DNA, 18 bp, Artificial Sequence
  Primer
tggtctatatttggaagc

SEQ ID NO: 69 - DNA, 18 bp, Artificial Sequence
  Primer
acctgcaatggtaacctg

SEQ ID NO: 70 - DNA, 19 bp, Artificial Sequence
  Primer
cctacacacacacacatac

SEQ ID NO: 71 - DNA, 18 bp, Artificial Sequence
  Primer
gtttattgaacagccctg

SEQ ID NO: 72 - DNA, 18 bp, Artificial Sequence
  Primer
ttccccaatttgggaagg

SEQ ID NO: 73 - DNA, 18 bp, Artificial Sequence
  Primer
ttagcaaaggtgcttttg

SEQ ID NO: 74 - DNA, 21 bp, Artificial Sequence
  Primer
ttggttactcttgcttgactc

SEQ ID NO: 75 - DNA, 18 bp, Artificial Sequence
  Primer
ctggcaacagagaaaagg

SEQ ID NO: 76 - DNA, 18 bp, Artificial Sequence
  Primer
tgcatagtattgatcatg

SEQ ID NO: 77 - DNA, 19 bp, Artificial Sequence
  Primer
agctccatgggactggaaa

SEQ ID NO: 78 - DNA, 22 bp, Artificial Sequence
  Primer
ttagcaccatcatttagtcttg

SEQ ID NO: 79 - DNA, 18 bp, Artificial Sequence
  Primer
aaagaccacccccagttc

SEQ ID NO: 80 - DNA, 19 bp, Artificial Sequence
  Primer
aattcctggcatttttttc

SEQ ID NO: 81 - DNA, 24 bp, Artificial Sequence
  Primer
cattttatagaaagaaagaagacc

SEQ ID NO: 82 - DNA, 18 bp, Artificial Sequence
  Primer
tatctatgcctgcctttc

SEQ ID NO: 83 - DNA, 20 bp, Artificial Sequence
  Primer
aatacctacccacctgaccc

SEQ ID NO: 84 - DNA, 20 bp, Artificial Sequence
  Primer
cactaatagaactgaaggac

SEQ ID NO: 85 - DNA, 20 bp, Artificial Sequence
  Primer
tgactgaattcccaccacgc

SEQ ID NO: 86 - DNA, 22 bp, Artificial Sequence
  Primer
taaccggagaggactgcaagtg

SEQ ID NO: 87 - DNA, 22 bp, Artificial Sequence
  Primer
tttgaggtaggcatgtgaccgg

SEQ ID NO: 88 - DNA, 25 bp, Artificial Sequence
  Primer
ttttccacacagcaagtctaccatc

SEQ ID NO: 89 - DNA, 24 bp, Artificial Sequence
  Primer
cattcttaggagaagggacaggtg

SEQ ID NO: 90 - DNA, 23 bp, Artificial Sequence
  Primer
caccccaacccagacgttaatac

SEQ ID NO: 91 - DNA, 22 bp, Artificial Sequence
  Primer
tcccaggatgttgttcccttgg

SEQ ID NO: 92 - DNA, 18 bp, Artificial Sequence
  Primer
tagtgtctgtgttttctg

SEQ ID NO: 93 - DNA, 20 bp, Artificial Sequence
  Primer
tccagggcagcaaatccatg

SEQ ID NO: 94 - DNA, 24 bp, Artificial Sequence
  Primer
ttcggttccatgacagaatttacc

SEQ ID NO: 95 - DNA, 23 bp, Artificial Sequence
  Primer
tgaaactggagcttgcacttagg

SEQ ID NO: 96 - DNA, 25 bp, Artificial Sequence
  Primer
cagcttgggagcacttcacatatac

SEQ ID NO: 97 - DNA, 22 bp, Artificial Sequence
  Primer
ttaagcccatctcagagccaag

SEQ ID NO: 98 - DNA, 20 bp, Artificial Sequence
  Primer
tgaagtaatatcactgagag

SEQ ID NO: 99 - DNA, 19 bp, Artificial Sequence
  Primer
acatactgtgagaccctcc

SEQ ID NO: 100 - DNA, 18 bp, Artificial Sequence
  Primer
cctgtatggttggtgatc

SEQ ID NO: 101 - DNA, 19 bp, Artificial Sequence
  Primer
gagaaagagatatgaaagg

SEQ ID NO: 102 - DNA, 18 bp, Artificial Sequence
  Primer
cccttaagtcagtcctac

SEQ ID NO: 103 - DNA, 18 bp, Artificial Sequence
  Primer
tttataggaccaatgctc

SEQ ID NO: 104 - DNA, 21 bp, Artificial Sequence
  Primer
ggggtgactgtgaatttaatc

SEQ ID NO: 105 - DNA, 21 bp, Artificial Sequence
  Primer
tgctagaccatcaaacaaatc

SEQ ID NO: 106 - DNA, 23 bp, Artificial Sequence
  Primer
atctctctctgtcagttatttcc

SEQ ID NO: 107 - DNA, 22 bp, Artificial Sequence
  Primer
aaatagaattgctggataattg

SEQ ID NO: 108 - DNA, 19 bp, Artificial Sequence
  Primer
tcttagttcagaatatcag

SEQ ID NO: 109 - DNA, 18 bp, Artificial Sequence
  Primer
tcatacatgaaggtgaag

SEQ ID NO: 110 - DNA, 18 bp, Artificial Sequence
  Primer
tgggctggcaacaggttc

SEQ ID NO: 111 - DNA, 18 bp, Artificial Sequence
  Primer
gccattatccgaggcatc

SEQ ID NO: 112 - DNA, 18 bp, Artificial Sequence
  Primer
atgggatttgctaatatg

SEQ ID NO: 113 - DNA, 18 bp, Artificial Sequence
  Primer
gacaggacttgcctcttc

SEQ ID NO: 114 - DNA, 18 bp, Artificial Sequence
  Primer
gaccacaccattctctgc

SEQ ID NO: 115 - DNA, 18 bp, Artificial Sequence
  Primer
taaaaaactgacaggtag

SEQ ID NO: 116 - DNA, 22 bp, Artificial Sequence
  Primer
gcaatgtaactttttttaatgc

SEQ ID NO: 117 - DNA, 18 bp, Artificial Sequence
  Primer
tcctatgtgataccaaag

SEQ ID NO: 118 - DNA, 18 bp, Artificial Sequence
  Primer
agctcatccggtgcattg

SEQ ID NO: 119 - DNA, 18 bp, Artificial Sequence
  Primer
ataactcttgaggtgaac

SEQ ID NO: 120 - DNA, 18 bp, Artificial Sequence
  Primer
gggagtaccacgtcagag

SEQ ID NO: 121 - DNA, 18 bp, Artificial Sequence
  Primer
tccagaagtgaaaggagc

SEQ ID NO: 122 - DNA, 23 bp, Artificial Sequence
  Primer
gatcaagaacttgtacctttgtc

SEQ ID NO: 123 - DNA, 22 bp, Artificial Sequence
  Primer
ttaaccaaagaatatcatttcc

SEQ ID NO: 124 - DNA, 18 bp, Artificial Sequence
  Primer
ttctttcctaatggtgac

SEQ ID NO: 125 - DNA, 19 bp, Artificial Sequence
  Primer
tttgtggggaagttcaggg

SEQ ID NO: 126 - DNA, 18 bp, Artificial Sequence
  Primer
taagattgatgacacccc

SEQ ID NO: 127 - DNA, 18 bp, Artificial Sequence
  Primer
gctgtttgaatcagtctg

SEQ ID NO: 128 - DNA, 19 bp, Artificial Sequence
  Primer
aaccaaccaacccaccaac

SEQ ID NO: 129 - DNA, 18 bp, Artificial Sequence
  Primer
tattaccaacctacaaac

SEQ ID NO: 130 - DNA, 18 bp, Artificial Sequence
  Primer
taagccttatcctcccag

SEQ ID NO: 131 - DNA, 18 bp, Artificial Sequence
  Primer
acttcatttcctctgatc

SEQ ID NO: 132 - DNA, 18 bp, Artificial Sequence
  Primer
tctggacaacttttcctc

SEQ ID NO: 133 - DNA, 18 bp, Artificial Sequence
  Primer
tcttccatgtcaacttag

SEQ ID NO: 134 - DNA, 19 bp, Artificial Sequence
  Primer
tgatgtcctcagttctatc

SEQ ID NO: 135 - DNA, 18 bp, Artificial Sequence
  Primer
ttgctcattagactttcc

SEQ ID NO: 136 - DNA, 18 bp, Artificial Sequence
  Primer
atgctttaggttctctgg

SEQ ID NO: 137 - DNA, 18 bp, Artificial Sequence
  Primer
tagtgccttaaacatggg

SEQ ID NO: 138 - DNA, 18 bp, Artificial Sequence
  Primer
aacagaatctcaggagcc

SEQ ID NO: 139 - DNA, 18 bp, Artificial Sequence
  Primer
tggggagaattcattgtg

SEQ ID NO: 140 - DNA, 20 bp, Artificial Sequence
  Primer
aagtgacataaaatatactc

SEQ ID NO: 141 - DNA, 19 bp, Artificial Sequence
  Primer
gacaattttaaatacactg

SEQ ID NO: 142 - DNA, 18 bp, Artificial Sequence
  Primer
gatcccctggatttgttg

SEQ ID NO: 143 - DNA, 18 bp, Artificial Sequence
  Primer
tcagttctggtcttcctg

SEQ ID NO: 144 - DNA, 18 bp, Artificial Sequence
  Primer
tatcatacatggggtaac

SEQ ID NO: 145 - DNA, 18 bp, Artificial Sequence
  Primer
aagacagccaaaacatag

SEQ ID NO: 146 - DNA, 20 bp, Artificial Sequence
  Primer
gttagaaacataaaaattgg

SEQ ID NO: 147 - DNA, 18 bp, Artificial Sequence
  Primer
aacaacaacaataacaac

SEQ ID NO: 148 - DNA, 18 bp, Artificial Sequence
  Primer
tcagacctttgctgtaac

SEQ ID NO: 149 - DNA, 18 bp, Artificial Sequence
  Primer
agcctaaaacaaccacac

SEQ ID NO: 150 - DNA, 20 bp, Artificial Sequence
  Primer
gtccagttttcttattttgc

SEQ ID NO: 151 - DNA, 19 bp, Artificial Sequence
  Primer
tcatctgttctgtctattc

SEQ ID NO: 152 - DNA, 25 bp, Artificial Sequence
  Primer
gtgccattgtgtaataatctttctg

SEQ ID NO: 153 - DNA, 18 bp, Artificial Sequence
  Primer
catcggcaagctaaaaag

SEQ ID NO: 154 - DNA, 18 bp, Artificial Sequence
  Primer
caaataatctctcaaccc

SEQ ID NO: 155 - DNA, 18 bp, Artificial Sequence
  Primer
gcagcataccaactaatg

SEQ ID NO: 156 - DNA, 18 bp, Artificial Sequence
  Primer
ggggttccttactaaatg

SEQ ID NO: 157 - DNA, 18 bp, Artificial Sequence
  Primer
gctctcaaaacattcatc

SEQ ID NO: 158 - DNA, 18 bp, Artificial Sequence
  Primer
ctttgtatcacatgcaag

SEQ ID NO: 159 - DNA, 18 bp, Artificial Sequence
  Primer
ttctgccatactagacac

SEQ ID NO: 160 - DNA, 22 bp, Artificial Sequence
  Primer
ggaagttatcacaatggattag

SEQ ID NO: 161 - DNA, 18 bp, Artificial Sequence
  Primer
gattcatctcttgggtag

SEQ ID NO: 162 - DNA, 18 bp, Artificial Sequence
  Primer
ttgtttttgtgacatatc

SEQ ID NO: 163 - DNA, 18 bp, Artificial Sequence
  Primer
tcaacatgctcgcaatcc

SEQ ID NO: 164 - DNA, 19 bp, Artificial Sequence
  Primer
ctctctttcttttaatttc

SEQ ID NO: 165 - DNA, 18 bp, Artificial Sequence
  Primer
cacaatacacacacatgc

SEQ ID NO: 166 - DNA, 18 bp, Artificial Sequence
  Primer
ctatccaactgttactcc

SEQ ID NO: 167 - DNA, 18 bp, Artificial Sequence
  Primer
ccaagaaaaagccctaag

SEQ ID NO: 168 - DNA, 20 bp, Artificial Sequence
  Primer
cactgttagtatatccaatg

SEQ ID NO: 169 - DNA, 18 bp, Artificial Sequence
  Primer
cattcacttaccttaacc

SEQ ID NO: 170 - DNA, 20 bp, Artificial Sequence
  Primer
gtttttttttcccacaactc

SEQ ID NO: 171 - DNA, 19 bp, Artificial Sequence
  Primer
aggctccaactggtaatgg

SEQ ID NO: 172 - DNA, 18 bp, Artificial Sequence
  Primer
aggaaagtacctgatgac

SEQ ID NO: 173 - DNA, 18 bp, Artificial Sequence
  Primer
gaaccacaaggttattag

SEQ ID NO: 174 - DNA, 18 bp, Artificial Sequence
  Primer
atattgtgtttggcacag

SEQ ID NO: 175 - DNA, 18 bp, Artificial Sequence
  Primer
agtccactttccttatag

SEQ ID NO: 176 - DNA, 18 bp, Artificial Sequence
  Primer
gaactgctttggtctgac

SEQ ID NO: 177 - DNA, 18 bp, Artificial Sequence
  Primer
atgactgaattcctaagc

SEQ ID NO: 178 - DNA, 18 bp, Artificial Sequence
  Primer
aaaattccgtcaaaaaag

SEQ ID NO: 179 - DNA, 18 bp, Artificial Sequence
  Primer
atggatgtatgaaatggc

SEQ ID NO: 180 - DNA, 18 bp, Artificial Sequence
  Primer
tggctggtggtttatatg

SEQ ID NO: 181 - DNA, 18 bp, Artificial Sequence
  Primer
gtacttcataaatatggc

SEQ ID NO: 182 - DNA, 19 bp, Artificial Sequence
  Primer
catgaaatgaaagagttgc

SEQ ID NO: 183 - DNA, 18 bp, Artificial Sequence
  Primer
accacaggcattgcattc

SEQ ID NO: 184 - DNA, 18 bp, Artificial Sequence
  Primer
tatcacttgttttgcttc

SEQ ID NO: 185 - DNA, 18 bp, Artificial Sequence
  Primer
gaggtacttttgttcccc

SEQ ID NO: 186 - DNA, 18 bp, Artificial Sequence
  Primer
aagtctgcttcatggatc

SEQ ID NO: 187 - DNA, 18 bp, Artificial Sequence
  Primer
gttagttagtctttcgag

SEQ ID NO: 188 - DNA, 18 bp, Artificial Sequence
  Primer
agtcttagaaaaaggctg

SEQ ID NO: 189 - DNA, 20 bp, Artificial Sequence
  Primer
gtaatttgttacttgataag

SEQ ID NO: 190 - DNA, 18 bp, Artificial Sequence
  Primer
caacagtaaggagcactg

SEQ ID NO: 191 - DNA, 18 bp, Artificial Sequence
  Primer
atggacctaaaaaatcag

SEQ ID NO: 192 - DNA, 25 bp, Artificial Sequence
  Primer
ggattgtggaaaattgctaccatag

SEQ ID NO: 193 - DNA, 25 bp, Artificial Sequence
  Primer
ggctgaatgctacatgctacttagc

SEQ ID NO: 194 - DNA, 25 bp, Artificial Sequence
  Primer
tctgaatccaactttttcttcctcc

SEQ ID NO: 195 - DNA, 22 bp, Artificial Sequence
  Primer
gctgcaaacattttctgtgcag

SEQ ID NO: 196 - DNA, 24 bp, Artificial Sequence
  Primer
ttcgcagaagacatgaagacattg

SEQ ID NO: 197 - DNA, 21 bp, Artificial Sequence
  Primer
cacagaataaaagcacactgt

SEQ ID NO: 198 - DNA, 20 bp, Artificial Sequence
  Primer
ttatattagcatcttattaa

SEQ ID NO: 199 - DNA, 22 bp, Artificial Sequence
  Primer
gacttttttttcagaaattttc

SEQ ID NO: 200 - DNA, 23 bp, Artificial Sequence
  Primer
gctgatggtcccacttacaactg

SEQ ID NO: 201 - DNA, 21 bp, Artificial Sequence
  Primer
ccatccacagtgggtctctcc

SEQ ID NO: 202 - DNA, 25 bp, Artificial Sequence
  Primer
tgaaaactaaatccatttcttcccc

SEQ ID NO: 203 - DNA, 22 bp, Artificial Sequence
  Primer
atctgagcaactgctcttggcc

SEQ ID NO: 204 - DNA, 22 bp, Artificial Sequence
  Primer
cctgcaagagactgggaactgg

SEQ ID NO: 205 - DNA, 21 bp, Artificial Sequence
  Primer
gaacattctgcctttcaggcc

SEQ ID NO: 206 - DNA, 13 bp, Homo sapien
tacgtcggtctgt

SEQ ID NO: 207 - DNA, 25 bp, Artificial Sequence
  Primer
acttcacacacctttaatgtgcact

SEQ ID NO: 208 - protein, 5 aa, Homo sapien
LSLHI

SEQ ID NO: 209 - DNA, 635 bp, Homo sapien
gtaggttggggttttaccaagtgcacatttcttatgtattgacccacaatcctaaagactatgagtctttggttttcttccttttctaaggacaggtgtaaaagtgggcctgaagggagacgaacagactgaccaacttctgtctttctagatactggcttgctaatgtcacagaagcaaagcacagcataccatgacaactctgtgtccactttcccattccaggctgtgggatgggactctgggatttctacatgagatgatatggaagtgtgtagcatagtgctcagcaatgcatctcccagaatcttggctatcttccttaaagacagtcctctacttgggagcttctgcaccattgcttagcatatccatccccagcagagtaaagagtaaatctgagggctgaaaaattcttttctggatttaggtgaacctgaagagagaaaactctttcataaaatcataaatgagaaaactcctttatacagcatctatgaggaaatgaggagtattaatttaatccttgggcaaatttaatgtcttacattacccccaggatcttagcacagtttcacactgtcctgtgtcaatgacaattctatgcagcctgagatttctttcccttgtgttag

SEQ ID NO: 210 - DNA, 1273 bp, Homo sapien
gtaagtgtgtgtttgtttttgtgtttaactagttacatacatgctgcatggcttcctgggaccagcttccctgatggcaataaattgtaagcagttggcacagaattgctattttcctttttgcagtttatggtccctcatgagctgggcttcagcaaactttgttaatgtaattgcaactggtcaattaagttggtccatttatttactgacaactagtcactctgggctcagtgtgaaatgaactgtaattgcatctgtggattttctttttgaattctcacccttccttcctttgtttctcagcctgaaatagattctttataaaatgatgtgtaaacccaaaagcccattataactttcttatttggttgttatccatttgtgctttaaaaattgtattagtggggtcagaggaaaacatggacacaggtattaaaagccatagagcttttaatgttttctgggaatgattaggatgtcaacatgactttcttcactggagacgtggtggaaagtgttactattaaatcatccacactggagagagagaacaagacaaaaagtgttgaaattgtagaggctgtaggaagctctaagcaactgtaatcattcaacaaatgaagcaattgtactgagcatttataaggtagaaggaaaatgatcttactatatactcagctgcatagattccatacatggtgtctaatttttccttcactctggatgggttgggggcatcctcttctgttctactccatctgtctgattttgccctagttgaagagaatatgatcttattactctcaggattgacatttctggcattaagcaatggtcaacttaccttacaagaaaactactcttcttgtggggggtagaaactcctactacatccttgggaatgtgccatattttgctaggtgatcccatctcagccttggtctgggatacagcctttgaaagggggaataaaggcataaatgcaatattatctctgtgtagaatcaatatagccagtgtggagatgatgaaggttttttgggaagggagaactattttctgtttagtgtgaaagcatgagccatgagtgcatccctactctctgctgagaatttgacttgacatccgccttcagccacctgcaatagtgcagaaaaatccagaggagagtgttcatagccatgtttcctctgagttttgtgaggaatgtttattgggagtattgcacaattatctcttgtcttttgttttcatttttttttaaatccag

SEQ ID NO: 211 - DNA, 2476 bp, Homo sapien
gtatgttgtaggaaaagtgggagcccacatcaaaagcacctttgctaacctaggtgaggctaggagacagataaggaaggtatcaagatttccaggaatttataacactagccaattcaacaacaacagagtttgacagtattctcaatcagtgttggtcatgctagtgcagggacaaattttgatcaatatgtaatatgtatattttccacaacttagtttacaatctgttttggagatgattatcaactagaacattgagctgagcaggccaggagactggaaagacagcagagtaaaacatgctttaggtacctggttaagaggtaaaactaacatctggttgatgccaaagaaagatccaagaatttgccctttgggtacatgagggagcaaagcctaggcagcatgctgtaggaacagccttccagtgctgaaggtcaacccaatggcccttgtatagtaggggaagctcttgcgggttctgtgagacattgttttcagtagtagtgatggcatcatgattctaattttcctgtttaatggtgtgtaattcctttagtctttttagtcccttcaaggatgtagccaaaatttctagtattctaaagtaaacttaaaaatataaaagcttgaaaattgcttagacacactgtttccttagtgtgttctcagtgtatatgttgaaggatggatcctttactgaatgatggatctcttttccctttgaaatccttcttagagaggatgactgtgactcatgagtgtgaatttcagtcagtctgtgacctcctccaaatattttaccattttattaggttggtgcaaaagtaattgcggtttttaccgttgaaggtaatggcaaaaaccgcaattacttttgcaccaatctaatagatttcaactcagttgtgtaaagaaaagcttagagaatttttaggcattgaataagccaagtagggttggtaatctgtgagcaggaaagtgtctcctgaagccttacttactaggtacttaaccatgagtttatggttttaaagtggcagttaaggctggatgctatggcttgagcctgtaatcccatctacttgggaggctgagacaggaggatcgcctgagtctaggagtttgggactaggccctgtctctaaaacgtaaaaaataaaaaattagccaggtgtgatggtgtgcacctgtagctacttgagaggctgaagtggagaattgtttgagctcaggagttagggctgcagcgagccatgatcacaccactgcactccagcctgggcaacagagtgagactctgtctttaaacaaagaaacaaataaacaaaaatgtggcagttaatttgtggtccattgtagcacctaataattcatatttatcatagtttttaacgaccatttaataatgttcaaaggaaaatctttcagctgcatttggtgtatttgggagtgttaaggagtgagtcagcctaccccaaaaggcaaaagaacattttgacttctatagtaagtgttgcagggtatgtccctttaactgccattttgaggaaccacaagccaggtagtaactaattttccattccctctctcattaatctctttaaaatctaaaaattttaaattaaaaaattaaaatattcatttattggtaagttaatataattatattgtggacttttaaagtagttatgtttgaagctatttttaggtatgaaattatctcaaaaataaagataatgtatgtacattaaagcaaaatttaaaaacatctttatattgatcatctgaagatttaagaaaatagcattatcagaaaatattcttggtatttatttacagagtctaatcagttctttaactatatctgcctataatattaggttgctggaatttaaatattaaaaatattatcttttaaaaaatgttttatgaaattgcctgtaatcttcttcaaagttgcatttgatgggttttaatttgaaattgttgacattttgaattgacgcttatctgtggatcataatatttttaattagaaaaatgctctctgcaggtgttgaggtagctggtaagctcagagcaaattctcataagcaatctgtctttatttctaataataagaatagtatttactatagattgagcttctaccatgatccagggcttgcaatagctgtgttaggtggttttgtcagctaagcttccgtatagctgtgtgctgtggctacaactttcccaaacatgacacaacagcaagggtatttgtgtccagtgtagagatgtattgggctactgagactaggcaggtgctatatctctttctagaatattctggacaattgtgtggcataagaatcaggttgttcttctttattaccataaaagaagacaaagtacaagggcagtcatttgttggttcagtgattcaggccttggttactcttgcttgactctatgttctactttacag

SEQ ID NO: 212 - DNA, 1537 bp, Homo sapien
gtaaggagtggcttttttacacctgtcccttctcctaagaatgcttgttggccttgagggacacaggtgtgacttttttcaggaaaagctacctgtcttgttgcttaaagcaatgttctcaatcttgagaatgcagcaccagattcacctggaagtattgttaaaattactgggcctactgtcagagtttctaaggcagtaggtttggggaaaaacctgacaatttgcatttttaataagttccttggtgatgctgattctgctggtctggggactacgctttgagaactaattccgagtcagttagtttaaaaatagtggcattttgtgctgttcagaggttcacatcaccactgttttgttttgttgttgttcttttcattgcattcctcatgtgttaatcttgttgaaatcctttgaaatgaaatgtctagagaagaaaaatagctatttctaagaactcaccaccagatggcactaaagccccataagcattgcttcataataaccatttctagccacagtttacaaagcagtaactaaggatcaggcttcttctaagcttctggaacacatgtatactctaatttgttcttttaatttttcacctaaaccccagtagaatgggaagaattgagtgctctgcctttgatgtatgtatatgtatatgtgggtgtagggatatatatattcacagtgtatatatgtgtatacacatatatgtctaaatatacagtcatgcaccacataacaatgtatctgtcggtgatggaccaaatatatgatggtggtcctataagattatactaccatatttttactgtagcttttgtatgtttagatatgtttggatacacaaatatgtagcattgtgttacaattgcctacagtattcagtacagcagcaggcttgtagcctaggagcaacaggctataccatatagcctaggtgtgtggtaggctgtaccatctaggtttgtgtaagtaaactgcatgatggttgcacaatgatgagactgtctaaggatgcatttcttaaaatgtatcctcactgttaagtgatgcataactgtataggtttctttctctcatctttttctcatatgttccaaaatgagttctcgtacctacagcatgctgcaatggcatattaaaggcaggtgcagaaagtttgaacccgtgctttactttccttgactaaatgtttgttccatggtcctctgggcaggaaaagcaataaaaccatccattgaaaatgaagaggtgagacatcttcagtggcagagaggaagcccaccatatcggtaggctctaaatgccaacgtttttagctctgaaatgatagacacgaattaagttactctgaaatgattctcacatcgagtggtcctgaaaactcccttactgagtttcctcaggtcccttagctctttctggaaggctgcagcctaagcaagacaggacatccttcagacaaggtcctcctaggtgcacaccccaacccagacgttaatactttgtttctgacgtgccacag

SEQ ID NO: 213 - DNA, 2303 bp, Homo sapien
gtaaaggggtgattggggttgcaacaattctcactcacatattctcactcacattcccaagggaacaacatcctgggaggttgtcagacaggtgataatattaaagctattttatttattttccagtgtctgaaagtgacattcttaaagtgaaagatttggaaagggacttattgttaatgctaccccaacctttacattttgtagatgcggaagatgtatagttgtggaaactgagtggatcagcttcctgggactgttgtaaggagttaaccacaaacttggtggcttcaaacagcaaaaaatgattctcccccagttctggggccagaagtccaaaatcaagttgtcggcagggccacactcctgtgaagtatttgctcctgcctcttctggcttctggtggtggctagcatgtcttggctgtggccatatcactccaaccaccgcctctgtggtcacgttgcttcttcctcttctatctcaaaattttctctgcctctctcttataagggtacctattatataattgtatttagggtccacctgggataatctaggacagatttttcctctcaagacccttagccacatcttttgccatatgcagtttattattttgctttataagttaacattcatattttctggggattaggccattaggccatcttttagggggttcaccgttcagccaactacactgaagcttgccaaaaggtcatacaaatagctgatgacagggtccagttgtgaacccaggtatttgattcctgggtcagtcctcttctttctagagttttatagaccaagatacatggtagccatggtccgtcagagttagacttaatggtgacaccatgtactatggaggatttcagtggtcccaacaaatgtagaaagaactttcagatgcaattagagtagtgtttgtaaatcttcaagaatacatacaatgcacaaagaacaaagattagaagtgataatgatacgttacttatttggtagaaacatggggaatcagaagcaaggctcagtagaacttgagaggggctgtataaaagttggaacatggtccaaaagtcttttgccctcccaagcagaaagtccctaggcctgcacaggaccttcttgggtgagaggaacagagtggtatttttattataatagggtgactcaggagaagtcgaagatccctaggtgttgattctggtaaatgggggaaaaaacatcaatcgtgccctgcctctatgaggctcattactgtgctatttatatttgattttacttctcatggaagacagctcatccgtagagacttcctgctactgatttctatcactgatgatcctcagatcaccgcctgcaaaacaactgggaaagtcagccttggcctggccttttgaatgatgatggatctgaaactctgtataaatgtagaaacatgatttgtaaaaggataacaatttcgtttgcaatttagaaacgcagtgatattttattagcccttcagggcagcgtggaattcaagagaaatgctcttgagtaggtagaaggcaacctgtgttttcatgtaaacctgtctttctgcattggcaagcttctgcatatggttgggtttcattttcttcatccctaaagtagacattaggatacccaatgacccacctcacagggctgctgggagcagctaatgaaatgggaacttctggtagagcacaaagcagggttcaaatacagagagctgttattcttctgtggggacatggaatggggaagcaggtggttgggcttaaagaaaattctaatagagaaactgaagttaaatgccaacattcctcagctgggctggccaagtttttatgggtgtcagctgctgtacgcctccctcactccctgtgaaaagtggccaatttttctctgttcacagggccagctaaatttttccagggaagatctaatatttaattatgctttcaggcattacaagggtgtgtggagtttacattttgagttgacagttgagatggttttatctgacatggctgtgctctctgattgctgtgtgatatggtatgagtgaaagatagtgtaatataaaaagtgggcatctacttgtctttaccttaggaaaaaatggtttaattatggctggggattctactgaagttctactttgacagaagggtctcctgaaaatttgataggcatcctagtatgtatcgtgttatccttgaggatgaaactctgtaaggtggattaattagtgtctgtgttttctgtatcaaaaccttctctcacag

SEQ ID NO: 214 - DNA, 2303 bp, Homo sapien; identical to SEQ ID NO: 213

SEQ ID NO: 215 - DNA, 1629 bp, Homo sapien
gtaagtcaagcaaataagacagcacactttctttatgtaaatgaattggtagctcctttcacttctggaaagtagaaaattaacacaatctgcctgatggaaggaaagagaagcccactggcttcttacaatcccaattccttcctgaggcttgtctttgttatgctctgtttatgaagattttttttatttttatcaaggaaactgtgcatattctctaaatgtttgattgtattttaatttgatgcattaactggatttgtattttaaattaaataacaccatttgtaagagctcataatttgatcatttaagaacgtaaatgccttcacctagttttagaccctatctgccaatttgcgttgtttaaaatttattaagtatgattaaatttttatcattttgtaatatttcacaggtggactcatatttcaacaataataagccaaatgcacagaacaattaaattggttggactgttttctccgtttagatataccaaaaattgcctagcctttccctccagtgaagaaaagctttcaatttctattcacttaaagccttgttgcacttgagatagttcaaatttggttgtgatttagaaagtaaggaaaaaaactatagaaacagaatcttagactgttggagtactaactaagacatttcttagaaaaaatttggtctcagcttggtggaacactgtggggactaggatttggctctagtctatcactaattttgctaatttatgcttgacttttctgagactcagcttccttatttgataaaggcactactccctaagattccatgtttttttatgatggaagagcagggaaataagaaatgcatcttaaaccaaaagcattatttttgagttagtcaagtggctaagtaccacctagacctggtatgaaggcagtagtgaggggaactctgtatctcagtgtaaactagaaatgtggactggttttactcatacaaatgtcccaaaacatgtacagttattgcatcactattaacaatagcaaaagaggagaaaaaatggaaatttcaataaataggaaaattgtcaaataaattgtagcagggccctataatggaattctaagtagctactaaaatgaactggatccatagagttttgaaaaaacatctatgctatactttgaagcaaaaaagcaagttccagaataatatggatagaataatgctttgaatcagaaacatagatgcttaaaaattctggaaggagccataagaaactctatacctgggtacctccggggagggagtcagatggttgggggaatttttattttcattttatggctctttgaataaaaagtttaaaaactataaataagcattaattttccatataaaaattgttacattaaaaaaaaacctctttaacacaataggatattgaatgtgattggagtcagacaaggccagagcctaagtggagaggaaactgaggccagcagccacctcaagccagttgtgcggattctgaggatgtgaaaagaagaatgggtagtgaggaagcaaatgtttgaattttgagttgagtaaagaagggagatttgcctgtatgatcaagaacttgtacctttgtctttaaag

SEQ ID NO: 216 - DNA, 5171 bp, Homo sapien
gtgagtaaagatgccacacacatatattttaaagcatatatatggaaatgatattctttggttaattctatttttgtgattaatgctgtactgactactaatttctctatgtgtgcctaggaaaagaatttctctactcctcatctaaaatgatagttagaatctgaggccacctgttaaaatgattcatcataacttttgtcatattaatatgtcaatgttagatgaatattgaaacacttatcacctgtgttcttaactgcagtcttaggatgacagaggaagtaattctgaattctgaatattagtttcaatgacagctataggagtcatggaggaaacaagctcttggctttgttctgaggtgaggttgctgaagacatctgcttattggaggtaccagtaaagggatggagattttcaagctggatatcacttgaaaattagctttgtgtgttcagttttcggtttcttattataggcaaaagagccaacactcatgttgtcttcttgttttgcaatactcacataattacttatttcttattcagtaaaacaccacaattgctcactactttaagctgcgtagtgatcatataaattgtggtatgagaagccgtgtctttgtggggaaagagatgagagagtaagttgcaaagcttggggattcacaacacatggtaataccccactgagcttcaggaaataaagtcaaaaggaagaaattactgttattactacaggaccttactacatttgagatagctcacatttagccaggtgattaccttatttgcctaatcataaatcttgtacctgtgcacttaaccgtttacatacctcctttctgtgtagtgtttaagttttattcctaatttatttgtctgcaacttgaatgtgggctaaagatttacaaaatgataaaagcaccacatcatctgagcttggatgagaatttaatgatggataatgctattgtgatggaaatttgtgctgtgttgtcttatacaaagaggagaaggaggggaaggaggagggggaagagggaagaggaagaagaaggggaggagaaaaaaatttttgttaaaatgctagaaattgagtttcctattatcatcatgtatataaacacattatggaagagagatagagtccacaatgggaaatttaagatacaatagaagattgaagttcaaaacattgtctgattcactgctctacctcctctgctaggaacagtgttgcagagggtatgtgcaaaattagtgcttggtgaggaataaatgagagatagcataaacagaaagctaagactgcacatttataggctctaatttttaactaatactgctacattttttaaaaaagctttcatatatacactgctcaaattaatatacttttaaaagccccgtgttattatttcaaaacacttaatttcaactagtataagataaaaatcaatgatgtttcaggatcaggtaagttgtgatattttaagttttataacttcttggggaagaataataaaataattattgtgtttagaaagatttagaaaagaactcaacattccatgattttttaaagaggaaaattagttggaaactgtaactatttatatggccactaggtgtcaccaaacactcattgattgtgcaaggcacacatttactctagaggaatgcataatttaactaattttcctttcataaaaaagtcttttaaaagactttcaatcacaatgtctaaagattgagaggaaattgtgagagccaactccagaggtttcttacttaagatccaagatgggatttggggattctgttaacccgctgaaactggatgaagcattttgagtgtatgactgtgtattttttttttttccctaggaagtaggtctataatggacatcaaatgtcaaaggggataggtcttagacattactagaaactactactaatttctccattttcagacaggattgagtacaaccaagttagctaaaaatcacttgtatttttaaagatttttcaggggagaatttcccagcagcaacccttacaagtgtttcgcagttgctcttttcagagaaatccatcctctgcctaatggatgctcccctactctaattttttcaaccttaatcagcgggtggttctttggaaagtggagtattcttagttcctctatggcagcagttctggaagtgtgcttcccagagcagtagctgcaagggcctcacccgggagcttgttagaaatgtgaattcttgggccctactgaatcagaaactttccaagtaaacattttagtatctcacttttcagccctctctgaaatcctatattctttcctaatttgaagccccaaatgaggaagctgttctatacaagcaagttctggccaaattgggtagcatcgtaagatgcctgtgtatcgtctgatcccatgtttgtttgtacaattatactgcagtcttgctggttaataagctttctgttacctactgttatcccaaggggtgtgtgtgtgcacgcacgcatttctaccatggctgtgcacagctggactttcctcatatttatatgatttctttttttcttttgtagtttactgtcttactcttttcatttgttcttttgtttatttctgttaacttctctcatttaccaaggtcgctttgaattctgacccgtgctttgagtttttttcttcagtttgactttgtaccatagagtgatgtttcacattccaggttacagataatgaccctgaactcaaacgttcttaattattgttttgtttatgcctgtgccacagaactaatatgttttctttacatgtgtggggcagggggagttaaagcagggggctctacaaatgtgcttttgctattttggaggtcacccatttttaccatccccacaaactgtacttctaaccataggagaaatttcctttttcttgtaatgagttgccaaaacatcctgttaggtgaaactaatactttgtgtttgtttaaaatgagcaaaatctttgatctttggctttcttgcatttataccttgaattataccacatgacattttcatggaaactttaacttttccataaggcatggaaagctaaagtgattaaggtcacgcagctatttagtggcagaactttgagtaaaacccatagattttgatccccagccctgggtgattttcataatcctgcttgtcaatttgcatctacttaatcacctttaatgaattcattattttataggtgtggagcttgtttttatttgccttagggcttactaactggaaagttcttatgttttgtctgttcatacaagagtcctactgtcagtcatttttattgctctaattcattcaaaacacattcacagccaacctcctaggtgccaggtgctttagaatataacagcaacaatagtactatttgggcatgattgcataactacaaaccctcccccacgtacttatctcatgtaacccttgccacagcactgtaatgaggcggggaagcacaggtctaagcccacggcccttgataacctctgttgcattacccacatgggcaggcttgctgcagaggaagcaggctacagagatttttttaaattttccctctttattaaggttttcagacagagctgctcctaaaccatctcatgtggagctttgttcatcttctagttaaagatcattcccagtctgcctcatgatgctttttatatgtgggtcatgactcagaaaaaaatggcctgagagtcagcatgcttcaaatacttgctttttatctctataacatgctaatgtttataattatgtgaaaatcaagaaattcataaacttatccaagtcaataagacaaacaacccaatagaaaaatgggcaaatgagagaaacaggaatttcacagaagagaaaacatgagttgttaccaaacataaaaaagttggtcaacttggccaggcacggtggctcatgcctgtaatcctagcactttttgaggctgaggcaggtggattgccagagctcaggaattcaagatcagcctgggcaacatggtgaaaccctctctctgctaaaatacaaaagatcatccggggtgtggtggtgtgcacctgtagtcccagctactcaggaggctgaggcaggggaattgcttgaacccgggaggcggaagttgcagtgaggtgaaattgtgccacaagcctgggcaacacagcgaaactctgtctcaaaaaataataataataataataattggtcaacctaaaagtaatgattgaaatgcagattaagacgacagtgaaatgccattttacacacctgattggcaaaaattaaaagtctgataatttcaagtgcttgtaattatatggagtaatgggaacttacaaatatggctggtaggaatacaaaatggcacaactactttggaaaacaatatggcactatcttgtaaagctgaacttgtacctactctttgaataagaaattttcttgataggcatgcaccctagatgaaattttgtacgtttttagtagcaaaagctaggaaataccaaaaagttcattgataagagaatagataaatttattgtggtagtttcatgcaatacaatattgaacagcagtgaaaatgtattaacaatagttatatgtagtaatgtggataaatcttgaaaatataatatggaatgaaaaaatcagacccaagaatattttatcatgtatcatattttataaagttcaaaaacaaggaaaactcaatctcttgtttaggaatacaaatgtttgtgataaaactatttaaaaaatgccaagtggttatttctgagaatgaggaggaaagggacgaacttggggagaaacacacagatttaagtgtattataagtgttttatatcttatgttggctgggaaattcacttgagttttttttatgatgattattcatattttacaaaaatgatacttgtatttttctgtgtacattacacatatacaatacattttaaaaaaagtaaactgctaacttcatattcctattggtcaaaggaggacacaggaacttcatttgtgaaaccgactagaggccttaggttctctctgtggttctttcctaatggtgactgtttcctgcag

SEQ ID NO: 217 - DNA, 16351 bp, Homo sapien
gtaagatgttcagctctaccaggaagaccagaactgagagggcataaagttctttctcaatcaggggtgtccaaccttttggcttccctgggccacattggaagaataattgccattggaagaattgggtcacacataaaatgcactaacgctaatgacagctgatgagctgaaaaaaaattgtaaaacaaatctcataatgtgttaagaaagttgatgaatttgtgtttggccacattcaaagccatcttgggctgcatacgacctgtgggctgccagttggacaagcttgctccaaaagttctttaaggtggcagcgttagtggtggtgtggtatgaaatgtttacttgctgcatattagtatcaagaaaataatttataattttgcattaaacaagtactttaggataaatgtaagcattcctctcaggatttctggaaacacttttttgaagcaataggtaatggagcaaaacaaagtagatattgatcgtttcctggtcatctaggtaatgcaaactaaaatatctccctgaactaccaactctgggtgctgatttttgtctcacttgattccaataccaatttctttttaaaccttcactatcttactatgttaatgtggccatattttgtcctttaagagtgttcaaaactgagacatgaggtgtataaggtcatgtcagattccaggaggatgaagtccacttcaataaccttgatttttaaaatcccattatttagtacaacttaacattggtttcccttggcacatttctgtttaaacatttaagaaattaatttttgaggggacaatgtagctgtagacctgagtgaacaagtgcatcagagtagtggcaagccatttcatttccccttttctaattattttatcccatggatcttctatcaaaggagttaagggcctcactgtttcaggaaactcctctcagcatccactatgtcattatgctgagtttctaaggaactcagaggatgagcttctttctcactgatcatctgtttttaattaccttcagctgaataaaccctagtaaaagtagtggttatctcttcttccttagattcttaattatgtttgttccaagcacagcactcacacaccattttaggtgcctaatgggagtggctaatatgccagtaagcaaaggaagcagaactacacggactgtaggaacccagatcccagatccaggacagctggagctgcatttaacttgttggtcactacaggccaaaaatcctaatgataattaggatttttttgttgttattttaaacttttaattttaaaataatttcagacttacaagaagctgcgtaaataaaagagttcctatgtgccctttctatgtctgtaagcttttgggcttaagaaaccatgtttttgtgtacttttctgggtagcataatgttgactacatcaaatgcctggaggaaagtaaaccctcagggttgcccagctcaccctgctgtgtgaagctggaatgtccttgtctcactgggtagtatgtggtctgctggacggagtgctggccactggctgacaagaagaagggttgagctggctgcaggtgcagagaaccagagggtaatctgaaaagctgtgggtggtgtgagcctttgcaaaatgacctgtagaataatacccagtcagcaatggaaaaattgagtctgcaactagctgcttgctctccctccaacgctttccatttcccttcatttactttcttactcttgtatctgctttacaaaattagaaaaaaaatacaatgcatatgttttgagaatggagttttaggttaaactggtaatgtagattcattaggtatattcctgacatatttatccttggtgaccttaaagttctttttttttttttttaagtttttttttcttttattattatactttaagttttagggtacatgagcacattgtgcatgttagttacatatgtatacatgtgccatgctggtgcgctgtacccactaacttgtcatctagcactaggtatatctcccagtgctattcctccccactccccccaccccacaacagtccccagagtgtgatgttccccttcctgtgtccatgtgatctcattgttcaattcccacctttgagtgagaatatgcggtgtttggttttttgttcttgcgatagtttactgagaatgatgatttccaatttcatccatgtccctacaaaggacatgaactcatcattttttatggctgcatagtattccacggtatatatgtgccacgttttcttgatgcagtctatcattgttggacatttgggttggttccaagtctttgctattgtgactaatgccgcaataaacatacgtgtgcatgtgtctttatagcagcatgatttatagtcctttgggtttatatacccagtaatgggatggctgggtcaaatggtatttctagttctagatccctgaggaatcaccacactgatttccacaatggttgaactagtttacagtcccaccaacagtgtaaaagtgttcctatttctccacatcctctccagcacctgttgtttcctgactttttaatgattgccattctaactggtgtgagatggtatctcattgtggttttgatttgcatttctctgatggccagtgatgatgagcatttttttatgtgttttttggctccataaatgtcttcttttgagaagtgtctgttcatgtccttcacccactttttgatggggttgtttttttcttgtaaatttgtttgagttcattgtagattctggatattagccctttgtcagatgagtaggttgtgaaaattttctcccattttgtaggttgcctgttcactctgatggtagtttcttttgctgtgcagaagctctttagtttaattagatcccatttgtcaattttggcttttgttgccattgcttttgatgttttagacatgaagtccttgcccatgcctatgtcctgaatggtaatgcctaggttttcttctagggtttttatggttttaggtctatcatttaagtctttaatccatcttgaattgatttttgaataaggtgtaaggaagtgatccagtttcagctttctacatatggctagccaattttcccagcaccatttattaaatagggaatcctttccccattgcttgtttttctcaggtttgtcaaagatcagatagttgtagatatgcagcgttatttctgagggctctgttctgttccattgatctacatctctgttttggtaccagtaccatgctgttttggttactgtagccttgtagtatagtttgaagtcaggtagggtgatgcctccagctttgttcttttggcttaggattgacttggcgatgcgggctctttttttggttccatatgaactttaaagtagttttttttccaattctgtgaagaaagtccttggtagcttgatggggatggcactgaatctgtaaattaccttgggcagtatggccattttcacgatattgattcttcctacccatgagcatggaatgttcttccatttgtttgtatcctctttgatttccttgagcagtggtttgtagttctccttgaagaggtccttcacatcccttgtaagttggattcctaggtattttattctctttgaagcaattgtgaatgggagttcactcatgatttggcactctgtttgtctgttgttggtgtataagaatgcttgtgatttttgtacattgatattgtatcctgagactttgctgaagttgcttatcaggttatggagattttgggctgagacaatggggctttccagatatacaatcatgtcttctgtaaacagggacaatttgacttcgtcttttcctaattgaataccctttatttccttctcctgcctaattgccctggccagaacttccaacactatgttgaataggagtggtgagagagggcatccctgtcttgtgccagttttcaaagggaatgcttccagtttttgcccattcagtatgatattggctgtgggtttgtcatagatagctcttattattttgaaatacatcccatcaatacctaatttattgagagtttttagcatgaagggttgttgaattttgtcaaaggccttttctgcatctattgagataattctgtggtttttgtctttggctctgtttatatgctggattacatttattgatttgcgtatattgaaccagccttgcatcccagggatgaagcccacttgatcatggtggataagctttttgatgtgctgctggattcgttttgccagtattttattgaggatttttgcatcaatgttcatcaaggatattggtgtaaagttctcttttttggttgtgtctctgcccggcttaggtatcagaatgatgctggcctcataaattgagttagggaggattccctctttttctatggattggaatagtttcagaaggaatggtaccagttcctccttgtacctctggtagaattcggctgtgaatctgcctggtcctggactctttttggttggtaagctattgattattgccacaatttcagatcctgttattggtctattcagagattcaacttcttcctggtttagtctctggagagtgtatgtgttcaggaatttatccatttcttctaaattttctagtttatttgcgtagaggtgtttgtggtattctctgatggtagtttgtatttctgtgggatcggtggtgatatcccctttatcattttttattgcgtctatttgattcttctctcttttttttctttattagtcttgctagcagtctatctattttgttgatcctttcaaaaaaccagctcctggattcattaattttttgaagggttttttgtgtctccatttccttcagttctgctctgattttagttatttcttgccttctgctagcttttgaatgtgtttgctcttgcttttctagttcttttaattgtgatgttagggtgtcaattttggatctttcctgctttctcctgtgggcatttagtgctataaatttccctctacacactgctttgaatgtgtcccagagattctggtatgttgtgtctttgttctcgttggtttcaaagaacatctttatttctgccttcatttcattatgtacccagtagtcattcaggagcaggttgttcagtttccatgtagttgagtggttttgagtgagattcttaatcctgagttctagtttgattgcactgtggtctgagagatagtttgttataatttctgttcttttacatttgctgaggagagctttacttccaagtatgtggtcaattttggaataggtgtggtgtggtgctgaaaaaaatgtatattctgttgatttgaggtggagagttctgtagatgtctattaggttcgcttggtgcagagctgaattcaattcctgggtatccttgttgactttctgtctcgttgatctgtctaatgttgacagtggggtgttaaagtctcccattattaatgtgtgggagtctaagtctctttgtaggtcactcaggacttgctttatgaatctgggtgctcctgtattgggtgcatatatatttaggatagttaactcttcttgttgaattgatccgtttaccattatgtaatggtcttctttgtctcttttgatctttgttggtttaaagtctgttttatcagagactaggattgcaacccctgcctttttttgttttccatttgcttggtagatcttcctccatccttttattttgagcctatgtgtgtctctgcacatgagatgggtttcctgaatacagcacactgatgggtcttgactctttatccaatttgccagtctgtgtcttttaattggagcatttagtccatttacatttaaagttaatattgttatgtgtgaatttgatcctgtcattatgatgttagctggtgattttgctcgttagttgatgcagtttcttcctagtctcaatggtctttacattttggcatgattttgcagtggctggtaccggttgttccattccatgtttagtgcttccttcaggagctcttttagggcaggcctggtggtgacaaaatctctcagcatttgcttgtctgtaaaggattttatttctccttcacttatgaggcttcgtttggctggatatgaaattctgggttgaaaattcttttctttaagaatgttgaatattggcccccacactcttctggcttgtagggtttctgccaagagatccgctgttagtctgatgggcttccctttgagggtaacccgacctttctctctggctgcccttaacatttttcccttcatttcaactttggtgaatctgacaattatgtgtcttggagttgctcttctcgatgaatatctttgtggcgttctctctatttcctgaatctgaacgttggcctgccttgctagattggggaagttctcctggataatatcctgcagagtgttttccaacttggttccattctccccatcactttcaggtacaccaatcagacgtagatttggtcttttcacatagtcccatatttcttggaggctttgctcatttctttttattcttttttctctaaacttcccttctcgcttcatttcattcacttcatcttccatcgctgataccctttcttccagttgatcgcatggctcctgaggcttctgcattcttcacgtagttctcgagccttggttttcagctccatcagctcctttaagcacttctctgtattggttattctagttatacattcttctaaatttttttcaaagttttcaacttctttgcctttggtttgaatgtcctcccgtagcttggagtaatttgatcgtctgaagccttcttctctcagctcgtcaaagtcattctccgtccagctttgttccgttgctggtgaggaactgcgttcctttggaggaggagaggtgctctgctttttagagtttccagtttttctgttctgttttttccccatctttgtggttttatctacttttggtctttgatgatggtgatgtacagatgggtttttggtgtggatgtcctttctgtttgttagttttccttttaacagacaggaccctcagctgcaggtctgttggagtaccctgccgtgtgaggtgtcagtgtgcccctgttggggggtgcctcccagttaggctgctcgggggtcaggggtcagggacccacttgaggaggcagtctgcccgttctcagatctccagctgcgtgctgggagaaccactgctctcttcaaagctgtcagacagggacatttaagtctgcagaggttactgttgtctgtttgtctgtgccctgcccccagaggtggagcctacagaggcaggcaggcctccttgagctgtggtgggctccacccagttcgagcttccgggctgctttgtttacctaagcgagcctgggcaatggtgggcgcccctcctccagcctggctaccgacttgcagtttgatctcagactgctgtgctagcaatcagcgagactccgtgggcataggaccctccgagccaggtgcgggatataatcttgtggtgtgccgttttttaagcccgtcggaaaagcgcagtattcgggtgggagtgacccgattttccaggtgccgtccatcacccctttgtttgactaggaaagggaactccctgaccccttgagcttcccgagtgaggcaatgcctcaccctgcttcggcttgtgcacggtgggcgcacccactgacctgcgcccactgtctggcactccctagtgagatgaacccggtacctcagatggaaatgcagaaatcgcctgtcttctgtgtcgctcacactgggagctgtagaccggagctgttcctattcggccatcttggctcctccggtgaccttaaagttctaattctattttcattagcctttagacactagcacaatttctaaaacatggtagttgctcaaaaaaaaatgtatttaatataacgaatgaaggtataaacctataaccaaattatgtgggggaagaaaaaggaaagtaaagaacaagttccaaagttctaaaatcattttgatgactgaaaaataagtttagtcagtggaagcatttagattctgttgggctatcagcattaaatcttgcaaagtctagtccagtttttcagttgtgccattggctttctgttattcagtctttttttgcattctattgtctgtgatgtgccattacttaaagacataggtggtaactgggtatacgtgaaaaaaacctgtgttccctaggcgagtctcaaactcttggtctcaagtgatctccccactttggcttcccaaaggattggaattacaggtgtgagccaccattcttggctgagttagtgttaattgtgtcctgtatggatgatatacagagtactttgaagaacatattttagaaatatacctttattccaaagggataataaggaggcttccacaataacaaggtgtgtgtgtatgtatgtgcatgtatttgtgtgtatgtgtataatatgtggaacagaaagagatgtgatagaaaattgtgattaattaggatttttctttgttgttattttaaacttttaattttaaaataatttcagacttacgagaagctgcataaataagagaattcctatgtgctcttcatctagctccctccaatgataacataacgataaccatagtcatggagctgtaccaaggccaggacattgacattggtatgatagtattaattaaacaaatgtatttatttagacttgaagaagatggcctaatcttcatgtttgggcaaccaactctgaaagcctaaactctctctcctcacatacctgagtctgaccttgaaatctctgacctggaaggtaggtaggcaggggaaatgattcttaagtctgtgcaatggtttgtggttttatattattctcaaattgccctgatgcagagctgggaccaaactctcagccacaacataaggaattcagctgggcttgcagtcagtcaccttgctagttcacttacattcaacctggcatgatgcaagtaaatttcaaatatgatattcttttttagttttgatgttcacaggtggtctgcttctctccctgcccagcctgcagctgaggtgagtccagggcagtttcactgagaagcaaaacctgagctttgtaccttcttgccagtgaggacccatgggtgataagtgcttgagtctccaggggcctgtggcattccctggtacatttcttaaataggcgtttgccatgctttccaaaagcaagttggtggtggttcttttgtgtatcacctgtagcttcttagagcgcttcaccctcaggctgtcttccctcagcagctccagctccggagtctaggacttctttgacaggagctccttgttatctgtcaagaggctgcatctttccagaagacataacctgtgtgaatctgttctcattctctctggttgggaagtctttgctctcaaacagcaagggactgttcacttccagagctcagagtctcccatgtttatgtaggtgttggaaatttcctataaccagacccctgtttaggatagtccgtggaaaaactctgaagaagagccctttggagacatgcatttaatttgaggagaatttgttctggtttgtgaatcaattttgtaaggaattctgtttaaaggcccaacctcagttctcaagctttccccactcaataggtaatgcatatttaagagctgcctgaagggttaactcactttttttctagtgactatagatgtacacctcaaggagctggcttacttgggcagttaatttctctctggaatttctcttgcttgaatgtccataatacattgacttccgcaaataattgatttccacacaaaattccctgcttgccttaaaccttccccgcaagctgccagctgcctgcataaatatgtggtttttcctcatgaatcatttgtagcatacacaattggagttgagagtaagaagggccaggaaggaatgcgatctgactgttaaagtgcttcttttgtaagaaaagtgagtcttatcagctaacagaggaccaaagttgcagtctcctgcccacctataaatgcgcttggaaagtgtttatatgtgatcacccagatgtgccataaatgctaatggcactagggcagatgattcaacaaacccatttattcagggaaacacgtgctttctgagcatctgtgctggcaaaggtgatgaataaggatgcagtccttggaatatattttttaaatttaagaatcttttctttcctcataaaatcttggtaatgaaatagcatttccttggtaatgaaatagagctgtgtggacaagtgagatgatcacttttagcttccagtccacatttctcattgtatacttgcagacaaggtgggtgtttaagaagcccccttctctggccctcggttcttatgtaccgctgacctttcccaaggctcgtggatatagagagacacacctagagaaggtggagtttcacttccctggggaaagtttgtgttgtgtaatcgtgactgagaacaacagctgtggtatttctttgtatatttacaagggctgaagagatacactgggcacttatatttgccaactaatagacagtgattcttcttgaggccaagaagcaggagtgcatactctgttttctagcacaagagaaaaattgagatagctgatttaatgctaataaatgtttttttaatgggaatgcctgatgcctcatctaatggttttaaatgatatcaatagtttcctagtaaagactattaaagcattcttattgtaattaaccatgttttcatatttaggcttgtctttccatcaaaatttggagatgggatactggaaaatgagtgtgggttattttgctaatatgtgactctagtgtagactatagaccaaggtttctcaactacgtcaatattgacattttgggctgggtaattccttgttgtcgagcctatcctctgtataacagcatccctggtatctatgcactgaataccagtagccctcttctctcttcttcatcctccaagttgttacaataaaaaatgtatccagaggctgttaaatgttccgtagggaacaaagtcatccctgattgacaaccaccactctagactaggccaccctaagaccattgaagtcttagaaatggacctgattctttgaagtcgtgttggcatctttgatagtgattcatcttaatattcagtgatatccaaaagtcacttttggtgagcaccctgtggtaggtgagacatgtgattagaattctaagtgcttttaggaaattggagaattgctcagactccaaaataatgaagtgcaggagtgactcggccaattagtccacaaaggaacaaaacatcctgtaaccgaatgaaaaatgaggaagaaatggctgtctatttacaaatacccagggtaagaacctttgggacccacagtgaattgaagctaaagctaaataaacagcccggggcaattgtgtcagtatgtgctggcctccctgaccaggtcctggctgctgcctgctggtcaggcttctagtttcagggggaactctggccctgactcacacttcccccaaaactgaaggcctccaggggttgaggcattggttctttctgtcacacaaggcagggaattgggcgttattgggttaagagcaatctttttcagttttctcccccatactgtctttgcttggctgaatgtgtgtgtgtgtgtgtgtgtgtgtgtgtgtgtgtgttccttgtggaacatgtggtacattcaggaaggaatagggtctttaagttcttctcctgttgggggtgccgctatattatcactgccagtggatcagccatgttcagagagttacccttaataatacctaaagttggctgggcacggtgcctcatgcctataatcccagcactttgggaggccaaggtgggcggatcacctgaggtcgggagtttgagaccagcttgaccaacatggagaaaccccatttctactaaaaatacaaaattagccaggcatggtggtgcatgcctgtaatcccagctactcaggaggctaaggcaggagaatcgcttgaatccgggaggtggaggttgcggtgagccgagatcgtgccattgcactccagcctgggcaacaagagtgaaactccatctcaaaaaagaaaacaaacaaacaaacaaaaaaacaaaaccccaaaacctgaagttttcctttcatttggaaattaaagtcaataaaaatattagtctagaatgactgaagccataggatgaaatgagtttacttagtctaaggataaagaaatggagaatagctacaagtaaaagggtatggagtcctcactctcaatccatccttactgagaatgtagtaaaactccctgagctgtgtgaaaggctagtgcaagaccctgtccttggatgggtcagaacttcctcattaaattctaacccttgtctgcattcagtccatagccctactgtctgcactgatagtagatgacaactgcagataatctcagtcttttcatctgtcattgctcaaaattcctgtgtcagaagccagtcctcatcaacatgtctcagccttaataacacttgatctccataatgctgaaattccagaaccctcatcctccttaaatgtactcttcaaaatgtcatgtcttcttcctactcttgagaaatgtcccacacccctacccatgtatctctctgtgcacgctaagctgtgcatgcttttttccccatgtggtgagaatgagaaggattttacattctctggatttggggtgtgaaaagagcaactccttagttaaatctggcctgcaagatgctctccagatttggagaaatcaaacactacctccggtgccttaaggattaattcactttttttttttttctggtgactatagatgcacacctctaggagctggcttacttggacagttaatttctctctggaatttcccttgcgtgaatgtccatgatacattgacttctgcaaaggatgttgttcaatatgttgtctccactactggggaggccacagcaaatgcctgttctgtccgtgaccgggtgacaagacttttctttgtactctcttgtttatgtttcttcacttgcaccctattattgggcatcctgcctttgggtcctaccttcatttctctactctagtctctaacttttccagacttggacttggatggagctggctctgacaactatttgctctgtgactttgggaaagttactcaacatctctgagcttcagtttctttgctatgaaactgtaataataatacaaaactcataaaactattgtaaaaataaatgagactctaaaatccagtacctggactgtagtgccttctctgtagattttcactatgagtgttattattctactcatcattacttaaaaaaaaaatacttctaagggaaaaaaaaaatccaccataaatataacttgcactgagctgggcagcatttttgctgggtacctggcaatggattctccccctgatttctaggtctctatcctccttcagttttcagcagtggcttccatttgcagtgggttcccaaagcaactcatggcaaaaaacttcaccaggaagtggtttttctagtgtctctctcctctttgaatgtctcctaggaacgttagtaacccttagcgttactctctcaatcacctgtagagaagtagttctcaaaatggggtcaaaatatcacctgcagcagacctatcagagcaccttgtgatggccaagagtaaagaatactgtccctcatcccttcctcatcacccccctaataaatcaaaatatctaaaaacctttggctaatatacccccaggtaaagcctgccagataaaatacagcatatttggtcaaatttaaatttcagatagtgtttgcgacacacttatactaaaatattttattttttatctgaaatttaaatttcactgggcattctgtgtttttatttgctaaatatgacaactctaccaggttgcacacgaaagtttaagaaactgttccaaggggatgctcacagcgtagtccccactgagccctgcatagagggctgcactgaagtattgttttttaaagaaaggcatttaatgtcttgttgagtggcacagtgtcatggtccttatgactccccattctctttactgtgaccctaatgccagtgttcaggatgagctctccaagccccatagtttgtgttcccgggattgagtgacttccaaggaggatactccagacattccttgtctcgcaccagccaggccatggaaaacccccaggtgatcaggattgaatcatgtgggagccttggagactcccttcatctatgtagcacagggcttacacctgagcagcctattccttaacttctgttctccaggtagacatgggcatcttgactttggacaaccactcagactgagagaggtcagatgcacgtgggcacataggaagtagtctcatagcttgatcgattggccctttgttcctcccactaatagcatcactgcagtgcagcagcctgatagggccccgatttttctcagagttctttctccctgatgataaagagaaggcttgggatccttaagatgtgagcgtgtgacactgagagaactcagatgtgattcaggaaagcagaaatagaaactttcatagaatagccaacatgttatttactttgttgcctgtagttgaggttgcaaattcaaatactgcagagactaggcagggatcataaatgagtgaaccaggaagggtgaggactgtggaaaaatcatccacagtccaagaaggaaggcccttcttatcttcacctgacctgtggctacttcacaggaatttgggctcagaaaagctgaaagtctgaattttttattaaatctcttggtttgaacatgttggaaccaaaaaaatatgaacattgggtaggccaaaaaaaaatacatctgtgtacagtcttaaacctttaggaagtcagttttttaatttgtcttaagtctatgttacatagggatgaacatattgtaggtacttcattgaatttataaagataacctcattgtgttcagtttgatcagaattagatccccaaactcatatggtccaagagagagaagtcagattttatatgaacacataaaatacatgaaattatttagatagtaagacctataaagaaatatcaaggcgacttaatagtctattcaggacaagagcactttgaagagatttgagttgtagttgtactcagtcaccctgtcttggtactaagtcatgactggtccaatcatgtggccttcagccaatagacaaatcgttgagttgttcaaagagccagagagaacacattgtgtaggcttgacttcaaggtacacctttgccttggagagctaccatatactgttgcattcaccaaagaggagaattttagatgtcatgagtaaactcactgtggaagcttagaaagaggcttgagcatgagataccagttattaagatgggcaaattgcagtctagttgagctggctggaggaagaagaatgacaatatttttattagttcattctcgcattgctataaagaaatacctgaggctgggtaacttataaagaaaagaggtttaattggctcatggttctgcaggctatacaggaaacatggaagcatcaatttctggggaggtctcaggtctcagggagcctttactcacggcagaaggcaaagcaggagcaggcaacttacatggcaggaacagaggcaagacaaggtgggagccacaaggtgctgtacacttttaaactaccagatctcatgagaactcacttactatggtgaggacagtactaaggggggacagtgctaaaccattcacgagaaactgaccccgtgatccagtcacctcccaccaggccctacctccaatactggggattacaattcaacctgagatatgggcagggggacaaatccaaaccatatcactatttttgttcccaaaaggaaaaaaaaagaataaatgagtctataatagagttttaatgtactttggcagaaagtgaatataaaattgggatgagtacaaacaagagtctttctttccagcaaccttatcatacatggggtaactattttttaactcttcttaatttctgtag

SEQ ID NO: 218 - DNA, 15314 bp, Homo sapien
gtgagtaaaggcacaaggattgcgagcatgttgagaattatattctgtgagaaggggctctgggaaacagatggtatcatcaagcaggttgcttaacagagtttagggagggttaaggtagtagtgagtgcaccagtaaagttgctcttcggcaagctttacatgtggtcagaggtggcttgtattggtcattctttcatgcccgtgtactatcaggctgtttctggtccgcctgctactgctcattccagggtgattttttacctcctccaggctacagatcaattcttcctttaggttttctttgtgtcagaatatgcctttaactcgctctttcagaagatatcataggatggatttgatatcatctgatcaactaactcacaataaaatttcaattgttgtcttaacataatttcctgggaaaacagacttgaaagcaaagctatgtgctagcactttactgggacaaataatcccaaagatgtgtgagtgaagggaaaagaaaagtgaggaggaaagggagggaaagcaaatacaaggtagccacagcttctcaaagaagcatgcttggtttgcttggttatgtgggatatcttgagagaagccttattagaaccaccatatcttgggacaatatggaagaggtagtgaggagaaggcaagcatctcacctggcccattccatgccattaactcctctgactttgttatttttgtggctccctgggtagctagtggaaggaccagagaatcagtaggtgctcggagatgaggctcctcttgctgggaagtggatcatgttgttgaggccacaggacaggcagaatggaatggatctagggtagtgtattaaccagatttggcttacttaaataaaaaaacctgaaatgttatgggtagactatatacttagaactgttgtaagcaataggtataaatgctgaaaagacatgtaccctgtctccaaaggaactaatttagaactttagtaaggcattttactttgccccaggaagaagattgaaagagaatgcccagaaaaaaatcatatcttattccaaagaaatatatctctggtggtaaggaaatatttagatatccttgttaggtagcatgaaaattttagaatctcagttgggagaaaattctaggtctaattacctgaacaaaggttaagttactgtaaaaatatttctgaaaaaatatggtcaatccttttcttgaatacttccagtgacaaaaaaatcaaaacctttcaaggcaacctgttcccattgtttaaaatcctattagaattctttttgttctattaagctaaaatctatcttcctctaatttctataggttcaggctcttccccttgagccattcaggacaagtatcttactccttttaattctatagcccttcattcacatctttttgaatcttttctgttttaggtcatacagtttataaccatggttcatagttattgctgttttaagtatttctttttagtctatggtacaaaactgcaaacacagaaaaacaaagagaattatgtggtggaaatgaagtgatcactcaaagtttccaagtgcaaatatttccaagtgcaaatataaccacaattaatattttggtgttttatctcaaagtcattttttttcttacacatacatacacatgcacacatgtatttcaaaatgtagggtacatactttgatttttttgatcctgtataaacaatgtaagaatttccacaactcattatatttcaaaggcccaacttcaataatttcaacattatataatatatgggaatatatagatatacatgtatatataataatttagccatgtttctattgttaagcacctaagttatttcagatttttgtccttgtaactcactctgcaatgaacattcaataattaaatcattattcatatcttcatttccttaggatgggcttttaaaaatgaaattaatgggcctaaggataagaacatattttaagatttttaatacatgctgccaaattgcttttcagaaaaattttacaaatttgtacaccccatttcccatagttatgagattccctctttcttattggcattgcctctaaacacaactttgttttgtttaattcctctgaaagccagggagaaagaactattcccaggtgggatctgactattctgaacagagtgagatgagtatagcagttaacagaatgggctctaagtcagatgttgggggtccaaatccaccttggccacctgttggctgtttgaccttaagcaagtcaacctctgggcaccttcatttcttcatctgcaacatatgtgggataagagtagaaccttcttcagagggatattaaacataactcataggaagtgcttggtatgtagtaggtgttcaataaatgtttatcagccagggtgcccacagaaaatatatgtcatatgcattagacagtttgaagggatttaaataaagaaatgatttacaaagatccaaatgggatgaggtataccctagggctcataaagccagttatccttactacctcttgccataaaggattgagaaaagaatgttataggaacccagagataaagagggaggtttagagatggttgtcagaccatcccagaggcatggcctttgtatggctgggatttaaccagtacatggaaggaactataggaataaacactcttacctcattcttttgtttccctcctgtcttctgcttgtgctaccatttggccacatccgactggaagtcagaataaaggagaagtcagaataaaggagaactccttgatgcagtacacataggtcagccacatcctggggcactgggtagggtggagaagcacagagtttggaaatggaggtgtcaacagaagctctccagcatagtcagcctccagctatcagaacagatatgattcttctgttaatacagtcaaatattaaactaacttctttttccagccaggtcatgccattagtcctgccaactacactgtcagctctaattcctgctctttatatttgtgctgccacaaatacatatctctacattgtacagttgtattgggctgattttgctttcccttgggttttgagggaatctaagggcaatactttttacctatcccttcatttaattatttcagttcatctttctacactgcagatattctcttgaagtctggtcttttcctcacagattactgccatttgcccatttgtttactattctgtaaaatcctcatccaattaattgagagatattaaatacagttgagccctgttcttgttactacaaaacttatttccagttgatatcttttattgctaagattgatctcttttttcagtaagttacatttagatttctaaggtcactcattactataatatatagcttgctgactgaaaaatttgtacctttgaaggtaagtgacactgtgccactttccagttttactttatatatttcaggtctcatttaaaaatttctccattcccaattctattttctacccatcactggatagtggtgtattctttttctctttgtttcatccatgtgttccaggcctaaaggtaggttattggaacccatctgcccaatgctatgtgtattgtgcctgaaggaagactttcaacccatctgtccaattttaccacatcctaaagaaatcccaacatgaaacagataataagactagttagtaaacaaacttaatagatttggacattggcacagagtgctgccagagtatgcctgctatctgatgccatttgatccaagtcccaacattgtggaattatattttttggtgatcctgcttttttttttttttcttgagatggagttttgctcttattgcccaggctggagtgcagtggcgccatctcagctcattgcaacctctgccttccagttttaagtaattctcctgcctcagcctcccaagtagctgggattatagctgctcgccaccatgcctggctaatttttgtagttttattagagacagggtttcaccatgttggccaggctggtcttgaactcctgaactcgtgatccgcctgccttggcctcccaaagtgctgggattataggcatgagccaccatgcccagccggtggtcctgcttgtaaggcaactggtgctggcaagggtatcaatggacttttaatagagttgaatcatagaatgctagaattagaaaggaagctcaagatcaaagctttgcttagagggccatagctgcatcacaaatgccaaactcaatttcattcttcgttcagtgcttgtctcattagtctgataaaacagttaagataagcttaaagaatttcacatgtgtgtccattctttcatttaacatgggtttactcagcgtctgccatgtgccaagtaccatgggggatactgggcatatagaaacagaaatcatgaacctgaactttcaagaggtgtctcaataaaatagtgtgacaaatgagtaaaaacatgattggaaggatcatgtcttccaacctagagggagcttaaagataaaggaagagatgtgaacaggtacatattttagatgagcattatacttttaggaaactgtaggggatgggtatgtctggattgtaggctttgtatgaaatggtggtagtagatgaagcaccttgacctgatatactatgccaaggagtttggattttcctatgaaggcagtgggtagtcactggatagttttaagcaaggaagtgacatgattatatttattcttagaaaaattagtgctggaagtgtagagaatgcacttaaaagggacacagtgaaaagtgagaagcctactgagatgtttcaggaaagaaatcgtcagggagtgaagtaaggtacaggagttaaagagtaaagaattaattagagaagcattgaggaggtaaaaatcactagaacctagtgagaattttgatctgaggcaaggaagagttatttgggatatatccagggtttcgggttgggagatctgggccaatggggatgccaggaatgagtgccctttccatccctcattagttgaatggccctcggcaagttatatgaactagctgtgtctcagtactcttatctccaaagtagaaataataatagtacgtatttcatagagtttttaaaaaagtaaattagttaatgtttattaaaatgcttgaacaatgttgagtatacagtaaactttatacattggtttgttaagtacccagactcataaatttcaaagttagatttcagtgaaagatctaagtgataatatctagagaacaagcagatttatgaataaagaaaccagtgagagaggtctgcgctaaatgtacatatttgaggatcatcagctcaaatgtgaaattaaaagtgtgaataaaactggccaaacagattttgtggatagaaattttgaggagcgcaagtctttgaagggtggtagaggaagctgagttcaggaaaagaatgaaaaagaaacagttatataggtggaaagaaaatgaaatgacaggaaaataatatcttgaaacaaaagaggcaatttacaagaggaagtagtcagtaatatgagatacagtggaaatggcaaagaaaatagaaaagaaaattggccatcttattccagctttttgttttttataatggctgataaatacagttttgacacaaattattacttttcatgtaaaacaaataaaaatgcaacacttactgcttcaggagcaataacttttcataaactcaataccatttatctattctttaacaacttcattttttggctttagtatatagcctgaggtgtcattgaacatattgaatttcatggttgaaaatgaatagttgtccatattgagtcatggcatcaaacctttataggattgctttcatgaataactggttttcttttggatggtgaaatgaaatgaatagatcgatactaaggagaaaataattcctctgatatggtgattttccaaagtttcctttgtagatttgtcccttcctcctttacctcctcccacctccctctttcattgaatgccaactcagtttcaggcattctttctgtcatttgaatattccacagtgaacaagacattcagtagtgaacaattcctgcttgcattattttattgggggaaacagacaattaaatagtaaaataaatgaggaagataagtttagatggtaatacattctctgaaagaaattaaataggaaaagtaattaatgagaggtccaggttggatgctggcaaccatatgttagatagtaaagacctttctaacaagtcaatgtgacatcagactatagagacaagaaaaagctagacctgcgaagacttgaacaatagcatttcaggctgagggagagctagtgcaaaggtcccgagggagaaatgtgctttgtgtggtagtcgtgaggcagaaaggcagcccctgtagctaaagaatagtgaatagtagtgagtttatagtgaacgtcatgagtttcatgggctagattttgagatatacagtgttgctttctaaatgagtccaaattatgctgagaattgctacttattgatagtctcttgagtatggctttaacaagacagaaattagcttcataaatatggaaattccagagacatgaattgtgattttagtttagtaactcattgtacaatttctggaaaattccccaatagaagattcataactgtgatgttgcaaatctctgtgattgtttgaaacgtctgtctcgttttattggtaaggaaactgaaatccagagagataattgatttgtttcaaggtctcagagctgacaaaaaacaggaccaggacaaatggctgttttagctatcttaaatatcatccttcaagcctttgcccaaggactacctttattatttaatggcaaaaaccacaattacttttgcaccaacctatatgaagcctctcctgtctccatgcagtagatttcagttacctttccttctgatccgtgctactcattcatgttgccattactgcattcggtaatgatatgaatagcatgttttaattagtgtcctatctccattatatgatagacttaatcagaacattttataaccctagtacctactgcattatagaattctgataatatgctgttatgactgacaatgtatatataagtgaatgggcatgtttaggtttgtctttcagctcactgcctttttcttggaaaacattcaatctccttacacttaaatgtctacatctgtgaggaagaaattatggtgttaatgtttaaatgactacaatatatttatcccctatgtgaaaggaaaaaaggagttacaaaaacaaatttaaaaagttgatgtgttgtaggattcaaagtattttcatttttacaatctgttagaacaggaaactgacttaaaaatgtcccccactcaaagcttgttcaataaacatttgtttgttatttgatttaaatgtttttttaaacatataaaagctttgcccatgaatagctgtctgagcccatccctttcatcaatgtctcctcttatatgagacattgttctcatgctttaacaaatatgttccttgttgttttagatatcaatgctgttaacttgttattgacccaaacagtcaactcaataaagactgttttaatattgtacttttaaaactgcaaatgaaaaatcttcttttaatatttttagaaggacttcagataggggttaccaataatacatttgtattaattattaaaataatataaaataaatatagtatttaaatattataaaataataaatttatataattttataaaatttatttgtattgtaaaaatacatttttgaaacacatatcctaatggatatttaaagtttaatagaaacctccttgatccctagtcaacaagatatgccccaaccattggtatccctggggacacagcagttcattgtttctttggcctgtcacaaccaggtcacatgatgcactgtctagttgctcagtaattatcctaggcactcgggcttttatcctaaaacttgtcacatattgtttttcagggtcagtgatgggtatttgtcagccctgtctcacatttgtagccagaagcctaaggtcatcattagcacgtgcctttgattgtcaggaaacgaagtactaggtatatggaagtttctttcattgcttccatgattgggactttctcaatcctaaagaagaagaaaaatccatacacagtttattgcctttcaaactgacagttctgtaaggctcattggatgattaagtaataagaaggagacaaacctgagaaaagcgtagattaaaacatatcctccagcttaaagccacaatacccagtagtcagacccattgagcagccagagagaatcttgtatgtgatgttcatttaaaaaaatatacaaagttaaaaaagtatgtcctccttcagatttaactatgattagaaaatgacacaaatacccacatatgcaatgtgtataatataagtgtacatagatacctgcaaaatagatggttagttcagtttatttagatgcacagttccctaatcaaactaacggttgtttgataaaataaaaaggtgacataaaagggatgtgacttttgaagctgttacttatttaaccatttaaccatttgctccatttaaccaggagcaaaagtgtaaaacctttgccccttagaggttcaggaaacactaaaactcatttctgtgcttggagagtccctaataaggctggcagccaacagttaaataatacttatttcaaaatgatattaagaatagaaagatctgtttattttctgatggatattagttctctcacaacatcacattatccttaaatctatatgtgtgtgtgtagttttaatatattttttaagccaaacactaatacaaataaatattcttctgtcctattgggacatcagttagtctttactaaaggcatttgcagtaatcatctttcctattttaataatttttaaatcagtctttcttaaactgtacttttgattcatgaatccatataacagtaatataatatttcttaaggatttatttagttattaaaataattaatcatataaatttattataaactatttaattttgcatacaaacaaaataattttaggaaagctagtgaataaatgaatgaatgatttttttttttagttagaaaacttacaggaatgtttcagtttaaggtaaactttgtgttttctgaagttccttgggctggccattgaaataagtagtcacttaatatttttgctttgaaatgtctagtgtctagctagtactttaggaattagatattacttgctaggaagagcttgccttatttttttaagttttcaaatttaaaggttaattttaaataagtgtgatacctccatggttactattactgtttttttaactactattattaatattgctaagagccaacattttttaaaaacatgtattatgtacagacactgtgccatatactattagcatggactaaatcatttaacctcacaaaaaatggatgctattattgtgcctattttatcattgaggaagctgaggcttaaagaaatgaggtaatttgctcgaccttatggaggaagttttcagaatttttgtacattttgcaagtagtgaatgaataacatattttcatgtttattttaatattccattaattgaaaaattgagaggcctcattaaaaatgaaaatttgttagcctgaataaaatcactttatgaggggttggacattataatttgataatgcctactgatttttataaccaatttttatcttactggactgcaagccccattctaccctacatttattttcagatggaatcagttctactggagaggaatgttttaggaataagcgagtcaaatatacactgggaattaaaagctaattcagaaaaaaatatgtgctgcaatttacttggttgagtgctatttaccagcttgggtttatatgatagtaagaaggttctagaggctctggtcttcagaaaattgctattgcagggcatgagaaaaatggttatgctttgggtagaagctcaacttccaaagtaaacaccatttaacccacttctgaggacaagggaaaaatgtacgaaatttattttgggggtaatttctttatctttcatgtgcagtcgtgatatttatagagttttaagattaactcagataaaaatacaaattatataaaaatacaatttgttgctttggattcagtgactagcacctggaaaatagtatattcagaagatgtgactaaaattttggccagtttcccctcttttgatcaacaaaactcattgaagagtattctcggaacatagtcatttaacatatttttattaattgtggaaaatgacacacatatacaacaaattttataaaatgaaaaatatatgcttaattaaacattataaatccagtctactggatatgtagtgagatctcattcttgttttaatttttatttcttttttatgatatgattgagcatagtagaatgtatttattatttatttgagagtatcttgttttgtgaaatagctgttctcttactcttttttaatgatgaatgtacctgttttatattgatttatcaaagtcatttatatattttggaatgtatcttgtgttggttataagtagtacaaatgtcttttcccactctgtggcttgcttttttactttcttttttttttttctttgagatggagtctttctctgtcaccaggctggagtgcagtgtggtgcgatctcggctcactgcaacctccgcaactgggattcaagctattctcctgcctcagcctcttgagtagctgagactacaggtgcgtcgcaccacgccaagctaatttttgtatttttagtagagacaaggtttcaccatgttggccagaatggtctagatctcttgaccttgtgatctgcctgcctcggcctcccagagtgctgggattacaggaatgagccaccacatctggctgcttttttactttcttaatggtattctttgataattggaaagatctttaatatagctcacttttgaaatttttttattatgaattgtaaattttgatggaactatagtcagaaaacaaatgctgtataattccaatttttaaaaatttgttaaactgctttatcatcatcatatgatcagtttttttaaatgtcttgtgcaaggtttaaaagaatgcgcattctttgtgggtctagttttccatctatgcctgttatgtcaaatatgcaaattgcattattcaaaactgctgtatcattattgtatttttttttttagcaacagttgtgttggctttactacaagtattataggtatatccatttgtccttgtagttctgttgatttttgcagtatatatttttaagctataatggtaggtacataaatgcttaaaagttgtaatttactggtgaaattagtcttttattactagggggtatttttctttattccaattgttttctccaggagggttggtaaaaattaccaagttaaacatttctggaataaaatatcttcaaataaactctgctgggaatacatagtttcttcttgcgctcactgagagaaggaggcaatcaggtaaatggcccttacagtatagttttagaagtattgtagcaaaacatggcattctgtgatagcccttagaaggtcacttaacccagacttagttgacaagggcatttccagaagaggtggcatctgaactgggtttgaaagtatgtaagagttggccgtgacaaaaacaggaatgaaggcagaggggggcagaggcaagggagtatatggttgcttggggacttgtatgtttcagctattaagtgtgtcaaaaagtctgggaagaaaggaaggagtttaaaatggacaaaaatatttctaattgtttgctatttcaaggtgtttttaaatgttgccaattataaataaataacactgcagtggagaactttgagcattattctttgtccttgtcccctactagttcttgaggacaagtttcttgaagtagaattactttagcaaggggtacagaagattttacacacaaaaggggacatctaattcaagatcatagaaggcttttaggaataaattacggtggagccaagttttgagggatgaatcagagttagttaagatgaaggtaaaacataaatgctgggaaatctcatgagcacagaggagagtgagttgaaggaagggtaaataatttcttacgggttggcttggtacaaggtctggtgagtcatgttgacagagtgggcttaagtgcaaactaggattatacttgattccatcaaagagctttagttaataaaataatgaaatgcctttttttcttattcttaccttttgaagttcctggaacccatttgtaccatattccccaaataattataatgattatctttcatatgccagaactcattttctaccatctctccaggtgattttagcatctggaacacattgtttccccttcactgcccaccatcatactaagtaatgcctgcttccctttacttcttttctattcctccatatccacacgtcctaggatggtacttattaagtctaaggcccaacttcgccatttaacagctgtgtgattcctgacaagctacttaacctctctttgcctcagtttcctcatcagaacctcaactgtattcatcataaaattgtgagaattaaattgtgcatgtgaaaatatccggaatgtctttgcagcacaatgacaattatttttcctcccattatgaatacataaatttgcccaggtttaatatgcctcttagtatagtttagagaagctttacaaaggtaaaccaaggatgagctgcgtatcctgagcatcttctgtctcagaggtggctaccctcatttaggggcaaagtccacgctggagagagcacctcacctttctttgattgctagattgagaagaaaatgaggatctcagcctttaaagctttccatttatcagcttttacacattcaagaagcatttcatttttatgttgcaatagcatgtatttccttctcttaggtcacctttgccttctttgtacttagtccctattcttcatctttcgttttctctcagttacaaaaaggaagccaaattctggccattgcagatagcaaaatatcacggcaggctaactaattttgcccagactttctgcgaaagctccacatgtgctgcagtgttgggttgcataattgatttaatctctaattttagaggaacgctgaactctttttgtgggactctgcatacatttctctagggcaaaaactgtcctacttttttctctctggtatctctttagtgctaggccacttttttttttttttttttttttttttttttttttcccaaagactctaaactaccctccgtgaaataaatgaaatcacagttggaaaacagcttactggacagacttgtaatggcaagataacagctacgccatacacatttactaaacattttcatcctcccctgcttcccttctttctgctctagcacatctttattttcactaagatgatgcatcaggctctcagctgaggagtggcattacctacacagaggagcaacacacctttgcatattattggaaatacataggcttgctcacccattgacctactatatgcctctaggcagatttacttacctgtctgaatcttgcttgctttcagcagagtactagagtaatggctgtgaaggctgaactataaaaataggtatgtagcaagctttagacttaccttgtgccttttggctatccaggtatataggaagtgtcagcaaggggggatgattttatgttgctcaaaagaccagcaaccaagatgaaaaccaggcaattgtaagtttactagttatttcacattgtatttagcaatgtcatttttggttatcttttcgggaatttatatactgggaagtcgcatttgatagccggtggtttttattaacctgtctgatttggaatataattttgggattaccttcccaccagttacttctcaaacaactgactttaataactttagtatttttgtactctaagacatatgttccattgaaactaaccttcctcaggtccatactcactaacgctaatttgacaaattccctgaacaattctcataccatagaagtatgaattttcataccaaactccactgttaacatcaataaaattaggtcagaaagaattccaactgctgtctgagtatcatagccttatgtctttatgagtctttatgctgctaaaagaatttagttttagaaaataacctcttatctgttaaataaacacttagcataaacagatctgtgttcaaatagattctgttacttaatagttggatcaatttagacaagtcccttaacctgtaagcctctttatgtgcaaatatttaaaactgggataataattatactgacttctcaggtttgttgtgaggagtaaatgctaaaatgcatataaagagatttgaaggttatctagccatagacagtgtatcaggcaggttttgccataataatggtgtgtaacaatccccaaatctcagtggtttacatcaagaaatatttgtgttagacttagttgaacttggctccagcttgtgcattctattcaggtttgctctgcatatctcttcattcagataccacaggctatttaagaaattctgtattcatggtagagaacaacattgaaagagatctggtaaaaaatgaaataaaacaaaacaaaacaacttgccaagtcccttaaagcatcaactgaaaactaatattcctaccactttcacacaaatttcattggtcaaagcaagtcatatgaccaggataaatattagtaggtcaggaaagtttacactgccctaataaagaaagaagagtgaatacctggtgaataatacatgagtctccttcaaagaagtttactgaatcttttttatcctactcttatttcatctgcatgaccattgactcctccatatagtctacttcacagtatctacattgcattattgaaattatactgcaatttctccctctctttcttttaatttcaaattgatttgaatgatatacttttctttgtaaacag

SEQ ID NO: 219 - DNA, 9730 bp, Homo sapien
gtaagtactaattatattcacatccaaatgagggctgattttttaggtccatattgacatatgttgcaaatgtcaatctacttacactgaatcatataactcatctgctttttaagataagaataaaaattcaaagagtcatttaaagacttaaattcttagtttttagattctatattaaattcatgtttttcatgattattctatttccaaggtggtagaaaacattatattttgattccttaaggtgaaaagcatgtaggaagagatatttttgaagcaacttgtttttagttcatttgattgtttatttatttatttaatgaatttaagcaatgggctaggttctagggttggtaaagtgaaatagatgttattctgttgctactgctatatcattttatttgtgatagtgctaataagctattagtatttaatgggtagtgactacttctatacaaccatctccagtgaatgtggaatttggtagaacttaaataaaaagcaaaactaaaaagaaaaccacgtcctcacacttggagaaatattcagctaatacattgattgtctttttgtacacagagcaacatatccaagtgaaagtttagctagtgtttttgtgtttataatagagatggatgttcctctttcctgttatacattgatatataaagaggatgagaaaaaatgcaagcaaatttttctggatctggttgatttaaggcctttgggaattgttaacttaaaattaaaaggtactcatagcttattattattcaaaatttattcagttttggatatttttgtcaactaccaaaccttcaccatgttagtgacattattataatattgtgtatggtggtatgagaattagaatgcttaaagaaatgatgctgatgcatcttagcttacattgaaatatctttccttttgcccaattttatttagatcccactgagttcttgtaagtatattataagtatacatacatgaataatttttgtctagagagaaagagagtcagtttattgcttaattttatttagattttacagtactgagctttctaataggtcgtaattccaaaatgactgattttatattatatataaaagacgactcagttaatattgtgcttttaactagaaacagagaaaagtcaatgagtagaagcaatagaggtacatcagaagaaaaccagcagagagtggatccagcacaagtttagatatatacacacatatatgtggggcatgccattctatcatgaggaagtgaaacagcttcagagaaaagatcacttttatttatcacccaccatttgtcatagccatcatgtttttttacttttgactcagttatttctgatttaataagaaacagttcaaagtgtataatatagcattgattttgtgttggaatgtgttctatccgcctatctctctctctttttctttagcattttaaaagacattttctagacataaggcaaagagtaaatcttctgaagataaagaagggctgcttcttaagtatgtctgaacaaatttctcccgaataagaaatgccatatcctatggcattcaatgagaattagcaaggtttctcatgttttctagtgttgcacagtatgataaaaaaccaaaccaaaatgaaacaaaaccctaaagcccgacacagtctccagagccagccttgtttctttcagctttgatcacagtcagttgggtcttccattgaactcactggagagccattgacgagcccatgaactgaccttaagccagcagaaatgagtctaggaatgcagttctttcttttttttttttttattattattatactttaagttttttcatggaaaagtaccaaggatgttctccagtggtttgctctcgttaatttaatgagcttatatgcatttcaacaaattcaggccatactggacacatttttctaagagagtactagctactttcaaaagctaccaactttgggccaggcgtgatggctcacacctgtaatctcagcactttgggaggtggaggtaggtggatagcttgagcgcaggagtttgagaccagcctaggcaacatggtgaaaccccgtctctacaaaaacacaaaaattagctggatgtgatggtgcatgcctgtagtcccagctacttggaaggctgaggtggattgttttagcctgggagagagaggttgcaatgagctgagattatgccactacacaccagcctgggtgacagagccagatactgtcttaaaaaaaaaaaaaaagaaagaaaagctataagctaccaactctgttttgtgggaagaaacggagtagccatctacttttaaagtaaataatacacagtgaactagcctcttctggtctttctctagaggcatctggattcaagcatcctctgcttagctaagtttccatcaccatgttttccaacttccaaaactttgttgatctctgttgtcatttgttgtcttcttgcatgtggcatggtggtttgctgcacctatcaacctgccatctaggttttcagccccacatgcgttaggtatttgtcctaatgctctcccttcccttgctccccacccgtcaacaggccccggtgtgtgatgttcccctcactgtgtccatgtattgtcattattcaactcccacttatgagtgagaacatgcggtgtttggttttctgttcctgtgttagtttgctgagaatgatggcttccagctttgtccttgtctctgcaaagggcatgaactgattcttttttatgactgcatagcattccatggtgtatatgtgccacatttctttatccagtctatcattgatgggcatttgggttggttccaagtctttgctattgtgaatagtgctgcaataaacatatgtgtgcatgtgtctttatagtagaatgatttataaataatcctttgggtatatccccagtagtgggattgctgagtcaaatggtatttcttgttctagatccttgaggaatcgccacactctcttccacaatggttgaactaatttacactcccaccaacagtgtaaaagtgttcctatttctccacaggctcaccagcatctgttatttcctgactttttaataattgcctttctgactggcatgagatagtatctcattgtggtttttcagggacctcaattctgtagtcataggaaatgcattcttccaacaacctgtttggaagtgaactcttcagtaaacccagccaagcctgtctggactcctggcccactccctggagataacacatttgtgttactgtaagccactgactgtggtaatttgttatacagcaatagaaagtaatatggcatatcacataaagctctttgtttgtgacattgccatgaattttgggccatgaaaacagttactgtgagggtgagcatgacactgtgggagtgtgtgtctgggtgtgtgcaatgtgtatatacttatcatattagtagctttttaagttcccccattctgcataaggagcaccctatcaggttgctttggaatagagaagaaaaccaggaaagccaatcaattatacaagtcttttggcattcagtcacctttagcattttctcttctgcatcttctcctctttatgtatcctgggcacctgctttctagaagagagctgtttggagcatgaaagagtagaatctttctatcagcaaggaagtatgttcttttccagaccggcagaatgttcatcctagaagagattcctatattgccagcttgtctcttttcttccctatgacaaaaatcacaactttgccattaaagagtgtttttacatcatttcttcttttcactacagtacaaattcactactttgtttaattgggtcgatggcaaaattattgtaatcttatttcatatgtgtggctttgtgggaaagtgagatgttctacgtaagcaagctttccatacaattaaaaattgttccattaataacaagcattttaaaaagtcttcaactcttaatcgttaaaaaaagacaaagattgcataatacccaggcttattcaatgggaaataatgtagactgttcacatttgttatgacgattcatggtcatcattaaatctgtcatttacttcacaatcctgtttttgctgtgatgcccttgggtactatttttcgtatactgaatggtgccagaccactaaatctttagttcttgtcctgcggtttgtattttttctgctattttgctgtgaaaaggatgctaattgtaatctattgtatttttttttttgctattttgctatgaaaaggatgctaattgtaatctattgttgctgttgacatatccacatccagaaaagttctaagtccaaaatcagacgacactaacttaaaatcaattaatttaagaattatttgtacacatagtgaactcataggtaaatataatagactcttgaaagcttacatgccaatgttttttattccccagtgttaaccgaacagctattatgttcaaaatatcatatggtccctataagaatctagagatttgaaaattaatggatatgaaaaacaacatttattcaactatttaatgagaagatgttatactaaatctcattccgtatctattacaaataaatcattaattctctatgaagctgcttagccattaatccagttttttgtaacattaacttgagtataaaatgttaatctttataaaatttattaggtggtcaaggtttcgacttatgattatattctcaagtgtgcctaagcacattgtaaatcacaataaattatcattcaatatatcacatacagtgaaaggtagcctgttttacttttctactattctaaaactcctgtgcaaccaatatcttgaattaaatctcttccattagaaagaccttgagtgatttctgttttcctgtacagacttatgactgctacagctccccaggctaaacaggaaaatttgattattttgcgtatggtctttaaaaatgcagagttttcattctcctagataaaatactcatgctagatttattataaaatcttgttttaaatgatttaccaattaaaatacttaggtttttgtcccctgatttttgcttataattgattttacttatctttcattgtccttgtgccctgttaaacccacctaggcttacttaacagctagtcaactcctaagaggtgtagttgtcccctgaattgttggaagatgtaccgtgtttattctgtgtctttatatacttctgatccactgcaagttatacctaaggttactggaatctcatttttaaatgaataattccatggaatcacaagagtttaatcagaagtatcacagaatagaagtaagcacactgcttgaggagtgccccagaatgtgaagttgaaggcaaagatgagtggtagtttttcagaggcaattagtgacctaagaacagaggggatgcatagtaggtactgatttggcaagtgttactataacaacatgtactgggtgcttacaacatgccaggcttggaactactagaccacagtgccagaaaaaaaccgtcagatcataagacatttcttccctcttaggaattggctatttgctaagaaagcctagataggcttaaaagggagcatcaggaccatgaaaggtaagaaaattgtatctatcaactcagaaggataaccagaaaagtcattcttgtcatacaaagtatacagaaggttattcttgagggggagagatcacagtgttctatatctttgcatatctcacagtgccttgaacaacatttgtacatagtagaagggttaacatatacttgttaactatatagaaatatgttgatattagatttgcattttccctgtatataaacataatatatttaatttagaattctttagaattgactaaagaatttacttttttctttcagagtacaaaattgtccaaaatcattttatatttcaagtccccttgtggcacatatagaatatttggccttatctagcctatttaaaacaagagagataggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaaaaaaaaaaaaaaaaaaaaaaaaaaaacaagagagataaaaaatgctcaggacatcatgaagcattcaatcaaaacatgaggctttttataggaactcggcttagtagctttgagaagaaggttgaagagagagtatctctgatttccaccccaacttcacctcaccccaccacagtaggtcttgttgagagccagttttcactaataaaacttacttgctttctgagttttccatgcaaatagaaataattccatcttctacagggtaaacatttcagatctggtggaatcgaaaagcaagaataatgacatgaataggagtgatctcttaattgggaaatttctgtattttcttgacttttctcctatagcctgcatttaagcattgcagaaaaaaaaatctctcacatacctaactgggtattggaagctattaaataatggtaaaaactgggatgaatggagggaacttttctcccccattttttaaagtttggctgtaggtccttctgcctgcaggctacagtcacattagcagttggaaattggataacgacagcacaatgacaaatccaggttgcttgcaataagttgctaatccctttgctttaatattggagcagcatgtaatgatgtttggacatcactccagtttactgacgaccccactggggccagcaatagaaccttacaactgtctaacaaggcatcaggtgactcccgtcgtcacagcaacggacactggaatctaatcttccctccatcccttctagcacccaataacgcctagattatataagtggcccatcattgcttggtaacttgaatcaattaacgttaattcgcacaatgctatgatgtattgtgatcactttcatttcagggaagggggagaaattgctataagtcacctaaaatgaggttgtctgtggtgctgaggtattaattgggtgtccatattaaatgcaaaaggagcccatcatgaaaggaaagtggatgagtgactctcttggctgctattgacctatcagaagaagacagtgcccttttcctttgcttttatctattacagttttccctattgcgtgtcaagttgatgtattataaaaaattcatttgatgacctttcacccccttagcagattaaacaatttcacttcgatggcatcattaacaggaccctgcatttaatgatgatttctctaaaaggccacggagattcgattttaagctaacagatttattgcactattataacatatcgtaaaaaacagtaccacctacggtcaggttttaggatagaaactgcttaagagtttatgtggagcttaaagtggtattaccttgggatatgctgcagcagcaagtctgtgacctccagtgagatgctgtttacatttaacaagagatgtttgctcacaataaactttaaagtgtgtcaggaatggaccttttagcgggtgttcaggcagttggtttccctctcttcgtctcaagacgtcaatgtacttatgtgagaatctttgatggggtctttcagtctctttaacccagactgcttgcctgcatcctagcccctatcagaaagaatttaaggaaaaggttaggggagaaataggatctgataacaacattagaattttctaataacctggaacccacagaaggatagaattaatagcatggatttaaaaaatctggtttaaattgttccctttaaaaaaggaaagaagccctagccactcaggctaaacaaactaagaaatgatgatggggctagtgtgagttgaattcccagggagtgattccagccaaaaagccagggtcttagctggtagatgactaaagagcttcttaggaccacatgtttgcactctgcccgcatttcctaggattcccaggggagaagaaaattgcttcctatggaaacttcatatttagtgatccattgtatttgctcttccactatttcctctttcagaacaaatcttcactttttcctgacatgcaccctccatattaaataaatactttatcctttcctcctcacgtggggaaggggaggtggttaggaaatagaatagttgacttacatttaatgggagaaagcaaagtaggctactttaaaaataaataaataaataaatacctcatgagacctggcctaggctattgaggataactcaccaagggttgctctatgtcagggtcccatatatatgtgatgtccaggtgtctgcatttagttgccaaacatctgttgcatgcctatgatatgtaattcacaataggcttaatgggacaataggtatataattttattttaatgaagcaggctctgtcatcaaaaataccgaaactctgagtgaaatatgttttacactgtcatgaattatgtaactattttctatgtattccattttgttgctaagtcatgtcagcacttaacatgttcaaaggaactaagaagtaatataggtcattggttaaccactctagatcttggtcaacacttactatttgtaggacctttggcaagttatttaagcctttttggacctcagtttcttcatatataaataagagtacctaccacatagtgtcattttaaagatgcaatgaatttatatatgaacactttatcatggaacttggtatatagtatgtgctcagtaaatatcaattctaaatattattctcctcagatgcttcatgcaactgaagtaaattaattcattaatgctattaatagttattgaatccatttagtacaaacacactcggaaaatataaatatggcttgttgtgaagattaaatgagctattctgtgtaaacagtggtgtactggagtttgctgttgggcatctcttcccagctctgctcagtgatattatcttgtcatctcagaattagctatgttggtggtatttatatcacagaattggaacacatccacaaatcagggcattatttgggaagctgatttaatagcttactattggacgtgtccatgtcctgtcattttagtctgaggcttttattattattatctatattagtctgaggctaattttcactgaggcttttattttttggctgattttaataaatgatgataaaaatatatgctcataatttgtgattttccctaccccctagtccattttcattatagctaagaaaacatatcaaatacatacacaattaacctttgctataaagccatgttcactacacaatgtattattttctagaaacacaggcaaatgcaaatattttcactggtaataatggattgtggaaaattgctaccatagggatatgtgtaatcctaaggatgtattttgtttgtttttatatctgtca

SEQ ID NO: 220 - protein, 314 aa, Homo sapien
LNLLDDVSSWKPGDQIVVASTDYSMYQAEEFTLLPCSECSHFQVKVKETPQFLHMGEIIDGVDMRAEVGILTRNIVIQGEVEDSCYAENQCQFFDYDTFGGHIMIMKNFTSVHLSYVELKHMGQQQMGRYPVHFHLCGDVDYKGGYRHATFVDGLSIHHSFSRCITVHGTNGLLIKDTIGFDTLGHCFFLEDGIEQRNTLFHNLGLLTKPGTLLPTDRNNSMCTTMRDKVFGNYIPVPATDCMAVSTFWIAHPNNNLINNAAAGSQDAGIWYLFHKEPTGESSGLQLLAKPELTPLGIFYNNRVHSNFKAGLFI

SEQ ID NO: 221 - protein, 316 aa, Homo sapien
LNLEDNVQSWKPGDTLVIASTDYSMYQAEEFQVLPCRSCAPNQVKVAGKPMYLHIGEEIDGVDMRAEVGLLSRNIIVMGEMEDKCYPYRNHICNFFDFDTFGGHIKFALGFKAAHLEGTELKHMGQQLVGQYPIHFHLAGDVDERGGYDPPTYIRDLSIHHTFSRCVTVHGSNGLLIKDVVGYNSLGHCFFTEDGPEERNTFDHCLGLLVKSGTLLPSDRDSKMCKMITEDSYPGYIPKPRQDCNAVSTFWMANPNNNLINCAAAGSEETGFWFIFHHVPTGPSVGMYSPGYSEHIPLGKFYNNRAHSNYRAGMII

(19) United States
(12) Patent Application Publication
(10) Pub. No.: US 2006/0088828 A1
(43) Pub. Date: Apr. 27, 2006

(54) Polycystic kidney disease nucleic acids and proteins

(76) Inventors: Peter C. Harris, Rochester, MN (US); Christopher J. Ward, Rochester, MN (US); Sandro Rossetti, Rochester, MN (US); Vicente E. Torres, Rochester, MN (US)

Correspondence Address: FISH & RICHARDSON P.C., PO BOX 1022, MINNEAPOLIS, MN, 55440-1022, US

(21) Appl. No.: 10/501,834

(22) PCT Filed: Jan. 23, 2003
(86) PCT No.: PCT/US03/02038

§ 371 Date: May 26, 2005

Related U.S. Application Data
(60) Provisional application No. 60/351,110, filed on Jan. 23, 2002.

(51) Int. Cl. C12Q 1/68; C07H 21/04; C12P 21/06; C07K 14/705

(52) U.S. Cl. 435/6; 435/69.1; 435/320.1; 435/325; 536/23.5; 530/350

(57) ABSTRACT

Nucleic acids and polypeptides are provided that are associated with autosomal recessive polycystic kidney disease. Antibodies against the polypeptides, vectors and host cells containing the nucleic acids, methods for using the nucleic acids, and compositions and articles of manufacture also are provided.

### TECHNICAL FIELD

This invention relates to disease-associated nucleic acids and polypeptides, and more particularly to nucleic acids and polypeptides associated with autosomal recessive polycystic kidney disease.

### BACKGROUND

Autosomal recessive polycystic kidney disease (ARPKD) is an important cause of renal associated infantile morbidity and mortality. ARPKD in the infant is characterized by greatly enlarged, echogenic polycystic kidneys and fusiform dilatation of the collecting duct. Presentation in later childhood usually is associated with less massive renal enlargement and more variability in cyst size. Approximately 50% of patients who survive the neonatal period progress to end stage renal disease within the first decade of life (Roy et al. (1997) Pediatr: Nephrol. 11:302-306; and Cole et al. (1987) J. Pediatr. 111:693-699). ARPKD also is characterized by liver involvement, including hepatomegaly, with approximately 45% of infants showing signs of liver disease; liver disease often is the major feature in older patients (Roy et al., supra; and Zerres (1996) Acta Paediatr: 85:437-445). The basic defect in ARPKD may be a failure of terminal differentiation in the collecting ducts and bilary systems (Guay-Woodford, in Polycystic Kidney Disease, Watson and Torres, eds. (Oxford University Press, New York, 1996)).

ARPKD presentation is highly variable. Typically, patients have been separated into groups based on age at presentation and severity of disease, which suggested different genetic entities. More recent evidence of intra-familial phenotypic variability, along with genetic linkage studies, have suggested that allelic heterogeneity rather than genetic heterogeneity, as well as genetic modifiers and the environment, may explain much of the observed variability (Kaplan et al. (1988) Am. J. Med. Genet. 29:639-647). Although linkage between ARPKD and chromosome 6 was first described in 1994 (Zerres (1994) Nat. Genet. 7:429-432), identification of the gene itself has proven difficult.

### SUMMARY

This invention is based on the identification and characterization of a gene associated with ARPKD in rats and humans. The sequences of the rat, mouse, and human transcripts are described, as well as the sequence of the “fibrocystin” polypeptide encoded by each species. In rats and mice, the gene is called Pkhd1, while the human gene is called PKHD1. The identification of genetic mutations in ARPKD patients provides methods by which to conduct diagnostic tests for ARPKD, allows for genetic screening of potential carriers, and provides methods for therapeutic intervention.

The invention features an isolated nucleic acid containing a sequence encoding a fibrocystin polypeptide. The fibrocystin polypeptide can be encoded by SEQ ID NO:1, SEQ ID NO:3, or SEQ ID NO:4. The fibrocystin polypeptide can have the amino acid sequence of SEQ ID NO:2, SEQ ID NO:6, or SEQ ID NO:7. The sequence of the isolated nucleic acid can contain a nucleotide sequence variant associated with autosomal recessive polycystic kidney disease.

The fibrocystin polypeptide can contain an amino acid sequence variant at a position selected from the group consisting of: position 17, position 36, position 222, position 739, position 757, position 805, position 1249, position 1389, position 1407, position 1664, position 1741, position 1833, position 1838, position 1867, position 1917, position 1942, position 1995, position 2331, position 2688, position 2869, position 2957, position 3018, position 3177, position 3346, position 3468, position 3502, position 3529, position 3553, and position 3622 of SEQ ID NO:2. The amino acid sequence variant can be selected from the group consisting of: Val at position 17, Met at position 36, Val at position 222, Leu at position 739, Leu at position 757, Leu at position 805, Trp at position 1249, Thr at position 1389, Arg at position 1407, Phe at position 1664, Met at position 1741, Leu at position 1833, Cys at position 1838, Asn at position 1867, Arg at position 1917, Gly at position 1942, Gly at position 1995, Lys at position 2331, Phe at position 2688, Lys at position 2869, Thr at position 2957, Phe at position 3018, Thr at position 3177, Arg at position 3346, Val at position 3468, Val at position 3502, Gln at position 3529, Thr at position 3553, and Tyr at position 3622.

The fibrocystin polypeptide can contain an amino acid sequence variant at a position selected from the group consisting of: position 25, position 752, position 760, position 830, position 852, position 1262, position 1709, position 1870, position 2938, position 3139, position 3505, position 3899, position 3960, and position 4048 of SEQ ID NO:2. The amino acid sequence variant can be selected from the group consisting of: Val at position 25, Met at position 752, Cys at position 760, Ser at position 830, Arg at position 852, Val at position 1262, Phe at position 1709, Val at position 1870, Met at position 2938, Tyr at position 3139, Arg at position 3505, Arg at position 3899, Ile at position 3960, and Arg at position 4048.

The fibrocystin polypeptide can conatin the amino acids from position 1 to 3299 of SEQ ID NO:2, position 1 to 2578 of SEQ ID NO:2, or position 1 to 3779 of SEQ ID NO:2.

The sequence can contain a nucleotide sequence variant with respect to SEQ ID NO:1, SEQ ID NO:214, SEQ ID NO:216, or SEQ ID NO:217. The nucleotide sequence variant with respect to SEQ ID NO: 1 can be at a position selected from the group consisting of: position 50, position 107, position 657, position 664, position 2216, position 2269, position 2414, position 3747, position 3761, position 4165, position 4220, position 4991, position 5221, position 5498, position 5513, position 5600, position 5750, position 5825, position 5984, position 6992, position 8011, position 8063, position 8606, position 8870, position 9053, position 9530, position 10036, position 10174, position 10402, position 10505, position 10585, position 10658, position 10865, and position 11612 of SEQ ID NO:1. The nucleotide sequence variant with respect to SEQ ID NO:1 can be selected from the group consisting of: T at position 50, T at position 107, T at position 657, G at position 664, T at position 2216, C at position 2269, T at position 2414, G at position 3747, G at position 3761, A at position 4165, G at position 4220, T at position 4991, A at position 5221, T at position 5498, G at position 55i3, A at position 5600, G at position 5750, G at position 5825, G at position 5984, A at position 6992, T at position 8011, T at position 8063, A or T at position 8606, C at position 8870, T at position 9053, C at position 9530, C at position 10036, T at position 10174, G at position 10402, T at position 10505, C at position 10585, C at position 10658, A at position 10865, and A at position 11612. The nucleotide sequence variant with respect to SEQ ID NO: 1 can be an A inserted at position 5895 or 5896, a deletion of the nucleotides at positions 1624, 1625, 1626, and 1627, a deletion of the nucleotide at position 10637, a deletion of the nucleotide at position 9689, a deletion of the nucleotide at position 3762, a deletion of the nucleotide at position 1529, a deletion of the nucleotide at position 383, a deletion of the nucleotide at position 6383, a deletion of the nucleotide at position 10856, or a deletion of the nucleotide at position 10364. The nucleotide sequence variant with respect to SEQ ID NO:214 can be at position −2 relative to the splice acceptor site of intron 28 (e.g., a C at position −2 relative to the splice acceptor site of intron 28). The nucleotide sequence variant with respect to SEQ ID NO:216 can be at position −9 relative to the splice acceptor site of intron 33 (e.g., a G at position −9 relative to the splice acceptor site of intron 33). The nucleotide sequence variant with respect to SEQ ID NO:217 can be at position +4 relative to the splice donor site of intron 43 (e.g., a T at position +4 relative to the splice donor site of intron 43).

The nucleotide sequence variant with respect to SEQ ID NO:1 can be at a position selected from the group consisting of: position 73, position 214, position 234, position 1185, position 1587, position 2046, position 2196, position 2255, position 2278, position 2489, position 2554, position 2853, position 3537, position 3756, position 3785, position 4920, position 5125, position 5608, position 7587, position 7764, position 8813, position 9237, position 9415, position 10515, position 10521, position 11340, position 11196, position 11878, and position 12143 of SEQ ID NO:1. The nucleotide sequence variant can be selected from the group consisting of: A at position 73, T at position 214, T at position 234, C at position 1185, C at position 1587, C at position 2046, T at position 2196, T at position 2255, T at position 2278, G at position 2489, C at position 2554, T at position 2853, C at position 3537, C at position 3756, T at position 3785, G at position 4920, T at position 5125, G at position 5608, A at position 7587, G at position 7764, T at position 8813, A at position 9237, T at position 9415, T at position 10515, T at position 10521, C at position 11340, G at position 11196, A at position 11878, and G at position 12143.

The fibrocystin polypeptide can be encoded by nucleotides 276 to 10174 of SEQ ID NO:1, nucleotides 276 to 8011 of SEQ ID NO:1, or nucleotides 276 to 11612 of SEQ ID NO:1. The isolated nucleic acid can contain nucleotides 1 to 192 of SEQ ID NO:1, nucleotides 193 to 328 of SEQ ID NO:1, or nucleotides 329 to 406 of SEQ ID NO: 1.

The isolated nucleic acid can contain a nucleotide sequence variant with respect to SEQ ID NO:5, SEQ ID NO:209, SEQ ID NO:210, SEQ ID NO:211, SEQ ID NO:212, SEQ ID NO:213, SEQ ID NO:215, SEQ ID NO:218, or SEQ ID NO:219. The nucleotide sequence variant can be at a position selected from the group consisting of: position −47 relative to the splice acceptor site of SEQ ID NO:5, the position just 5′ to the splice donor site of SEQ ID NO:209, position +19 relative to the splice donor site of SEQ ID NO:210, position +23 relative to the splice donor site of SEQ ID NO:21 1, position +13 relative to the splice donor site of SEQ ID NO:212, position +50 relative to the splice donor site of SEQ ID NO:213, position +53 relative to the splice donor site of SEQ ID NO:213, positions +42 through +45 relative to the splice donor site of SEQ ID NO:215, position −32 relative to the splice acceptor site of SEQ ID NO:218, and position +9 relative to the splice donor site of SEQ ID NO:219. The nucleotide sequence variant can be a T at position −47 relative to the splice acceptor site of SEQ ID NO:5, an A inserted just 5′ to the splice donor site of SEQ ID NO:209, a C at position +19 relative to the splice donor site of SEQ ID NO:210, a T at position +23 relative to the splice donor site of SEQ ID NO:211, a G at position +13 relative to the splice donor site of SEQ ID NO:212, a T at position +50 relative to the splice donor site of SEQ ID NO:213, a G at position +53 relative to the splice donor site of SEQ ID NO:213, deletion of the nucleotides at positions +42 through +45 relative to the splice donor site of SEQ ID NO:215, a G at position −32 relative to the splice acceptor site of SEQ ID NO:218, or a G at position +9 relative to the splice donor site of SEQ ID NO:219.

In another aspect, the invention features an isolated nucleic acid encoding a fibrocystin polypeptide, wherein the nucleic acid comprises at least 300 contiguous nucleotides of SEQ ID NO:1 or a sequence variant thereof. The invention also features a vector containing the isolated nucleic acid, and host cells containing the vector.

In another aspect, the invention features an isolated nucleic acid 10 to 1650 nucleotides in length, the nucleic acid containing a sequence, and the sequence containing one or more nucleotide sequence variants relative to the sequence of SEQ ID NO:1. The sequence can be at least 80% identical over its length to the corresponding sequence in SEQ ID NO:1. The nucleotide sequence variant can be at position 50, 107, 383, 657, 664, 1529, 1624, 1625, 1626, 1627, 2216, 2269, 2414, 3747, 3761, 3762, 4165, 4220, 4991, 5221, 5498, 5513, 5600, 5750, 5825, 5895, 5896, 5984, 6383, 6992, 8011, 8063, 8606, 8870, 9053, 9530, 10036, 10174, 10364, 10402, 10505, 10585, 10658, 10856, 10865, or 11612 of SEQ ID NO:1. The nucleotide sequence variant can be at position 73, 214, 234, 1185, 1587, 2046, 2196, 2255, 2278, 2489, 2554, 2853, 3537, 3756, 3785, 4920, 5125, 5608, 7587, 7764, 8813, 9237, 9415, 9689, 10515, 10521, 10637, 11340, 11196, 11878, or 12143 of SEQ ID NO:1.

In yet another aspect, the invention features a plurality of oligonucleotide primer pairs, wherein each primer is 10 to 50 nucleotides in length, and wherein each primer pair, in the presence of mammalian genomic DNA and under polymerase chain reaction conditions, produces a nucleic acid product corresponding to a region of an ARPKD nucleic acid molecule. The nucleic acid product can be 30 to 1650 nucleotides in length. The nucleic acid product comprises a nucleotide sequence variant relative to SEQ ID NO:1. The plurality can contain at least three primer pairs, at least thirteen primer pairs, at least sixteen primer pairs, or at least twenty-three primer pairs.

The invention also features a composition containing a first oligonucleotide primer and a second oligonucleotide primer, wherein the first oligonucleotide primer and the second oligonucleotide primer are each 10 to 50 nucleotides in length, and wherein the first and second primers, in the presence of mammalian genomic DNA and under polymerase chain reaction conditions, produce a nucleic acid product corresponding to a region of an ARPKD nucleic acid molecule. The nucleic acid product can be 30 to 1650 nucleotides in length. The nucleic acid product can contain a nucleotide sequence variant relative to SEQ ID NO:1.

In another aspect, the invention features an isolated nucleic acid containing the nucleotide sequence of SEQ ID NO:1 or its complement.

In still another aspect, the invention features an antibody having specific binding affinity for a fibrocystin polypeptide. 1. In yet another aspect, the invention features a method for determining the susceptibility of a subject to autosomal recessive polycystic kidney disease. The method can include providing a nucleic acid sample from the subject and determining whether the nucleic acid sample contains one or more nucleotide sequence variants within the PKHD1 gene of the subject relative to a wild-type PKHD1 gene. The presence of one or more nucleotide sequence variants can be associated with increased susceptibility of the subject to autosomal recessive polycystic kidney disease. The nucleic acid sample can be genomic DNA. The determining step can be performed by denaturing high performance liquid chromatography. The method can further include identifying the nucleotide sequence variant by DNA sequencing. The nucleotide sequence variant can be a deletion of the nucleotides at positions 1624, 1625, 1626, and 1627, and an A at position 6992 of SEQ ID NO:1. The nucleotide sequence variant can be a G at position 664 and a T at position 10174 of SEQ ID NO:1, a G at position 4220 and an A inserted at position 5896 of SEQ ID NO:1, a T at position 8011 and a C at position 10658 of SEQ ID NO:1, a G at position 5984 and an A at position 11612 of SEQ ID NO:1, or a deletion at position 10637, and a C at position 8870 of SEQ ID NO:1. The nucleotide sequence variant can be a T at position 4991 and a T at position 9053 of SEQ ID NO:1, a G at position 3747 and a G at position 5750 of SEQ ID NO:1, an A at position 5221 of SEQ ID NO:1, a T at position 107 of SEQ ID NO:1, or a deletion at position 9689 of SEQ ID NO:1. The nucleotide sequence variant can be a deletion at position 9689 and a G at position 3761 in combination with a deletion at position 3762 of SEQ ID NO:1, a deletion at position 9689 and an A at position 10865 of SEQ ID NO:1, a deletion at position 9689 and a T at position 50 of SEQ ID NO:1, an A inserted at position 5895, a T at position 8063, and a G at position 10402 of SEQ ID NO:1, or a deletion at position 1529, a T at position 657, and an A at position 8606 of SEQ ID NO:1. The nucleotide sequence variant can be a G at position 664 and a G at position 3761 in combination with a deletion at position 3762 of SEQ ID NO:1, an insertion at position 5895 and a C at position 10036 of SEQ ID NO:1, a deletion at position 383 and a G at position 5513 of SEQ ID NO:1, a deletion at position 6383 and a G at position 664 of SEQ ID NO:1, or a deletion at position 383 and a G at position 664 of SEQ ID NO:1. The nucleotide sequence variant can be a deletion at position 10856 of SEQ ID NO:1 and a G at position −9. relative to the splice acceptor site of SEQ ID NO:216, a T at position 10505 and an A at position 8606 of SEQ ID NO:1, and a C at position −2 relative to the splice acceptor site of SEQ ID NO:214, a T at position 107 of SEQ ID NO:1 and a T at position +4 relative to the splice donor site of SEQ ID NO:217, a G at position 5825, a T at position 8606, and a T at position 2216 of SEQ ID NO:1, a T at position 107, a T at position 2414, and a C at position 9530 of SEQ ID NO:1 or a C at position 2269 and a C at position 9530 of SEQ ID NO:1. The nucleotide sequence variant can be a C at position 2269 and a C at position 9530 of SEQ ID NO:1, a deletion at position 1529 of SEQ ID NO:1, an A inserted at position 5895 of SEQ ID NO:1, an A at position 5600 of SEQ ID NO:1, or a C at position 10585 of SEQ ID NO:1. The nucleotide sequence variant can be an A at position 4165 of SEQ ID NO:1, a deletion at position 9689 and an A at position 8606 of SEQ ID NO:1, a deletion at position 10364 and a G at position 10402 of SEQ ID NO:1, an A at position 5221 and a T at position 5498 of SEQ ID NO:1, or an A at position 8606 and a C at position 8870 of SEQ ID NO:1. The one or more variants can be on separate alleles.

In yet another aspect, the invention features a method for diagnosing autosomal recessive polycystic kidney disease in a subject. The method can include providing a nucleic acid sample from the subject and determining whether the nucleic acid sample contains one or more disease-associated sequence variants within the PKHD1 gene of the subject compared to a wild-type PKHD1 gene. The presence of the one or more disease-associated sequence variants can be diagnostic of autosomal recessive polycystic kidney disease.

The invention also features an article of manufacture containing a substrate, wherein the substrate contains a population of isolated nucleic acid molecules, wherein each nucleic acid molecule is 10 to 1000 nucleotides in length. Each nucleic acid molecule can contain a different nucleotide sequence variant relative to the sequence of SEQ ID NO:1, and each nucleic acid molecule can be at least 80% identical over its length to the corresponding sequence in SEQ ID NO:1.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 (top) is a genetic map of rat chromosome 9 showing the genetic distances (cM; brackets) of markers from the Pkhd1 locus (shown as “Pck”). FIG. 1 (middle) is a map of the rat region syntenic to the human ARPKD interval, showing the recombination fraction and genetic distances of markers (cM; brackets) from Pkhd1. FIG. 1 (bottom) depicts the corresponding region of the human genome, showing some of the human genes in the interval (solid bars) and markers flanking the ARPKD candidate region.

FIG. 2 (top) is a map of the ARPKD candidate region on human chromosome 6 showing the positions of known genes and the PKHD1 gene (boxes), microsatellite markers (above the line), and markers (arrows) used to localize the rat Pkhd1 gene. FIG. 2 (middle) shows the intron/exon structure of the PKHD1 gene. FIG. 2 (bottom) depicts the PKHD1 transcript (coding regions are black, untranslated regions are unshaded). RT-PCR products and cDNAs used for cloning are indicated by the lines below the transcript, and the position of mutations are shown above the transcript.

FIG. 3 is the nucleotide sequence of the wild-type human PKHD1 coding region (SEQ ID NO:1).

FIG. 4 is the amino acid sequence of the wild-type human fibrocystin polypeptide (SEQ ID NO:2).

FIG. 5 is a series of aberrant DHPLC profiles from segregation analysis of two ARPKD pedigrees, M54 and M57. A heteroduplex with the mutant allele is seen as an aberrant peak(s) eluted before the homoduplex product. The probands and affected siblings are compound heterozygotes, having inherited one mutant allele from each parent.

FIG. 6 is the nucleotide sequence of the rat Pkhd1 transcript (SEQ ID NO:3). Start and stop codons are in bold.

FIG. 7 is the nucleotide sequence of the mouse Pkhd1 transcript (SEQ ID NO:4). Start and stop codons are in bold.

FIG. 8 is an alignment of the amino acid sequence of human fibrocystin (SEQ ID NO:2) with the amino acid sequences of mouse and rat fibrocystin (SEQ ID NO:6 and SEQ ID NO:7, respectively).

FIGS. 9A, 9B, 9C, and 9D are alignments of regions within fibrocystin that demonstrate homology to other proteins. FIG. 9A shows the alignment of the 7 TIG domains of human fibrocystin (TIG 1 to TIG 7; SEQ ID NO:8 to SEQ ID NO:14, respectively) with the 5 TIG-like domains of human fibrocystin (TIGL-A to TIGL-E; SEQ ID NO:15 to SEQ ID NO:19, respectively) and TIG domains from D86 (SEQ ID NO:20), hepatocyte growth factor receptor (HGFR; SEQ ID NO:21), Plexin 1 (SEQ ID NO:22) and macrophage-stimulating protein receptor (Ron; SEQ ID NO:23), as well as a consensus TIG domain (SEQ ID NO:24). Amino acids 31-41 and 87-90 have been removed from the TIG consensus to match the other receptor TIG domains. Capital letters indicate highly conserved amino acid residues, and lower case letters indicate less conserved sites. FIG. 9B shows the alignment of human fibrocystin (SEQ ID NO:25; residues 1 to 1776 of SEQ ID NO:2) with the mouse D86 protein (SEQ ID NO:26). FIG. 9C is an alignment of two regions of human fibrocystin (SEQ ID NO:27 and SEQ ID NO:28; residues 1930 to 2375 and 2882 to 3069, respectively, of SEQ ID NO:2) with TMEM2 (SEQ ID NO:29) and XP051857 (SEQ ID NO:30). FIG. 9D is an alignment of human fibrocystin (SEQ ID NO:31; residues 3461 to 3949 of SEQ ID NO:2) with DKFZp586C1021 (DKFZ; SEQ ID NO:32). Black boxes indicate identity and shaded boxes indicate amino acid similarity.

FIG. 10 is a model of the fibrocystin protein, showing conserved domains and regions of homology with other proteins.

FIGS. 11A, 11B, 11C, and 11D are examples of mutation analysis of ARPKD families. FIG. 11A contains data from pedigree PRR-9 segregating the mutations 6384delT and I222V. FIG. 11B shows that mutations I222V and 383delC segregate in family PRR-15. FIG. 11C shows that proband PRR-17 is a compound heterozygote for two truncating mutations, 3761CC→G and 9689deLA. FIG. 11D shows that mutations T36M and IVS43+4A→T segregate in pedigree OV-7 with two affected children, 2537 (brother 2) and 2538 (brother 3).

FIG. 12 is a diagram depicting the open reading frame of PKHD1 and the location of mutations described herein. Mutations detected at least twice in this study are in bold. Missense mutations are shown above the diagram of the open reading frame. a, insertion/deletion mutations; b, splicing mutations; and c, nonsense mutations. Missense mutations that may lead to aberrant splicing are marked with asterisks.

FIG. 13 contains the nucleotide sequences of introns 1, 3, 7, 14, 22, 23, 28, 32, 33, 43, 53, and 61 (SEQ ID NOS:5, 209, 210, 211, 212, 213, 214, 215, 216, 217, 218, and 219, respectively).

### DETAILED DESCRIPTION

As described herein, the human PKHD1 gene and the rat and mouse Pkhd1 genes are associated with ARPKD, and are referred to as “ARPKD genes.” The term “associated with ARPKD,” with respect to a particular gene, refers to a gene which, when mutated in both alleles, leads to a phenotype of ARPKD. The mutation most likely leads to loss of production of functional protein, but may increase or decrease production of the encoded protein, or cause production of a protein with a sequence, structure, and/or function that differs from the wild-type protein. As described herein, the association of PKHD1 and Pkhd1 with ARPKD is indicated by the discovery that certain sequence variants within the genes are correlated with the presence of ARPKD.

1. Isolated ARPKD Nucleic Acid Molecules

As used herein, the term “nucleic acid” refers to both RNA and DNA, including cDNA, genomic DNA, and synthetic (e.g., chemically synthesized) DNA. The nucleic acid can be double-stranded or single-stranded (i.e., a sense or an antisense single strand). As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that flank an ARPKD gene). The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated ARPKD nucleic acid molecules are at least 10 nucleotides in length (e.g., 10, 20, 50, 100, 200, 300, 400, 500, 1000, or more nucleotides in length). In some embodiments, isolated ARPKD nucleic acid molecules are between 150 and 370 nucleotides in length (e.g., 150, 175, 200, 225, 250, 275, 300, 325, 350, or 370 nucleotides in length). As described in the Examples (below), the full-length human ARPKD transcript contains 67 exons and is 16,235 nucleotides in length, with a coding region that is 12,222 nucleotides in length (SEQ ID NO:1). The full-length rat transcript is 13,971 nucleotides in length (SEQ ID NO:3), with a coding region that is 12,153 nucleotides in length (nucleotides 206 to 12,358 of SEQ ID NO:3). The full-length mouse transcript is 12,819 nucleotides in length (SEQ ID NO:4), with a coding region that is 12,177 nucleotides in length (nucleotides 200 to 12,376 of SEQ ID NO:4). An ARPKD nucleic acid molecule therefore is not required to contain all of the coding region listed in SEQ ID NOS:1, 3, or 4, or all of the exons; in fact, an ARPKD nucleic acid molecule can contain as little as a single exon (as listed in Table 3, for example) or a portion of a single exon (e.g., 10 nucleotides from a single exon). In some embodiments, the ARPKD transcript is alternatively spliced, which can remove a portion of an exon, a single exon, or multiple exons from the transcript. See Table 8 for examples of splice forms of PKHD1. Alternatively spliced forms of PKHD1 thus can be less than 12,222 nucleotides in length (e.g., 12,281, 11,946, 12,026, 11,958, 11,261, 11,580, 12,119, 12,065, 12,009, 12,146, 12,175, or 11,204 nucleotides in length). Nucleic acid molecules that are less than full-length can be useful, for example, for diagnostic purposes.

Nucleic acid molecules of the invention may have sequences identical to those found in SEQ ID NO:1, SEQ ID NO:3, or SEQ ID NO:4. Nucleic acid molecules also can have sequences identical to those found in the complement of SEQ ID NO:1, SEQ ID NO:3, or SEQ ID NO:4. Alternatively, the sequence of an ARPKD nucleic acid molecule may contain one or more variants relative to the sequences set forth in SEQ ID NO:1, SEQ ID NO:3, or SEQ ID NO:4, or the complement of SEQ ID NO:1, SEQ ID NO:3, or SEQ ID NO:4. As used herein, a “sequence variant” refers to any mutation that results in a difference between nucleotides at one or more positions within the nucleic acid sequence of a particular nucleic acid molecule and the nucleotides at the same positions within the corresponding wild-type sequence set forth in SEQ ID NO:1, SEQ ID NO:3, and SEQ ID NO:4. Nucleotides are referred to herein by the standard one-letter designation (A, C, G, or T). Sequence variants can be found in coding and non-coding regions, including exons, introns, promoters, and untranslated sequences. The presence of one or more sequence variants in the ARPKD nucleic acid sequence of a subject can be detected as set forth below in subsection 7.

Sequence variants can be, for example, deletions, insertions, or substitutions at one or more nucleotide positions (e.g., 1, 2, 3, 10, or more than 10 positions), provided that the nucleic acid is at least 80% identical (e.g., 80%, 85%, 90%, 95%, or 99% identical) over its length to the corresponding region of the wild-type sequences set forth in SEQ ID NO:1, SEQ ID NO:3, or SEQ ID NO:4. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained from Fish & Richardson's web site (World Wide Web at fr.com/blast) or the U.S. government's National Center for Biotechnology Information web site (World Wide Web at ncbi.nlm.nih.gov). Instructions explaining how to use the Bl2seq program can be found in the readme file accompanying BLASTZ.

Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: =i is set to a file containing the first nucleic acid sequence to be compared (e.g., C:\seq1.txt); −j is set to a file containing the second nucleic acid sequence to be compared (e.g., C:\seq2.txt); −p is set to blastn; −o is set to any desired file name (e.g., C:\output.txt); −q is set to −1; −r is set to 2; and all other options are left at their default setting. The following command will generate an output file containing a comparison between two sequences: C:\Bl2seq −i c:\seq1.txt −j c:\seq2.txt −p blastn −o c:\output.txt −q −1 −r 2. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides from the target sequence presented in alignment with sequence from the identified sequence starting with any matched position and ending with any other matched position. A matched position is any position where an identical nucleotide is presented in both the target and identified sequence. Gaps presented in the target sequence are not counted since gaps are not nucleotides. Likewise, gaps presented in the identified sequence are not counted since target sequence nucleotides are counted, not nucleotides from the identified sequence.

The percent identity over a particular length is determined by counting the number of matched positions over that length and dividing that number by the length followed by multiplying the resulting value by 100. For example, if (1) a 1000 nucleotide target sequence is compared to the sequence set forth in SEQ ID NO:1, (2) the Bl2seq program presents 200 nucleotides from the target sequence aligned with a region of the sequence set forth in SEQ ID NO:1 where the first and last nucleotides of that 200 nucleotide region are matches, and (3) the number of matches over those 200 aligned nucleotides is 180, then the 1000 nucleotide target sequence contains a length of 200 and a percent identity over that length of 90 (i.e., 180÷200×100=90).

It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

Sequence variants that are deletions or insertions can create frame-shifts within the coding region that alter the amino acid sequence of the encoded polypeptide, and thus can affect its structure and function. Isolated nucleic acids can contain, by way of example and not limitation, a deletion of the nucleotide at position 383, 1529, 6383, 9689, 10364, 10637, or 10856 of SEQ ID NO:1, a deletion of the nucleotides at position 1624 through position 1627 of SEQ ID NO:1, or an insertion at nucleotide position 5895 or 5896 of SEQ ID NO:1.

Substitutions include silent mutations that do not affect the amino acid sequence of the encoded polypeptide, missense mutations that alter the amino acid sequence of the encoded polypeptide, and nonsense mutations that prematurely terminate and therefore truncate the encoded polypeptide. Non-limiting examples of silent mutations are included in Tables 7 and 12, below (e.g., T substituted for C at position 214 of SEQ ID NO:1, T substituted for C at position 234 of SEQ ID NO:1, C substituted for T at position 1587 of SEQ ID NO:1, C substituted for A at position 2046 of SEQ ID NO:1, C substituted for T at position 3537 of SEQ ID NO:1, and G substituted for A at position 4920 of SEQ ID NO:1). Non-limiting examples of missense mutations are included in Tables 6, 7, 10, and 12 below (e.g., A substituted for G at position 73 of SEQ ID NO:1, G substituted for A at position 5513 of SEQ ID NO:1, A substituted for T at position 6992 of SEQ ID NO:1, G substituted for A at position 5984 of SEQ ID NO:1, A or T substituted for C at position 8606 of SEQ ID NO:1, and C substituted for G at position 10585 of SEQ ID NO:1). Non-limiting examples of nonsense mutations are included in Table 6, below (e.g., T substituted for C at position 10174 of SEQ ID NO:1, T substituted for C at position 8011 of SEQ ID NO:1, and A substituted for G at position 11612 of SEQ ID NO:1).

Deletion, insertion, and substitution sequence variants can create or destroy splice sites and thus alter the splicing of an ARPKD transcript, such that the encoded polypeptide contains a deletion or insertion relative to the polypeptide encoded by the corresponding wild-type sequences set forth in SEQ ID NO:1, SEQ ID NO:3, or SEQ ID NO:4. Sequence variants that affect splice sites of ARPKD nucleic acid molecules can result in ARPKD polypeptides that lack the amino acids encoded by, for example, exon 27, exon 30, exons 27 and 30, exons 30 and 31, exon 32, exon 36, exon 37, exon 38, exon 43, or exon 61, or portions thereof. For example, a T substituted for an A at the second position within intron 35 of the rat Pkhd1 gene results in skipping of exon 36.

Polymorphisms in introns are numbered either as positive numbers relative to the guanine in the splice donor site (GT, guanine is +1), or as negative numbers relative to the guanine in the splice acceptor site (AG, guanine is −1). In the PKHD1 gene, for example, G can be substituted for T at position −9 relative to the splice acceptor site of intron 33 (SEQ ID NO:216), T can be substituted for A at position +4 relative to the splice donor site of intron 43 (SEQ ID NO:217), T can be substituted for C at position −47 relative to the splice acceptor site of intron 1 (SEQ ID NO:5), or the nucleotide at position +42 relative to the splice donor site of intron 32 can be deleted (SEQ ID NO:215). Other examples of sequence variants within intervening sequences can be found in Tables 10 and 12.

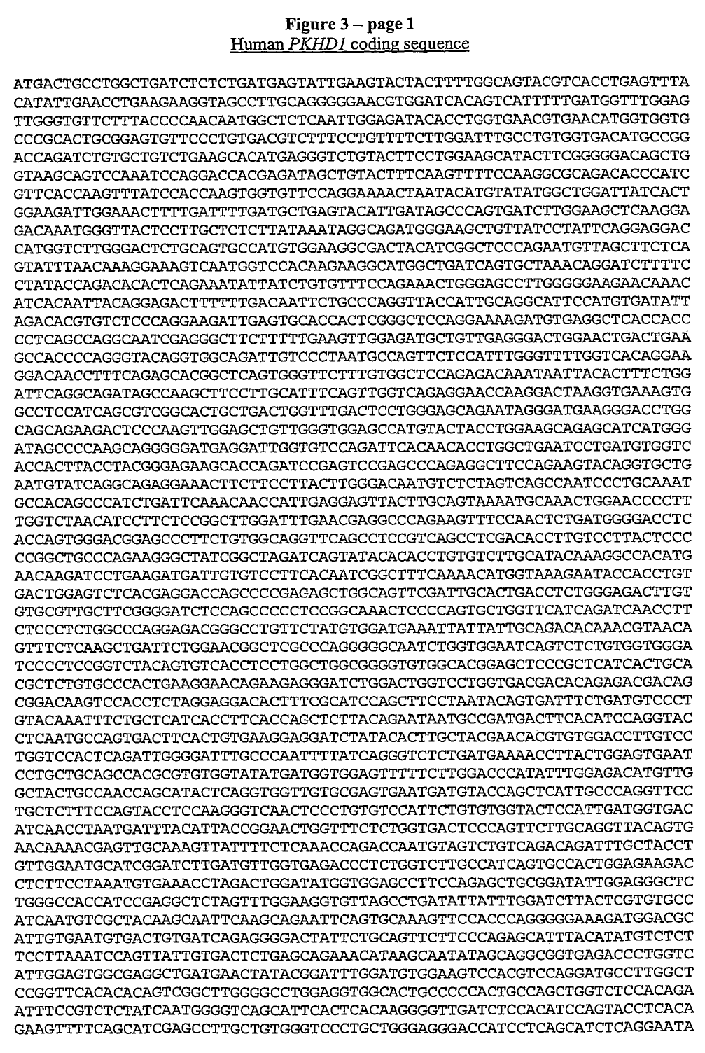

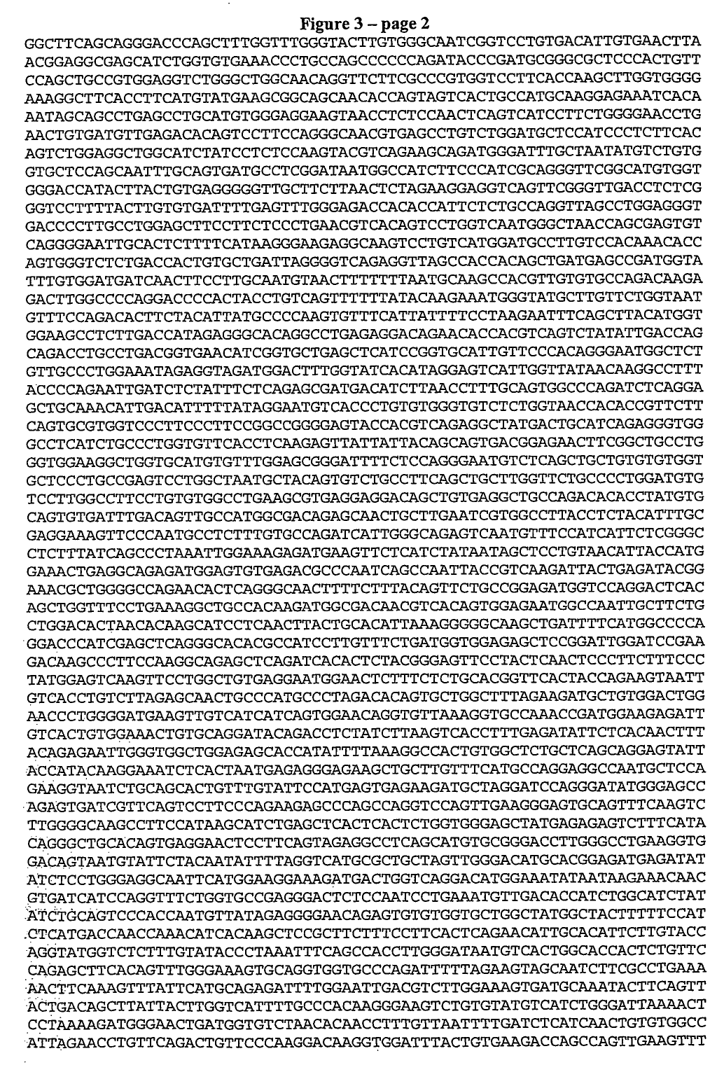

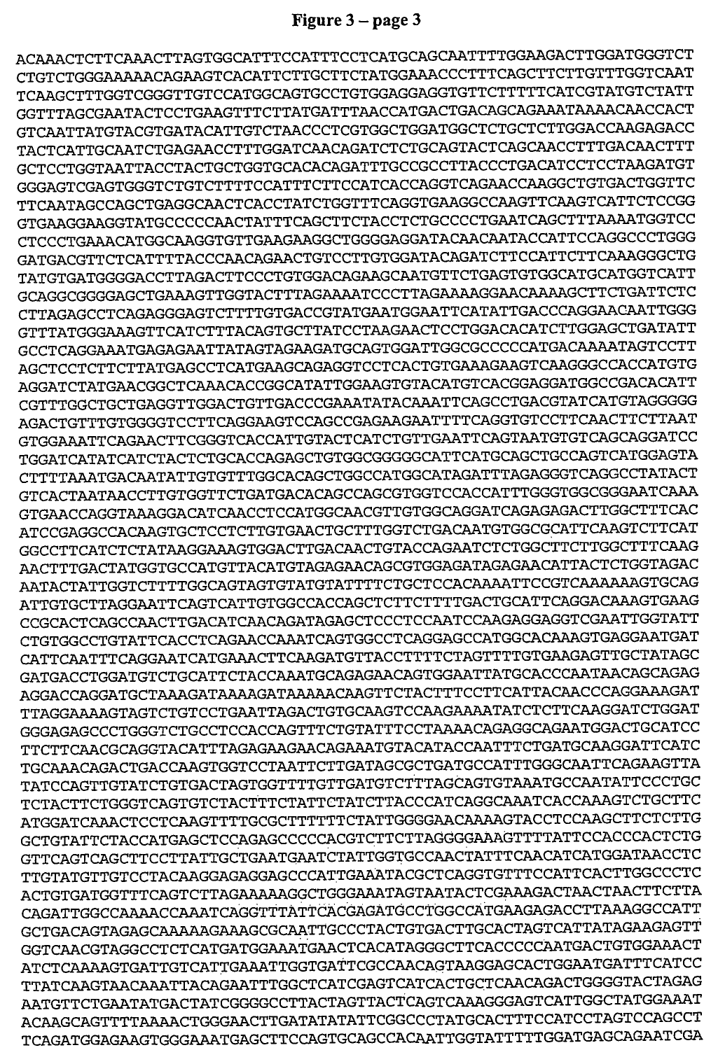

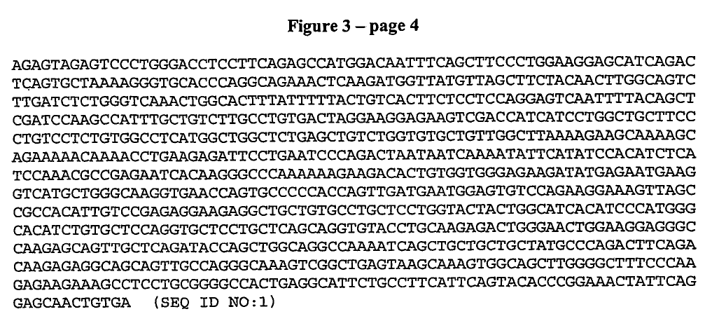

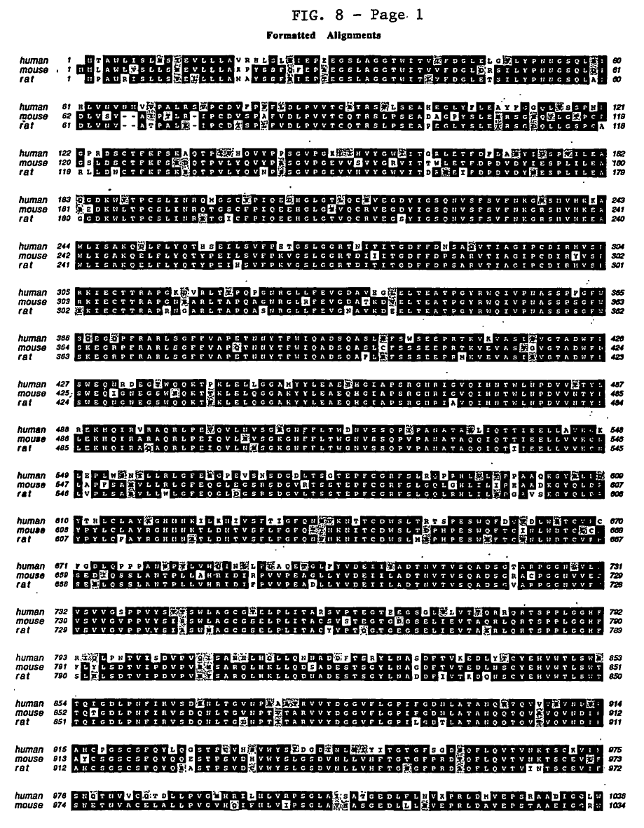

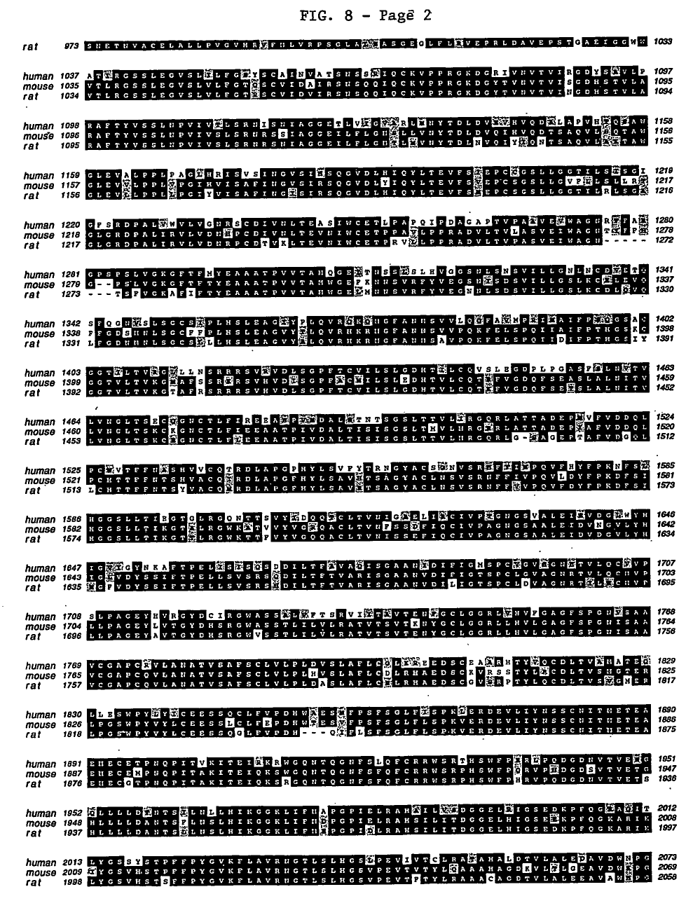

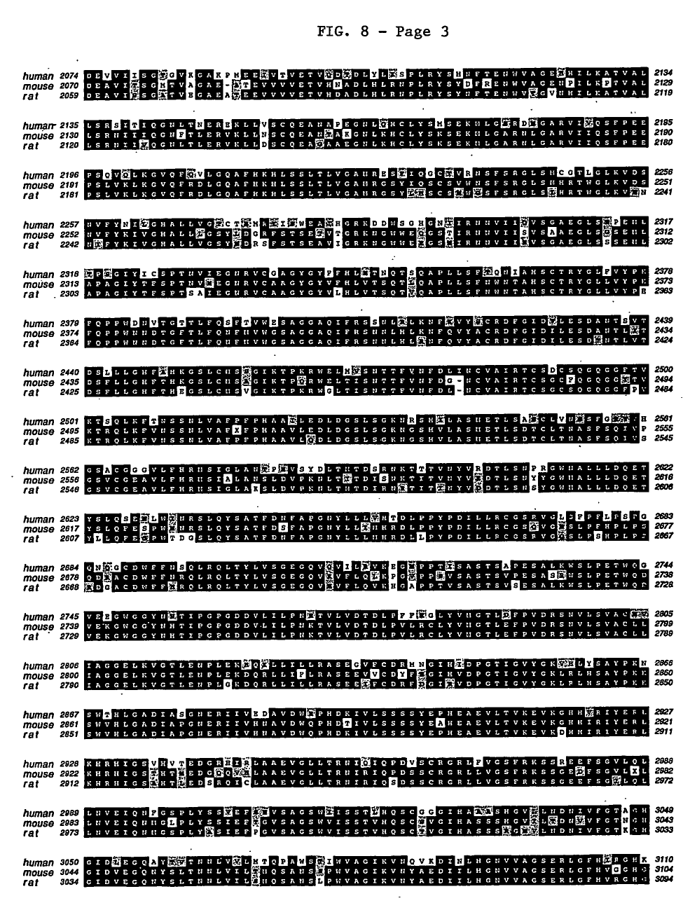

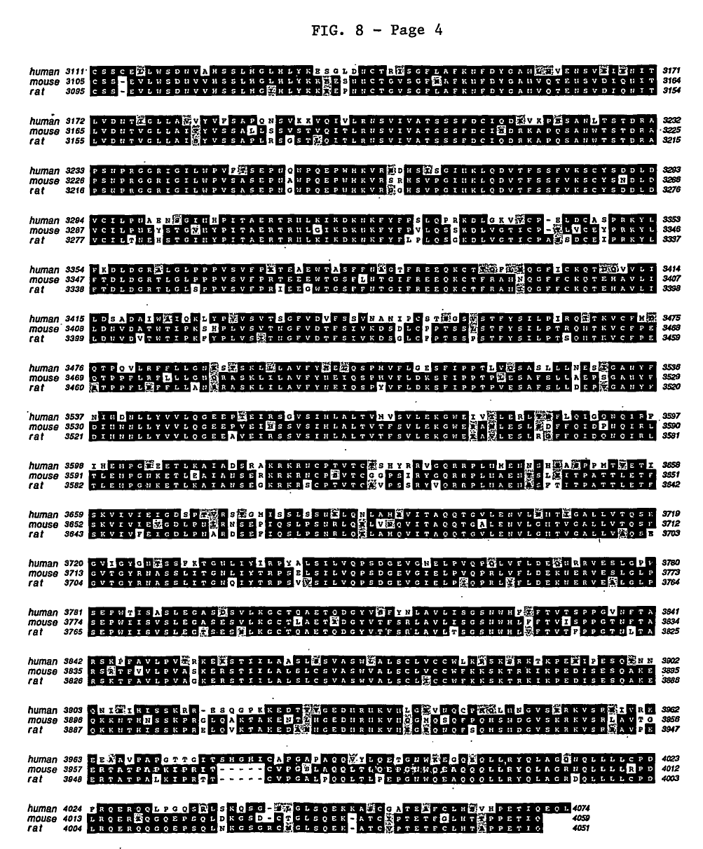

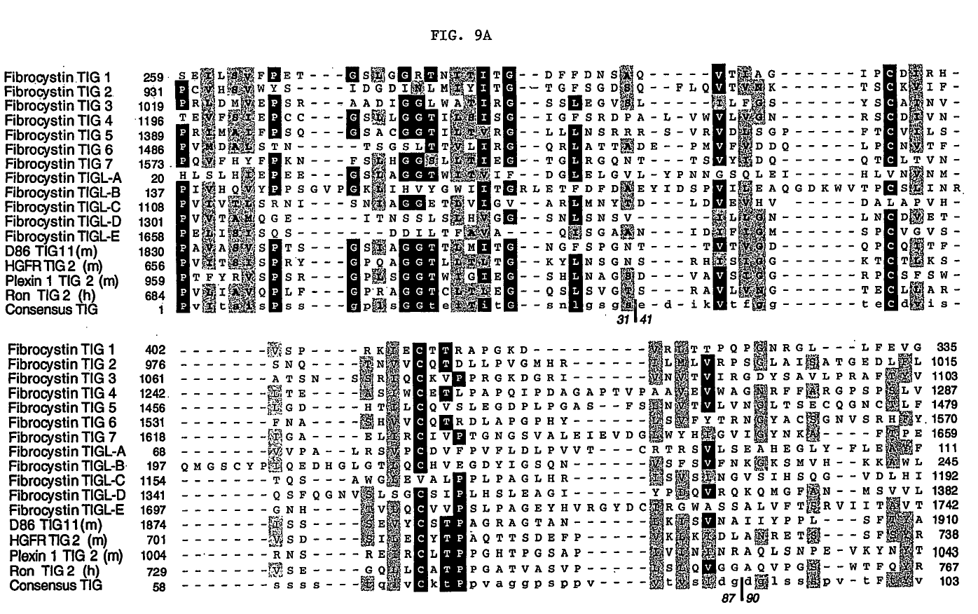

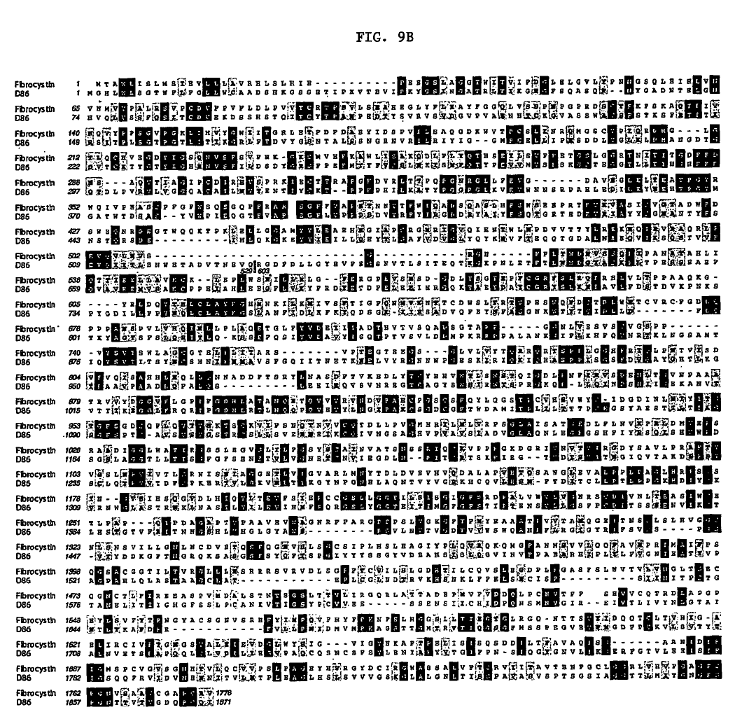

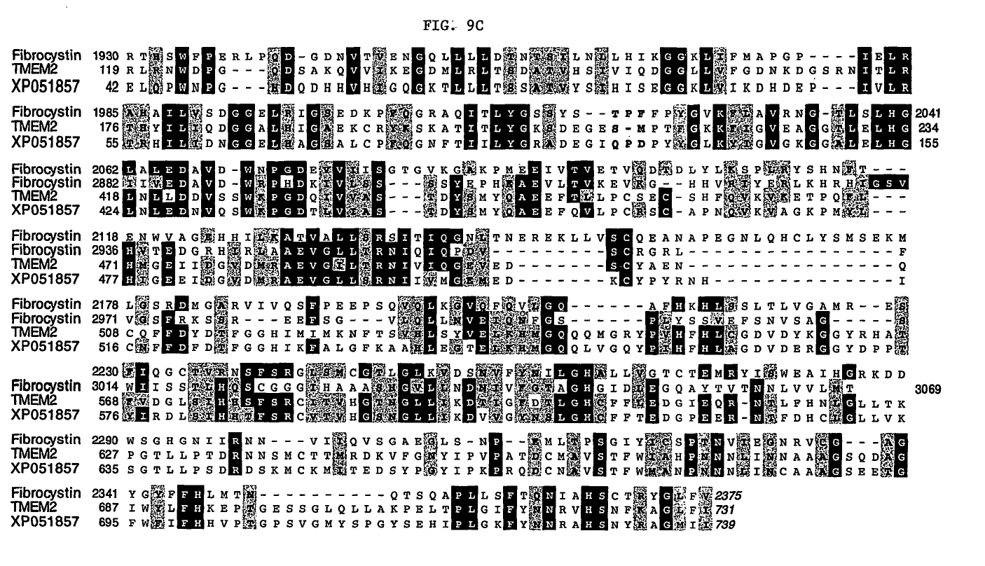

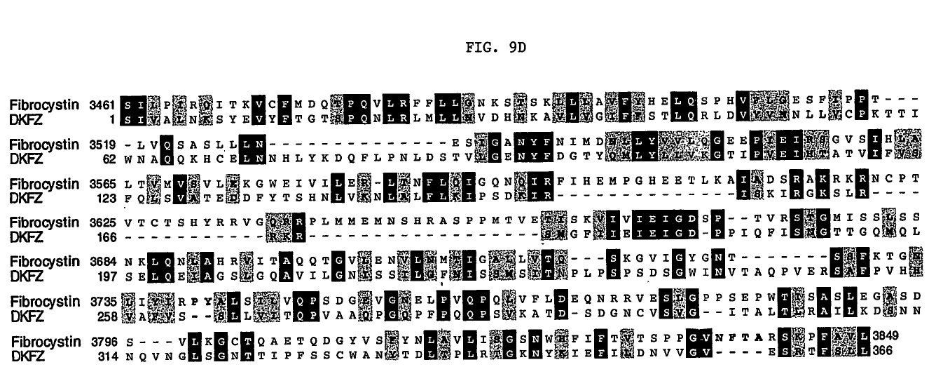

Certain sequence variants described herein are associated with ARPKD. Such sequence variants typically result in a change in the encoded polypeptide that can have a dramatic effect on the function of the polypeptide. These changes can include, for example, a truncation, a frame-shifting alteration, or a substitution at a highly conserved position. Conserved positions can be identified by inspection of a nucleotide or amino acid sequence alignment showing related nucleic acids or polypeptides from different species (e.g., the sequence alignments shown in FIGS. 8, 9A, 9B, 9C, and 9D). With respect to SEQ ID NO:1, sequence variants that can be associated with APRKD include, for example, deletion of the nucleotides at positions 1624 through 1627 of SEQ ID NO:1, an A at position 6992 of SEQ ID NO:1, a T at position 10174 of SEQ ID NO:1, a G at position 664 of SEQ ID NO:1, an A inserted at position 5896 of SEQ ID NO:1, a G at position 4220 of SEQ ID NO:1, a T at position 8011 of SEQ ID NO:1, a C at position 10658 of SEQ ID NO:1, an A at position 11612 of SEQ ID NO:1, a G at position 5984 of SEQ ID NO:1, a deletion at position 10637 of SEQ ID NO:1, a C at position 8870 of SEQ ID NO:1, a T at position 4991 of SEQ ID NO:1, a T at position 9053 of SEQ ID NO:1, a G at position 3747 of SEQ ID NO:1, a G at position 5750 of SEQ ID NO:1, an A at position 5221 of SEQ ID NO:1, and a T at position 107 of SEQ ID NO:1. Other examples of sequence variants that can be associated with ARPKD include, with respect to SEQ ID NO:1, deletion of the A at position 9689, an A at position 10865, a T at position 50, a T at position 8063, a G at position 10402, a deletion of the G at position 1529, a T at position 657, an A or a T at position 8606, a C at position 10036, a deletion of the C at position 383, a G at position 5513, a deletion of the T at position 6383, a deletion of the A at position 10856, a T at position 10505, a G at position 5825, a T at position 2216, a T at position 2414, a C at position 9530, a C at position 2269, an A at position 5600, a C at position 10585, an A at position 4165, a deletion of the C at position 10364, and a T at position 5498. It should be noted that these cited nucleotide positions are numbered relative to the ATG at the beginning of the coding sequence (see FIG. 3). Other sequence variants that can be associated with ARPKD can be located within intervening sequences. Such variants include, for example, a G at position −9 relative to the splice acceptor site of intron 33 (SEQ ID NO:216), a C at position −2 relative to the splice acceptor site of intron 28 (SEQ ID NO:214), and a T at position +4 relative to the splice donor site of intron 43 (SEQ ID NO:217).

In some ARPKD patients, the same ARPKD-associated sequence variant can be found on both alleles. In other patients, a combination of ARPKD-associated sequence variants can be found on separate alleles of an ARPKD gene. Examples of ARPKD-associated combinations are shown in Tables 6 and 10, and include, without limitation, deletion of the nucleotides at positions 1624 through 1627 and an A at position 6992 of SEQ ID NO:1, a T at position 10174 and a G at position 664 of SEQ ID NO:1, an A inserted at position 5896 and a G at position 4220 of SEQ ID NO:1, a T at position 8011 and a C at position 10658 of SEQ ID NO:1, an A at position 11612 and a G at position 5984 of SEQ ID NO:1, a deletion at position 10637 and a C at position 8870 of SEQ ID NO:1, a T at position 4991 and a T at position 9053 of SEQ ID NO:1, and a G at position 3747 and a G at position 5750 of SEQ ID NO:1. Additional examples of ARPKD-associated combinations of variants include a deletion at position 9689 and a G at position 3761 in combination with a deletion at position 3762 of SEQ ID NO:1, a deletion at position 9689 and an A at position 10865 of SEQ ID NO:1, a deletion at position 9689 and a T at position 50 of SEQ ID NO:1, an A inserted at position 5895, a T at position 8063, and a G at position 10402 of SEQ ID NO:1, a deletion at position 1529, a T at position 657, and an A at position 8606 of SEQ ID NO:1, a G at position 3761 with a deletion at position 3762 and a G at position 664 of SEQ ID NO:1, an insertion at position 5895 and a C at position 10036 of SEQ ID NO:1, a deletion at position 383 and a G at position 5513 of SEQ ID NO:l, a deletion at position 6383 and a G at position 664 of SEQ ID NO:1, a deletion at position 383 and a G at position 664 of SEQ ID NO:1, a deletion at position 10856 of SEQ ID NO:1 and a G at position -9 relative to the splice acceptor site of intron 33 (SEQ ID NO:216), a C at position -2 relative to the splice acceptor site of intron 28 (SEQ ID NO:214) together with a T at position 10505 and an A at position 8606 of SEQ ID NO:1, a T at position 107 of SEQ ID NO:1 and a T at position 4 relative to the splice donor site of intron 43 (SEQ ID NO:217), a G at position 5825, a T at position 8606, and a T at position 2216 of SEQ ID NO:1, a T at position 107, a T at position 2414, and a C at position 9530 of SEQ ID NO:1, and a C at position 2269 and a C at position 9530 of SEQ ID NO:1.

Other sequence variants described herein include polymorphisms that occur within a normal population and typically are not associated with ARPKD. Sequence variants of this type can be, for example, nucleotide substitutions (e.g., silent mutations) that do not alter the amino acid sequence of the encoded ARPKD polypeptide, or alterations that alter the amino acid sequence but that do not affect the overall function of the polypeptide. With respect to SEQ ID NO:1, sequence variants that are polymorphisms can include, for example, an A at position 73 of SEQ ID NO:1, a T at position 234 of SEQ ID NO:1, a C at position 1587 of SEQ ID NO:1, a T at position 2255 of SEQ ID NO:1, a T at position 2278 of SEQ ID NO:1, a C at position 2554 of SEQ ID NO:1, a T at position 2853 of SEQ ID NO:1, a C at position 3756 of SEQ ID NO:1, a T at position 3785 of SEQ ID NO:1, a G at position 4920 of SEQ ID NO:1, an A at position 7587 of SEQ ID NO:1, a G at position 7764 of SEQ ID NO:1, a T at position 8813 of SEQ ID NO:1, an A at position 9237 of SEQ ID NO:1, a T at position 9415 of SEQ ID NO:1, a T at position 10515 of SEQ ID NO:1, a T at position 10521 of SEQ ID NO:1,a C at position 11340 of SEQ ID NO:1, a G at position 11196 of SEQ ID NO:1, an A at position 11878 of SEQ ID NO:1, and a G at position 12143 of SEQ ID NO:1. Other examples of sequence variants that are polymorphisms include, with respect to SEQ ID NO:1, a T at position 214, a C at position 1185, a C at position 2046, a T at position 2196, a G at position 2489, a C at position 3537, a T at position 5125, and a G at position 5608. Still other sequence variants that are polymorphisms can be located within intervening sequences. Such variants include, for example, a T at position −47 relative to the splice acceptor site of intron 1 (SEQ ID NO:5), an A inserted at the splice donor site of intron 3 (SEQ ID NO:209), a C at position +19 relative to the splice donor site of intron 7 (SEQ ID NO:210), a T at position +23 relative to the splice donor site of intron 14 (SEQ ID NO:211), a G at position +13 relative to the splice donor site of intron 22 (SEQ ID NO:212), a T at position +50 relative to the splice donor site of intron 23 (SEQ ID NO:213), a G at position +53 relative to the splice donor site of intron 23 (SEQ ID NO:213), a deletion of the nucleotides at positions +42 through +45 relative to the splice donor site of intron 32 (SEQ ID NO:215), a G at position −32 relative to the splice acceptor site of intron 53 (SEQ ID NO:218), and a G at position +9 relative to the splice donor site of intron 61 (SEQ ID NO:219).

2. Production of Isolated ARPKD Nucleic Acid Molecules

Isolated nucleic acid molecules of the invention can be produced by standard techniques, including, without limitation, common molecular cloning and chemical nucleic acid synthesis techniques. For example, polymerase chain reaction (PCR) techniques can be used to obtain an isolated ARPKD nucleic acid molecule. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. General PCR techniques are described, for example in PCR Primer: A Laboratory Manual, Ed. by Dieffenbach, C. and Dveksler, G, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication or nucleic acid sequence-based amplification also can be used to obtain isolated nucleic acids. See, for example, Lewis (1992) Genetic Engineering News 12(9):1; Guatelli et al. (1990) Proc. Natl. Acad. Sci. USA 87:1874-1878; and Weiss (1991) Science 254:1292-1293.

In one embodiment, a primer is a single-stranded or double-stranded oligonucleotide that typically is 10 to 50 nucleotides in length, and when combined with mammalian genomic DNA and subjected to PCR conditions, is capable of being extended to produce a nucleic acid product corresponding to a region of an ARPKD nucleic acid molecule. Typically, an ARPKD PCR product is 30 to 1650 nucleotides in length (e.g., 30, 35, 50, 100, 250, 500, 1000, 1500, or 1650 nucleotides in length). Primers such as those listed in Table 5 are particularly useful for producing ARPKD PCR products. Specific regions of mammalian DNA can be amplified (i.e., replicated such that multiple exact copies are produced) when a pair of oligonucleotide primers is used in the same PCR reaction, wherein one primer contains a nucleotide sequence from the coding strand of an ARPKD nucleic acid and the other primer contains a nucleotide sequence from the non-coding strand of an ARPKD nucleic acid. The “coding strand” of a nucleic acid is the nontranscribed strand, which has the same nucleotide sequence as the specified RNA transcript (with the exception that the RNA transcript contains uracil in place of thymidine residues), while the “non-coding strand” of a nucleic acid is the strand that serves as the template for transcription.

A single PCR reaction mixture may contain one pair of oligonucleotide primers. Alternatively, a single reaction mixture may contain a plurality of oligonucleotide primer pairs, in which case multiple PCR products can be generated. Each primer pair can amplify, for example, one exon or a portion of one exon. Intron sequences also can be amplified.

Oligonucleotide primers can be incorporated into compositions. Typically, a composition of the invention will contain a first oligonucleotide primer and a second oligonucleotide primer, each 10 to 50 nucleotides in length, which can be combined with genomic DNA from a mammal and subjected to PCR conditions as set out below, to produce a nucleic acid product that corresponds to a region of an ARPKD nucleic acid molecule. A composition also may contain buffers and other reagents necessary for PCR (e.g., DNA polymerase or nucleotides). Furthermore, a composition may contain one or more additional pairs of oligonucleotide primers (e.g., 3, 13, 16, or 23 primer pairs), such that multiple nucleic acid products can be generated.

Specific PCR conditions typically are defined by the concentration of salts (e.g., MgCl₂) in the reaction buffer, and by the temperatures utilized for melting, annealing, and extension. Specific concentrations or amounts of primers, templates, deoxynucleotides (dNTPs), and DNA polymerase also may be set out. For example, PCR conditions with a buffer containing 2.5 mM MgCl₂, and melting, annealing, and extension temperatures of 94° C., 44-65° C., and 72° C., respectively, are particularly useful. Under such conditions, a PCR sample can include, for example, 60 ng genomic DNA, 8 mM each primer, 200 pM dNTPs, 1 U DNA polymerase (e.g., AmpliTaq Gold), and the appropriate amount of buffer as specified by the manufacturer of the polymerase (e.g., 1× AmpliTaq Gold buffer). Denaturation, annealing, and extension each may be carried out for 30 seconds per cycle, with a total of 25 to 35 cycles, for example. An initial denaturation step (e.g., 94° C. for 2 minutes) and a final elongation step (e.g., 72° C. for 10 minutes) also may be useful.

Isolated nucleic acids of the invention also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Isolated nucleic acids of the invention also can be obtained by mutagenesis. For example, the reference sequence depicted in FIG. 3 can be mutated using standard techniques including oligonucleotide-directed mutagenesis and site-directed mutagenesis through PCR. See, Short Protocols in Molecular Biology, Chapter 8, Green Publishing Associates and John Wiley & Sons, Edited by Ausubel et al., 1992. Examples of positions that can be modified are described above and in Tables 6, 7, 10, and 12, as well as in the alignments of FIGS. 8, 9A, 9B, 9C, and 9D.

3. Vectors and Host Cells

The invention also provides vectors containing nucleic acids such as those described above. As used herein, a “vector” is a replicon, such as a plasmid, phage, or cosmid, into which another DNA segment may be inserted so as to bring about the replication of the inserted segment. The vectors of the invention can be expression vectors. An “expression vector” is a vector that includes one or more expression control sequences, and an “expression control sequence” is a DNA sequence that controls and regulates the transcription and/or translation of another DNA sequence.

In the expression vectors of the invention, the nucleic acid is operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

An expression vector can include a tag sequence designed to facilitate subsequent manipulation of the expressed nucleic acid sequence (e.g., purification or localization). Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide including at either the carboxyl or amino terminus.

The invention also provides host cells containing vectors of the invention. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Suitable methods for transforming and transfecting host cells are found in Sambrook et al., Molecular Cloning: A Laboratory Manual (2^{nd} edition), Cold Spring Harbor Laboratory, New York (1989), and reagents for transformation and/or transfection are commercially available (e.g., Lipofectin (Invitrogen/Life Technologies); Fugene (Roche, Indianapolis, Ind.); and SuperFect (Qiagen, Valencia, Calif.)).

4. Fibrocystin Polypeptides

The invention provides purified fibrocystin polypeptides that are encoded by the ARPKD nucleic acid molecules of the invention. A “polypeptide” refers to a chain of at least 10 amino acid residues (e.g., 10, 20, 50, 75, 100, 200, or more than 200 residues), regardless of post-translational modification (e.g., phosphorylation or glycosylation). Typically, a fibrocystin polypeptide of the invention is capable of eliciting a fibrocystin-specific antibody response (i.e., is able to act as an immunogen that induces the production of antibodies capable of specific binding to fibrocystin).

The full-length human, rat, and mouse fibrocystin polypeptides are 4074, 4051, and 4059 amino acids in length, respectively. The amino acid sequences of the wild type human, rat, and mouse fibrocystin polypeptides are set forth in SEQ ID NOS:2, 6, and 7, respectively. A fibrocystin polypeptide may have an amino acid sequence that is identical to that of SEQ ID NO:2, SEQ ID NO:6, or SEQ ID NO:7. Alternatively, a fibrocystin polypeptide can include an amino acid sequence variant. As used herein, an amino acid sequence variant refers to a deletion, insertion, or substitution at one or more amino acid positions (e.g., 1, 2, 3, 10, or more than 10 positions), provided that the polypeptide has an amino acid sequence that is at least 80% identical (e.g., 80%, 85%, 90%, 95%, or 99% identical) over its length to the corresponding region of the sequences set forth in SEQ ID NO:2, SEQ ID NO:6, and SEQ ID NO:7.

Percent sequence identity is calculated by determining the number of matched positions in aligned amino acid sequences, dividing the number of matched positions by the total number of aligned amino acids, and multiplying by 100. The percent identity between amino acid sequences therefore is calculated in a manner analogous to the method for calculating the identity between nucleic acid sequences, using the Bl2seq program from the stand-alone version of BLASTZ containing BLASTN version 2.0.14 and BLASTP version 2.0.14; see subsection 1, above. A matched position refers to a position in which identical residues occur at the same position in aligned amino acid sequences. To compare two amino acid sequences, the options of Bl2seq are set as follows: −i is set to a file containing the first amino acid sequence to be compared (e.g., C:\seq1.txt); −j is set to a file containing the second amino acid sequence to be compared (e.g., C:\seq2.txt); −p is set to blastp; −o is set to any desired file name (e.g., C:\output.txt); and all other options are left at their default setting. The following command will generate an output file containing a comparison between two amino acid sequences: C:\Bl2seq −i c:\seq1.txt −j c:\seq2.txt −p blastp −o c:\output.txt. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive amino acid residues from the target sequence presented in alignment with sequence from the identified sequence starting with any matched position and ending with any other matched position. A matched position is any position where an identical amino acid residue is presented in both the target and identified sequence. Gaps presented in the target sequence are not counted since gaps are not amino acid residues. Likewise, gaps presented in the identified sequence are not counted since target sequence amino acid residues are counted, not amino acid residues from the identified sequence.

The percent identity over a particular length is determined by counting the number of matched positions over that length and dividing that number by the length followed by multiplying the resulting value by 100. For example, if (1) a 1000 amino acid target sequence is compared to the sequence set forth in SEQ ID NO:2, (2) the Bl2seq program presents 200 amino acids from the target sequence aligned with a region of the sequence set forth in SEQ ID NO:2 where the first and last amino acids of that 200 amino acid region are matches, and (3) the number of matches over those 200 aligned amino acids is 180, then the 1000 amino acid target sequence contains a length of 200 and a percent identity over that length of 90 (i.e. 180÷200×100=90). As described for aligned nucleic acids in subsection 1, different regions within a single amino acid target sequence that aligns with an identified sequence can each have their own percent identity. It also is noted that the percent identity value is rounded to the nearest tenth, and the length value will always be an integer.

The deletion of amino acids from a fibrocystin polypeptide or the insertion of amino acids into a fibrocystin polypeptide can significantly affect the structure of the polypeptide. A deletion can result in a fibrocystin polypeptide that is truncated, for example, after the amino acid at position 3392, 2670, 3870, 3229, 509, 127, 2127, or 3618 of SEQ ID NO:2. Amino acids also may be deleted from a fibrocystin polypeptide as a result of altered splicing (see subsection 1, above).

Amino acid substitutions may be conservative or non-conservative. Conservative amino acid substitutions replace an amino acid with an amino acid of the same class, whereas non-conservative amino acid substitutions replace an amino acid with an amino acid of a different class. Conservative amino acid substitutions typically have little effect on the structure or function of a polypeptide. Examples of conservative substitutions include amino acid substitutions within the following groups: glycine and alanine; valine, isoleucine, and leucine; aspartic acid and glutamic acid; asparagine, glutamine, serine, and threonine; lysine, histidine, and arginine; and phenylalanine and tyrosine. Conservative substitutions within a fibrocystin polypeptide can include, for example, Val substituted for Be at amino acid position 25 of SEQ ID NO:2, Val substituted for Ala at amino acid position 1262 of SEQ ID NO:2, Ile substituted for Val at amino acid position 3960 of SEQ ID NO:2, Val substituted for Ile at amino acid position 3468 of SEQ ID NO:2, Leu substituted for Ile at amino acid position 757 of SEQ ID NO:2, Phe substituted for Leu at amino acid position 1709 of SEQ ID NO:2, and Val substituted for Leu at amino acid position 1870 of SEQ ID NO:2. Other non-limiting examples are provided in Tables 6, 7, 10, and 12, below.

Non-conservative substitutions may result in a substantial change in the hydrophobicity of the polypeptide or in the bulk of a residue side chain. In addition, non-conservative substitutions may make a substantial change in the charge of the polypeptide, such as reducing electropositive charges or introducing electronegative charages. Examples of non-conservative substitutions include a basic amino acid for a non-polar amino acid, or a polar amino acid for an acidic amino acid. Non-conservative substitutions within a fibrocystin polypeptide can include, for example, Arg substituted for Trp at amino acid position 852 of SEQ ID NO:2, Lys substituted for Ile at amino acid position 2331 of SEQ ID NO:2, Phe substituted for Ser at amino acid position 1664 of SEQ ID NO:2, Phe substituted for Cys at position 2688 of SEQ ID NO:2, Val substituted for Glu at amino acid position 3502 of SEQ ID NO:2, and Asn substituted for Ser at amino acid position 1867 of SEQ ID NO:2. Other non-limiting examples are provided in Tables 6, 7, 10, and 12, below.

The term “purified” as used herein with reference to a polypeptide refers to a polypeptide that either has no naturally occurring counterpart (e.g., a peptidornimetic), has been chemically synthesized and is thus uncontaminated by other polypeptides, or has been separated or purified from other cellular components by which it is naturally accompanied (e.g., other cellular proteins, polynucleotides, or cellular components). Typically, the polypeptide is considered “purified” when it is at least 70% (e.g., 70%, 80%, 90%, 95%, or 99%), by dry weight, free from the proteins and naturally occurring organic molecules with which it naturally associates.

Fibrocystin polypeptides typically contain multiple functional domains (e.g., two or more regions that are responsible for a specific function of the polypeptide.) A fibrocystin polypeptide may contain one or more transmembrane (TM) domains, such that part of the polypeptide is cytoplasmic and part is extracellular. A TM domain can be located, for example, between amino acid residues 3859 and 3881 of SEQ ID NO:2, such that the full length fibrocystin polypeptide has a large N-terminal extracellular region and a 192 amino acid C-terminal cytoplasmic region. In order to facilitate insertion of the polypeptide into the cellular membrane, a fibrocystin polypeptide also may include a hydrophobic signal peptide (e.g., the 19 amino acid residues at the N-terminus). Additionally, a fibrocystin polypeptide can contain one or more TIG/IPT domains (Transcription-associated ImmunoGlobulin domain/Immunoglobulin-like fold shared by Plexins and Transcription factors; referred to hereafter as TIG domains), similar to those found in the hepatocyte growth factor receptor, plexins, and the macrophage-stimulating protein receptor. TIG domains can be located anywhere within the polypeptide, although localization within the N-terminal 40% of a fibrocystin polypeptide is particularly comrnon. Furthermore, a fibrocystin polypeptide can contain one or more sites for N-glycosylation (e.g., 64 N-glycosylation sites in the N-terminal region). A fibrocystin polypeptide also may contain sites (e.g., in the C-terminal tail) for phosphorylation by protein kinase A (e.g., amino acid residue 3956 of SEQ ID NO:2) and/or protein kinase C (e.g., amino acid residues 3887, 3910, and 3951 of SEQ ID NO:2).

5. Production of Fibrocystin Polypeptides

Fibrocystin polypeptides can be produced by a number of methods, many of which are well known in the art. By way of example and not limitation, fibrocystin polypeptides can be obtained by extraction from a natural source (e.g., from isolated cells, tissues or bodily fluids), by expression of a recombinant nucleic acid encoding the polypeptide, or by chemical synthesis.

Fibrocystin polypeptides of the invention can be produced by, for example, standard recombinant technology, using expression vectors encoding fibrocystin polypeptides. The resulting fibrocystin polypeptides then can be purified. Expression systems that can be used for small or large scale production of fibrocystin polypeptides include, without limitation, microorganisms such as bacteria (e.g., E. coli and B. subtilis) transformed with recombinant bacteriophage DNA, plasmid DNA, or cosmid DNA expression vectors containing the nucleic acid molecules of the invention; yeast (e.g., S. cerevisiae) transformed with recombinant yeast expression vectors containing the nucleic acid molecules of the invention; insect cell systems infected with recombinant virus expression vectors (e.g., baculovirus) containing the nucleic acid molecules of the invention; plant cell systems infected with recombinant virus expression vectors (e.g., tobacco mosaic virus) or transformed with recombinant plasmid expression vectors (e.g., Ti plasmid) containing the nucleic acid molecules of the invention; or mammalian cell systems (e.g., primary cells or immortalized cell lines such as COS cells, Chinese hamster ovary cells, HeLa cells, human embryonic kidney 293 cells, and 3T3 L1 cells) harboring recombinant expression constructs containing promoters derived from the genome of mammalian cells (e.g., the metallothionein promoter) or from manmnalian viruses (e.g., the adenovirus late promoter and the cytomegalovirus promoter), along with the nucleic acids of the invention.

Suitable methods for purifying the polypeptides of the invention can include, for example, affinity chromatography, immunoprecipitation, size exclusion chromatography, and ion exchange chromatography. See, for example, Flohe et al. (1970) Biochlim. Biophys. Acta. 220:469-476, or Tilgmann et al. (1990) FEBS 264:95-99. The extent of purification can be measured by any appropriate method, including but not limited to: column chromatography, polyacrylamide gel electrophoresis, or high-performance liquid chromatography. Fibrocystin polypeptides also can be “engineered” to contain a tag sequence described herein that allows the polypeptide to be purified (e.g., captured onto an affinity matrix). Immunoaffinity chromatography also can be used to purify fibrocystin polypeptides.

6. Anti-Fibrocystin Antibodies

The invention also provides antibodies having specific binding activity for fibrocystin polypeptides. Such antibodies can be useful for diagnostic purposes (e.g., an antibody that recognizes a specific fibrocystin variant, could be used to diagnose ARPKD). “Antibody” or “antibodies” include intact molecules as well as fragments thereof that are capable of binding to an epitope of a fibrocystin polypeptide. The term “epitope” refers to an antigenic determinant on an antigen to which an antibody binds. Epitopes usually consist of chemically active surface groupings of molecules such as amino acids or sugar side chains, and typically have specific three-dimensional structural characteristics, as well as specific charge characteristics. Epitopes generally have at least five contiguous amino acids. The terms “antibody” and “antibodies” include polyclonal antibodies, monoclonal antibodies, humanized or chimeric antibodies, single chain Fv antibody fragments, Fab fragments, and F(ab)₂ fragments. Polyclonal antibodies are heterogeneous populations of antibody molecules that are specific for a particular antigen, while monoclonal antibodies are homogeneous populations of antibodies to a particular epitope contained within an antigen. Monoclonal antibodies are particularly useful.

In general, a fibrocystin polypeptide is produced as described above, i.e., recombinantly, by chemical synthesis, or by purification of the native protein, and then used to immunize animals. Various host animals including, for example, rabbits, chickens, mice, guinea pigs, and rats, can be immunized by injection of the protein of interest. Depending on the host species, adjuvants can be used to increase the immunological response and include Freund's adjuvant (complete and/or incomplete), mineral gels such as aluminum hydroxide, surface-active substances such as lysolecithin, pluronic polyols, polyanions, peptides, oil emulsions, keyhole limpet hemocyanin, and dinitrophenol. Polyclonal antibodies are contained in the sera of the immunized animals. Monoclonal antibodies can be prepared using standard hybridoma technology. In particular, monoclonal antibodies can be obtained by any technique that provides for the production of antibody molecules by continuous cell lines in culture as described, for example, by Kohler et al. (1975) Nature 256:495-497, the human B-cell hybridoma technique of Kosbor et al. (1983) Immunology Today 4:72, and Cote et al. (1983) Proc. Natl. Acad. Sci. USA 80:2026-2030, and the EBV-hybridoma technique of Cole et al., Monoclonal Antibodies and Cancer Therapy, Alan R. Liss, Inc. pp. 77-96 (1983). Such antibodies can be of any immunoglobulin class including IgM, IgG, IgE, IgA, IgD, and any subclass thereof. The hybridoma producing the monoclonal antibodies of the invention can be cultivated in vitro or in vivo.

A chimeric antibody is a molecule in which different portions are derived from different animal species, such as those having a variable region derived from a mouse monoclonal antibody and a human immunoglobulin constant region. Chimeric antibodies can be produced through standard techniques.

Antibody fragments that have specific binding affinity for fibrocystin polypeptides can be generated by known techniques. Such antibody fragments include, but are not limited to, F(ab′)₂ fragments that can be produced by pepsin digestion of an antibody molecule, and Fab fragments that can be generated by deducing the disulfide bridges of F(ab′)₂ fragments. Alternatively, Fab expression libraries can be constructed. See, for example, Huse et al. (1989) Science 246:1275-1281. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques, such as those disclosed in U.S. Pat. No. 4,946,778.

Once produced, antibodies or fragments thereof can be tested for recognition of a fibrocystin polypeptide by standard immunoassay methods including, for example, enzyme-linked immunosorbent assay (ELISA) or radioimmuno assay (RIA). See, Short Protocols in Molecular Biology eds. Ausubel et al., Green Publishing Associates and John Wiley & Sons (1992). Suitable antibodies typically have equal binding affinities for recombinant and native proteins.

7. Methods for Determining Susceptibility to ARPKD or Diagnosing ARPKD

Methods of the invention can be utilized to determine whether the ARPKD gene of a subject contains a sequence variant or combination of sequence variants (e.g., those identified herein as being associated with ARPKD). Furthermore, methods of the invention can be used to determine whether both ARPKD alleles of a subject contain sequence variants (either the same sequence variant(s) on both alleles or separate sequence variants on each allele), or whether only a single allele of a subject contains sequence variants. The identification of one or more ARPKD-associated sequence variants on each allele can be used to diagnose ARPKD in a patient, typically when known clinical symptoms of ARPKD also are present. The identification of other sequence variants (e.g., sequence variants not known to be associated with ARPKD) on both alleles can be used to support a potential diagnosis of ARPKD. The identification of sequence variants on only one allele can serve as an indicator that a subject is an ARPKD carrier.

Sequence variants within an ARPKD nucleic acid can be detected by a number of methods. Sequence variants can be detected by, for example, sequencing exons, introns, or untranslated sequences, denaturing high performance liquid chromatography (DHPLC; Underhill et al. (1997) Genome Res. 7:996-1005), allele-specific hybridization (Stoneking et al. (1991) Am. J. Hum. Genet. 48:370-382; and Prince et al. (2001) Genome Res. 11(1):152-162), allele-specific restriction digests, mutation specific polymerase chain reactions, single-stranded conformational polymorphism detection (Schafer et al. (1998) Nat. Biotechnol. 15:33-39), infrared matrix-assisted laser desorption/ionization mass spectrometry (WO 99/57318), and combinations of such methods.

Genomic DNA generally is used in the analysis of ARPKD sequence variants. Genomic DNA typically is extracted from a biological sample such as a peripheral blood sample, but can be extracted from other biological samples, including tissues (e.g., mucosal scrapings of the lining of the mouth or from renal or hepatic tissue). Routine methods can be used to extract genomic DNA from a blood or tissue sample, including, for example, phenol extraction. Alternatively, genomic DNA can be extracted with kits such as the QIAamp® Tissue Kit (Qiagen, Chatsworth, Calif.), the Wizard® Genomic DNA purification kit (Promega, Madison, Wis.), or the Puregene DNA Isolation System (Gentra Systems, Inc., Minneapolis, Minn.).

Typically, an amplification step is performed before proceeding with the detection method. For example, exons or introns of the ARPKD gene can be amplified and then directly sequenced. Dye primer sequencing can be used to increase the accuracy of detecting heterozygous samples.

ARPKD sequence variants can be detected by, for example, DHPLC analysis of ARPKD nucleic acid molecules. Genomic DNA can be isolated from a subject (e.g., a human, a mouse, or a rat), and sequences from one or more regions of an ARPKD gene can be amplified (e.g., by PCR) using specific pairs of oligonucleotide primers (e.g., as described above in subsection 2). The primer pairs listed in Table 5, for example can be used to collectively amplify all 66 coding exons of the human PKHD1 gene. After amplification, PCR products can be denatured and reannealed, such that an allele containing an ARPKD sequence variant can reanneal with a wild-type allele to form a heteroduplex (i.e., a double-stranded nucleic acid with a mismatch at one or more positions). The reannealed products then can be subjected to DHPLC, which detects heteroduplexes based on their altered melting temperatures, as compared to homoduplexes that do not contain mismatches. Samples containing heteroduplexes can be sequenced by standard methods to specifically identify the variant nucleotides. Examples of specific sequence variants are provided in Tables 6 and 7, below.

Allele specific hybridization also can be used to detect ARPKD nucleotide sequence variants, including complete haplotypes of a mammal. In practice, samples of DNA or RNA from one or more mammals are amplified using pairs of primers, and the resulting amplification products are immobilized on a substrate (e.g., in discrete regions). Hybridization conditions are selected such that a nucleic acid probe will specifically bind to the sequence of interest, e.g., the ARPKD nucleic acid molecule containing a particular sequence variant. Such hybridizations typically are performed under high stringency, as some nucleotide sequence variants include only a single nucleotide difference. High stringency conditions can include the use of low ionic strength solutions and high temperatures for washing. For example, nucleic acid molecules can be hybridized at 42° C. in 2×SSC (0.3M NaCl/0.03 M sodium citrate/0. 1% sodium dodecyl sulfate (SDS)) and washed in 0.1×SSC (0.015M NaCl/0.0015 M sodium citrate), 0.1% SDS at 65° C. Hybridization conditions can be adjusted to account for unique features of the nucleic acid molecule, including length and sequence composition. Probes can be labeled (e.g., fluorescently) to facilitate detection. In some embodiments, one of the primers used in the amplification reaction is biotinylated (e.g., 5′ end of reverse primer) and the resulting biotinylated amplification product is immobilized on an avidin or streptavidin coated substrate.

Allele-specific restriction digests can be performed in the following manner. For ARPKD nucleotide sequence variants that introduce a restriction site, restriction digest with the particular restriction enzyme can differentiate the alleles. For ARPKD sequence variants that do not alter a common restriction site, mutagenic primers can be designed that introduce a restriction site when the variant allele is present or when the wild type allele is present. A portion of an ARPKD nucleic acid can be amplified using the mutagenic primer and a wild type primer, followed by digestion with the appropriate restriction endonuclease.

Certain sequence variants, such as insertions or deletions of one or more nucleotides, change the size of the DNA fragment encompassing the variant. The insertion or deletion of nucleotides can be assessed by amplifying the region encompassing the sequence variant and determining the size of the amplified products in comparison with size standards. For example, a region of an ARPKD nucleic acid can be amplified using a primer set from either side of the sequence variant. One of the primers is typically labeled, for example, with a fluorescent moiety, to facilitate sizing. The amplified products can be electrophoresed through acrylamide gels with a set of size standards that are labeled with a fluorescent moiety that differs from the primer.

Other methods also can be used to detect variants. For example, conventional and field-inversion electrophoresis in conjunction with Southern blotting and hybridization can be utilized to detect larger rearrangements such as deletions and insertions.

The association of certain sequence variants with susceptibility to APRKD or a diagnosis of ARPKD can be determined. As defined above, an ARPKD-associated (or disease-associated) sequence variant is a sequence variant or combination of sequence variants within the ARPKD gene of a subject that is correlated with the presence of ARPKD in that subject. Sequence variants associated with the presence of ARPKD in a subject can include, for example, mutations that result in truncation of an ARPKD polypeptide or a substantial in-frame alteration within an ARPKD transcript from the subject, missense or small in-frame mutations found within a nucleic acid sample of a subject and not found at a significant level in the normal population, and mutations that segregate in ARPKD families in a fashion known in the art to be consistent with autosomal recessive inheritance. Other sequence variants may be identified that are not individually disease-associated, but which may be associated with ARPKD when combined with one or more additional sequence variants. Still other sequence variants can be identified that are simply polymorphisms within the normal population, and which are not associated with ARPKD.

8. Articles of Manufacture

ARPKD nucleic acid molecules (e.g., oligonucleotide primer pairs and probes) of the invention can be combined with packaging material and sold as kits for determining the susceptibility of a subject to ARPKD or for diagnosing a patient with ARPKD, based on the detection of ARPKD-associated sequence variants within the ARPKD gene of the subject. Components and methods for producing articles of manufacture such as kits are well known. An article of manufacture may include one pair of ARPKD oligonucleotide primers or a plurality of oligonucleotide primer pairs (e.g., 2, 3, 4, 10, or more than 10 primer pairs). In addition, the article of manufacture may include buffers or other solutions, or any other components necessary to assess whether the ARPKD gene of a subject contains one or more variants. Instructions describing how the ARPKD primer pairs are useful for detecting sequence variants within an ARPKD gene also can be included in such kits.

In other embodiments, articles of manufacture include populations of isolated ARPKD nucleic acid molecules immobilized on a substrate. Suitable substrates provide a base for the immobilization of the nucleic acids, and in some embodiments, allow immobilization of nucleic acids into discrete regions. In embodiments in which the substrate includes a plurality of discrete regions, different populations of isolated nucleic acids can be immobilized in each discrete region. Thus, each discrete region of the substrate can include an ARPKD nucleic acid molecule containing a different sequence variant (e.g., one or more of the variants described in Tables 6, 7, 10, and 12). Such articles of manufacture can include two or more nucleic acid molecules with different sequence variants, or can include nucleic acid molecules with all of the sequence variants known for ARPKD.

Suitable substrates can be of any shape or form and can be constructed from, for example, glass, silicon, metal, plastic, cellulose or a composite. For example, a suitable substrate can include a multiwell plate or membrane, a glass slide, a chip, or polystyrene or magnetic beads. Nucleic acid molecules or polypeptides can be synthesized in situ, immobilized directly on the substrate, or immobilized via a linker, including by covalent, ionic, or physical linkage. Linkers for immobilizing nucleic acids and polypeptides, including reversible or cleavable linkers, are known in the art. See, for example, U.S. Pat. No. 5,451,683 and WO98/20019. Immobilized nucleic acid molecules typically are about 20 nucleotides in length, but can vary from about 10 nucleotides to about 1000 or more nucleotides in length.

In practice, a sample of DNA or RNA from a subject is amplified, the amplification product is hybridized to an article of manufacture containing populations of isolated nucleic acid molecules in discrete regions, and hybridization can be detected. Typically, the amplified product is labeled to facilitate detection of hybridization. See, for example, Hacia et al. (1996) Nature Genet., 14:441-447; and U.S. Pat. Nos. 5,770,722 and 5,733,729.

The invention will be further described in the following examples, which do not limit the scope of the invention described in the claims.

### EXAMPLE 1

### Materials and Methods

Establishment of genetic crosses for mapping the rat Pkhd1 gene: PCK rats (rats having a polycystic kidney phenotype) in the Sprague-Dawley strain (PCK^{SD}/PCK^{SD}) were derived from breeding pairs of the F9 generation obtained from Charles River Japan (Yokohama, Japan). Normal Brown Norway rats (+^{BN}/+^{BN}) were obtained from Charles River USA (Wilmington, Mass.). PCK^{SD}/PCK^{SD} and +^{BN}/+^{BN} rats were crossed to generate 8 female and 5 male PCK^{SD}/+^{BN} F1 animals, which were interbred to generate 469 F2 animals. The F2 animals were sacrificed at 8 weeks; kidneys and livers were formalin fixed for histology and spleens were frozen for DNA isolation. Hematoxylin/eosin stained kidney and liver sections were typed for cysts by microscopic examination prior to genetic analysis.

Genomic mapping of the rat Pkhd1 gene: DNA was isolated from finely chopped rat spleen after 4 h of proteinase K digestion, using the salting out method (Puregene DNA Isolation Kit; Gentra Systems, Minneapolis, Minn.). Primers were generated for 76 markers selected from the Whitehead/MIT rat database (available on the internet) at ˜20 cM intervals to cover the entire rat genome, and were fluorescently labeled with FAM, HEX, or TET (Glen Research, Sterling, Va.). These markers were amplified from 39 of the affected F2 rats using: 50 ng DNA template, 0.2 mM each nucleotide, 8 pmole each primer, and 0.125 U AmpliTaq Gold (PE Applied Biosystems, Foster City, Calif.) in the supplied buffer. The PCR procedure included an initial denaturation at 95° C. for 12 min, followed by 10 cycles of 95° C. for 15 s, 55-65° C. for 15 s, and 72° C. for 30s, 20 cycles as above but with an 89° C., 15 s denaturation step, and a final elongation at 72° C. for 10 min. PCR products were assayed on multiplexed gels using an ABI 377 sequencer (Applied Biosystems) for genotyping. Allele sizes were assigned using the GeneScan® 3.1 and Genotyper® 2.5 software (Applied Biosystems). Linkage between markers and the Pkhd1 gene was assessed by calculating the level of SD/SD homozygotes. Once linkage was established, additional rat markers from the candidate interval were typed as above, or on 2.5% agarose gels, to precisely localize the gene. Calculated recombination fractions were assumed to be equivalent to centi-Morgan (cM) distances.

Rat polymorphisms within orthologs of genes from the human ARPKD candidate region: Rat orthologs of genes in the human ARPKD candidate region (PTD011, MCM3, and IL-17) were obtained by BLAST analysis of the NCBI EST database (est_others; available on the internet from the NCBI government web site). The rat sequence for ug8, another marker within the ARPKD candidate region, was successfully amplified by positioning primers within regions conserved in humans. To obtain an additional ARPKD marker (a rat USG cDNA), a mouse USG EST was used to screen 0.5×10⁶ plaques of an adult rat kidney cDNA library (Stratagene, La Jolla, Calif.), by standard methods. Accession numbers for these markers are provided in Table 1.

Using the human sequence as a guide, polymorphisms were identified within rat orthologs of genes in the ARPKD candidate region. Primers flanking small introns in the rat orthologs were used to amplify adjacent sequences by PCR. PCR samples contained 50 ng rat genomic DNA, 200 mM each dNTP, 8 pmole each primer, 1.5 mM MgCl₂ (supplied in the manufacturer's buffer), and 2.5 U AmpliTaq Gold. The PCR procedure included denaturation at 94° C. for 5 min, 30 cycles of 94° C. for 60 s, 53-64° C. for 60 s, and 72° C. for 120 s, and a final extension at 72° C. for 10 min. The introns were sequenced, and differences were detected using the Sequencher program (Gene Codes Corporation, Ann Arbor, Mich.). Each polymorphism was scored using a restriction enzyme (sites were created with a modified primer if necessary) with products resolved on 1-2% agarose gels. The sequence of the Pkhd1 IVS37 polymorphism was obtained by BLAST screening of the rat Trace database (available on the internet from the NCBI government web site) with the sequence of the human ortholog. Polymorphisms also were developed for rat orthologs of the following human chromosome 6 genes: KIAA0936, TFAP2B, GLP1R, KCNK5, KIAA0105, and KIAA0244. None of these polymorphisms mapped to rat chromosome 9 close to the Pkhd1 gene.

Northern Analysis: RNA was isolated from mouse, rat, and snap frozen human tissues by the method of Chomczynski and Sacchi (Chomczynski and Sacchi (1987) Anal. Biochem. 162:156-159), subjected to electrophoresis in denaturing formaldehyde gels with 0.5% agarose, northern blotted, and hybridized in Express Hyb (Clontech, Palo Alto, Calif.). Probes included the following: a 1.5 kb NotI/EcoRI fragment from murine USG (Image clone 4238864); a 0.65 kb 5′ PstI fragment of the 2 kb insert from rat USG; a 2.3 kb XmnI/EcoRI fragment from murine ug8 (Image clone 1432609) and a 0.8 kb EcoRI/NotI fragment from exons 23 to 26 of murine Pkd1 (Pritchard et al. (2000) Hum. Mol. Genet. 9:2617-2627). The human probes to PKHD1 were cDNA clones A2, C3, and D5 (see Table 2 for details). Commercial human northern blots were purchased from Clontech.

ARPKD Patients: All individuals involved in the study gave informed consent and the project had IRB approval. Clinical information either was extracted from medical records or was taken from interviews of patients and their families. Blood samples for DNA isolation were obtained from patients and family members wishing to participate in the study. Renal tissue from ARPKD patients was collected at nephrectomy, snap frozen in liquid N₂ and stored at −80° C.

RT-PCR Analysis: RNA for RT-PCR was isolated from normal human kidney obtained at nephrectomy, mouse and rat tissues, or cell lines using the SV Total RNA Isolation System (Promega, Madison, Wis.) or NucleoSpin (Clontech), both of which include a DNAse step to remove contaminating genomic DNA. RT was performed with 1-5 mg total RNA using the Powerscript Reverse Transcriptase Kit (Clontech) and 250 ng random primers (Invitrogen/Life Technologies).

Cloning of human PKHD1: Predicted exons in the human region syntenic to the Pkhd1 interval were identified with the NIX suite of programs (available on the internet from the UK Human Genome Mapping Project Resource Centre) and the GenomeScan program 28, and are available on the NCBI Human Chromosome 6 Map View (available on the internet from the NCBI government web site). Genomic sequence spanning the human PKHD1 candidate region was taken from the bacterial artificial chromosomes: RP3-335N17, (AL355997) RP11-442L12 (AL15774), RP3-357H1 (AL121946), RP11-347E4 (AL590391), and RP11-771D21 (AL391221). Primer pairs were generated 1-2 kb apart in the predicted transcript and matching genomic sequence in strongly predicted exons, and were used to amplify nine primary exon-linking clones: C3, D5, B2, A2, J21/23, F2, G4, H14, and I7. To link these clones and confirm the structure of the transcript, a second group of linking clones was amplified: M9, N10, O11, JH8, K7, P9, Q9, and R3. PCR samples contained 300 ng adult kidney cDNA, 200 mM each dNTP, 8 pmole each primer, 1.5 mM MgCl₂ (supplied in the manufacturer's buffer), and 2.5 U AmpliTaq Gold. The PCR procedure included denaturation at 94° C. for 5 min, 30 cycles of 94° C. for 60 s, 53-64° C. for 60 s, and 72° C. for 120 s, and a final extension at 72° C. for 10 min. Exon linking fragments were cloned using the TOPO TA Cloning Kit (Invitrogen) and grown in the E. coli XL-2MRF′ host (Stratagene).

The 5′ region of the gene was amplified by a 5′ RACE strategy using the SMART RACE cDNA Amplification Kit (Clontech). Human kidney RNA was reverse transcribed using PowerScript RT with the 5′ RACE-CDS and SMART-II primers from the kit. Touchdown PCR was carried out with a gene specific primer (5′-GCCTTCTTGTGGACCATTGACTTTCCTTTG-3′; SEQ ID NO:33), the Universal primer mix, and Advantage 2 polymerase, according to the manufacturer's protocol. The 3′ UTR was cloned using Image clones identified by BLAST analysis of the NCBI human EST database and the S3 linking clone.

Mutation analysis by Southern blotting: Genomic DNA was isolated from whole blood and kidney tissue using the DNA Isolation System (Gentra Systems). Probands and familial samples were genotyped with the microsatellites D6S465, D6S 1714, and D6S1344 as previously described (Harris et al. (1991) Lancet 338:1484-1487) to establish whether patients were heterozygous at PKHD1. To look for genomic rearrangements, genomic DNA was digested with BamHI or EcoRI, subjected to electrophoresis on 0.5% agarose gels, Southern blotted, and hybridized by standard methods to the following cDNA probes: 5′ RACE, C3, D5, B2, A2, JN8, F2, G4, H14, and I7.

Mutation analysis by denaturing high-performance liquid chromatography (DHPLC): To screen for polymorphisms and mutations, the coding region of PKHD1 (exons 2-67) was amplified as 79 PCR amplicons of 150-370 bp, using the primers listed in Table 5. PCR samples contained 60 ng genomic DNA, 8 pmol each primer, 200 pM dNTPs, 2.5 mM MgCl₂, and 1 U AmpliTaq Gold, and amplification involved initial denaturation at 94° C. for 2 min, 35 cycles of 94° C. for 30 s, 44-65° C. for 30 s, and 72° C. for 30s, and a final extension at 72° C. for 10 min. Heteroduplexes were generated by heating PCR products at 95° C. for 5 min, cooling to 65° C. at a rate of 0.1 ° C./s, incubating at 65° C. for 30 min, cooling to 37° C. at a rate of 0.1° C./s, and incubating at 37° C. for 10 min. Normal amplicon DNA (representing wild-type PKHD1) was not added prior to heteroduplex formation, since haplotype analysis had indicated that patients with mutations were expected to be compound heterozygotes rather than homozygotes.

Fragments were analyzed on the WAVE Fragment Analysis System (Transgenomics, Omaha, Nev.) using the Wavemaker 4.0.32 software to calculate melting profiles and the required elution gradient. Each fragment was analyzed at the predicted melting temperature and at temperatures 1° C. and 2° C. above and below the predicted melting temperatures (and at additional temperatures if necessary) to determine the optimal analysis conditions. Analysis was typically in the range of 50-75% helical fraction, and nucleic acids containing sequence variants detected during this study (see Table 9) were used as positive controls. Heteroduplex fragments (300-500 ng) were injected into a DNA Sep Cartridge column (Transgenomics) and eluted through a linear gradient of Buffer A (5% TEAA) and Buffer B (5% TEAA, 25% acetonitrile).

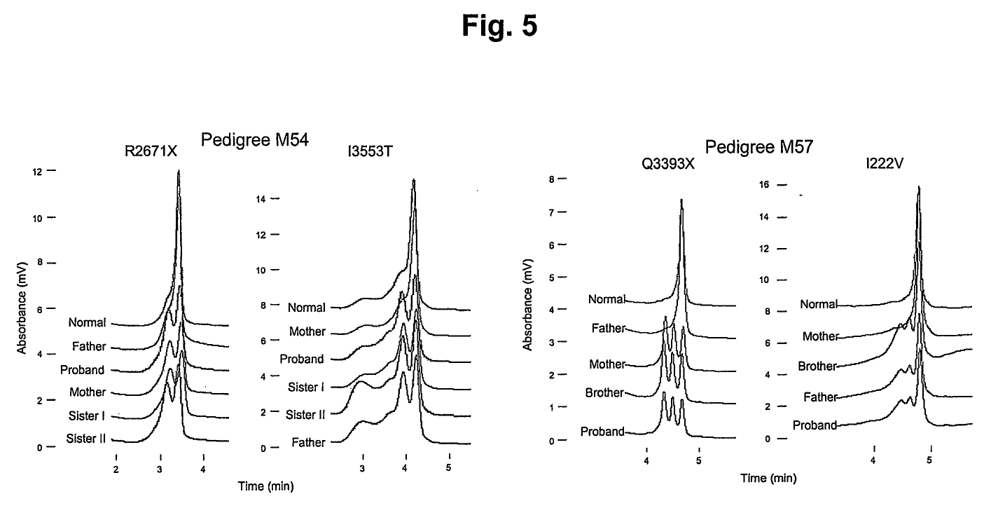

Exons showing an aberrant profile were characterized by direct sequencing using the Big-Dye Terminator Kit (PE Applied Biosystems) and analyzed on an ABI377 Sequencer. The significance of missense changes was determined by assaying the fragment in samples from 100 normal individuals by DHPLC. The segregation of potential missense mutations in families was tested by DHPLC (see FIG. 5).

Sequence Analysis: The sequence of the PKHD1 transcript was assembled from the sequenced, exon-linked clones and compared to the genomic template using the Sequencer 4.1.2 (Gene Codes Corporation, Ann Arbor Mich.) and MacVector 7.0 (Oxford Molecular, Oxford, UK) programs. The intron/exon structure was determined using MacVector to compare the genomic sequence with the cloned transcript. The genomic sequence of murine Pkhd1 was obtained by BLAST analysis of the NCBI Trace murine database (available on the internet from the NCBI government web site) using human cDNA and genomic fragments under conditions of 75% homology. Putative murine exons were identified for approximately 95% of the gene and were assembled into a putative mouse transcript.

Regions of homology between fibrocystin and other proteins were obtained by BLAST, including reiterative PSI-BLAST (available on the internet from the NCBI government web site) and FASTA analysis (available on the internet from the European Bioinformatics Institute web site) of the Genbank database. The Pfam program (available on the internet from the web site of Washington University, St. Louis, Mo.) was used to identify known domains within the protein. The program SignalP 2.0 (available on the internet from the Center for Biological Sequence Analysis web site of the Technical University of Denmark; Nielsen et al (1997) Protein Eng. 10:1-6) was used to assay for the presence of a signal peptide. The transmembrane structure was analyzed by the programs SOUSI (available on the internet at the web site for the Mitalcu laboratory in the Department of Biotechnology at the Tokyo University of Agriculture and Technology), TMHMM v2.0 [available on the internet from the Center for Biological Sequence Analysis web site of the Technical University of Denmark; Sonnhammer et al. Proceedings of Sixth International Conference on Intelligent Systems for Molecular Biology Glasgow et al., ed. (AAAI Press, Menlo Park, 1998)], TopPred2 (available on the internet from the Biological Software web page of the Institut Pasteur; Claros and von Heijne (1994) Comput. Appl. Biosci. 10:685-686), and PHDhtm (available on the internet from the Columbia University Bioinformatics Center; Rost et al. (1996) Protein Sci. 5:1704-1718). Alignments were made using the ClustalW (v1.4) program within MacVector 7.0. Accession numbers of related proteins are: D86 (mouse), BAB32449; HGFR (mouse), NP_{—}032617; Plexin 1 (mouse), NP_{—}032907; Ron (human), NP_{—}002438; TMEM2 (human), NP_{—}037522; XM051857 (human), XP_{—}051857; and DKFZp586C1021 (human), T43498.

### EXAMPLE 2

### PCK is a Rat Model of ARPKD

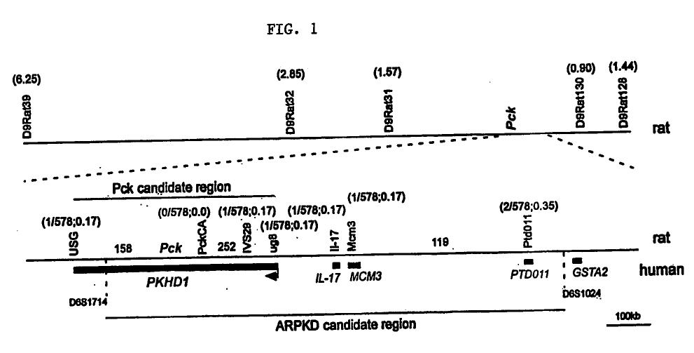

To map the Pkhd1 gene, a cross was established between PCK homozygotes in the Sprague-Dawley strain (PCK^{SD}/PCK^{SD}) and normal Brown Norway rats (+^{BN}/+^{BN}). F1 animals (PCK^{SD}/+^{BN}) were interbred and 469 F2 animals were generated. These were sacrificed at 8 weeks and typed for the PCK/PCK phenotype by histological analysis of kidneys and liver. One hundred and nine affected F2 animals were identified (23.2%). To localize the position of the Pkhd1 gene, 62 markers positioned at 20 cM intervals throughout the 20 rat chromosomes were typed in 39 of the affected F2 animals. Significant enrichment for SD/SD homozygotes, consistent with linkage, was found for just two markers positioned on rat chromosome 9, D9Rat39 and D9Rat128, with SD homozygosities of 84.2% and 94.6%, respectively. Haplotype analysis indicated that the Pkhd1 gene lay between these markers, and typing of all affected animals defined the interval as 7.7 cM. See FIG. 1, top. Further markers within the candidate interval were typed in recombinant animals and Pkhd1 was localized to 2.47 cM between D9Rat31 and D9Rat130.

One possible region syntenic to this interval of rat chromosome 9 was human chromosome 6, close to the ARPKD region, suggesting that the Pkhd1 gene may be an ortholog of PKHD1. To test this possibility, rat ESTs were obtained for genes that mapped to the ARPKD candidate interval (PTD011 and MCM3; Hofmann et al. (2000) Eur. J. Hum. Genet. 8:163-166; see FIG. 1, middle and bottom). To identify intragenic polymorphisms, small introns within the rat orthologs were amplified and sequenced in the SD and NB rat strains. A rat Ptd011 polymorphism was typed in all affected animals (Table 1), and complete correspondence was found between the SD/SD haplotype and the PCK/PCK phenotype. This indicated that Pkhd1 was likely a rat ortholog of PKHD1 and suggested that this model could be used to further localize the human ARPKD gene.

To utilize all genetic information within the cross, the 360 normal F2 rats were also typed for the Ptd011 polymorphism, with the assumption that normal animals could not have the SD/SD haplotype at the disease locus. The analysis revealed two normal animals that were SD/SD at Ptd011; this marker therefore was positioned 0.35 cM from the Pkhd1 gene (FIG. 1). Typing of an Mcm3 polymorphism (Table 1) in the informative animals showed that the marker was recombinant in only one rat and thus was closer to Pkhd1 (0.17 cM), with the disease gene located distal to Mcm3 (FIG. 1).

TABLE 1

Rat polymorphisms assayed in the ARPKD candidate area

| | | |
|---|---|---|
| Accession | SEQ | Fragments |

| | | | |
|---|---|---|---|
| Gene/ | No. | ID | (bp) |

| | | | | | | | |
|---|---|---|---|---|---|---|---|
| Marker | (Species) | Polymorphism | Primers (5′-3′) | NO | Enzyme | PKHD1SD | BN |

| | | | | | | | |
|---|---|---|---|---|---|---|---|
| Ptd011 | BF567520 | IVS4 + 119G/A | F: GCCAGCATTTCCGAGTGGTG | 34 | Tsp509I | 99 + 90 | 189 |
| | (Rat) | | R: TGGGTAAGCAAGGTCAAAGTTCC | 35 | | | |
| Mcm3 | AW520335 | IVS2 + 191A/G | F: ACCCTCAATACAAGAAGGAACCAC | 36 | HhaI | 157 + 21 | 178 |
| | (Rat) | | R: GGTGCTCCCGTCTCCCAGCG* | 37 | | | |
| IL-17 | L13839 | IVS2 + ˜640C/A | F: GGTCTTTGAAAGAAGGAA | 38 | TspRI | 167 | 151 + 16 |
| | (Rat) | | R: CAATCAATTCAAAGGTCTCAG* | 39 | | | |
| Pkhd1/USG | USG cDNA | 3′UTR 411C/T | F: GCAGGACTACAGAAATACTCAG | 40 | Tsp45I | 332 | 183 + 149 |
| | (Rat) | | R: TCCCACTTACCACAGAGAAG | 41 | | | |
| | from mUSG | | | | | | |
| | BF786063 | | | | | | |
| Pkhd1/ug8 | AI049325 | IVS3 + ˜210G/A | F: AGGAGGAACATGGATCACAGT | 42 | BbsI | ˜650 | ˜420 + 230 |
| | (Mouse) | | R: AGCCATTGTTGGGGTAAAGAA | 43 | | | |
| Pkhd1/IVS29 | Ti54702436 | IVS29 + 288A/T | F: TTATAATCATGCAAAATGGGC | 44 | Mfel | 128 | 105 + 23 |
| | (Rat) | | R: ACATCATTTTCATGAGGCAAT* | 45 | | | |
| Pkhd1/CA | | IVS37 + 42ins4 | F: GGCTGTGAGGAATGGAACTCT | 46 | - | 137 | 141 |
| | (Rat) | | R: ATGGTCATTGTCCTCATATTTGC | 47 | | | |

*Primers include engineered change to allow assay with restriction enzyme

Rat orthologs of several human loci positioned close to this interval were mapped in the PCK cross (see Example 1), but none mapped to rat chromosome 9 close to Pkhd1, indicating that the synteny between rat chromosome 9 and human chromosome 6 ended before the location of these markers. The interleukin-17 gene (IL-17) eventually was mapped to the candidate region in rat chromosome 9, and a rat polymorphism was identified and typed in the recombinant animals. IL-1 7 mapped to the same interval as Mcm3, so the proximal limit of the Pkhd1 gene was distally relocated by approximately 50 kb (FIG. 1). No other definite genes were identified in the next ˜1 Mb of corresponding human sequence, but several ESTs were available for a possible gene approximately 80 kb distal to D6S1714. The mouse USG EST was obtained and used to identify a rat clone, rUSG, by screening a rat kidney cDNA library. Sequencing suggested that rUSG contained the 3′ UTR of a gene, and comparison of SD and BN sequence revealed a polymorphism (Table 1). Analysis of this marker in the PCK cross showed no recombination in the rat that was recombinant for Mcm3, and a crossover in just one different affected animal. This result indicated that rUSG marked a distal limit for the Pkhd1 gene and localized the gene to 0.34 cM, corresponding to an ˜620 kb region of human sequence (FIG. 1). The mouse ug8 EST was identified within this interval approximately 150 kb distal to IL-17, and a polymorphism was identified in an intron of the corresponding rat gene (Table 1). Typing of this marker revealed one crossover with the Pkhd1 gene (in the same rat that was recombinant for Mcm3), and thus demonstrated that ug8 was the closest flanking proximal marker. The Pkhd1 gene therefore was localized to an area of 0.34 cM, and an equivalent human physical interval of approximately 470 kb (FIG. 1).

Although no genes had been definitively identified within the 470 kb interval, four different genes were predicted by GenomeScan analysis. Two of these genes were represented at the 5′ or 3′ end by the USG and ug8 ESTs that had been typed. To characterize the transcripts associated with USG and ug8, they were analyzed by northern blotting with mouse RNA. The 14.1 kb murine Pkd1 transcript was used as a size control. Hybridization with either the USG or ug8 probe revealed a large transcript of ˜13 kb, which was moderately expressed in adult kidney but not in brain tissue, indicating that these ESTs represented parts of the same large gene. A similar large transcript was visualized with rat RNA.

The human ortholog was cloned and characterized as the PKHD1 gene (see Example 3, below). This sequence was used to identify intragenic polymorphisms in Pkhd1. A crossover (in the rat that was recombinant for Mcm3 and ug8) with an IVS29 polymorphism (see Table 1) excluded the first 29 exons of Pkhd1 as the location of the PCK mutation, but an IVS37 microsatellite showed no crossovers with the PCK phenotype. The mutation was sought in the area surrounding the point of zero recombination by sequence comparison of the PCK^{SD}/PCK^{SD} and SD⁺/SD⁺ transcripts. No changes were found in exons 37-40, but amplification of exons 33-37 revealed a smaller product in the PCK rat. Genomic sequencing showed that the PCK mutation is a splicing change, IVS35-2A→T, which results in a frameshifting skipping of the 157 bp exon 36.

### EXAMPLE 3

### Cloning of Human PKHD1

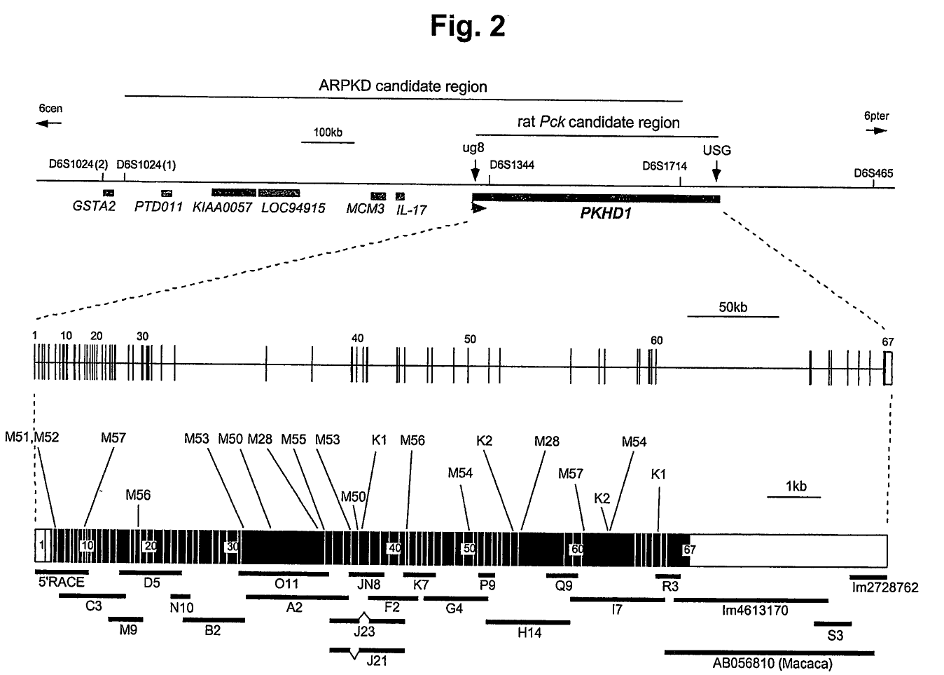

The location of the Pkhd1 gene and the similarity of the PCK phenotype to ARPKD indicated that Pkhd1 was a strong candidate for the PKHD1 gene. The human transcript therefore was cloned. Human and Macaque ESTs were available only from the 3′ UTR and immediate 3′ portion of the putative coding region. However, GenomeScan predictions of the genomic sequence from this interval suggested the presence of many exons (initially assembled as four different genes), represented at the 5′ and 3′ ends, respectively, by ug8 and USG (FIG. 2, top). This information was used as a guide to clone the gene by an exon-linking strategy, with primers positioned 1-2 kb apart in strongly predicted exons of the putative transcript. Human adult kidney RNA was used as the template for RT-PCR. The transcript was cloned as 9 overlapping fragments, and the structure of the mRNA was confirmed by amplifying 9 further linking clones (Table 2 and FIG. 2, middle and bottom).

TABLE 2
Details of PKHD1 cDNA clones

| Clone | Size (bp) | Exons | Position (nt)* | Comment |
|---|---|---|---|---|
| 5′RACE | 1003 | 1-11 | −276:728 | |
| C3 | 1277 | 4-16 | 147:1423 | |
| M9 | 666 | 14-18 | 1009:1674 | |
| D5 | 1186 | 16-24 | 1260:2445 | |
| N10 | 389 | 23-25 | 2284:2672 | |
| B2 | 1276 | 24-32 | 2445:3720 | |
| O11 | 1745 | 31-33 | 3582:5326 | |
| A2 | 1968 | 32-35 | 3735-5702 | |
| J21 | 1263 | 33-41 | 5357:6776 | no ex36 |
| J23 | 1207 | 33-41 | 5357:6776 | no ex37 |
| JN8 | 690 | 35-39 | 5719:6408 | |
| F2 | 971 | 37-44 | 6098:7068 | |
| K7 | 623 | 41-47 | 6753:7375 | |
| G4 | 1236 | 45-53 | 7148:8383 | |
| P9 | 296 | 52-54 | 8224:8519 | |
| H14 | 1606 | 53-59 | 8341:9946 | |
| Q9 | 612 | 58-60 | 9489:10100 | |
| I7 | 1810 | 59-66 | 9931:11740 | |
| R3 | 472 | 65-67 | 11533:12004 | |
| Im4613170 | 2934 | 67 | 12181:3′ 2556 | |
| S3 | 858 | 67 | 3′ 2243:3′ 3100 | |
| Im2728762 | 694 | 67 | 3′ 3044:3′ 3737 | |
| AB056810 | 4259 | 64-67 | 11485:3′ 3511 | Macaque |

*3′ = Position in 3′ UTR; negative number = position in 5′ UTR

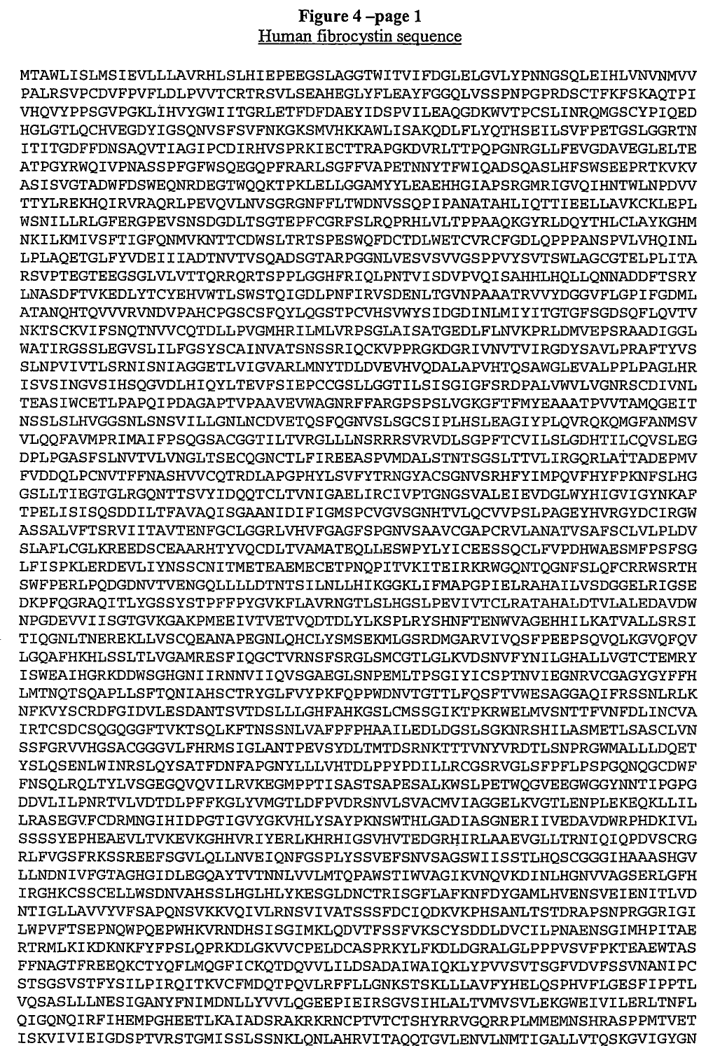

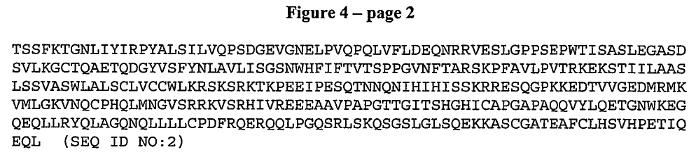

A number of different products were generated in some parts of the gene, especially within fragments A, I, and J, suggesting significant alternative splicing of the gene. The largest RT-PCR fragment in each case that had an open reading frame (ORF) was selected from each reaction for inclusion in the final product. A 5′ RACE strategy was used to clone the start of the gene, a position that was roughly equivalent to that found in the mouse ug8 EST. In total, a transcript of 16,235 bp was cloned. Comparison with genomic DNA showed that the transcript was encoded by 67 exons in a genomic region of approximately 472 kb (Table 3 and FIG. 2, middle and bottom). Analysis of PKHD1 revealed an ORF starting in exon two, with a putative start codon that was preceded by a sequence consistent with a Kozak consensus and an in-frame stop-codon 12 bp upstream; the ORF ended in exon 67. The ORF was 12222 bp in length, with 5V and 3′ UTRs of 275 bp and 3738 bp, respectively, and a typical polyadenylation signal 17 bp upstream from the site of polya addition. The sequence of the ORF is shown in FIG. 3. PKHD1 was predicted to encode a protein with 4074 amino acid residues; the amino acid sequence is shown in FIG. 4.

TABLE 3
Intron/exon structure of PKHD1

| Exon | | Transcript | Coding Region | | IVS |
|---|---|---|---|---|---|
| Number | Size | Position (nt) | Position (nt) | Position (aa) | Size |
| 1 | 192 | 1-192 | — | — | 2416 |
| 2 | 136 | 193-328 | 1-52 | 1-18 | 1626 |
| 3 | 78 | 329-406 | 53-130 | 18-44 | 635 |
| 4 | 151 | 407-557 | 131-281 | 44-94 | 2383 |
| 5 | 109 | 558-666 | 282-390 | 94-130 | 3566 |
| 6 | 58 | 667-724 | 391-448 | 131-150 | 2734 |
| 7 | 79 | 725-803 | 449-527 | 150-176 | 1273 |
| 8 | 75 | 804-878 | 528-602 | 176-201 | 1044 |
| 9 | 65 | 879-943 | 603-667 | 201-223 | 560 |
| 10 | 40 | 944-983 | 668-707 | 223-236 | 878 |
| 11 | 71 | 984-1054 | 708-778 | 236-260 | 3379 |
| 12 | 102 | 1055-1156 | 779-880 | 260-294 | 925 |
| 13 | 96 | 1157-1252 | 881-976 | 294-326 | 2294 |
| 14 | 142 | 1253-1394 | 977-1118 | 326-373 | 2476 |
| 15 | 115 | 1395-1509 | 1119-1233 | 373-411 | 1326 |
| 16 | 279 | 1510-1788 | 1234-1512 | 412-504 | 1343 |
| 17 | 90 | 1789-1878 | 1513-1602 | 505-534 | 101 |
| 18 | 91 | 1879-1969 | 1603-1693 | 535-565 | 968 |
| 19 | 143 | 1970-2112 | 1694-1836 | 565-612 | 1421 |
| 20 | 128 | 2113-2240 | 1837-1964 | 613-655 | 786 |
| 21 | 176 | 2241-2416 | 1965-2140 | 655-714 | 2781 |
| 22 | 139 | 2417-2555 | 2141-2279 | 714-760 | 1517 |
| 23 | 128 | 2556-2683 | 2280-2407 | 760-803 | 2303 |
| 24 | 185 | 2684-2868 | 2408-2592 | 803-864 | 915 |
| 25 | 123 | 2869-2991 | 2593-2715 | 865-905 | 1235 |
| 26 | 106 | 2992-3097 | 2716-2821 | 906-941 | 490 |
| 27 | 276 | 3098-3373 | 2822-3097 | 941-1033 | 7137 |
| 28 | 131 | 3374-3504 | 3098-3228 | 1033-1076 | 2425 |
| 29 | 136 | 3505-3640 | 3229-3364 | 1077-1122 | 4678 |
| 30 | 196 | 3641-3836 | 3365-3560 | 1122-1187 | 259 |
| 31 | 68 | 3837-3904 | 3561-3628 | 1187-1210 | 1647 |
| 32 | 1608 | 3905-5512 | 3629-5236 | 1211-1746 | 1630 |
| 33 | 144 | 5513-5656 | 5237-5380 | 1745-1794 | 5171 |
| 34 | 220 | 5657-5876 | 5381-5600 | 1794-1867 | 6950 |
| 35 | 151 | 5877-6027 | 5601-5751 | 1867-1917 | 50282 |
| 36 | 157 | 6028-6184 | 5752-5908 | 1918-1970 | 25547 |
| 37 | 213 | 6185-6397 | 5909-6121 | 1970-2041 | 21533 |
| 38 | 211 | 6398-6608 | 6122-6332 | 2041-2111 | 409 |
| 39 | 158 | 6609-6766 | 6333-6490 | 2111-2164 | 2324 |
| 40 | 192 | 6767-6958 | 6491-6682 | 2164-2228 | 2942 |
| 41 | 126 | 6959-7084 | 6683-6808 | 2228-2270 | 2172 |
| 42 | 57 | 7085-7141 | 6809-6865 | 2270-2289 | 258 |
| 43 | 131 | 7142-7272 | 6866-6996 | 2289-2332 | 16351 |
| 44 | 113 | 7273-7385 | 6997-7109 | 2333-2370 | 1160 |
| 45 | 106 | 7386-7491 | 7110-7215 | 2370-2405 | 2639 |
| 46 | 135 | 7492-7626 | 7216-7350 | 2406-2450 | 12453 |
| 47 | 136 | 7627-7762 | 7351-7486 | 2451-2496 | 2394 |
| 48 | 247 | 7763-8009 | 7487-7733 | 2496-2578 | 11792 |
| 49 | 178 | 8010-8187 | 7734-7911 | 2578-2637 | 7922 |
| 50 | 196 | 8188-8383 | 7912-8107 | 2638-2703 | 11305 |
| 51 | 66 | 8384-8449 | 8108-8173 | 2703-2725 | 5414 |
| 52 | 129 | 8450-8578 | 8174-8302 | 2725-2768 | 39487 |
| 53 | 138 | 8579-8716 | 8303-8440 | 2768-2814 | 15314 |
| 54 | 114 | 8717-8830 | 8441-8554 | 2814-2852 | 3018 |
| 55 | 88 | 8831-8918 | 8555-8642 | 2852-2881 | 17763 |
| 56 | 155 | 8919-9073 | 8643-8797 | 2881-2933 | 1430 |
| 57 | 153 | 9074-9226 | 8798-8950 | 2933-2984 | 4535 |
| 58 | 879 | 9227-10105 | 8951-9829 | 2984-3277 | 897 |
| 59 | 169 | 10106-10274 | 9830-9998 | 3277-3333 | 2178 |
| 60 | 158 | 10275-10432 | 9999-10156 | 3333-3386 | 84415 |
| 61 | 1018 | 10433-11450 | 10157-11174 | 3386-3725 | 9731 |
| 62 | 136 | 11451-11586 | 11175-11310 | 3725-3770 | 966 |
| 63 | 88 | 11587-11674 | 11311-11398 | 3771-3800 | 9074 |
| 64 | 108 | 11675-11782 | 11399-11506 | 3800-3836 | 6125 |
| 65 | 159 | 11783-11941 | 11507-11665 | 3836-3889 | 5448 |
| 66 | 120 | 11942-12061 | 11666-11785 | 3889-3929 | 7476 |
| 67 | 4174 | 12062-16235 | 11786-12222 | 3929-4074 | |

### EXAMPLE 4

### Screening the Human PKHD1 Gene for Mutations in ARPKD Patients

To determine whether the cloned transcript was the PKUD1 gene, a strategy was devised to screen the entire coding region for mutations in ARPKD patients. Blood samples were collected from 12 probands who either were clinically diagnosed with ARPKD or were suspected to have ARPKD (Table 4). Samples also were isolated from frozen renal tissue of two ARPKD patients and although clinical information was not available, histological analysis confirmed a diagnosis of ARPKD. Two strategies were employed to look for mutations: 1) Southern blots of genomic DNA from the 14 probands were hybridized with cDNA probes covering the entire ORF. Although polymorphisms were detected with one probe, no clear disease-associated rearrangements were identified. 2) DHPLC, a rapid and sensitive method for detecting base pair changes (Xiao and Oefner (2001) Hum. Mutat. 17:439-474; Underhill et al. 1997 Genome Res. 7:996-1005), was used to screen the 66 coding exons. Using the primers listed in Table 5, the exons were amplified as 79 different genomic fragments of 150-370 bp, an optimal size for DHPLC analysis. A significant number of base pair changes were detected by this method. Examples of aberrant profiles and sequences of some of the detected changes are shown in FIG. 5.

To differentiate likely pathogenic changes from neutral polymorphisms, segregation in families was tested where possible. Missense changes also were assayed in 200 normal chromosomes to determine whether the changes were found in the normal population. Table 6 contains a list of likely pathogenic changes, which either were predicted to truncate the protein or were missense changes not found in the normal cohort. The significance of the missense substitutions was further assessed by analyzing the conservation of the substitutions in the murine ortholog and in other homologs. A list of polymorphisms found in the normal population is shown in Table 7. In addition, a number of alternatively spliced forms of PKHD1 were detected; these are listed in Table 8.

TABLE 4
Clinical details of ARPKD patients

| Pedigree | Patient | Sex | Age at dx | Presentation | Investigation^{●} | Renal Imaging^{▪} |
|---|---|---|---|---|---|---|
| M52 | R955 | F | In utero | Mechanical | US | +++, cysts |
| | | | | ventilation, | | echogenic |
| | | | | hypertension at birth | | |
| M36 | R324 | F | Birth | Mechanical | US | +++, cysts |
| | | | | ventilation | | echogenic |
| M58 | R948 | M | Birth | Renal enlargement | ExU, US, CT | ++, cysts |
| | | | | | | echogenic |
| P244 | OX1431 | M | Birth | Renal enlargement | ExU, US, | +++, cysts |
| | | | | pneumothorax | liver bx | |
| M56 | R925 | M | 5 d | Hypertension | ExU, US, CT, MR | +++, cysts |
| | | | | | | echogenic, |
| M57 | R895 | F | 1 m | Renal enlargement | US, CT, MR, renal | +++, cysts |
| | | | | | bx | echogenic |
| | R973 | M | 2 m | Hypertension | US | +++, cysts |
| | | | | | | echogenic |
| M51 | R954 | F | 6 w | Palpable right | US, CT | ++, cysts |
| | | | | kidney and liver | | |
| | R953 | F | 2 d | Affected sibling | US | ++, cysts |
| | | | | | | echogenic |
| M28 | R272 | F | 9 m | Abdominal mass | US, CT | +++, cysts |
| | | | | | | echogenic |
| M50 | R328 | M | 1 y | Hepatosplenomegaly | ExU, US | +++, cysts |
| | | | | | renal bx | echogenic |
| M53 | R947 | F | 20 y | Cholangitis | US, CT, ERCP, | MSK, nephro- |
| | | | | | liver bx | calcinosis |
| M55 | R946 | M | 25 y | Flank pain | US, CT, ERCP | + |
| | | | | | | polycystic |
| M54 | R945 | M | 37 y | Elevated serum | ExU, US, CT, MR | MSK, |
| | | | | creatinine | | cysts |
| | R982 | F | 42 y | Proteinuria | MR | 1 renal cyst |
| | | | | splenomegaly | | |
| | R985 | F | 42 y | F/H CHF | US | renal cysts |

| | Hypertension | Renal function* | | | |
|---|---|---|---|---|---|
| Pedigree | (Y/N, age) | (age; years) | CHF | CD | Associated findings |
| M52 | Y (at birth) | Cl_{In} 37 (3) | Yes | No | Hematemesis at 3 y, |
| | | | | | variceal banding |
| M36 | Y (1 w) | SC 0.9 (3) | Yes | No | |
| M58 | Y(1 m) | SC 0.7 (4) | No | No | |
| P244 | Y (1 y) | CI_{EDTA} 81 (15) | Yes | Yes | |
| M56 | Y (1 w) | SC 0.6 (7) | Yes | Yes | |
| M57 | Y (1 y) | Renal Tx (13) | No | No | |
| | Y (2 m) | SC 1.6 (12) | Yes | No | Coloboma |
| M51 | Y (6 m) | SC 1.0 (16) | Yes | No | Splenomegaly |
| | Y (2 y) | SC 0.8 (10) | Yes | No | |
| M28 | Y (2 y) | Renal Tx (10) | Yes | Yes | Bilateral inguinal |
| | | | | | hernias, pyloric stenosis, |
| | | | | | very low uric acid |
| M50 | Transient | Renal Tx (30) | Yes | No | Hematemesis at 4 y, |
| | | | | | portocaval shunt, |
| M53 | N | SC 0.9 (20) | Yes | Yes | |
| M55 | N | SC 1.8 (41) | Yes | Yes | Esophageal varices, |
| | | | | | cholangitis, |
| | | | | | splenomegaly |
| M54 | Y (37 y) | SC 3.8 (40) | No | Yes | |
| | N | SC 0.7 (44) | Yes | No | |
| | N | SC 1.1 (42) | Yes | No | |

*Clᵢₙ = Inulin clearance, ml/min/1.73 m²; CL_{EDTA}, = EDTA clearance ml/min/1.73 m²; SC = Serum creatinine mg/dl;
^{▪}Renal enlargement; +++ marked, ++ moderate, + mild; MSK = Medullary sponge kidney;
^{●}US = Abdominal ultrasound; ExU = Excretory urogram; CT = Computer tomography; MRI = Magnetic resonance imaging; bx = Biopsy; ERCP = Endoscopic retrograde cholangio-pancreatography

TABLE 5

DHPLC primers

| | |
|---|---|
| SEQ | SEQ |
| ID | ID |

| | | | | | |
|---|---|---|---|---|---|
| EXON | Size, Anneal | Forward 5′-3′ | NO | Reverse 5′-3′ | NO |

| | | | | | |
|---|---|---|---|---|---|
| 2 | 269 bp, 50C | AGGTTTCAGAACAGCAAAATAATCG | 48 | TTCTCAAGGTAACCTATTGTGTTCTTA | 49 |
| 3 | 160 bp, 54C | TGGTTTGAATCTGACCTTCAAAACC | 50 | AAATGTGCACTTGGTAAAACCCC | 51 |
| 4 | 260 bp, 65C | TTTCACACTGTCCTGTGTCAATGAC | 52 | AAAATCCCTCATCCTGTCTGGTC | 53 |
| 5 | 189 bp, 54C | TTGGGAATTCATGGTTTTTGATTC | 54 | ACATACCTTCCTCCAGCCTTAGAAC | 55 |
| 6 | 178 bp, 52C | GAAAGGCTTGTGCCTCCTGTG | 56 | TGGCAAACAGATTCACAATTATTCC | 57 |
| 7 | 180 bp, 47C | CATTGAGTTTGAGCTAAGTCC | 58 | CATGCAGCATGTATGTAACTAG | 59 |
| 8 | 186 bp, 47C | GTTTATTGGGAGTATTGC | 60 | GTGGACGAACTTACAAGC | 61 |
| 9 | 157 bp, 47C | TGAGTTGTCTGGTCATTC | 62 | GAGAAAGAAATGGATAAGAC | 63 |
| 10 | 152 bp, 44C | ACTCGTGCAGATTCCTGAG | 64 | CAAGATGAGAGAGATAGG | 65 |
| 11 | 157 bp, 49C | CAATCCCAGTTGATATTTTC | 66 | ACAAGGGAAGGGGTACTTG | 67 |
| 12 | 151 bp, 46C | TGGTCTATATTTGGAAGC | 68 | ACCTGCAATGGTAACCTG | 69 |
| 13 | 170 bp, 49C | CCTACACACACACACATAC | 70 | GTTTATTGAACAGCCCTG | 71 |
| 14 | 247 bp, 52C | TTCCCCAATTTGGGAAGG | 72 | TTAGCAAAGGTGCTTTTG | 73 |
| 15 | 193 bp, 50C | TTGGTTACTCTTGCTTGACTC | 74 | CTGGCAACAGAGAAAAGG | 75 |
| 16 | 347 bp, 51C | TGCATAGTATTGATCATG | 76 | AGCTCCATGGGACTGGAAAG | 77 |
| 17 | 188 bp, 52C | TTAGCACCATCATTTAGTCTTG | 78 | AAAGACCACCCCCAGTTC | 79 |
| 18 | 185 bp, 49C | AATTCCTGGCATTTTTTTC | 80 | CATTTTATAGAAAGAAAGAAGACC | 81 |
| 19 | 228 bp, 53C | TATCTATGCCTGCCTTTC | 82 | AATACCTACCCACCTGACCC | 83 |
| 20 | 204 bp, 49C | CACTAATAGAACTGAAGGAC | 84 | TGACTGAATTCCCACCACGC | 85 |
| 21 | 289 bp, 57C | TAACCGGAGAGGACTGCAAGTG | 86 | TTTGAGGTAGGCATGTGACCGG | 87 |
| 22 | 245 bp, 57C | TTTTCCACACAGCAAGTCTACCATC | 88 | CATTCTTAGGAGAAGGGACAGGTG | 89 |
| 23 | 250 bp, 56C | CACCCCAACCCAGACGTTAATAC | 90 | TCCCAGGATGTTGTTCCCTTGG | 91 |
| 24 | 269 bp, 48C | TAGTGTCTGTGTTTTCTG | 92 | TCCAGGGCAGCAAATCCATG | 93 |
| 25 | 208 bp, 55C | TTCGGTTCCATGACAGAATTTACC | 94 | TGAAACTGGAGCTTGCACTTAGG | 95 |
| 26 | 229 bp, 54C | CAGCTTGGGAGCACTTCACATATAC | 96 | TTAAGCCCATCTCAGAGCCAAG | 97 |
| 27 | 363 bp, 48C | TGAAGTAATATCACTGAGAG | 98 | ACATACTGTGAGACCCTCC | 99 |
| 28 | 207 bp, 49C | CCTGTATGGTTGGTGATC | 100 | GAGAAAGAGATATGAAAGG | 101 |
| 29 | 253 bp, 46C | CCCTTAAGTCAGTCCTAC | 102 | TTTATAGGACCAATGCTC | 103 |
| 30 | 348 bp, 54C | GGGGTGACTGTGAATTTAATC | 104 | TGCTAGACCATCAAACAAATC | 105 |
| 31 | 164 bp, 50C | ATCTCTCTCTGTCAGTTATTTCC | 106 | AAATAGAATTGCTGGATAATTG | 107 |
| 32a | 312 bp, 50C | TCTTAGTTCAGAATATCAG | 108 | TCATACATGAAGGTGAAG | 109 |
| 32b | 365 bp, 56C | TGGGCTGGCAACAGGTTC | 110 | GCCATTATCCGAGGCATC | 111 |
| 32c | 345 bp, 52C | ATGGGATTTGCTAATATG | 112 | GACAGGACTTGCCTCTTC | 113 |
| 32d | 350 bp, 50C | GACCACACCATTCTCTGC | 114 | TAAAAAACTGACAGGTAG | 115 |
| 32e | 368 bp, 50C | GCAATGTAACTTTTTTTAATGC | 116 | TCCTATGTGATACCAAAG | 117 |
| 32f | 349 bp, 50C | AGCTCATCCGGTGCATTG | 118 | ATAACTCTTGAGGTGAAC | 119 |
| 32g | 171 bp, 50C | GGGAGTACCACGTCAGAG | 120 | TCCAGAAGTGAAAGGAGC | 121 |
| 33 | 239 bp, 52C | GATCAAGAACTTGTACCTTTGTC | 122 | TTAACCAAAGAATATCATTTCC | 123 |
| 34 | 288 bp, 51C | TTCTTTCCTAATGGTGAC | 124 | TTTGTGGGGAAGTTCAGGG | 125 |
| 35 | 231 bp, 49C | TAAGATTGATGACACCCC | 126 | GCTGTTTGAATCAGTCTG | 127 |
| 36 | 222 bp, 49C | AACCAACCAACCCACCAAC | 128 | TATTACCAACCTACAAAC | 129 |
| 37 | 293 bp, 51C | TAAGCCTTATCCTCCCAG | 130 | ACTTCATTTCCTCTGATC | 131 |
| 38 | 278 bp, 50C | TCTGGACAACTTTTCCTC | 132 | TCTTCCATGTCAACTTAG | 133 |
| 39 | 232 bp, 49C | TGATGTCCTCAGTTCTATC | 134 | TTGCTCATTAGACTTTCC | 135 |
| 40 | 288 bp, 51C | ATGCTTTAGGTTCTCTGG | 136 | TAGTGCCTTAAACATGGG | 137 |
| 41 | 250 bp, 50C | AACAGAATCTCAGGAGCC | 138 | TGGGGAGAATTCATTGTG | 139 |
| 42 | 152 bp, 43C | AAGTGACATAAAATATACTC | 140 | GACAATTTTAAATACACTG | 141 |
| 43 | 195 bp, 50C | GATCCCCTGGATTTGTTG | 142 | TCAGTTCTGGTCTTCCTG | 143 |
| 44 | 198 bp, 47C | TATCATACATGGGGTAAC | 144 | AAGACAGCCAAAACATAG | 145 |
| 45 | 190 bp, 45C | GTTAGAAACATAAAAATTGG | 146 | AACAACAACAATAACAAC | 147 |
| 46 | 199 bp, 47C | TCAGACCTTTGCTGTAAC | 148 | AGCCTAAAACAACCACAC | 149 |
| 47 | 213 bp, 46C | GTCCAGTTTTCTTATTTTGC | 150 | TCATCTGTTCTGTCTATTC | 151 |
| 48 | 324 bp, 51C | GTGCCATTGTGTAATAATCTTTCTG | 152 | CATCGGCAAGCTAAAAAG | 153 |
| 49 | 257 bp, 48C | CAAATAATCTCTCAACCC | 154 | GCAGCATACCAACTAATG | 155 |
| 50 | 277 bp, 50C | GGGGTTCCTTACTAAATG | 156 | GCTCTCAAAACATTCATC | 157 |
| 51 | 147 bp, 47C | CTTTGTATCACATGCAAG | 158 | TTCTGCCATACTAGACAC | 159 |
| 52 | 207 bp, 48C | GGAAGTTATCACAATGGATTAG | 160 | GATTCATCTCTTGGGTAG | 161 |
| 53 | 215 bp, 48C | TTGTTTTTGTGACATATC | 162 | TCAACATGCTCGCAATCC | 163 |
| 54 | 224 bp, 44C | CTCTCTTTCTTTTAATTTC | 164 | CACAATACACACACATGC | 165 |
| 55 | 153 bp, 46C | CTATCCAACTGTTACTCC | 166 | CCAAGAAAAAGCCCTAAG | 167 |
| 56 | 231 bp, 48C | CACTGTTAGTATATCCAATG | 168 | CATTCACTTACCTTAACC | 169 |
| 57 | 228 bp, 52C | GTTTTTTTTTCCCACAACTC | 170 | AGGCTCCAACTGGTAATGG | 171 |
| 58a | 283 bp, 48C | AGGAAAGTACCTGATGAC | 172 | GAACCACAAGGTTATTAG | 173 |
| 58b | 292 bp, 50C | ATATTGTGTTTGGCACAG | 174 | AGTCCACTTTCCTTATAG | 175 |
| 58c | 272 bp, 48C | GAACTGCTTTGGTCTGAC | 176 | ATGACTGAATTCCTAAGC | 177 |
| 58d | 307 bp, 50C | AAAATTCCGTCAAAAAAG | 178 | ATGGATGTATGAAATGGC | 179 |
| 59 | 265 bp, 45C | TGGCTGGTGGTTTATATG | 180 | GTACTTCATAAATATGGC | 181 |
| 60 | 227 bp, 50C | CATGAAATGAAAGAGTTGC | 182 | ACCACAGGCATTGCATTC | 183 |
| 61a | 350 bp, 48C | TATCACTTGTTTTGCTTC | 184 | GAGGTACTTTTGTTCCCC | 185 |
| 61b | 350 bp, 48C | AAGTCTGCTTCATGGATC | 186 | GTTAGTTAGTCTTTCGAG | 187 |
| 61c | 350 bp, 48C | AGTCTTAGAAAAAGGCTG | 188 | GTAATTTGTTACTTGATAAG | 189 |
| 61d | 218 bp, 48C | CAACAGTAAGGAGCACTG | 190 | ATGGACCTAAAAAATCAG | 191 |
| 62 | 270 bp, 53C | GGATTGTGGAAAATTGCTACCATAG | 192 | GGTGAATGCTACATGCTACTTAGC | 193 |
| 63 | 202 bp, 53C | TCTGAATCCAACTTTTTCTTCCTCC | 194 | GCTGCAAACATTTTCTGTGCAG | 195 |
| 64 | 213 bp, 49C | TTCGCAGAAGACATGAAGACATTG | 196 | CACAGAATAAAAGCACACTGT | 197 |
| 65 | 263 bp, 47C | TTATATTAGCATCTTATTAA | 198 | GACTTTTTTTTCAGAAATTTTC | 199 |
| 66 | 230 bp, 54C | GCTGATGGTCCCACTTACAACTG | 200 | CCATCCACAGTGGGTCTCTCC | 201 |
| 67a | 283 bp, 57C | TGAAAACTAAATCCATTTCTTCCCC | 202 | ATCTGAGCAACTGCTCTTGGCC | 203 |
| 67b | 292 bp, 57C | CCTGCAAGAGACTGGGAACTGG | 204 | GAACATTCTGCCTTTCAGGGC | 205 |

TABLE 6
Details of mutations to PKHD1

| Pedigree | Patient(s) | Mutations | Change* | Exon | Segregation^{§} | Normal Screen^{‡} | Mouse | Homology | Comments |
|---|---|---|---|---|---|---|---|---|---|
| M56 | R925 | 1624del4 | 541↓ | 18 | M | | | | |
| | | I2331K | 6992T→A | 43 | P | X | M | L (TMEM2) | |
| M57 | R895, R973 | Q3392X | 10174C→T | 61 | M | | | | |
| | | I222V | 664A→G | 9 | P | X | I | I (D86) | |
| M50 | R328 | 5896insA | 1965↓ | 35 | M | | | | |
| | | L1407R | 4220T→G | 32 | P | X | L | I (D86) | Splice change? |
| M54 | R945, R982 | R2671X | 8011C→T | 50 | M | | | | |
| | R985 | I3553T | 10658T→C | 61 | P | X | V | V(DKFZ) | |
| K1 | Ind 1 | W3871X | 11612G→A | 65 | NP | | | | |
| | | E1995G | 5984A→G | 37 | NP | X | E | A (TMEM2) | |
| K2 | Ind 2 | 10637delT | 3534↓ | 61 | NP | | | | |
| | | I2957T | 8870T→C | 57 | NP | X | I | I (TMEM2) | |
| M28 | R72 | S1664F | 4991C→T | 32 | M | X | S | S (D86) | |
| | | S3018F | 9053C→T | 58 | P | X | S | L (TMEM2) | |
| M53 | R947 | C1249W | 3747T→G | 32 | P | X | C | C (D86) | |
| | | Q1917R | 5750A→G | 35 | M | X | Q | — | Splice change? |
| M55 | R946 | V1741M | 5221G→A | 32 | NP | X | V | S (D86) | |
| M51 | R953, R954 | T36M | 107C→T | 3 | M | X | T | T (D86) | |
| M52 | R955 | T36M | 107C→T | 3 | M | X | T | T (D86) | |

*Nucleotide change or ↓ = frameshift after amino acid
^{§}Allele inheritance: M, maternal; P, paternal; NP, analysis not possible because samples unavailable
^{‡}X = substitution not found in 200 normal chromosomes

TABLE 7
Exonic polymorphisms of PKHD1

| | Amino Acid Position/ | | | |
|---|---|---|---|---|
| Designation | Nucleotide Change | Exon | Allele Frequency (%) | |
| I/V25 | 73G/A | 3 | 1/200 | (0.5) |
| 234C/T | D78 | 4 | 6/38 | (16) |
| 1587T/C | N529 | 16 | 2/38 | (5) |
| T/M752 | 2255C/T | 22 | 1/200 | (0.5) |
| R/C760 | 2278C/T | 22 | 28/106 | (26) |
| W/R852 | 2554T/C | 23 | 15/200 | (8) |
| 2853C/T | T951 | 27 | 1/38 | (3) |
| 3756G/C | L1252 | 32 | 2/38 | (5) |
| A/V1262 | 3785C/T | 32 | 3/50 | (6) |
| 4920A/G | V1640 | 38 | 1/200 | (0.5) |
| 7587G/A | G2529 | 48 | 8/38 | (21) |
| 7764A/G | L2588 | 49 | 6/38 | (16) |
| T/M2938 | 8813C/T | 57 | 1/200 | (0.5) |
| 9237G/A | A3079 | 58 | 7/38 | (18) |
| D/Y3139 | 9415C/T | 58 | 3/200 | (2) |
| S/R3505 | 10515C/T | 61 | 1/200 | (0.5) |
| 10521C/T | H3507 | 61 | 7/200 | (4) |
| 11340T/C | P3780 | 63 | 5/38 | (13) |
| Q/R3899 | 11196A/G | 66 | 10/46 | (22) |
| V/I3960 | 11878G/A | 67 | 2/46 | (4) |
| Q/R4048 | 12143A/G | 67 | 12/46 | (26) |

TABLE 8
Splice forms of PKHD1

| | | Size (bp) | Position | Reading | Strong |
|---|---|---|---|---|---|
| Name | Exon(s) lost | difference | (coding region) | frame* | product |
| B3 | 27 (part) | −59 | 2822-2881 | F/S | − |
| B5 | 27 | −276 | 2822-3098 | | − |
| | 30 | −196 | 3365-3561 | F/S | − |
| B4 | 30 | −196 | 3365-3561 | F/S | + |
| B1 | 30 + 31 | −264 | 3365-3629 | I/F | − |
| A1 | 32 (part) | −961 | 4102-5063 | F/S | − |
| A3 | 32 (part) | −642 | 4102-4744 | I/F | + |
| A4 | 32 (part) | −642 | 4102-4744 | | − |
| | | −103 | 4918-5021 | F/S | − |
| J21 | 36 | −157 | 5752-5909 | F/S | + |
| J23 | 37 | −213 | 5909-6122 | I/F | − |
| F3 | 38 (part) | −76 | 6257-6333 | F/S | − |
| F1 | 43 (part) | −47 | 6865-6913 | F/S | − |
| I2 | 61 | −1018 | 10157-11175 | F/S | + |

*F/S = frame-shifting; I/F = in-frame

Six probands were heterozygous for a truncating mutation (a frame-shifting deletion or insertion or a nonsense mutation; Table 4). In five of these cases, a second nonconservative missense mutation also was detected. In the sixth case, a more conservative change was identified (I222V; 664A→G), which may generate a new cryptic splice site (TAC/GTCG/GTCTGT; SEQ ID NO:206) four bases upstream from the normal IVS9 5′ site. Unfortunately, an mRNA sample was not available from PKHD1-expressing tissue of this proband, so the effect of the mutation could not be assessed. In the four cases where material was available, segregation was consistent with autosomal recessive inheritance, and in two families both mutations were found in an affected sib(s) (FIG. 5 and Table 4). Two other nonconservative missense mutations were detected in each of two other cases, which also exhibited segregation that was consistent with autosomal recessive inheritance. A mutation in one of these cases (Q1917R) may also disrupt splicing at the normal IVS35 5′ splice site, which was mutated from CAG/GTATAA to CGG/GTATAA. A single possible pathogenic missense mutation was found in three additional cases, and in one of these pedigrees (M51), the same mutation was found in an affected sib (Table 4).

The evidence presented here, including the positioning of the gene within the ARPKD candidate interval, the finding of a mutation in the rat ortholog that causes an ARPKD-like phenotype, the identification of truncating and non-conservative missense mutations in ARPKD patients, and the segregation analysis, demonstrates that this is the PKHD1 gene.

### EXAMPLE 5

### Expression of Pkhd1 and PKHD1

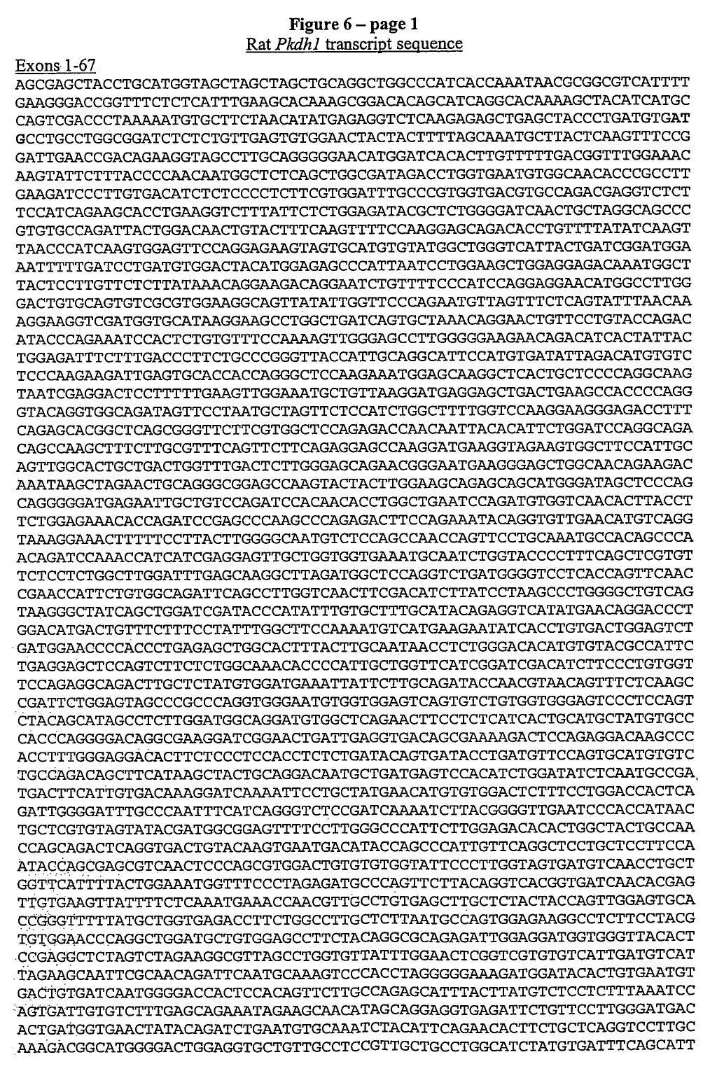

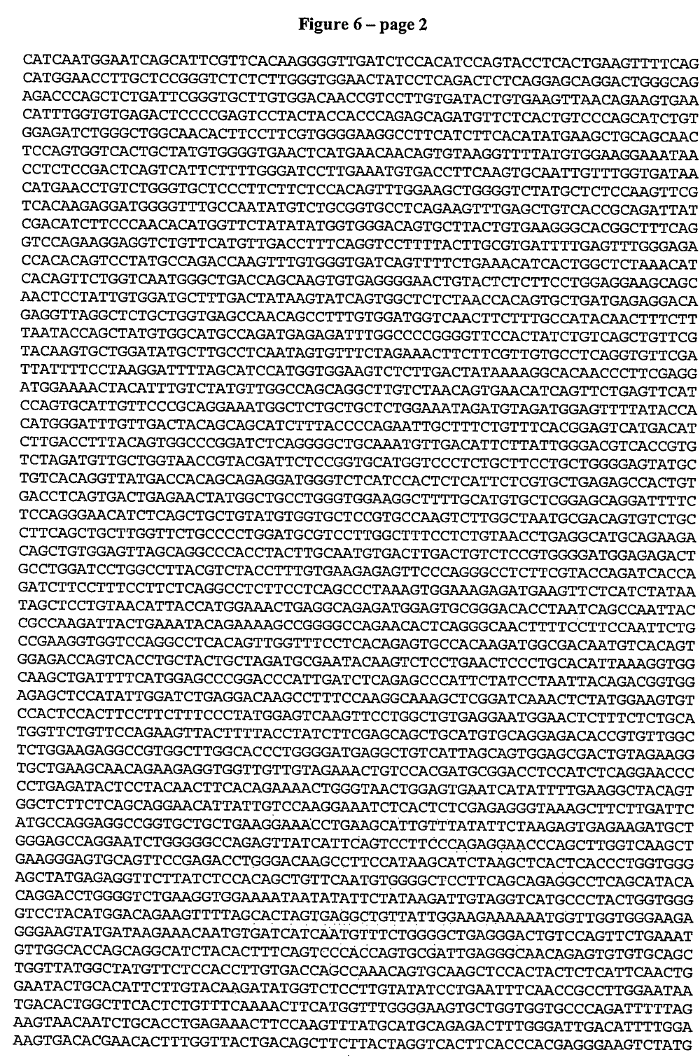

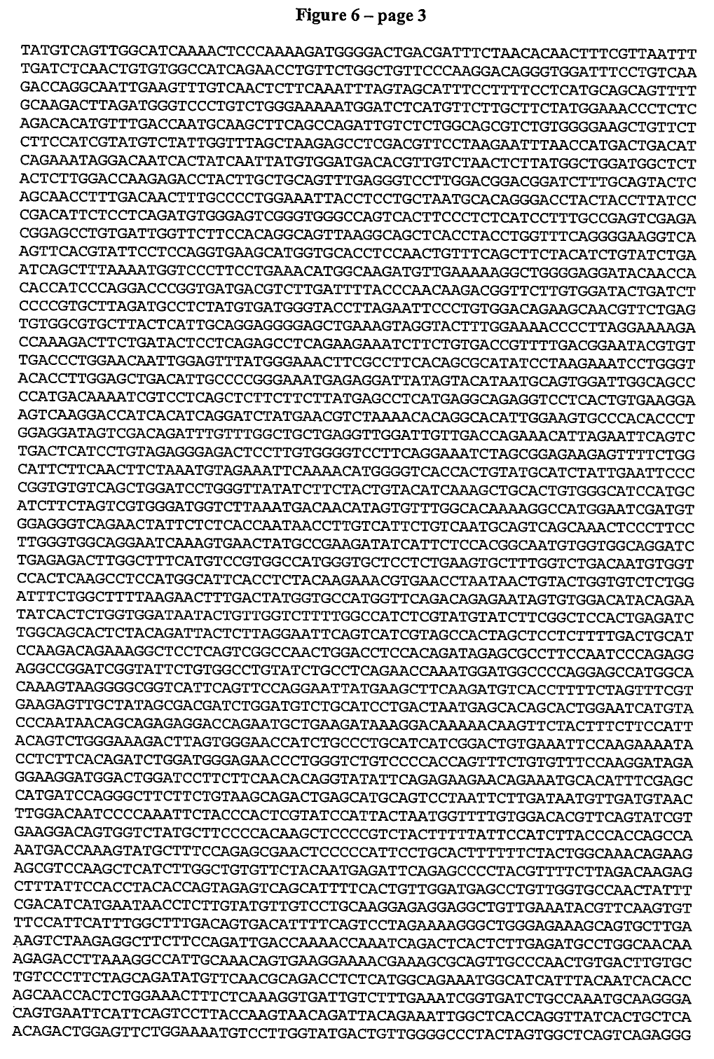

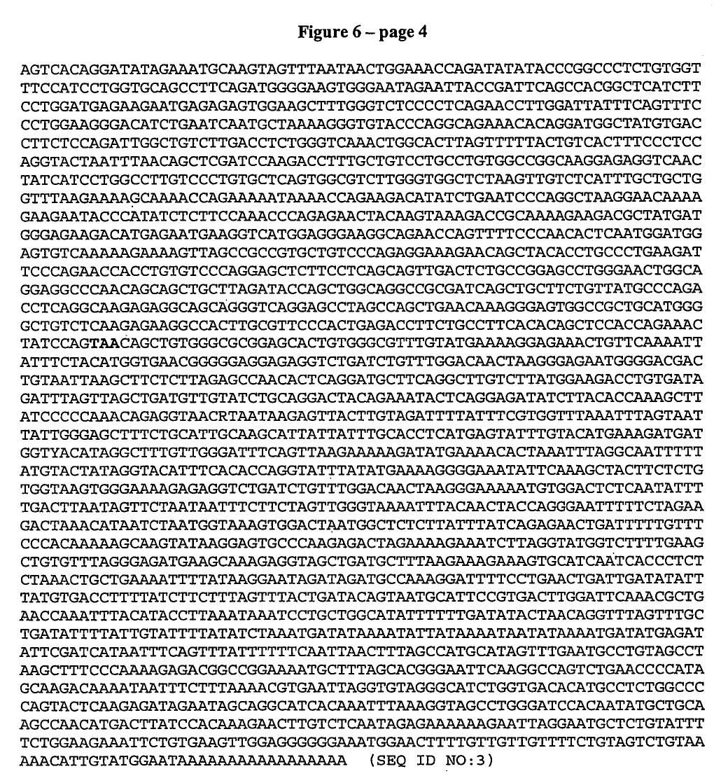

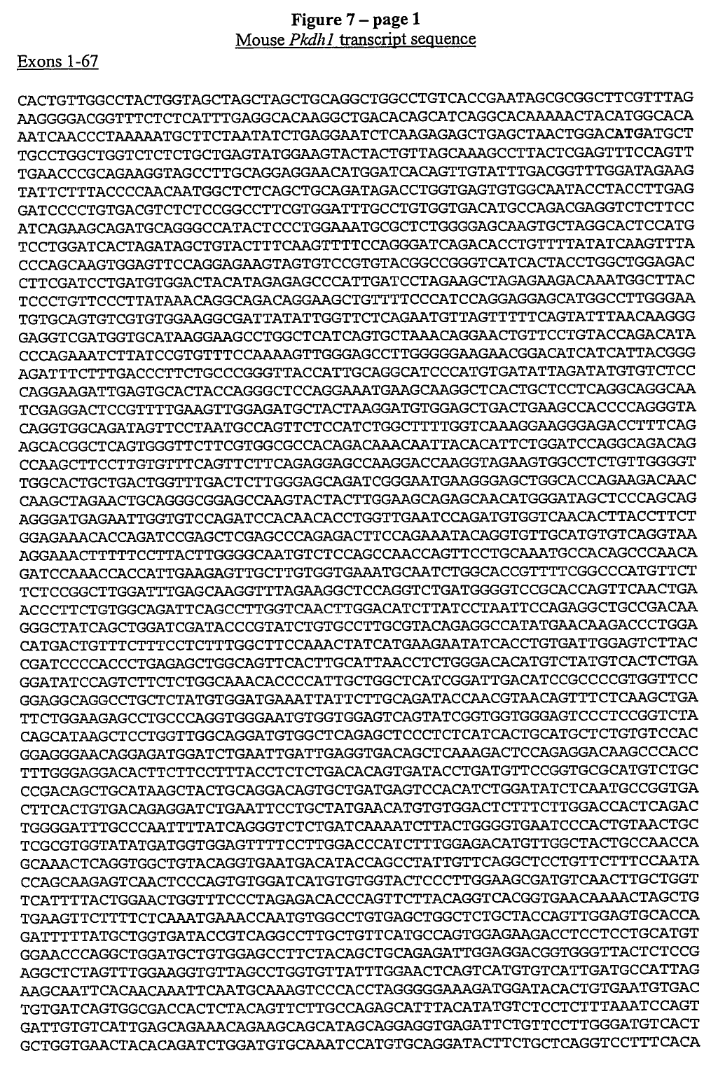

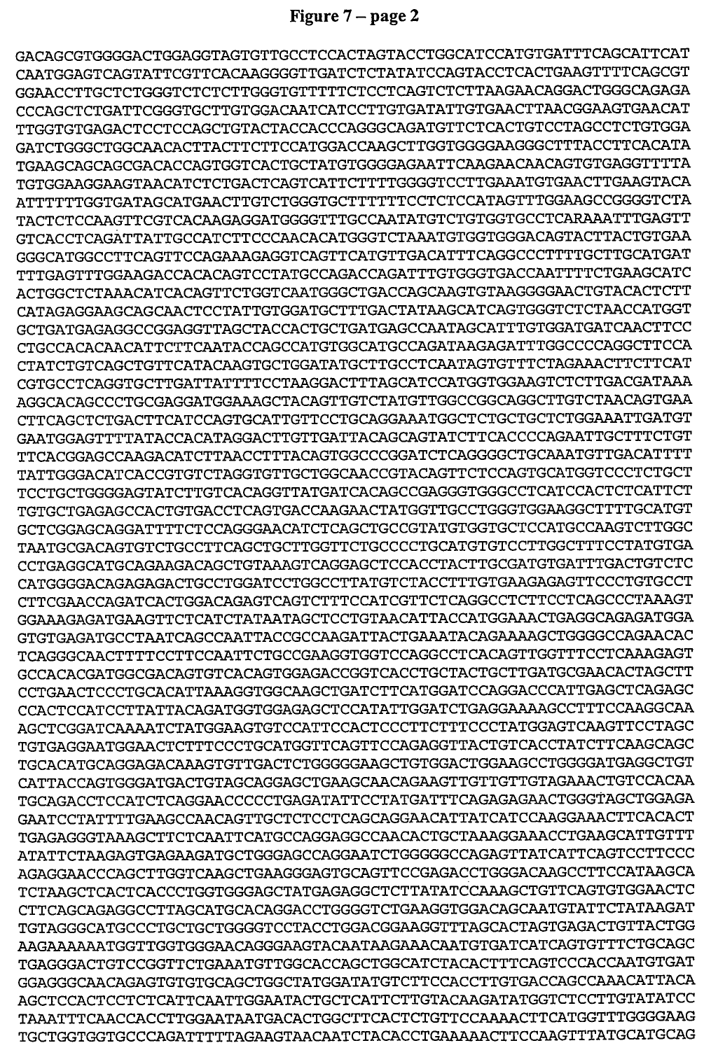

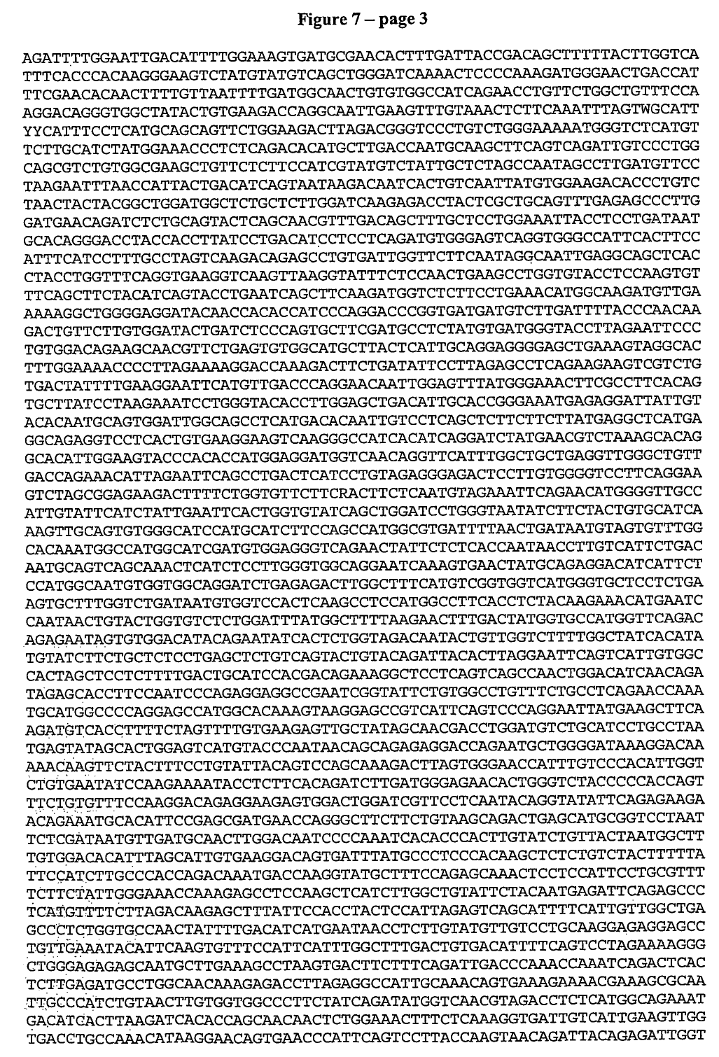

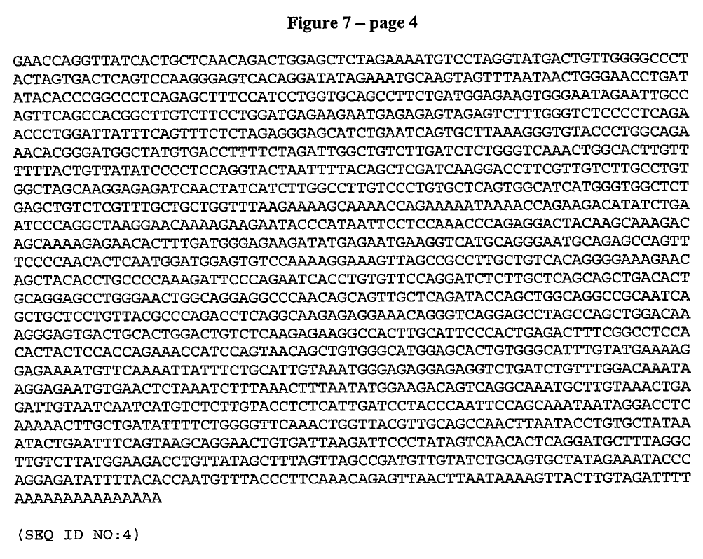

Rat Pkhd1 was visualized by northern blotting as a ˜14 kb transcript. The sequence of the rat transcript (SEQ ID NO:3) is shown in FIG. 6. A mouse transcript of ˜13 kb also has been detected. The sequence of exons 1-67 (SEQ ID NO:4) is shown in FIG. 7. The human transcript is larger, approximately 16.2 kb, due to a long 3′ UTR, and has proven difficult to visualize as a discrete band by northern analysis. Northern blotting revealed only a smear originating around the predicted transcript size, with similar results obtained using either commercial filters or filters generated in-house. The PKHD1 mRNA therefore may be particularly prone to degradation. Based on the intensity of the smears, moderate expression was observed in adult kidney and pancreas, with lower levels in liver. Moderate expression was also detected in fetal kidney using the same criteria.

Northern analysis of mouse RNA showed a moderate level of expression in adult, newborn and fetal kidney, with no signal detected in brain, spleen, colon or heart. RT-PCR was used to more accurately assess the level of Pkhd1 expression in mouse tissues. The strongest expression was observed in kidney, with lower levels detected in liver, pancreas, and lung, and no expression was detected in brain, heart, spleen, colon, thymus, and skeletal muscle. Similar results were obtained with newborn tissues, which also exhibited the highest expression in kidney. Preliminary analysis of cell lines (MacKay et al. (1988) Kidizey Int. 33:677-684) revealed the highest level of expression in the mouse cortical collecting duct cell line, M1. This expression pattern is consistent with the ARPKD phenotype, with the major sites of disease in the collecting duct of the kidney and the liver.

### EXAMPLE 6

### Structure of Fibrocystin

The protein encoded by PKHD1 has been termed fibrocystin, reflecting the hepatic and renal changes associated with ARPKD. The amino acid sequences of human, rat, and mouse fibrocystin (SEQ ID NO:2, SEQ ID NO:6, and SEQ ID NO:7, respectively) are aligned as shown in FIG. 8. The full-length human fibrocystin protein contains 4074 amino acids, and has a predicted unglycosylated molecular weight of 447 kDa. Analysis with the Pfam program revealed the presence of seven TIG domains. These domains typically contain 80 to 100 amino acids, and all seven domains identified in fibrocystin were located within the N-terminal 40% of the protein. TIG domains are found in a wide range of different proteins, both in extracellular and cytoplasmic locations, but the fibrocystin TIG domains are most similar to those found in the extracellular regions of receptor proteins such as the hepatocyte growth factor receptor (HGFR), plexins, and the macrophage-stimulating protein receptor (Ron). FIG. 9A shows the alignment of fibrocystin TIG domains with TIG domains from these other proteins.

The protein with the closest overall homology to fibrocystin is a lymphocyte-secreted murine protein, D86. Similarity between fibrocystin and D86 extends over approximately 1800 amino acids from the N-terminus of both proteins; the two proteins are 24% identical and 41% similar at the amino acid level. The aligmnent of fibrocystin with D86 is shown in FIG. 9B. D86 is predicted to have 11 TIG domains; the homology of these regions with fibrocystin, along with PSI-BLAST analysis, suggests that fibrocystin may have five additional TIG-related regions that do not fully meet the TIG domain criteria. These are included in the alignment shown in FIG. 9A.

BLAST analysis of the fibrocystin sequence revealed three further regions with significant homology to other proteins. The first two regions, between amino acids 1930-2375 and 2882-3069, display homology to the protein TMEM2 (Scott et al. (2000) Gene 246:265-274) and the related predicted protein XP051857. The TMEM2 gene is widely expressed, and the protein is predicted to have a single transmembrane domain, indicating a cytoplasmic N-terminus and large extracellular C-terminal region. The predicted homology between fibrocystin (amino acids 1930-2375), TMEM2, and XP051857 is shown in FIG. 9C. Fibrocystin also contains a region that is significantly homologous to a predicted protein of unknown function (DKFZp586C1021), with identity of 24.7% and similarity of 43.7%; the alignment is shown in FIG. 9D.

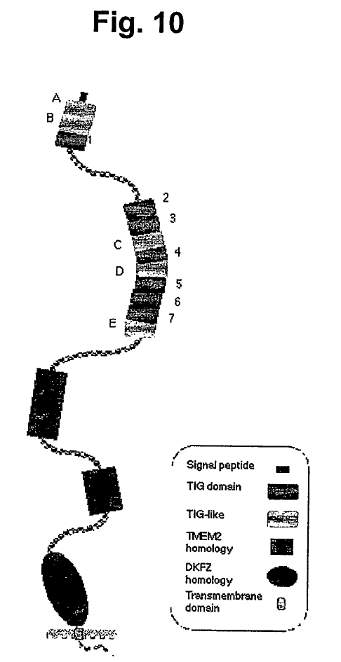

Analysis of the N-terminal region of fibrocystin indicated the presence of a hydrophobic signal peptide, with predicted cleavage after position 19. Inspection for potential transmembrane (TM) domains was complicated by the hydrophobic nature of much of the protein, and the programs PHDhtm and TopPred2 predicted multiple TM domains. Some of these predicted TM domains, however, were within the TIG domain region or in areas predicted to be extracellular based on homology to other proteins such as TMEM2. A more likely prediction was generated by two other programs, SOSUI and TMHMM, which indicated two transmembrane regions: one associated with the signal peptide and the other between residues 3859 and 3881. This model therefore predicted that most of the fibrocystin protein is extracellular, with just a short cytoplasmic tail of 192 amino acids (FIG. 10). This structure is consistent with both the presence of the TIG domains and the homology to TMEM2, which also has a large extracellular domain. In addition, the predicted external region of fibrocystin contains 64 potential N-glycosylation sites, suggesting that the protein may be highly glycosylated. Potential clues as to the function of fibrocystin were provided by the identification of potential phosphorylation sites for PKA (residue 3956) and PKC (residues 3887, 3910, and 3951) within the C-terminal tail, which are consistent with the predicted structure and are conserved in the mouse Pkhd1 gene.

### EXAMPLE 7

### Monoclonal Antibodies to the C-Terminal Tail of Fibrocystin

A 583 bp fragment of human PKHD1 encoding the 192 aa C-terminal tail of fibrocystin was amplified by PCR. The product was subcloned into the pET-43^{a+} vector (Novagen) to generate a fusion protein containing the soluble Nus A protein and the C-terminal polypeptide of fibrocystin. The recombinant protein was purified by cobalt affinity chromatography (TALON™, BD Clontech, Palo Alto, Calif.), and the fibrocystin segment was cleaved using TEV protease and purified by ion exchange chromatography.

Four mice were immunized with the purified fibrocystin protein and monoclonal antibodies were generated by standard methods. Five hundred clones were screened by ELISA and positive clones were analyzed by western blotting of the purified protein. Western positive clones were further assayed by immunostaining of human kidney tissue, and five clones that detected fibrocystin in all assays were used to characterize the endogenous protein. Two of the monoclonals (FB1 and FB2) were IgMs and three (FB5, FB6 and FB7) were IgGs.

Endogenous fibrocystin was assayed by western blotting of human, mouse, and rat kidney and other tissues. Membrane preparations of tissues were isolated by standard sucrose cushion methods. For western analysis, 1-5 mg of membrane protein was separated on 3-8% Tris-acetate NuPAGE gradient gels (Invitrogen) and transferred to Immobilon-P membrane (Millipore, Billerica, Mass.). Fibrocystin was detected with the primary antibody and an appropriate isotype specific, peroxidase conjugated, secondary antibody. In the kidney of all species tested, a large molecular weight protein (>400 kDa) was strongly detected with each of the monoclonal antibodies. Analysis of ARPKD tissue showed no such large protein, although the large epithelial membrane antigen (EMA) was detected. These results indicate that a large, full-length fibrocystin protein is detected with the monoclonal antibodies in renal tissue and that this product is lost in ARPKD kidney. Analysis of other murine tissues showed that the large fibrocystin product was only found in kidney, but smaller fibrocystin products (e.g., ˜200 kDa) were detected in other tissues such as liver. Analysis of various renal derived cell-lines has not revealed the >400 kDa product, suggesting that some characteristics of intact tissue are required for expression of the full-length product.

Immunostaining of formalin fixed tissues was performed to determine the cellular distribution of fibrocystin. Samples were obtained from fetal and childhood kidneys, liver, pancreas, spleen, adrenal gland, heart, bowel, and testes ranging between 30 weeks gestation and five years of age, as well as adult kidney and liver. In the fetal kidneys, staining for fibrocystin was detected in the epithelial cells of the branching ureteric bud and in the elongating tubules within the renal cortex and nephrogenic zone. At later stages of gestation and early childhood, staining for fibrocystin was noted not only in the collecting ducts but also in the distal tubules, loops of Henle, and proximal tubules. No staining was detected in the glomeruli. In the adult kidneys, immunostaining for fibrocystin was weaker and mostly confined to the collecting ducts. Staining for fibrocystin also was detected in epithelial cells of the bile ducts in the portal tracts, with a weaker signal in hepatocytes. Strong fibrocystin staining also was evident in cardiac myocytes and in the adrenal gland. Weak-to-moderate staining was detected in testes and bowel, while the pancreas and spleen were mostly negative.

### EXAMPLE 8

### Materials and Methods for Mutation Screen of Pedigrees

Details of the study cohort: A family history and clinical information was collected from 66 pedigrees containing one or more patients with a phenotype consistent with ARPKD. These families came from Spain (designated OV, PRR or HEP), the United States (M), and the United Kingdom (P). The proband and all family members wishing to participate gave a blood sample for DNA isolation. For purposes of analysis, the cohort was divided into two groups: those that presented as neonates or children with predominantly kidney disease (typical ARPKD) and those presenting during childhood or later with liver disease (CBF) being the major disease manifestation.

Amplification of the PKHD1 gene by PCR: Genomic DNA was isolated from a peripheral blood sample by standard methods. In a few perinatal cases the QIAmp DNA Mini Kit (QIAGEN Inc.) was used to extract genomic DNA from formalin fixed, paraffin embedded kidney tissue blocks. In these cases, three slices of tissue 20 millimeters thick were obtained using a Biocut 2030 microtome (Leica Instruments), and were dissolved in xylene to release the tissue, followed by a 100% ethanol wash. Samples were digested with proteinase K at 56° C. overnight and the DNA isolated using the standard QIAmp DNA kit extraction protocol.

All coding exons of the PKHD1 gene were amplified from genomic DNA as fragments of 150-370 bp. Primers generally were positioned within introns 25-30 bp from the exon boundary, in order to detect mutations of the canonic splice sites but to minimize detection of non-pathogenic intronic changes. PCR was performed as described in Example 1 herein (see subsection entitled Mutation analysis by denaturing high-performance liquid chromatography (DHPLC)), except that PCR mixes contained 6 pmole of each primer. This protocol was used for all exons except 2 and 3, where a DMSO-based PCR buffer (Dodé et al. (1990) Brit. J. Haematol. 76:275-281; exon 2) and a hot start protocol with a high annealing temperature (exon 3) was employed due to persistent nonspecific amplification and primer dimerization. PCR primers and conditions were as summarized in Table 5. Heteroduplexes also were generated as described in Example 1. This analysis assumed that patients were compound heterozygotes for mutations. To screen for homozygous changes, an equal quantity of normal amplicon was added to the patient product before heteroduplex formation.

Mutation analysis of the PKHD1 gene by DHPLC: DHPLC was performed using the Wave system (Transgenomic Inc.) as described previously (Ward et al. (supra); and Rossetti et al. (supra). Briefly, 300-600 ng of crude PCR product was injected into a chromatographic column (DNASep cartridge, Transgenomic Inc.) and eluted through an 8.6 min linear gradient of Buffer A (5% TEAA) and Buffer B (5% TEAA and 25% Acetonitrile). A 2% Buffer B slope per minute was used. Melting profiles were analyzed using Wavemaker 4.0.32 software and each amplicon was run at the predicted melting temperature ±1 and 2° C. (and additional temperatures when needed) to optimize conditions. Due to the initial lack of positive controls, a set of 8 samples was used to test the analysis conditions. The optimal temperature was considered to be the one immediately before a significant decrease in the retention time (before 3 minutes) and/or excessive broadening of the peak occurred, indicating excess denaturation. This typically was located in the range of 50-75% helical fraction. When a sequence change was found, that sample was used to refine the optimal analysis temperature, at which the best resolution of the mutant amplicon occurred. Where more than one positive control was available, the most subtle change was chosen as an internal control. Samples showing an aberrant elition profile typically were re-amplified and subjected to direct sequencing. The DHPLC conditions and positive controls available for each amplicon are summarized in Table 9.

Validation of mutations: Missense mutations and subtle splicing mutations were validated by analyzing 50 normal controls (100 normal chromosomes) using DHPLC and including the candidate mutation for comparison with the normal samples. Whenever DNA from other members of the pedigree was available, missense and subtle splicing mutations also were confirmed by family segregation analysis. This was performed by DHPLC or by direct sequencing when more than one sequence change was present in the fragment being analyzed.

Restriction assays were developed to facilitate the detection of the four most common mutations: 5895insA, 9689delA, T36M, and I222V. The restriction enzyme HpyCH4 IV (New England Biolabs) was used for mutations T36M and I222V. The T36M mutation abolishes a restriction site, such that the normal restriction pattern of exonic fragment 3 (54+52+52 bp) is changed to 106+52 bp. The I222V mutation creates a new restriction site, such that the exon 9 amplicon (which is not cut by the enzyme in the absence of the mutation) generates fragments of 102+55 bp. A restriction generating PCR (RG-PCR) approach was designed to detect 5896insA with a modified reverse primer (5′-ACTTCACACACCTTTAATGTGCACT-3′; SEQ ID NO:207; underlined base modified) and the forward exon 36 primer (Table 5). The normal 206 bp fragment was resolved as 179+28 bp when the mutant was amplified and digested with Afl II (New England Biolabs). The restriction enzyme HpyCH4 III (New England Biolabs) was predicted to digest the mutant exon 58d amplicon as fragments of 119+188 bp. As this digestion was inconsistent, DHPLC with the undigested fragment added was used to analyze for homozygotes. Restriction digestions typically were performed in a total volume of 20 μl, using 10 μl (˜1 mg) PCR product and 10-20 units of restriction enzyme in the supplied buffer. Reactions were incubated at 37° C. for 2 hours. BSA (1%) was added to Afl II. Restriction bands were visualized on 3% agarose gels after ethidium bromide staining.

Positions of mutations are described using the PKHD1 cDNA sequence, AY074797, with the A of the start codon designated as first nucleotide. The program SignalP 2.0 (World Wide Web at cbs.dtn.dk/services/SignalP/) was used to analyze the consequence of the A17V mutation on cleavage of the protein.

TABLE 9
DHPLC conditions and positive controls

| | DHPLC Conditions | | |
|---|---|---|---|
| Exonic fragment | Temp (° C.) | Initial % buffer B | Positive control |
| 2 | 53, 54 | 53 | IVS1 − 47C/T |
| 3 | 57 | 48 | T36M |
| 4 | 60 | 53 | V/M65 |
| 5 | 60 | 50 | 383delC |
| 6 | 58 | 49 | None |
| 7 | 55 | 49 | IVS7 + 19T/C |
| 8 | 56 | 49 | None |
| 9 | 55, 57 | 47 | I222V |
| 10 | 53 | 49 | None |
| 11 | 54 | 49 | None |
| 12 | 55, 57 | 47 | None |
| 13 | 56, 58 | 48 | None |
| 14 | 59 | 52 | IVS14 + 23G/T |
| 15 | 58 | 50 | 1185T/C |
| 16 | 61 | 55 | None |
| 17 | 58 | 50 | 1587T/C |
| 18 | 58 | 49 | 1624del4 |
| 19 | 61 | 52 | None |
| 20 | 60 | 50 | None |
| 21 | 61 | 54 | 2046A/C |
| 22 | 59 | 52 | R/C760 |
| 23 | 59 | 53 | IVS23 + 50C/T |
| 24 | 58 | 53 | W/R852 |
| 25 | 60 | 51 | None |
| 26 | 56, 58 | 52 | R/Q909 |
| 27 | 52, 57 | 56 | 2853C/T |
| 28 | 59 | 51 | None |
| 29 | 53, 56 | 53 | IVS28 − 2A→C |
| 30 | 58 | 55 | 3537T/C |
| 31 | 56, 58 | 48 | None |
| 32a | 54, 58 | 55 | A/V1262 |
| 32b | 61 | 56 | None |
| 32c | 60 | 55 | L1407R |
| 32d | 60 | 56 | None |
| 32e | 58, 59 | 56 | None |
| 32f | 59 | 55 | S1664F |
| 32g | 58 | 48 | IVS32 + 42del4 |
| 33 | 60 | 52 | None |
| 34 | 60 | 54 | 1833L |
| 35 | 58 | 52 | Q1917R |
| 36 | 61 | 51 | D1942G |
| 37 | 61 | 54 | E1995G |
| 38 | 59 | 54 | None |
| 39 | 58 | 52 | 6383delT |
| 40 | 59 | 54 | None |
| 41 | 56, 58 | 53 | None |
| 42 | 53 | 47 | None |
| 43 | 57, 58 | 50 | I2331K |
| 44 | 56, 57 | 50 | None |
| 45 | 56 | 50 | None |
| 46 | 56 | 50 | None |
| 47 | 56 | 51 | None |
| 48 | 57 | 55 | 7587G/A |
| 49 | 57 | 53 | 7764A/G |
| 50 | 58, 60 | 54 | R2671X |
| 51 | 56 | 49 | P/S2720 |
| 52 | 53, 55 | 51 | None |
| 53 | 59 | 51 | None |
| 54 | 53 | 51 | R/C2840 |
| 55 | 54 | 49 | T2869K |
| 56 | 56, 58 | 52 | None |
| 57 | 56, 58 | 52 | I2957T |
| 58a | 58 | 54 | S3018F |
| 58b | 60 | 54 | 9237G/A |
| 58c | 58 | 53 | D3139Y |
| 58d | 57 | 54 | 9689delA |
| 59 | 52 | 53 | None |
| 60 | 58 | 52 | C3346R |
| 61a | 55, 57 | 56 | Q3392X |
| 61b | 58 | 56 | I3553T |
| 61c | 58 | 56 | 10856delA |
| 61d | 57 | 51 | IVS61 + 9A/G |
| 62 | 55, 56 | 53 | R/W3739 |
| 63 | 56, 57 | 50 | 11340T/C |
| 64 | 56, 57 | 51 | None |
| 65 | 54 | 53 | W3871X |
| 66 | 56, 57 | 52 | Q/R3899 |
| 67a | 60 | 54 | V/I3960 |
| 67b | 61 | 54 | Q/R4048 |

### EXAMPLE 9

### Mutation Screening of a Large Novel Cohort of ARPKD and CHF Patients

The large size of the PKHD1 open reading frame (12,222 bp) and multiple exon structure indicated that a rapid semi-automated method for mutation screening was required. The 66 coding exons of PKHD1 were amplified from genomic DNA as 79 PCR amplicons, ranging from 150-370 bp (see Table 5 for details), a size previously found optimal for DHPLC analysis (Rossetti et al., supra). Most exons were amplified as a single fragment, but multiple overlapping fragments were required for the larger exons 32, 58, 61, and 67 (see Table 5). To establish appropriate conditions for DBPLC, each fragment was analyzed using the Wavemaker software and idealized conditions were determined emperically. Most amplicons were analyzed at a single temperature but, because of different distinct melting domains, 22 amplicons were analyzed at two different temperatures (see Table 9 for details). Fragments that generated an aberrant profile were sequenced to determine the DNA change and, if samples were available, segregation was analyzed in the family. Changes predicted to truncate the protein and missense and splicing changes that segregated with the disease (if analysis was possible) and were not found in 100 normal controls were considered to be mutations. An initial test of the screening system employed a small group of ARPKD patients as part of a study to identify the disease gene (Ward et al., supra), and 18 different mutations were identified throughout PKHD1.

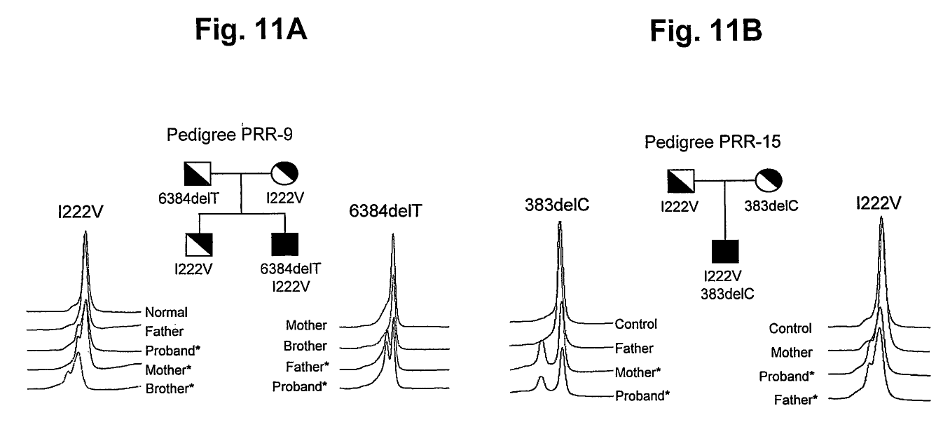

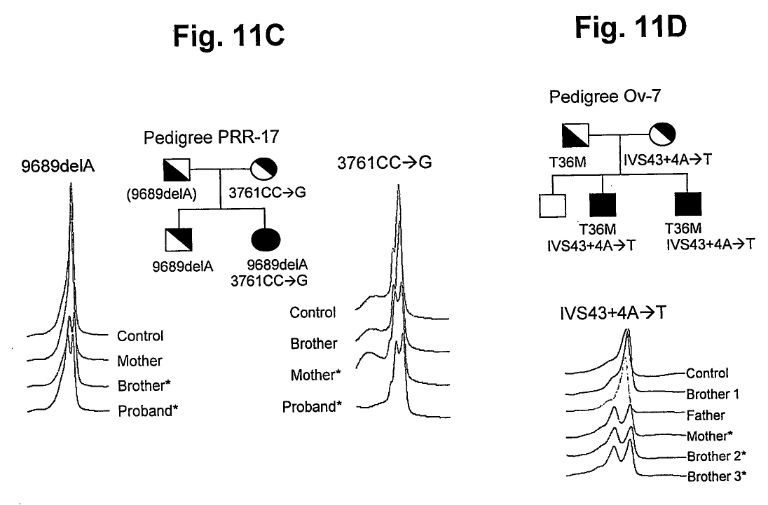

To establish a clearer view of the types of mutations associated with ARPKD, and the prospects for gene-based diagnostics, a larger cohort of patients was analyzed. DNA samples were collected from Spanish, American and British pedigrees. As the preliminary analysis (Ward et al., supra) indicated that PKHD1 mutations were found in patients with a primary diagnosis of CHF and/or CD, as well as typical ARPKD, patients with a wide range of phenotypes were analyzed. A total cohort of 66 families was screened for mutations, 47 with a primary diagnosis of ARPKD and 19 with a diagnosis of CHF and/or CD. DNA from the proband was analyzed in 61 cases, while parental DNA was screened in the remaining 5 families since the patients had died in the perinatal period and no samples were available. Examples of mutant DHPLC profiles and segregation analysis are shown in FIGS. 11A-11D. Mutant heteroduplexes are visualized as an earlier elution peak or shoulder on the homoduplex peak. Asterisks indicate individuals with mutations. In each case the affected individuals are compound heterozygotes for mutations inherited from both parents, consistent with a recessive disease. Affected individuals are shown as filled shapes and carrier individuals are shown as half-filled shapes. Details of the mutations identified in this cohort are shown in Table 10, as are clinical characteristics of these patients. Mutation details of the population are summarized in Table 11.

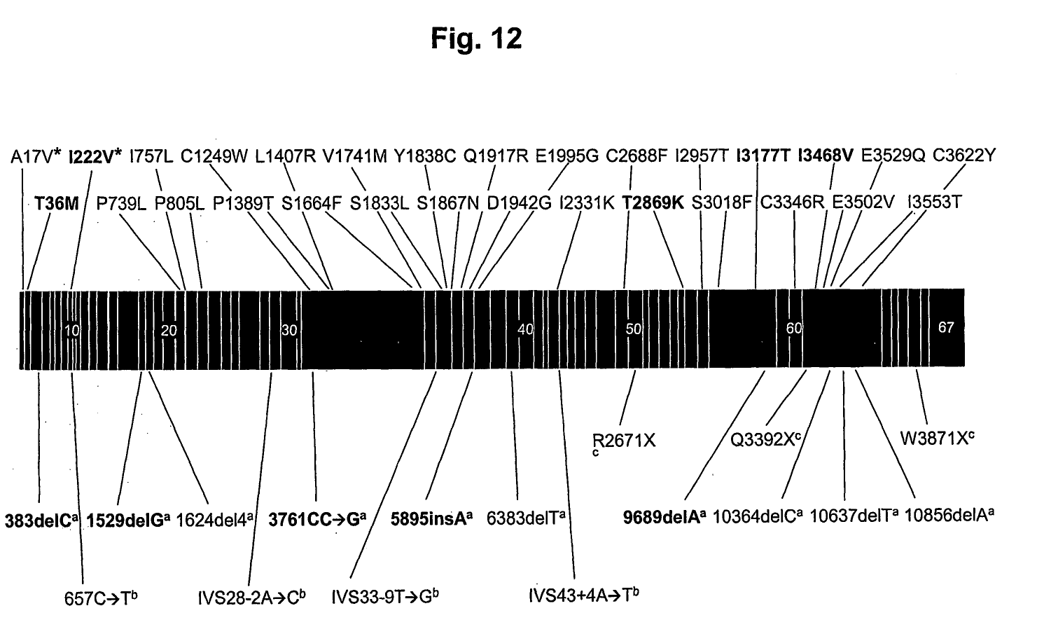

The mutations identified herein are spread throughout the gene (FIG. 12). A total of 33 different mutations were characterized on 57 mutant alleles; mutations on both alleles were identified in 22 families and mutation(s) on only one allele were identified in a further 13 families (see Table 11 for details). Ten mutations were found on more than one allele, and two of these were particularly frequent: 9689delA (9 alleles) and 5895insA (8 alleles). Three missense changes, T2688K (5 alleles), T36M (4 alleles) and I222V (3 alleles), also were common. Eight different insertion or deletion mutations were identified that were predicted to cause a frame-shifting change, accounting for 26 of the mutant alleles, while 21 missense changes were found on 27 alleles. In addition, four potential intronic or exonic splicing mutations were identified. IVS28-2A→C is a clear splicing mutation. The remainder may change or create splice sites: IV33-9T→G may weaken the polypyrimidine tract; IVS43+4A→T may lower the strength of the splice acceptor site; and 657C→T may generate a cryptic splice site, AAG/G(T)GACT, close to the end of exon 9. Two of the missense mutations also may cause aberrant splicing. The conservative substitution, I222V, may cause cryptic splicing at the end of exon 9, while the A17V mutation may generate a cryptic splice site, TGG/G(T)AGGT, four bp 5′ to the normal IVS2 splice site, resulting in a frameshifting change. The A17V mutant also may cause disease by disrupting cleavage of the protein. This change is predicted to move the site of cleavage of the signal peptide four residues C-terminal to the sequence LSL-HI (SEQ ID NO:208).

In the 22 pedigrees where mutations were identified on both alleles, 20 were compound heterozygotes. These included one pedigree with two truncating changes; ten with one truncating and one missense mutation; five with two missense mutations; three with one missense and one splicing mutation, and one with one truncating and one splicing mutation (see FIG. 11 for examples). The probands in families PRR-1 and PRR-12 were predicted to be homozygous for 9689delA (from analysis of the parental alleles) but patient material was not available for confirmation. Restriction assays were developed to rapidly screen for the four most common changes, 5895insA, 9689delA, T36M and 1222V. FIG. 11D shows how this assay was used to trace T36M in pedigree OV-7. To test whether other homozygous cases were missed by the screening method used herein, the four common mutations were analyzed by restriction assays or by DHPLC analysis after adding normal DNA. In addition, the entire gene was screened by DHPLC with normal DNA added in six mutation negative patients, but neither of these methods identified any further homozygous mutations.

In addition to the putative pathogenic changes, a total of 34 polymorphic changes were detected, 24 of which are exonic and 10 of which are intronic (Table 12). These changes were defined as polymorphisms because they were detected in the normal population or did not segregate appropriately with the disease phenotype. Several of the changes involve non-conservative amino acid substitutions.

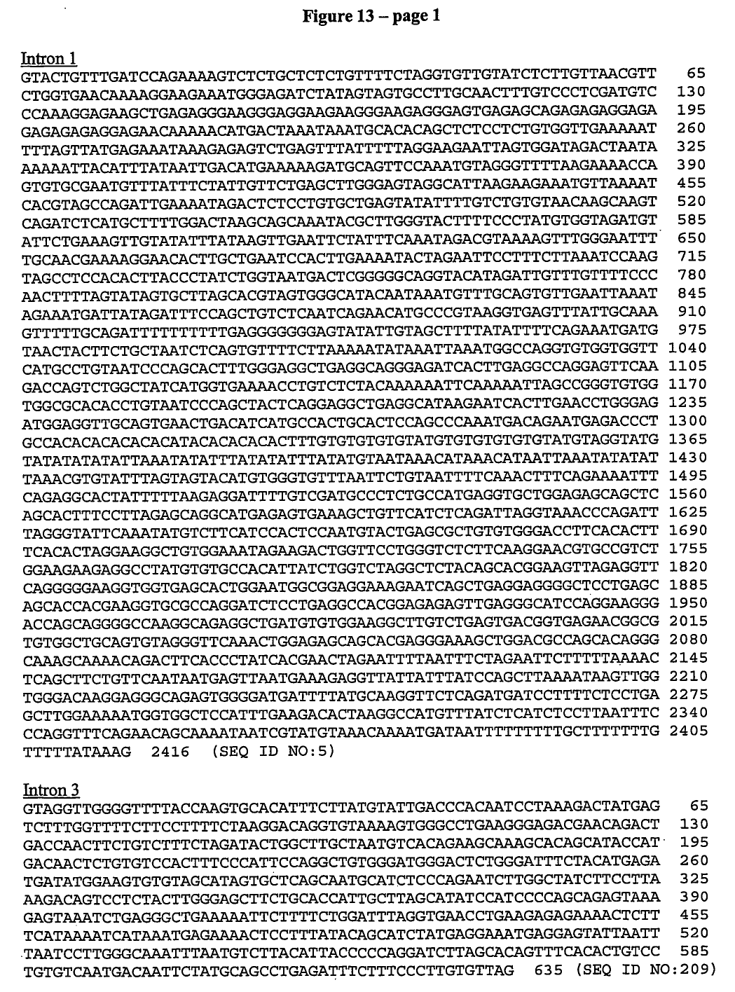

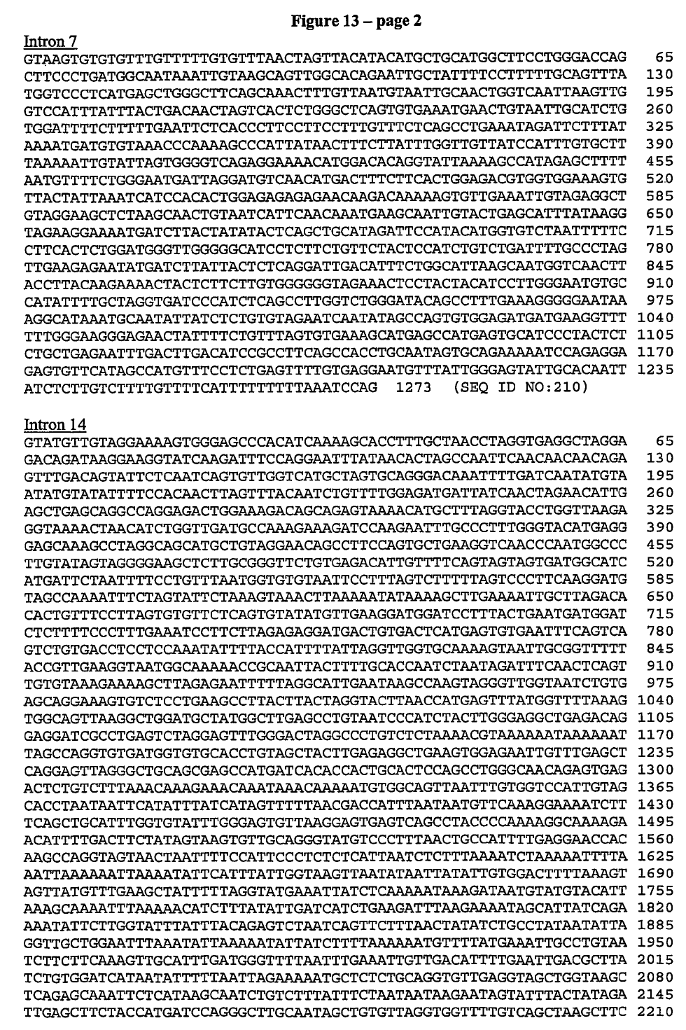

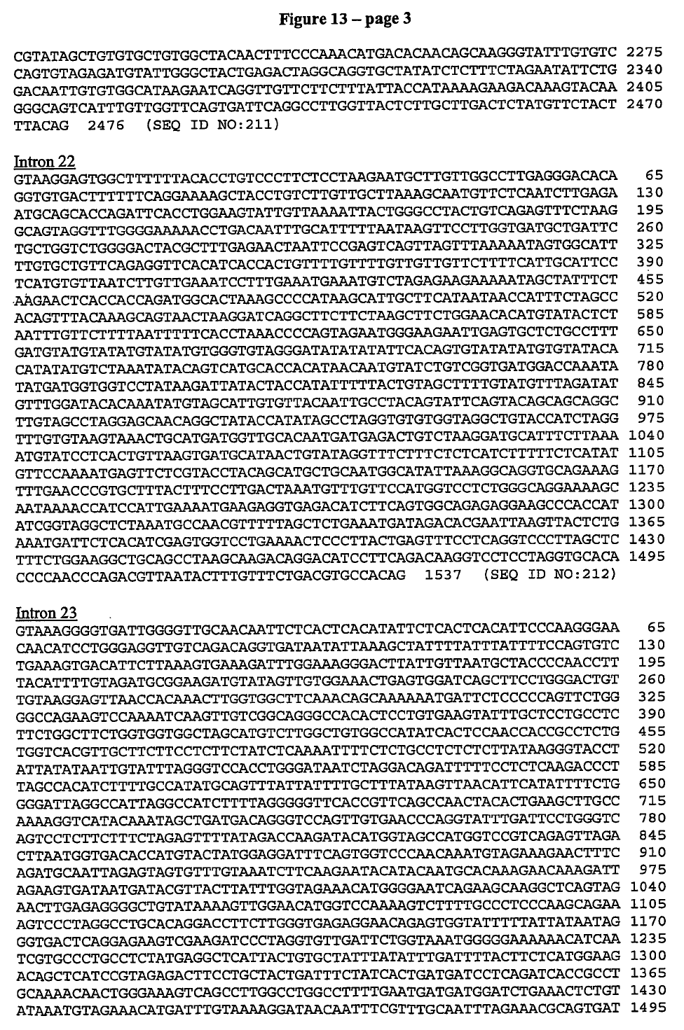

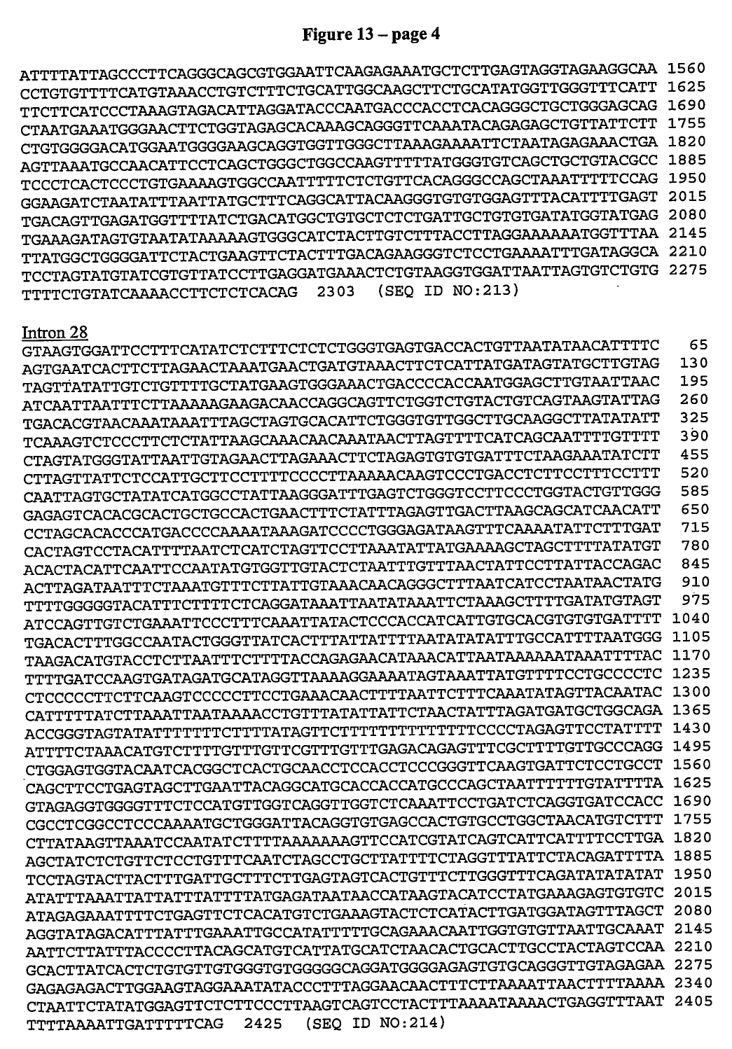

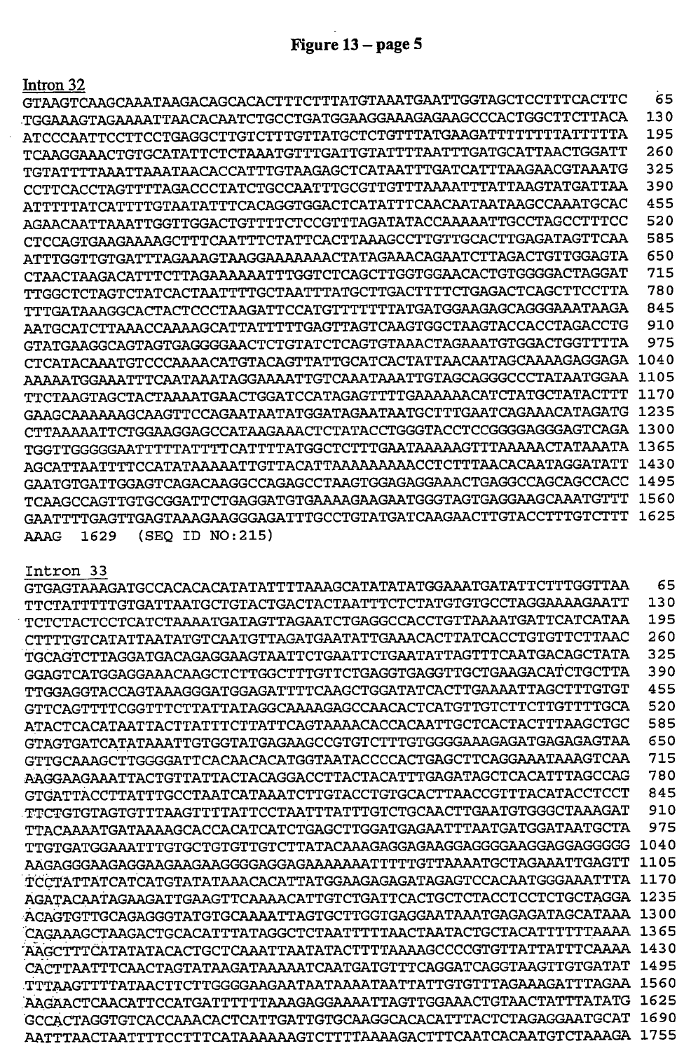

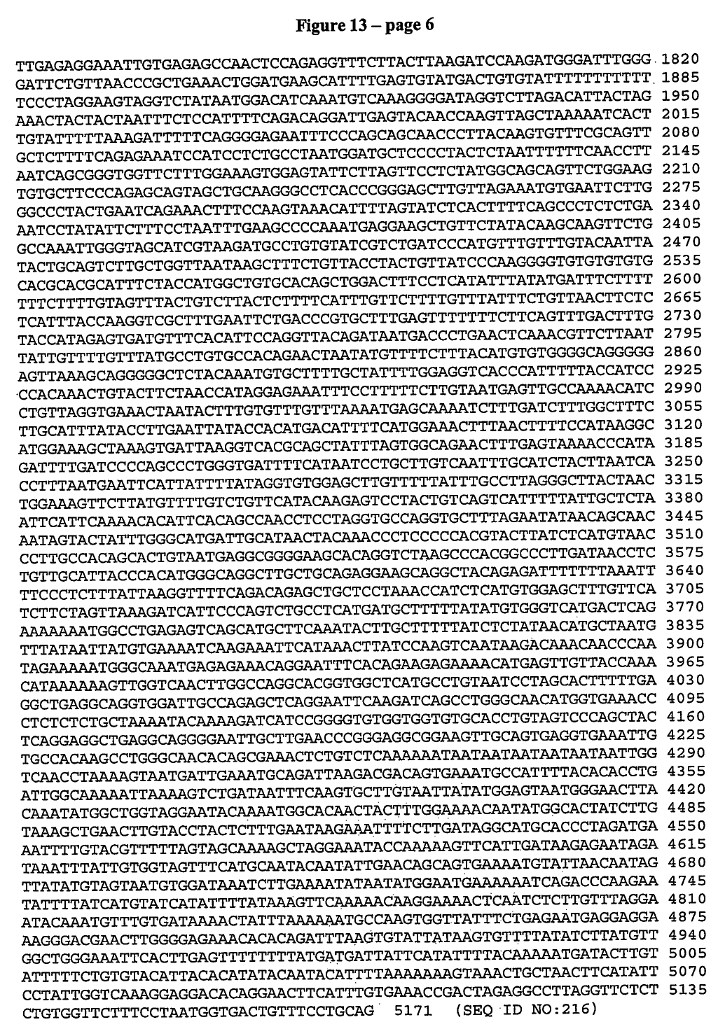

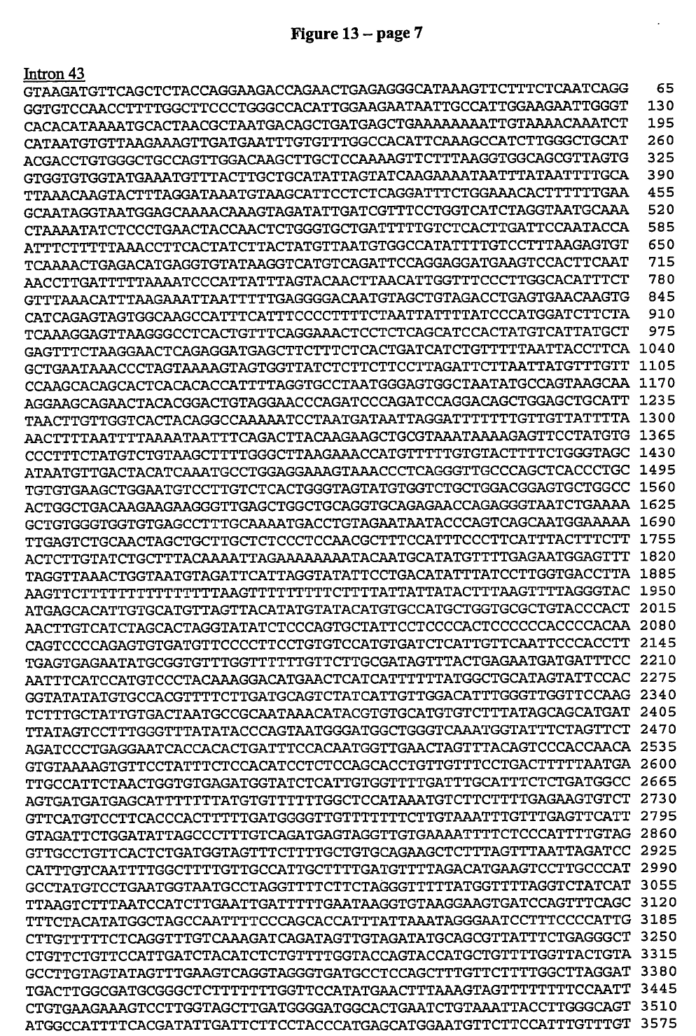

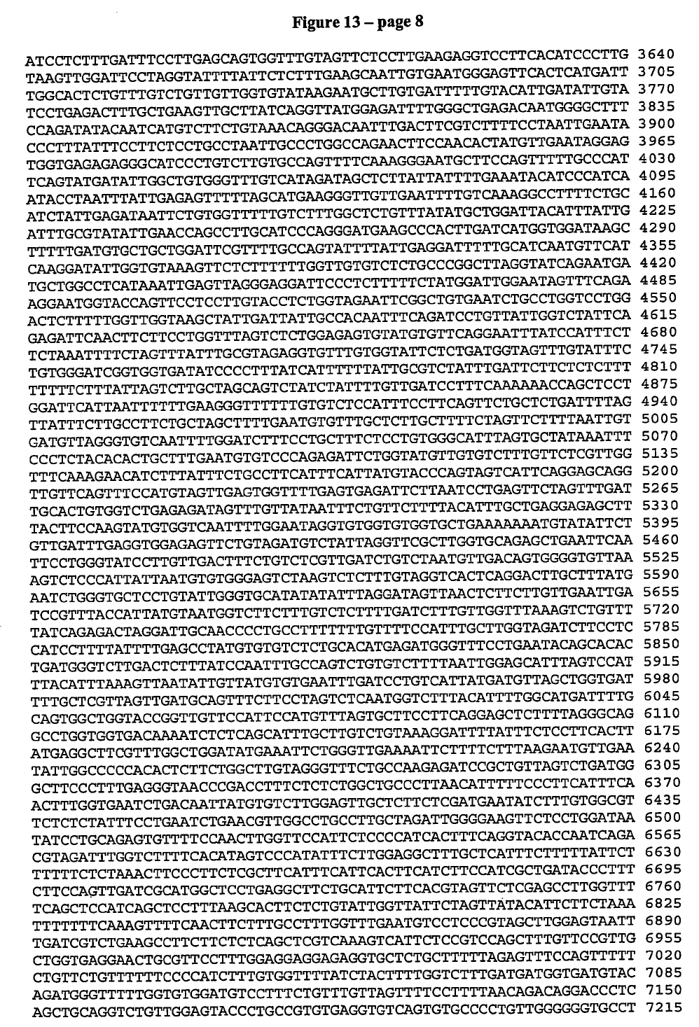

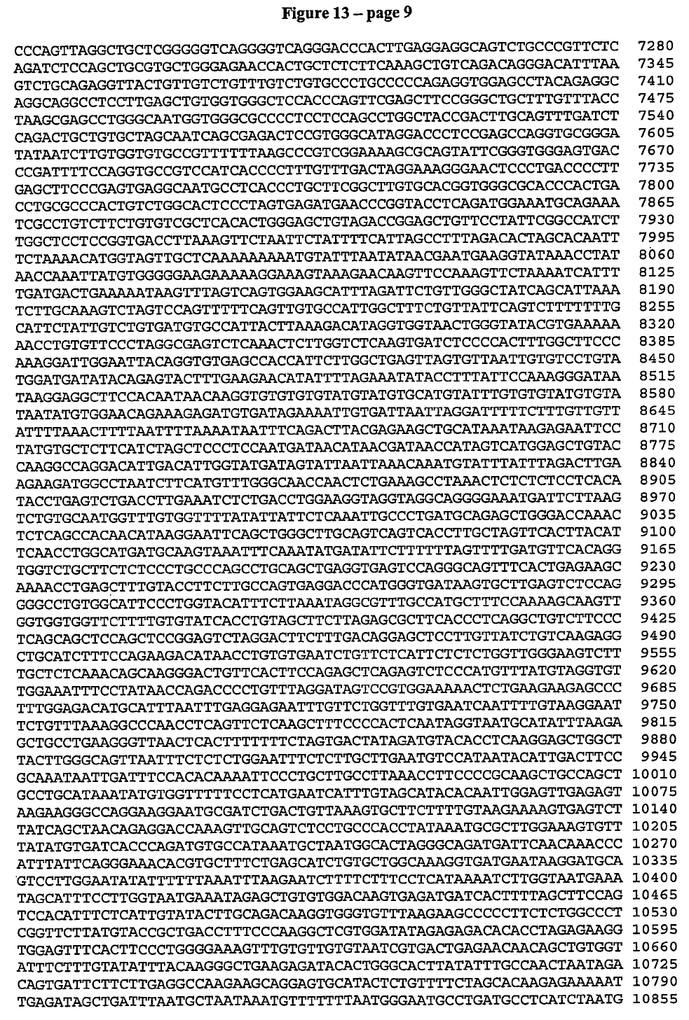

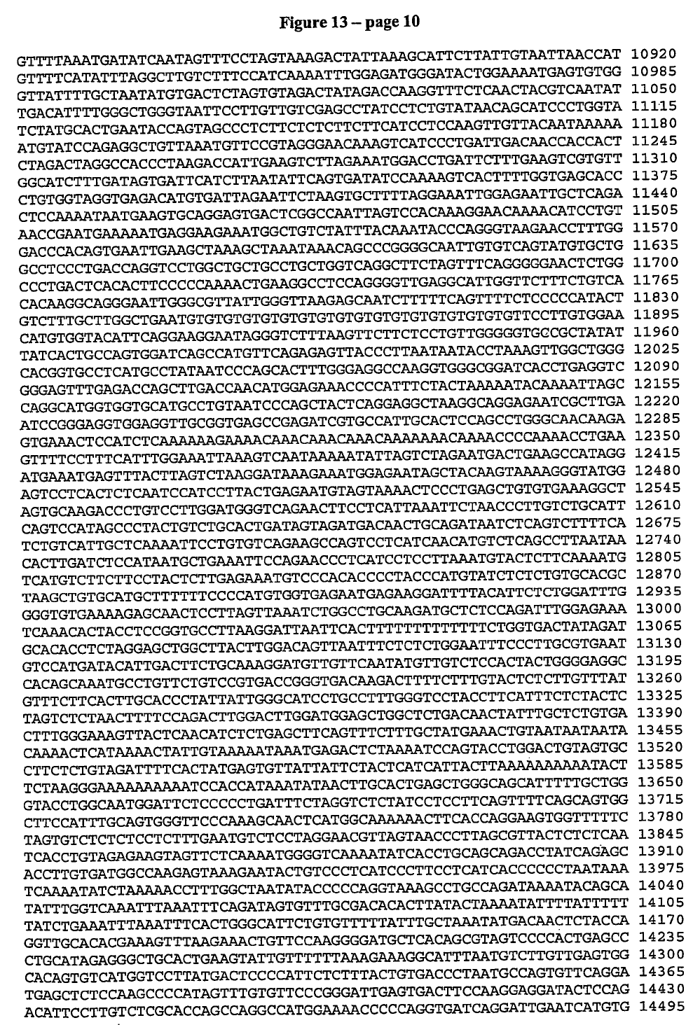

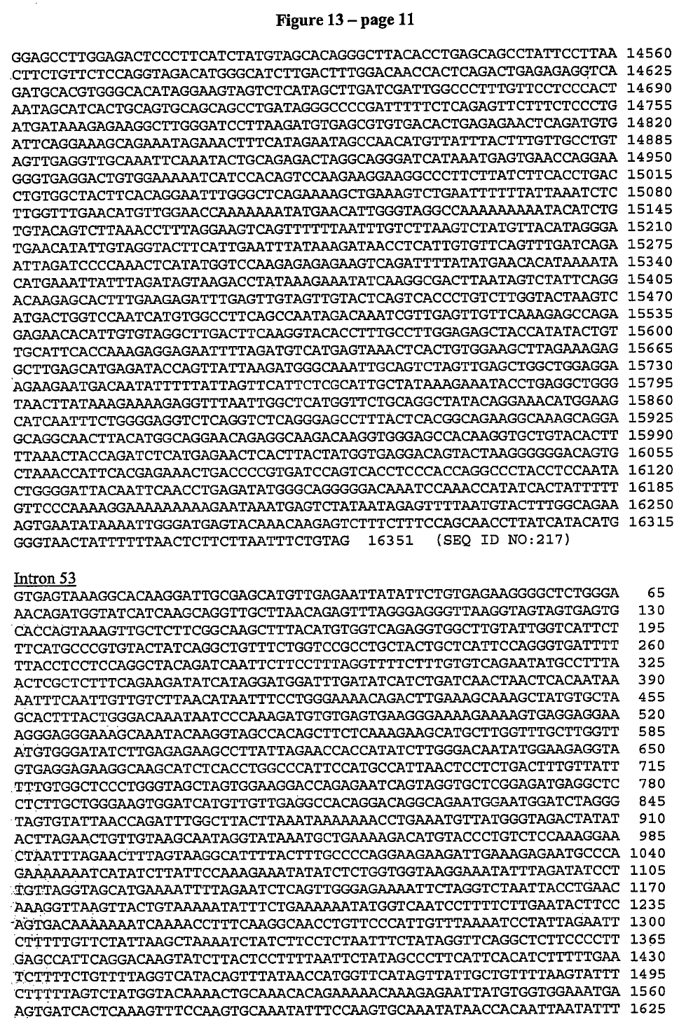

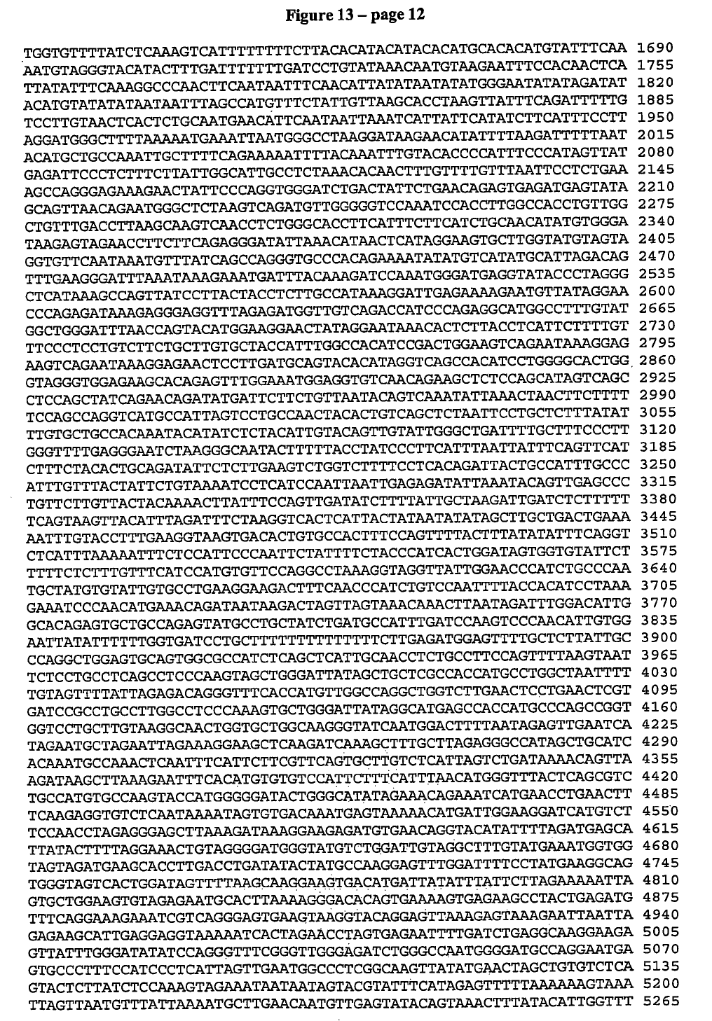

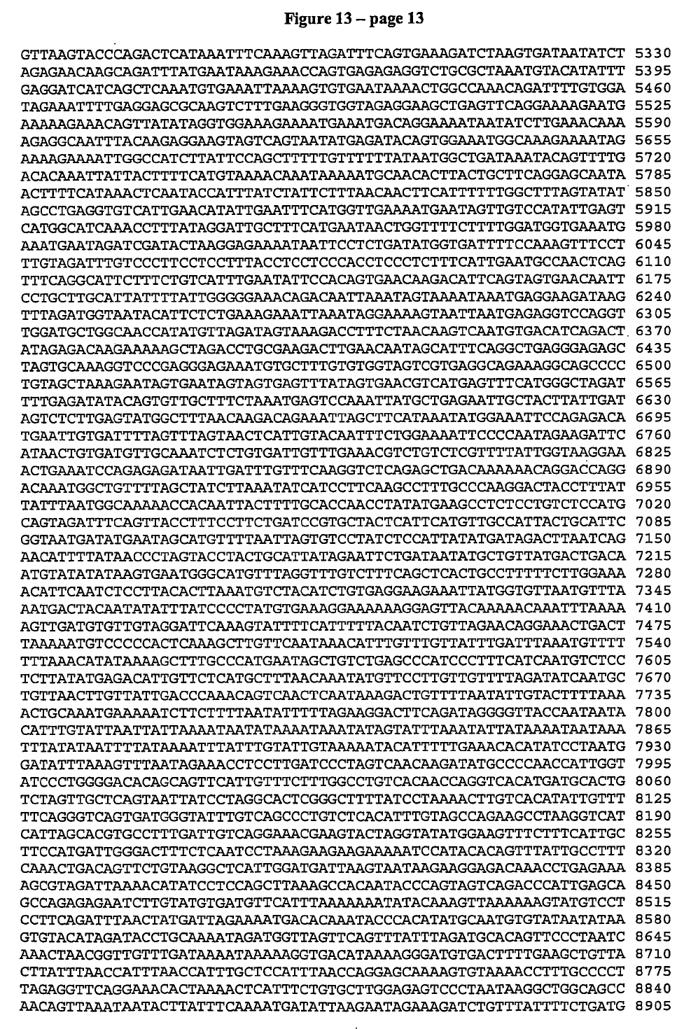

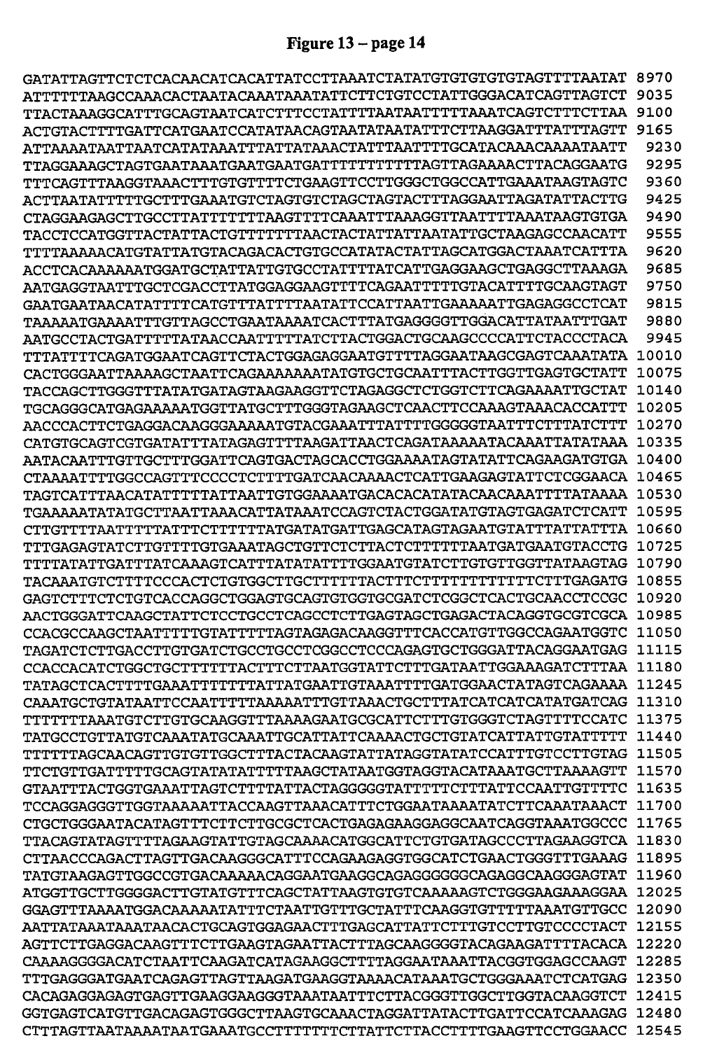

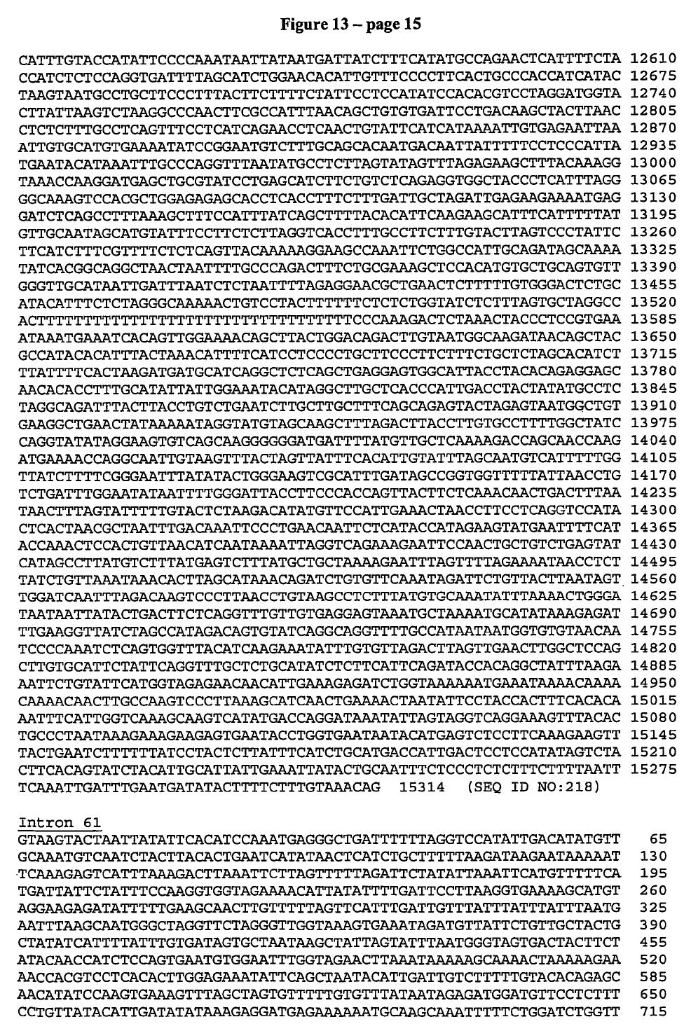

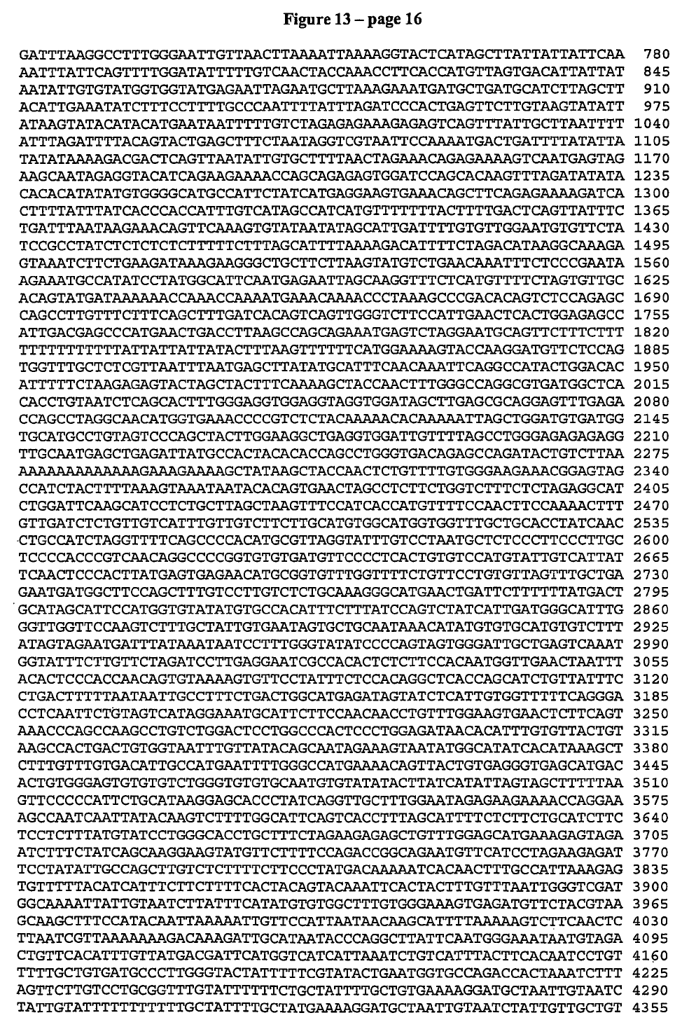

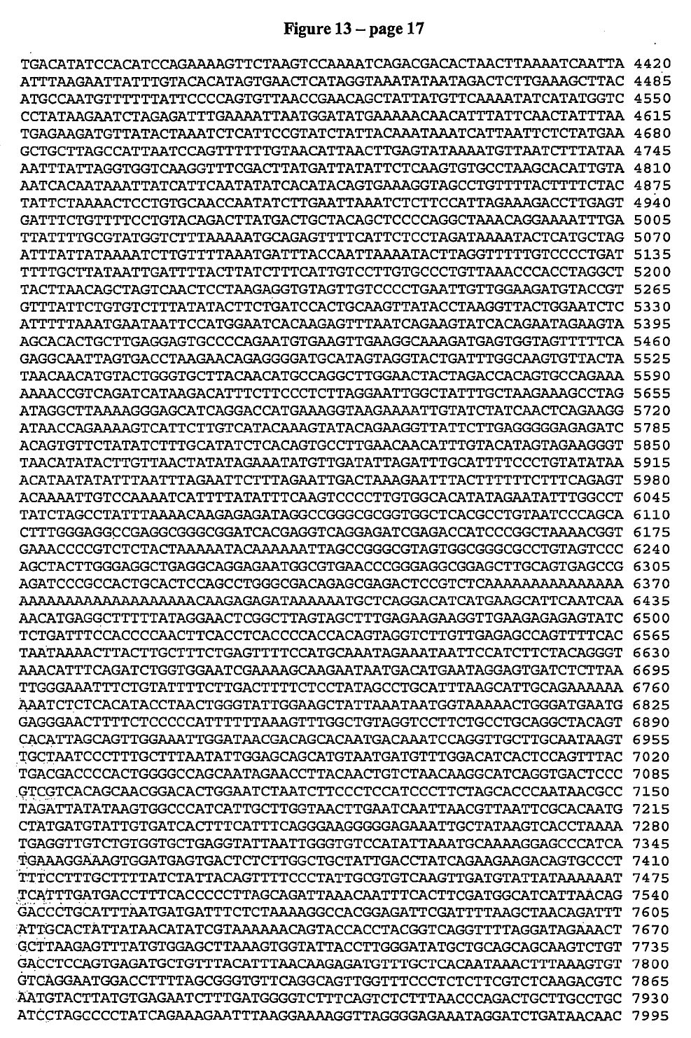

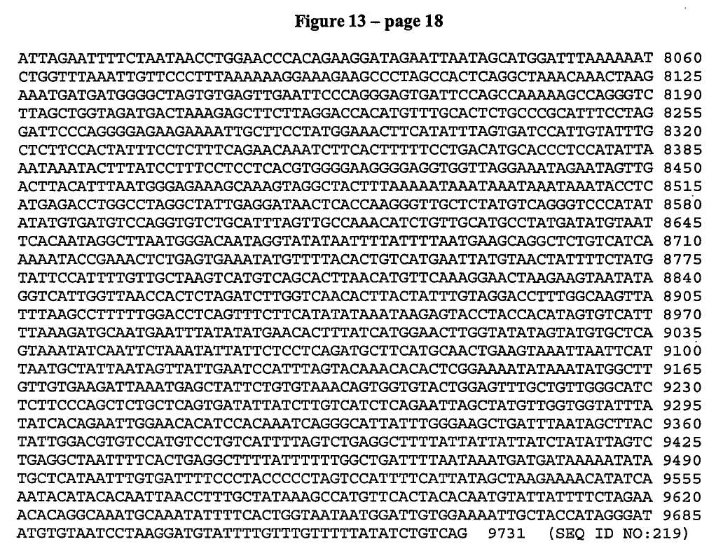

Nucleotide sequences of introns in which nucleotide sequence variants were detected are shown in FIG. 13. These included introns 1, 3, 7, 14, 22, 23, 28, 32, 33, 43, 53, and 61 (SEQ ID NOS:5, 209, 210, 211, 212, 213, 214, 215, 216, 217, 218, and 219, respectively). Variants in several of these introns were determined to be associated with ARPKD, while others were determined to be polymorphisms (see Tables 10 and 12).

TABLE 10
Clinical phenotype and mutations

| | | | | | | | | | | | | |
|---|---|---|---|---|---|---|---|---|---|---|---|---|
| | | | | | | | | | Exon/ | | Segre- | Normal |
| Pedigree | Patient^{a} | Sex | Age at dx^{b} | Presentation^{c} | Renal^{d} | Liver^{e} | Mutations | Change^{f} | IVS | Mouse^{g} | gation^{h} | screenⁱ |

ARPKD

| | | | | | | | | | | | | |
|---|---|---|---|---|---|---|---|---|---|---|---|---|
| PRR-1 | (1F) | ? | Birth, | Potter's | +++cystic | CHF | 9689delA | 3229↓ | 58 | | M | |
| | | | died PN | | | | 9689delA | 3229↓ | 58 | | P | |
| PRR-12 | (12F) | F | Birth, | Potter's | +++cystic | CHF | 9689delA | 3229↓ | 58 | | M | |
| | | | died PN | | | | 9689delA | 3229↓ | 58 | | P | |
| PRR-17 | 2655 | F | Birth, | Enlarged | +++cystic | CHF | 9689delA | 3229↓ | 58 | | (P) | |
| | | | died PN | kidneys | | | 3761CC→G | 1253↓ | 32 | | M | |
| PRR-7 | 2634 | F | Birth | Enlarged | +++cystic | CHF | 9689delA | 3229↓ | 58 | | S | X |
| | | | | kidneys | | | C3622Y | 10865G→A | 61 | C | S | |
| OV-10 | 2433 | M | 7 m | Enlarged | +++cystic | CHF | 9689delA | 3229↓ | 58 | | M | |
| | | | | kidneys | | | A17V | 50C→T* | 2 | A | (P) | X |
| OV-16 | 2439 | M | Birth | Echogenic | +++cystic | ? | 5895insA | 1965↓ | 36 | | P | |
| | | | | kidneys | | | C2688F | 8063G→T | 50 | C | M | X |
| | | | | | | | I3468V | 10402A→G | 61 | M | M | X |
| OV-18 | 2441 | M | 23 m | Echogenic | ++cystic | CHF | 1529delG | 509↓ | 16 | | M | |
| | | | | kidneys | | | 657C→T | G219* | 9 | | P | X |
| | | | | | | | T2869K | 8606C→A | 55 | V | P | X |
| OV-33 | 2453 | F | Birth | Enlarged | +++cystic | CHF | 3761CC→G | 1253↓ | 32 | | P | |
| | | | | kidneys | | | I222V | 664A→G* | 9 | I | M | X |
| PRR-2 | (2F) | ? | Birth, | Potter's | +++cystic | ? | 5895insA | 1965↓ | 36 | | P | |
| | | | died PN | | | | C3346R | 10036T→C | 60 | C | M | X |
| PRR-4 | (4F) | ? | Birth, | Potter's | +++cystic | CHF | 383delC | 127↓ | 5 | | M | |
| | | | died PN | | | | Y1838C | 5513A→G | 34 | Y | P | X |
| PRR-9 | 2644 | M | 19 y | ESRD | ++cystic | CHF | 6383delT | 2127↓ | 39 | | P | |
| | (9F) | M | Birth, | Potter's | +++cystic | CHF | I222V | 664A→G* | 9 | I | M | X |
| | | | died PN | | | | | | | | | |
| PRR-15 | 2651 | M | In utero | Enlarged | +++cystic | ? | 383delC | 127↓ | 34 | | M | |
| | | | | kidneys | | | 1222V | 664A→G* | 9 | I | P | X |
| OV-30 | 2450 | M | Birth | OH | Enlarged, | CHF | 10856delA | 3618↓ | 61 | | P | |
| | | | | | +++cystic | | IVS33 − 9T→G | ? | IVS33 | | M | X |
| OV-35 | 2455 | F | 18 m | Splenomegaly | +cystic | CHF | IVS28 − 2A→C | (1076↓) | IVS28 | | P | |
| | | | | | | | E3502V | 10505A→T | 61 | E | M | X |
| | | | | | | | T2869K | 8606C→A | 55 | V | M | X |
| OV-7 | 2537 | M | Birth | Enlarged | +++cystic | No | T36M | 107C→T | 3 | T | P | X |
| | | | | kidneys | | | | (2332↓) | | | M | X |
| | 2538 | M | Birth | Enlarged | +++cystic | No | IVS43 + 4A→T | | IVS43 | | | |
| | | | | kidneys | | | | | | | | |
| OV-23 | 2444 | M | 3 m | Echogenic | +++cystic | CHF | D1942G | 5825A→G | 36 | D | P | X |
| | | | | kidneys | | | T2869K | 8606C→T | 55 | V | P | X |
| | 2581 | F | 6 m | Echogenic | +++cystic | CHF | P739L | 2216C→T | 22 | P | M | X |
| | | | | kidneys | | | | | | | | |
| P728 | 2689 | M | Birth, | Enlarged | +++cystic | CHF | T36M | 107C→T | 3 | T | NP | X |
| | | | died PN | kidneys, | | | P805L | 2414C→T | 24 | P | NP | X |
| | | | | OH | | | I3177T | 9530T→C | 58 | V | NP | X |
| OV-20 | 2443 | ? | In utero | Echogenic | Enlarged, | CD, | I757L | 2269A→C | 22 | I | P | X |
| | | | | kidneys | +++cystic | CHF | I3177T | 9530T→C | 58 | V | M | X |
| OV-14 | 2437 | M | Birth | Echogenic | +++cystic | ? | 1529delG | 509↓ | 16 | | M | |
| | | | | kidneys | | | | | | | | |
| PRR-3 | 2624 | M | 18 y | ESRD | ++cystic | CHF | 5895insA | 1965↓ | 36 | | P | |
| OV-17 | 2440 | M | 14 m | Enlarged | +++cystic | CHF | 5895insA | 1965↓ | 36 | | M | |
| | | | | kidneys | | | | | | | | |
| OV-29 | 2449 | M | Birth | OH | Echogenic | CHF | 5895insA | 1965↓ | 36 | | P | |
| | | | | | +++cystic | | | | | | | |
| PRR-5 | 2816 | ? | Birth, | Potter's | +++cystic | CHF | 5895insA | 1965↓ | 36 | | M | |
| | | | died PN | | | | | | | | | |
| PRR-18 | (18F) | ? | Birth, | Enlarged | +++cystic | ? | 5895insA | 1965↓ | 36 | | P | |
| | | | died PN | kidneys | | | | | | | | |
| OV-1 | 2427 | F | Birth | Echogenic | +++cystic | CHF | 9689delA | 3229↓ | 58 | | P | |
| | | | | kidneys | | | | | | | | |
| M94 | R1101 | M | In utero, | Enlarged | +++cystic | CHF | S1867N | 5600G→A | 34 | S | P | X |
| | | | died PN | kidneys, | | | | | | | | |
| | | | | OH | | | | | | | | |
| M96 | R1050 | F | 2 y | Hematuria | +++cystic | CHF | E3529Q | 10585G→C | 61 | E | NP | X |
| PRR-6 | 2633 | F | Birth | Enlarged | Echogenic | CHF | T36M | 107C→T | 3 | T | NP | X |
| | | | | kidneys | | | | | | | | |
| OV-13 | 2436 | F | 21 m | Echogenic | Enlarged | No | P1389T | 4165C→A | 32 | P | M | X |
| | | | | kidneys | +cystic | | | | | | | |
| OV-27 | 2447 | M | Birth | Enlarged | +++cystic | CHF | T36M | 107C→T | 3 | T | M | X |
| | | | | kidneys | | | | | | | | |

CHF

| | | | | | | | | | | | | |
|---|---|---|---|---|---|---|---|---|---|---|---|---|
| HEP-3 | 2666 | F | 18 y | Splenomegaly | +cystic | CD, | 9689delA | 3229↓ | 58 | | NP | |
| | | | | CD, CHF | | CHF | T2869K | 8606C→A | 55 | V | NP | X |
| HEP-13 | 2675 | F | | | | | 10364delC | 3454↓ | 61 | | NP | |
| | | | | | | | I3468V | 10402A→G | 61 | M | NP | X |
| M83 | R1046 | F | 38 y | Cholangitis | MSK, | CD, | V1741M | 5221G→A | 32 | V | NP | X |
| | | | | | 1 cyst | CHF, | S1833L | 5498C→T | 34 | S | NP | X |
| M84 | R1051 | F | 18 y | Cholangitis | 1 cyst | CD, | T2869K | 8606C→A | 55 | V | M | X |
| | | | | | | CHF | I2957T | 8870T→C | 57 | I | P | X |
| HEP-1 | 2659 | M | | | | CD | 5895insA | 1965↓ | 36 | | M | |
| | 1661 | F | | | | | | | | | | |

^{a}( ) = DNA from the patient is unavailable and the parents were screened for mutations
^{b}Age at diagnosis; died PN = died in perinatal period
^{c}Potter's = Potter's phenotype, consisting of pulmonary hypoplasia, characteristic facies and skeletal abnormalities (Potter (1964) Obstet. Gynecol. 51: 885-888); OH = oligohydramnios
^{d}Cystic +++, multiple dilated collecting ducts fill kidney; cystic ++, moderately cystic, cystic +; some cysts; MSK = medullary sponge kidney
^{e}Liver phenotype; CD = Caroli's Disease; CHF = congenital hepatic fibrosis
^{f}Nucleotide change or ↓ = frameshift after indicated amino acid; * = possible cryptic splicing change; ( ) = predicted consequence of aberrant splicing
^{g}Corresponding residue in murine Pkhd1
^{h}P = paternal; M = maternal; NP = samples not available for segregation analysis; S = segregation indicates the mutations are on separate alleles but the parental origin cannot be determined; ( ) = parental origin inferred
ⁱScreen of 100 normal chromosomes; X = negative

TABLE 11
Details of mutations in ARPKD and CHF patient populations

| | Clinical phenotype | | |
|---|---|---|---|
| Population and mutation details | ARPKD | CHF | Total |
| Pedigrees^{a} | 47 (94) | 19 (38) | 66 (132) |
| Both mutations detected^{b} | 18 (17) | 4 (1) | 22 (18) |
| Single mutation detected^{c} | 12 (10) | 1 (1) | 13 (11) |
| Mutant alleles^{d} | 48 (51.1) | 9 (23.7) | 57 (43.2) |
| Mutant pedigrees^{d,e} | 30 (63.8) | 5 (26.3) | 35 (53.0) |
| Different mutations | 29 | 8 | 33 |
| Ancestral mutations^{a,f} | 10 (34) | 6 (7) | 12 (41) |

^{a}( ) = Disease alleles
^{b}( ) = Segregation demonstrated
^{c}( ) = Parental origin known
^{d}( ) = % of total
^{e}At least one mutation detected
^{f}Detected at least twice in this study or previously described (Ward et al., supra; and Onuchic et al. (2002) Am. J. Hum. Genet. 70: 1305-1317)

TABLE 12
Polymorphisms found in the study population

| | Nucleotide change/ | | |
|---|---|---|---|
| Designation | amino acid position | Exon/IVS | Allele frequency |
| IVS1 − 47C/T | | IVS1 | 40/128 |
| IVS30insA* | | IVS1 | 2/238 |
| 214C/T | L72 | 4 | 19/128 |
| 234C/T | D78 | 4 | 8/128 |
| IVS7 + 19T/C | | IVS7 | 32/128 |
| IVS14 + 23G/T | | IVS14 | 1/238 |
| 1185T/C | D395 | 15 | 2/128 |
| 1587T/C | N529 | 17 | 8/128 |
| 2046A/C | P682 | 21 | 21/128 |
| 2196C/T | V732 | 22 | 1/238 |
| R/C760 | 2278C/T | 22 | 37/128 |
| IVS22 + 13T/G | | IVS22 | 16/128 |
| IVS23 + 50C/T | | IVS23 | 53/128 |
| IVS23 + 53A/G | | IVS23 | 14/128 |
| N/S830 | 2489A/G | 24 | 10/128 |
| 3537T/C | N1179 | 30 | 2/238 |
| 3756G/C | L1252 | 32 | 2/238 |
| A/V1262 | 3785C/T | 32 | 9/238 |
| 4920A/G | V1640 | 32 | 3/238 |
| L/F1709 | 5125C/T | 32 | 1/338 |
| IVS32 + 42del4 | | IVS32 | 5/128 |
| L/V1870 | 5608T/G | 35 | 4/128 |
| 7587G/A | G2529 | 48 | 33/128 |
| 7764A/G | L2588 | 49 | 30/128 |
| IVS53 − 32C/G | | IVS53 | 33/128 |
| 9237G/A | A3079 | 58 | 33/128 |
| D/Y3139 | 9415G/T | 58 | 2/128 |
| S/R3505 | 10515C/T | 61 | 5/128 |
| 10521C/T | H3507 | 61 | 5/128 |
| IVS61 + 9A/G | | IVS61 | 5/128 |
| 11340T/C | P3780 | 63 | 4/128 |
| Q/R3899 | 11196A/G | 66 | 37/128 |
| V/I3960 | 11878G/A | 67 | 3/238 |
| Q/R4048 | 12143A/G | 67 | 42/128 |

*A is inserted just 5′ to the splice donor site of intron 3, i.e., A is inserted between the nucleotide at the 3′ end of exon 3 and the guanine at the 5′ end of intron 3.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated nucleic acid comprising a sequence encoding a fibrocystin polypeptide:

2. The isolated nucleic acid of claim 1, wherein said fibrocystin polypeptide is encoded by SEQ ID NO:1, SEQ ID NO:3, or SEQ ID NO:4.

3-4. (canceled)

5. The isolated nucleic acid of claim 1, wherein said fibrocystin polypeptide comprises the amino acid sequence of SEQ ID NO:2, SEQ ID NO:6, or SEQ ID NO:7.

6-7. (canceled)

8. The isolated nucleic acid of claim 1, wherein said sequence comprises a sequence variant associated with autosomal recessive polycystic kidney disease.

9. The isolated nucleic acid of claim 1, wherein said fibrocystin polypeptide comprises an amino acid sequence variant at a position selected from the group consisting of: position 17, position 36, position 222, position 739, position 757, position 805, position 1249, position 1389, position 1407, position 1664, position 1741, position 1833, position 1838, position 1867, position 1917, position 1942, position 1995, position 2331, position 2688, position 2869, position 2957, position 3018, position 3177, position 3346, position 3468, position 3502, position 3529, position 3553, and position 3622 of SEQ ID NO:2.

10. The isolated nucleic acid of claim 9, wherein said amino acid sequence variant is selected from the group consisting of: Val at position 17, Met at position 36, Val at position 222, Leu at position 739, Leu at position 757, Leu at position 805, Trp at position 1249, Thr at position 1389, Arg at position 1407, Phe at position 1664, Met at position 1741, Leu at position 1833, Cys at position 1838, Asn at position 1867, Arg at position 1917, Gly at position 1942, Gly at position 1995, Lys at position 2331, Phe at position 2688, Lys at position 2869, Thr at position 2957, Phe at position 3018, Thr at position 3177, Arg at position 3346, Val at position 3468, Val at position 3502, Gln at position 3529, Thr at position 3553, and Tyr at position 3622.

11. The isolated nucleic acid of claim 1, wherein said fibrocystin polypeptide comprises an amino acid sequence variant at a position selected from the group consisting of: position 25, position 752, position 760, position 830, position 852, position 1262, position 1709, position 1870, position 2938, position 3139, position 3505, position 3899, position 3960, and position 4048 of SEQ ID NO:2.

12. The isolated nucleic acid of claim 11, wherein said amino acid sequence variant is selected from the group consisting of: Val at position 25, Met at position 752, Cys at position 760, Ser at position 830, Arg at position 852, Val at position 1262, Phe at position 1709, Val at position 1870, Met at position 2938, Tyr at position 3139, Arg at position 3505, Arg at position 3899, Ile at position 3960, and Arg at position 4048.

13. The isolated nucleic acid of claim 1, wherein said fibrocystin polypeptide comprises the amino acids from position 1 to 3299 of SEQ ID NO:2, from position 1 to 2578 of SEQ ID NO:2, or from position 1 to 3779 of SEQ ID NO:2.

14-15. (canceled)

16. The isolated nucleic acid of claim 1, wherein said sequence comprises a nucleotide sequence variant with respect to SEQ ID NO:1, SEQ ID NO:214, SEQ ID NO:216, or SEQ ID NO:217.

17. The isolated nucleic acid of claim 16, wherein said nucleotide sequence variant with respect to SEQ ID NO:1 is at a position selected from the group consisting of: position 50, position 107, position 657, position 664, position 2216, position 2269, position 2414, position 3747, position 3761, position 4165, position 4220, position 4991, position 5221, position 5498, position 5513, position 5600, position 5750, position 5825, position 5984, position 6992, position 8011, position 8063, position 8606, position 8870, position 9053, position 9530, position 10036, position 10174, position 10402, position 10505, position 10585, position 10658, position 10865, and position 11612 of SEQ ID NO:1.

18. The isolated nucleic acid of claim 17, wherein said nucleotide sequence variant with respect to SEQ ID NO:1 is selected from the group consisting of: T at position 50, T at position 107, T at position 657, G at position 664, T at position 2216, C at position 2269, T at position 2414, G at position 3747, G at position 3761, A at position 4165, G at position 4220, T at position 4991, A at position 5221, T at position 5498, G at position 5513, A at position 5600, G at position 5750, G at position 5825, G at position 5984, A at position 6992, T at position 8011, T at position 8063, A or T at position 8606, C at position 8870, T at position 9053, C at position 9530, C at position 10036, T at position 10174, G at position 10402, T at position 10505, C at position 10585, C at position 10658, A at position 10865, and A at position 11612.

19. The isolated nucleic acid of claim 16, wherein said nucleotide sequence variant with respect to SEQ ID NO:1 comprises an A inserted at position 5895 or 5896, a deletion of the nucleotides at positions 1624, 1625, 1626, and 1627, a deletion of the nucleotide at position 10637, a deletion of the nucleotide at position 9689, a deletion of the nucleotide at position 3762, a deletion of the nucleotide at position 1529, a deletion of the nucleotide at position 383, a deletion of the nucleotide at position 6383, a deletion of the nucleotide at position 10856, or a deletion of the nucleotide at position 10364.

20-28. (canceled)

29. The isolated nucleic acid of claim 16, wherein said nucleotide sequence variant with respect to SEQ ID NO:214 is at position -2 relative to the splice acceptor site of intron 28.

30. The isolated nucleic acid of claim 29, wherein said nucleotide sequence variant is a C at position -2 relative to the splice acceptor site of intron 28.

31. The isolated nucleic acid of claim 16, wherein said nucleotide sequence variant with respect to SEQ ID NO:216 is at position −9 relative to the splice acceptor site of intron 33.

32. The isolated nucleic acid of claim 31, wherein said nucleotide sequence variant is a G at position −9 relative to the splice acceptor site of intron 33.

33. The isolated nucleic acid of claim 16, wherein said nucleotide sequence variant with respect to SEQ ID NO:217 is at position +4 relative to the splice donor site of intron 43.

34. The isolated nucleic acid of claim 33, wherein said nucleotide sequence variant is a T at position +4 relative to the splice donor site of intron 43.

35. The isolated nucleic acid of claim 16, wherein said nucleotide sequence variant with respect to SEQ ID NO:1 is at a position selected from the group consisting of: position 73, position 214, position 234, position 1185, position 1587, position 2046, position 2196, position 2255, position 2278, position 2489, position 2554, position 2853, position 3537, position 3756, position 3785, position 4920, position 5125, position 5608, position 7587, position 7764, position 8813, position 9237, position 9415, position 10515, position 10521, position 11340, position 11196, position 11878, and position 12143 of SEQ ID NO:1.

36. The isolated nucleic acid of claim 35, wherein said nucleotide sequence variant with respect to SEQ ID NO:1 is selected from the group consisting of: A at position 73, T at position 214, T at position 234, C at position 1185, C at position 1587, C at position 2046, T at position 2196, T at position 2255, T at position 2278, G at position 2489, C at position 2554, T at position 2853, C at position 3537, C at position 3756, T at position 3785, G at position 4920, T at position 5125, G at position 5608, A at position 7587, G at position 7764, T at position 8813, A at position 9237, T at position 9415, T at position 10515, T at position 10521, C at position 11340, G at position 11196, A at position 11878, and G at position 12143.

37. The isolated nucleic acid of claim 1, wherein said fibrocystin polypeptide is encoded by nucleotides 276 to 10174 of SEQ ID NO:1, nucleotides 276 to 8011 of SEQ ID NO:1, or nucleotides 276 to 11612 of SEQ ID NO:1.

38-39. (canceled)

40. The isolated nucleic acid of claim 1, said nucleic acid comprising nucleotides 1 to 192 of SEQ ID NO:1, nucleotides 193 to 328 of SEQ ID NO:1, or nucleotides 329 to 406 of SEQ ID NO:1.

41-42. (canceled)

43. The isolated nucleic acid of claim 1, wherein said nucleotide sequence comprises a sequence variant with respect to SEQ ID NO:5, SEQ ID NO:209, SEQ ID NO:210, SEQ ID NO:21 1, SEQ ID NO:212, SEQ ID NO:213, SEQ ID NO:215, SEQ ID NO:218, or SEQ ID NO:219.

44. The isolated nucleic acid of claim 43, wherein said nucleotide sequence variant is at a position selected from the group consisting of: position −47 relative to the splice acceptor site of SEQ ID NO:5, the position just 5′ to the splice donor site of SEQ ID NO:209, position +19 relative to the splice donor site of SEQ ID NO:210, position +23 relative to the splice donor site of SEQ ID NO:21 1, position +13 relative to the splice donor site of SEQ ID NO:212, position +50 relative to the splice donor site of SEQ ID NO:213, position +53 relative to the splice donor site of SEQ ID NO:213, positions +42 through +45 relative to the splice donor site of SEQ ID NO:215, position −32 relative to the splice acceptor site of SEQ ID NO:218, and position +9 relative to the splice donor site of SEQ ID NO:219.

45. The isolated nucleic acid of claim 44, wherein said nucleotide sequence variant is a T at position −47 relative to the splice acceptor site of SEQ ID NO:5, an A inserted just 5′ to the splice donor site of SEQ ID NO:209, a C at position +19 relative to the splice donor site of SEQ ID NO:210, a T at position +23 relative to the splice donor site of SEQ ID NO:211, a G at position +13 relative to the splice donor site of SEQ ID NO:212, a T at position +50 relative to the splice donor site of SEQ ID NO:213, a G at position +53 relative to the splice donor site of SEQ ID NO:213, deletion of the nucleotides at positions +42 through +45 relative to the splice donor site of SEQ ID NO:215, a G at position −32 relative to the splice acceptor site of SEQ ID NO:218, or a G at position +9 relative to the splice donor site of SEQ ID NO:219.

46. An isolated nucleic acid encoding a fibrocystin polypeptide, wherein said nucleic acid comprises at least 300 contiguous nucleotides of SEQ ID NO:1 or a sequence variant thereof.

47. A vector comprising the isolated nucleic acid of claim 46.

48. Host cells comprising the vector of claim 47.

49. An isolated nucleic acid 10 to 1650 nucleotides in length, said nucleic acid comprising a sequence, said sequence comprising one or more nucleotide sequence variants relative to the sequence of SEQ ID NO:1, and wherein said sequence is at least 80% identical over its length to the corresponding sequence in SEQ ID NO:1.

50. The isolated nucleic acid of claim 49, wherein said one or more nucleotide sequence variants are at position 50, 107, 383, 657, 664, 1529, 1624, 1625, 1626, 1627, 2216, 2269, 2414, 3747, 3761, 3762, 4165, 4220, 4991, 5221, 5498, 5513, 5600, 5750, 5825, 5895, 5896, 5984, 6383, 6992, 8011, 8063, 8606, 8870, 9053, 9530, 9689, 10036, 10174, 10364, 10402, 10505, 10585, 10637, 10658, 10856, 10865, or 11612 of SEQ ID NO:1.

51. The isolated nucleic acid of claim 49, wherein said one or more nucleotide sequence variants are at position 73, 214, 234, 1185, 1587, 2046, 2196, 2255, 2278, 2489, 2554, 2853, 3537, 3756, 3785, 4920, 5125, 5608, 7587, 7764, 8813, 9237, 9415, 10515, 10521, 11340, 11196, 11878, or 12143 of SEQ ID NO:1.

52. A plurality of oligonucleotide primer pairs, wherein each primer is 10 to 50 nucleotides in length, and wherein each said primer pair, in the presence of mammalian genomic DNA and under polymerase chain reaction conditions, produces a nucleic acid product corresponding to a region of an ARPKD nucleic acid molecule, wherein said product is 30 to 1650 nucleotides in length.

53. The plurality of primer pairs of claim 52, wherein said nucleic acid product comprises a nucleotide sequence variant relative to SEQ ID NO:1.

54. The plurality of primer pairs of claim 52, wherein said plurality comprises at least three primer pairs.

55. The plurality of primer pairs of claim 52, wherein said plurality comprises at least thirteen primer pairs.

56. The plurality of primer pairs of claim 52, wherein said plurality comprises at least sixteen primer pairs.

57. The plurality of primer pairs of claim 52, wherein said plurality comprises at least twenty-three primer pairs.

58. A composition comprising a first oligonucleotide primer and a second oligonucleotide primer, wherein said first oligonucleotide primer and said second oligonucleotide primer are each 10 to 50 nucleotides in length, and wherein said first and second primers, in the presence of mammalian genomic DNA and under polymerase chain reaction conditions, produce a nucleic acid product corresponding to a region of an ARPKD nucleic acid molecule, wherein said product is 30 to 1650 nucleotides in length.

59. The composition of claim 58, wherein said nucleic acid product comprises a nucleotide sequence variant relative to SEQ ID NO:1.

60. An isolated nucleic acid comprising the nucleotide sequence of SEQ ID NO:1 or its complement.

61. An antibody having specific binding affinity for a fibrocystin polypeptide.

62. A method for determining the susceptibility of a subject to autosomal recessive polycystic kidney disease, said method comprising providing a nucleic acid sample from said subject, and determining whether said nucleic acid sample contains one or more nucleotide sequence variants within the PKHD1 gene of said subject relative to a wild-type PKHD1 gene, wherein the presence of said one or more sequence variants is associated with increased susceptibility of said subject to autosomal recessive polycystic kidney disease.

63. The method of claim 62, wherein said nucleic acid sample is genomic DNA.

64. The method of claim 62, wherein said determining step is performed by denaturing high performance liquid chromatography.

65. The method of claim 62, further comprising identifying said one or more sequence variants by DNA sequencing.

66. The method of claim 62, wherein said one or more nucleotide sequence variants are selected from the group consisting of:
(a) a deletion of the nucleotides at positions 1624, 1625, 1626, and 1627, and an A at position 6992 of SEQ ID NO:1;
(b) a G at position 664 and a T at position 10174 of SEQ ID NO:1;
(c) a G at position 4220 and an A inserted at position 5896 of SEQ ID NO:1;
(d) a T at position 8011 and a C at position 10658 of SEQ ID NO:1;
(e) a G at position 5984 and an A at position 11612 of SEQ ID NO:1;
(f) a deletion at position 10637, and a C at position 8870 of SEQ ID NO:1;
(g) a T at position 4991 and a T at position 9053 of SEQ ID NO:1;
(h) a G at position 3747 and a G at position 5750 of SEQ ID NO:1;
(i) an A at position 5221 of SEQ ID NO:1,
(j) a T at position 107 of SEQ ID NO:1;
(k) a deletion at position 9689 of SEQ ID NO:1; and
(l) a deletion at position 9689 and a G at position 3761 in combination with a deletion at position 3762 of SEQ ID NO:1.

67-77. (canceled)

78. The method of claim 62, wherein said one or more nucleotide sequence variants are selected from the group consisting of:
(a) a deletion at position 9689 and an A at position 10865 of SEQ ID NO:1
(b) a deletion at position 9689 and a T at position 50 of SEQ ID NO:1;
(c) an A inserted at position 5895, a T at position 8063. and a G at position 10402 of SEQ ID NO:1;
(d) a deletion at position 1529, a T at position 657, and an A at position 8606 of SEQ ID NO:1;
(e) a G at position 664 and a G at position 3761 in combination with a deletion at position 3762 of SEQ ID NO:1;
(f) an insertion at position 5895 and a C at position 10036 of SEQ ID NO:1;
(g) a deletion at position 383 and a G at position 5513 of SEQ ]ID NO:1;
(h) a deletion at position 6383 and a G at position 664 of SEQ i) NO:1;
(i) a deletion at position 383 and a G at position 664 of SEQ ID NO:1;
(j) a deletion at position 10856 of SEQ ID NO:1 and a G at position −9 relative to the splice acceptor site of SEQ ID NO:216;
(k) a T at position 10505 and an A at position 8606 of SEQ ID NO:1, and a C at position −2 relative to the splice acceptor site of SEQ ID NO:214; and
(l) a T at position 107 of SEQ ID NO:1 and a T at position +4 relative to the splice donor site of SEQ ID NO:217.

79-89. (canceled)

90. The method of claim 62, wherein said one or more nucleotide sequence variants are selected from the group consisting of:
(a) a G at position 5825, a T at position 8606, and a T at position 2216 of SEQ ID NO:1;
(b) a T at position 107, a T at position 2414, and a C at position 9530 of SEQ ID NO:1;
(c) a C at position 2269 and a C at position 9530 of SEQ ID NO:1;
(d) a deletion at position 1529 of SEQ ID NO:1;
(e) an A inserted at position 5895 of SEQ ID NO:1;
(f) an A at position 5600 of SEQ ID NO:1;
(g) a C at position 10585 of SEQ ID NO:1;
(h) an A at position 4165 of SEQ ID NO:1;
(i) a deletion at position 9689 and an A at position 8606 of SEQ ID NO:1;
(j) a deletion at position 10364 and a G at position 10402 of SEQ ID NO:1;
(k) an A at position 5221 and a T at position 5498 of SEQ ID NO:1; and
(l) an A at position 8606 and a C at position 8870 of SEQ ID NO:1.

91-101. (canceled)

102. The method of claim 62, wherein said one or more nucleotide sequence variants are on separate alleles.

103. A method for diagnosing autosomal recessive polycystic kidney disease in a subject, said method comprising providing a nucleic acid sample from said subject, and determining whether said nucleic acid sample contains one or more disease-associated sequence variants within the PKHD1 gene of said subject compared to a wild-type PKHD1 gene, wherein the presence of said one or more disease-associated sequence variants is diagnostic of autosomal recessive polycystic kidney disease.

104. An article of manufacture comprising a substrate, wherein said substrate comprises a population of isolated nucleic acid molecules, wherein each said nucleic acid molecule is 10 to 1000 nucleotides in length, wherein each said nucleic acid molecule comprises a different nucleotide sequence variant relative to the sequence of SEQ ID NO:1, and wherein said nucleic acid molecule is at least 80% identical over its length to the corresponding sequence in SEQ ID NO:1.
